SEQUENCE LISTING (91 sequences)

SEQ ID NO: 1 - DNA, 3160 bp, H. sapiens
cctcccctcgcccggcgcggtcccgtccgcctctcgctcgcctcccgcctcccctcggtcttccgaggcgcccgggctcccggcgcggcggcggagggggcgggcaggccggcgggcggtgatgtggcaggactctttatgcgctgcggcaggatacgcgctcggcgctgggacgcgactgcgctcagttctctcctctcggaagctgcagccatgatggaagtttgagagttgagccgctgtgaggcgaggccgggctcaggcgagggagatgagagacggcggcggccgcggcccggagcccctctcagcgcctgtgagcagccgcgggggcagcgccctcggggagccggccggcctgcggcggcggcagcggcggcgtttctcgcctcctcttcgtcttttctaaccgtgcagcctcttcctcggcttctcctgaaagggaaggtggaagccgtgggctcgggcgggagccggctgaggcgcggcggcggcggcggcggcacctcccgctcctggagcgggggggagaagcggcggcggcggcggccgcggcggctgcagctccagggagggggtctgagtcgcctgtcaccatttccagggctgggaacgccggagagttggtctctccccttctactgcctccaacacggcggcggcggcggcggcacatccagggacccgggccggttttaaacctcccgtccgccgccgccgcaccccccgtggcccgggctccggaggccgccggcggaggcagccgttcggaggattattcgtcttctccccattccgctgccgccgctgccaggcctctggctgctgaggagaagcaggcccagtcgctgcaaccatccagcagccgccgcagcagccattacccggctgcggtccagagccaagcggcggcagagcgaggggcatcagctaccgccaagtccagagccatttccatcctgcagaagaagccccgccaccagcagcttctgccatctctctcctcctttttcttcagccacaggctcccagacatgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctgaatttttttttatcaagagggataaaacaccatgaaaataaacttgaataaactgaaaatggacctttttttttttaatggcaataggacattgtgtcagattaccagttataggaacaattctcttttcctgaccaatcttgttttaccctatacatccacagggttttgacacttgttgtccagttgaaaaaaggttgtgtagctgtgtcatgtatatacctttttgtgtcaaaaggacatttaaaattcaattaggattaataaagatggcactttcccgttttattccagttttataaaaagtggagacagactgatgtgtatacgtaggaattttttccttttgtgttctgtcaccaactgaagtggctaaagagctttgtgatatactggttcacatcctacccctttgcacttgtggcaacagataagtttgcagttggctaagagaggtttccgaaaggttttgctaccattctaatgcatgtattcgggttagggcaatggaggggaatgctcagaaaggaaataattttatgctggactctggaccatataccatctccagctatttacacacacctttctttagcatgctacagttattaatctggacattcgaggaattggccgctgtcactgcttgttgtttgcgcatttttttttaaagcatattggtgctagaaaaggcagctaaaggaagtgaatctgtattggggtacaggaatgaaccttctgcaacatcttaagatccacaaatgaagggatataaaaataatgtcataggtaagaaacacagcaacaatgacttaaccatataaatgtggaggctatcaacaaagaatgggcttgaaacattataaaaattgacaatgatttattaaatatgttttctcaattgtaaaaaaaaaa

SEQ ID NO: 2 - DNA, 26 bp, Artificial Sequence
  Primer
aatggctaagtgaagatgacaatcat

SEQ ID NO: 3 - DNA, 25 bp, Artificial Sequence
  Primer
tgcacatatcattacaccagttcgt

SEQ ID NO: 4 - DNA, 30 bp, Artificial Sequence
  Probe
ttgcagcaattcactgtaaagctggaaagg

SEQ ID NO: 5 - DNA, 21 bp, Artificial Sequence
  Synthetic Oligonucleotide
tugucucugguccuuacuuaa

SEQ ID NO: 6 - RNA, 21 nt, Artificial Sequence
  Synthetic Oligonucleotide
uugucucugguccuuacuuaa

SEQ ID NO: 7 - DNA, 23 bp, Artificial Sequence
  Synthetic Oligonucleotide
acaaacaccattgtcacacacca

SEQ ID NO: 8 - DNA, 20 bp, Artificial Sequence
  Synthetic Oligonucleotide
ctgctagcctctggatttga

SEQ ID NO: 9 - DNA, 14 bp, Artificial Sequence
  Synthetic Oligonucleotide
cttagcactggcct

SEQ ID NO: 10 - RNA, 19 nt, Artificial Sequence
  Synthetic Oligonucleotide
uugucucugguccuuacuu

SEQ ID NO: 11 - RNA, 19 nt, Artificial Sequence
  Synthetic Oligonucleotide
cgagaggcggacgggaccg

SEQ ID NO: 12 - DNA, 21 bp, Artificial Sequence
  Synthetic Oligonucleotide
cgagaggcggacgggaccgtt

SEQ ID NO: 13 - DNA, 21 bp, Artificial Sequence
  Synthetic Oligonucleotide
ttgctctccgcctgccctggc

SEQ ID NO: 14 - DNA, 19 bp, Artificial Sequence
  Synthetic Oligonucleotide
gctctccgcctgccctggc

SEQ ID NO: 15 - RNA, 19 nt, Artificial Sequence
  Synthetic Oligonucleotide
aaguaaggaccagagacaa

SEQ ID NO: 16 - RNA, 21 nt, Artificial Sequence
  Synthetic Oligonucleotide
aaguaaggaccagagacaauu

SEQ ID NO: 17 - DNA, 21 bp, Artificial Sequence
  Synthetic Oligonucleotide
uugucucugguccuuacuutt

SEQ ID NO: 18 - DNA, 27 bp, Artificial Sequence
  Synthetic Oligonucleotide
gcgtttgctcttcttcttgcgtttttt

SEQ ID NO: 19 - DNA, 20 bp, Artificial Sequence
  Synthetic Oligonucleotide
ctgctagcctctggatttga

SEQ ID NO: 20 - RNA, 23 nt, Artificial Sequence
  Synthetic Oligonucleotide
agcagcacguaaauauuggcgaa

SEQ ID NO: 21 - RNA, 22 nt, Artificial Sequence
  Synthetic Oligonucleotide
uagcagcacguaaauauuggcg

SEQ ID NO: 22 - RNA, 22 nt, Artificial Sequence
  Synthetic Oligonucleotide
ccaauauuuacgugcugcgaaa

SEQ ID NO: 23 - RNA, 24 nt, Artificial Sequence
  Synthetic Oligonucleotide
uggcagugucuuagcugguuguaa

SEQ ID NO: 24 - RNA, 24 nt, Artificial Sequence
  Synthetic Oligonucleotide
ugagaacugaauuccauggguuaa

SEQ ID NO: 25 - RNA, 25 nt, Artificial Sequence
  Synthetic Oligonucleotide
uuaaugcuaaucgugauagggguaa

SEQ ID NO: 26 - RNA, 22 nt, Artificial Sequence
  Synthetic Oligonucleotide
uugucucugguccuuacuuaac

SEQ ID NO: 27 - RNA, 20 nt, Artificial Sequence
  Synthetic Oligonucleotide
uugucucugguccuuacuua

SEQ ID NO: 28 - RNA, 22 nt, Artificial Sequence
  Synthetic Oligonucleotide
uugucucugguccuuacuuaca

SEQ ID NO: 29 - DNA, 21 bp, Artificial Sequence
  Synthetic Oligonucleotide
uugucucugguccuuacutaa

SEQ ID NO: 30 - DNA, 21 bp, Artificial Sequence
  Synthetic Oligonucleotide
uugucucugguccuuactuaa

SEQ ID NO: 31 - DNA, 21 bp, Artificial Sequence
  Synthetic Oligonucleotide
uugucucugguccutacuuaa

SEQ ID NO: 32 - DNA, 21 bp, Artificial Sequence
  Synthetic Oligonucleotide
uugucucuggucctuacuuaa

SEQ ID NO: 33 - DNA, 21 bp, Artificial Sequence
  Synthetic Oligonucleotide
uugucucuggtccuuacuuaa

SEQ ID NO: 34 - DNA, 21 bp, Artificial Sequence
  Synthetic Oligonucleotide
uugucuctgguccuuacuuaa

SEQ ID NO: 35 - DNA, 21 bp, Artificial Sequence
  Synthetic Oligonucleotide
uuguctcugguccuuacuuaa

SEQ ID NO: 36 - DNA, 21 bp, Artificial Sequence
  Synthetic Oligonucleotide
uugtcucugguccuuacuuaa

SEQ ID NO: 37 - DNA, 21 bp, Artificial Sequence
  Synthetic Oligonucleotide
utgucucugguccuuacuuaa

SEQ ID NO: 38 - RNA, 22 nt, H. sapiens
ugagguaguagguuguauaguu

SEQ ID NO: 39 - RNA, 22 nt, H. sapiens
ugagguaguagguugugugguu

SEQ ID NO: 40 - RNA, 22 nt, H. sapiens
ugagguaguagguuguaugguu

SEQ ID NO: 41 - RNA, 22 nt, H. sapiens
ugagguaguaguuugugcuguu

SEQ ID NO: 42 - RNA, 22 nt, H. sapiens
uggaauguaaagaaguauguau

SEQ ID NO: 43 - RNA, 23 nt, H. sapiens
uacccuguagauccgaauuugug

SEQ ID NO: 44 - RNA, 22 nt, H. sapiens
uagcagcacauaaugguuugug

SEQ ID NO: 45 - RNA, 22 nt, H. sapiens
uagcagcacguaaauauuggcg

SEQ ID NO: 46 - RNA, 22 nt, H. sapiens
uagcaccaucugaaaucgguua

SEQ ID NO: 47 - RNA, 23 nt, H. sapiens
uagcaccauuugaaaucaguguu

SEQ ID NO: 48 - RNA, 22 nt, H. sapiens
uagcaccauuugaaaucgguua

SEQ ID NO: 49 - RNA, 22 nt, H. sapiens
uggcagugucuuagcugguugu

SEQ ID NO: 50 - RNA, 22 nt, H. sapiens
caaucacuaacuccacugccau

SEQ ID NO: 51 - RNA, 23 nt, H. sapiens
aggcaguguaguuagcugauugc

SEQ ID NO: 52 - RNA, 23 nt, H. sapiens
caaagugcuguucgugcagguag

SEQ ID NO: 53 - RNA, 21 nt, H. sapiens
uacaguacugugauaacugaa

SEQ ID NO: 54 - RNA, 22 nt, H. sapiens
uggagugugacaaugguguuug

SEQ ID NO: 55 - RNA, 20 nt, H. sapiens
uaaggcacgcggugaaugcc

SEQ ID NO: 56 - RNA, 24 nt, H. sapiens
ucccugagacccuuuaaccuguga

SEQ ID NO: 57 - RNA, 22 nt, H. sapiens
ucccugagacccuaacuuguga

SEQ ID NO: 58 - RNA, 22 nt, H. sapiens
ucguaccgugaguaauaaugcg

SEQ ID NO: 59 - RNA, 22 nt, H. sapiens
uaacagucuacagccauggucg

SEQ ID NO: 60 - RNA, 22 nt, H. sapiens
uuugguccccuucaaccagcug

SEQ ID NO: 61 - RNA, 22 nt, H. sapiens
uuugguccccuucaaccagcua

SEQ ID NO: 62 - RNA, 22 nt, H. sapiens
ugagaacugaauuccauggguu

SEQ ID NO: 63 - RNA, 22 nt, H. sapiens
ucucccaacccuuguaccagug

SEQ ID NO: 64 - RNA, 23 nt, H. sapiens
uuaaugcuaaucgugauaggggu

SEQ ID NO: 65 - RNA, 23 nt, H. sapiens
aacauucaacgcugucggugagu

SEQ ID NO: 66 - RNA, 23 nt, H. sapiens
aacauucauugcugucggugggu

SEQ ID NO: 67 - RNA, 22 nt, H. sapiens
ugggucuuugcgggcgagauga

SEQ ID NO: 68 - RNA, 22 nt, H. sapiens
uagguaguuucauguuguuggg

SEQ ID NO: 69 - RNA, 22 nt, H. sapiens
gugaaauguuuaggaccacuag

SEQ ID NO: 70 - RNA, 22 nt, H. sapiens
uggaauguaaggaagugugugg

SEQ ID NO: 71 - RNA, 22 nt, H. sapiens
cugugcgugugacagcggcuga

SEQ ID NO: 72 - RNA, 21 nt, H. sapiens
agggcccccccucaauccugu

SEQ ID NO: 73 - RNA, 23 nt, H. sapiens
ucaagagcaauaacgaaaaaugu

SEQ ID NO: 74 - RNA, 23 nt, H. sapiens
uggaagacuagugauuuuguugu

SEQ ID NO: 75 - RNA, 22 nt, H. sapiens
uagcuuaucagacugauguuga

SEQ ID NO: 76 - RNA, 22 nt, H. sapiens
aagcugccaguugaagaacugu

SEQ ID NO: 77 - RNA, 22 nt, H. sapiens
uucaaguaauccaggauaggcu

SEQ ID NO: 78 - RNA, 21 nt, H. sapiens
uucaaguaauucaggauaggu

SEQ ID NO: 79 - RNA, 22 nt, H. sapiens
uaacacugucugguaaagaugg

SEQ ID NO: 80 - RNA, 21 nt, H. sapiens
ugagaugaagcacuguagcuc

SEQ ID NO: 81 - RNA, 23 nt, H. sapiens
guccaguuuucccaggaaucccu

SEQ ID NO: 82 - RNA, 21 nt, H. sapiens
uagcagcacagaaauauuggc

SEQ ID NO: 83 - RNA, 22 nt, H. sapiens
uaacacugucugguaacgaugu

SEQ ID NO: 84 - RNA, 22 nt, H. sapiens
uaauacugccugguaaugauga

SEQ ID NO: 85 - RNA, 23 nt, H. sapiens
uaauacugccggguaaugaugga

SEQ ID NO: 86 - RNA, 22 nt, H. sapiens
uccuucauuccaccggagucug

SEQ ID NO: 87 - RNA, 22 nt, H. sapiens
auaagacgagcaaaaagcuugu

SEQ ID NO: 88 - RNA, 22 nt, H. sapiens
auaagacgaacaaaagguuugu

SEQ ID NO: 89 - RNA, 23 nt, H. sapiens
agcuacauugucugcuggguuuc

SEQ ID NO: 90 - RNA, 21 nt, H. sapiens
agcuacaucuggcuacugggu

SEQ ID NO: 91 - RNA, 22 nt, H. sapiens
ugucaguuugucaaauacccca

(19) United States
(12) Patent Application Publication
(10) Pub. No.: US 2013/0156845 A1
(43) Pub. Date: Jun. 20, 2013

(54) LIPID FORMULATED SINGLE STRANDED RNA

(71) Applicants: Muthiah Manoharan, Cambridge, MA (US); Sayda Elbashir, Cambridge, MA (US); Kallanthottathil G. Rajeev, Cambridge, MA (US); Thazha P. Prakash, Carlsbad, CA (US); Walter F. Lima, Carlsbad, CA (US); Eric E. Swayze, Carlsbad, CA (US)

(72) Inventors: Muthiah Manoharan, Cambridge, MA (US); Sayda Elbashir, Cambridge, MA (US); Kallanthottathil G. Rajeev, Cambridge, MA (US); Thazha P. Prakash, Carlsbad, CA (US); Walter F. Lima, Carlsbad, CA (US); Eric E. Swayze, Carlsbad, CA (US)

(73) Assignees: ALNYLAM PHARMACEUTICALS, INC., Cambridge, MA (US); ISIS PHARMACEUTICALS, INC., Carlsbad, CA (US)

(21) Appl. No.: 13/643,940

(22) PCT Filed: Apr. 29, 2011
(86) PCT No.: PCT/US2011/034648

§ 371 Date: Mar. 4, 2013

Related U.S. Application Data
(60) Provisional application No. 61/329,466, filed on Apr. 29, 2010.

(51) Int. Cl. A61K 9/127; A61K 31/7088

(52) U.S. Cl. CPC A61K 9/1272; A61K 31/7088; A61K 9/1275
USPC 424/450; 514/44 A; 536/24.5

(57) ABSTRACT

The present invention provides compositions comprising a nucleic acid lipid particle and an oligomeric compound and uses thereof. In certain embodiments, such compositions are useful as antisense compounds. Certain such antisense compounds are useful as RNase H antisense compounds or as RNAi compounds.

### STATEMENT OF GOVERNMENT SUPPORT

This invention was made with United States Government support under contract #5R44GM076793-03 awarded by the NIH. The United States Government has certain rights in the invention.

### SEQUENCE LISTING

The present application is being filed along with a Sequence Listing in electronic format. The Sequence Listing is provided as a file entitled ALNIS0002WOSEQ.txt, created on Apr. 28, 2011, which is 19 Kb in size. The information in the electronic format of the sequence listing is incorporated herein by reference in its entirety.

### FIELD OF THE INVENTION

The present invention provides compounds, compositions, and methods for modulating nucleic acids and proteins. Provided herein are modified oligomeric compounds and compositions prepared therefrom. In certain embodiments, modified nucleosides are provided having at least one 5′-substituent and a 2′-substituent, oligomeric compounds comprising at least one of these modified nucleosides and compositions comprising at least one of these oligomeric compounds. In some embodiments, the oligomeric compounds provided herein are expected to hybridize to a portion of a target RNA resulting in loss of normal function of the target RNA. In certain embodiments, such compounds are formulated with lipid particle herein to form compositions. Certain such compositions modulate expression of a target nucleic acid.

### BACKGROUND OF THE INVENTION

Antisense compounds have been used to modulate target nucleic acids. Antisense compounds comprising a variety of modifications and motifs have been reported. In certain instances, such compounds are useful as research tools and as therapeutic agents. Certain double-stranded RNA-like compounds (siRNAs) are known to inhibit protein expression in cells. Such double-stranded RNA compounds function, at least in part, through the RNA-inducing silencing complex (RISC). Certain single-stranded RNA-like compounds (ssRNAs) have also been reported to function at least in part through RISC.

Targeting disease-causing gene sequences was first suggested more than thirty years ago (Belikova et al., Tet. Lett., 1967, 37, 3557-3562), and antisense activity was demonstrated in cell culture more than a decade later (Zamecnik et al., Proc. Natl. Acad. Sci. U.S.A., 1978, 75, 280-284). One advantage of antisense technology in the treatment of a disease or condition that stems from a disease-causing gene is that it is a direct genetic approach that has the ability to modulate (increase or decrease) the expression of specific disease-causing genes. Another advantage is that validation of a therapeutic target using antisense compounds results in direct and immediate discovery of the drug candidate; the antisense compound is the potential therapeutic agent.

Generally, the principle behind antisense technology is that an antisense compound hybridizes to a target nucleic acid and modulates gene expression activities or function, such as transcription or translation. The modulation of gene expression can be achieved by, for example, target degradation or occupancy-based inhibition. An example of modulation of RNA target function by degradation is RNase H-based degradation of the target RNA upon hybridization with a DNA-like antisense compound. Another example of modulation of gene expression by target degradation is RNA interference (RNAi). RNAi generally refers to antisense-mediated gene silencing involving the introduction of dsRNA leading to the sequence-specific reduction of targeted endogenous mRNA levels. An additional example of modulation of RNA target function by an occupancy-based mechanism is modulation of microRNA function. MicroRNAs are small non-coding RNAs that regulate the expression of protein-coding RNAs. The binding of an antisense compound to a microRNA prevents that microRNA from binding to its messenger RNA targets, and thus interferes with the function of the microRNA. Regardless of the specific mechanism, this sequence-specificity makes antisense compounds extremely attractive as tools for target validation and gene functionalization, as well as therapeutics to selectively modulate the expression of genes involved in the pathogenesis of malignancies and other diseases.

Antisense technology is an effective means for reducing the expression of one or more specific gene products and can therefore prove to be uniquely useful in a number of therapeutic, diagnostic, and research applications. Chemically modified nucleosides are routinely used for incorporation into antisense compounds to enhance one or more properties, such as nuclease resistance, pharmacokinetics or affinity for a target RNA. In 1998, the antisense compound, Vitravene® (fomivirsen; developed by Isis Pharmaceuticals Inc., Carlsbad; Calif.) was the first antisense drug to achieve marketing clearance from the U.S. Food and Drug Administration (FDA), and is currently a treatment of cytomegalovirus (CMV)-induced retinitis in AIDS patients.

New chemical modifications have improved the potency and efficacy of antisense compounds, uncovering the potential for oral delivery as well as enhancing subcutaneous administration, decreasing potential for side effects, and leading to improvements in patient convenience. Chemical modifications increasing potency of antisense compounds allow administration of lower doses, which reduces the potential for toxicity, as well as decreasing overall cost of therapy. Modifications increasing the resistance to degradation result in slower clearance from the body, allowing for less frequent dosing. Different types of chemical modifications can be combined in one compound to further optimize the compound's efficacy.

The synthesis of 5′-substituted DNA and RNA derivatives and their incorporation into oligomeric compounds has been reported in the literature (Saha et al., J. Org. Chem., 1995, 60, 788-789; Wang et al., Bioorganic & Medicinal Chemistry Letters, 1999, 9, 885-890; and Mikhailov et al., Nucleosides & Nucleotides, 1991, 10(1-3), 339-343; Leonid et al., 1995, 14(3-5), 901-905; and Eppacher et al., Helvetica Chimica Acta, 2004, 87, 3004-3020). The 5′-substituted monomers have also been made as the monophosphate with modified bases (Wang et al., Nucleosides Nucleotides & Nucleic Acids, 2004, 23 (1 & 2), 317-337).

A genus of modified nucleosides including optional modification at a plurality of positions including the 5′-position and the 2′-position of the sugar ring and oligomeric compounds incorporating these modified nucleosides therein has been reported (see International Application Number: PCT/US94/02993, Published on Oct. 13, 1994 as WO 94/22890).

The synthesis of 5′-CH₂ substituted 2′-O-protected nucleosides and their incorporation into oligomers has been previously reported (see Wu et al., Helvetica Chimica Acta, 2000, 83, 1127-1143 and Wu et al. Bioconjugate Chem. 1999, 10, 921-924).

Amide linked nucleoside dimers have been prepared for incorporation into oligonucleotides wherein the 3′ linked nucleoside in the dimer (5′ to 3′) comprises a 2′-OCH₃ and a 5′-(S)—CH₃ (Mesmaeker et al., Synlett, 1997, 1287-1290).

A genus of 2′-substituted 5′-CH₂ (or O) modified nucleosides and a discussion of incorporating them into oligonucleotides has been previously reported (see International Application Number: PCT/US92/01020, published on Feb. 7, 1992 as WO 92/13869).

The synthesis of modified 5′-methylene phosphonate monomers having 2′-substitution and their use to make modified antiviral dimers has been previously reported (see U.S. patent application Ser. No. 10/418,662, published on Apr. 6, 2006 as US 2006/0074035).

There remains a long-felt need for agents that specifically regulate gene expression via antisense mechanisms. Disclosed herein are oligomeric compounds such as antisense compounds useful for modulating gene expression pathways, including those relying on mechanisms of action such as RNaseH, RNAi and dsRNA enzymes, as well as other antisense mechanisms based on target degradation or target occupancy. One having skill in the art, once armed with this disclosure will be able, without undue experimentation, to identify, prepare and exploit antisense compounds for these uses.

### SUMMARY OF THE INVENTION

In certain embodiments, provided herein are compositions comprising oligomeric compounds and lipid particles wherein the oligomeric compounds comprise a modified nucleoside having at least one 2′ substituent group and either a 5′ substituent group, a 5′ phosphorus moiety or both a 5′ substituent group and a 5′ phosphorus moiety. In certain embodiments, the compositions provided herein that incorporate one or more modified nucleosides are expected to hybridize to a portion of a target RNA resulting in loss of normal function of the target RNA. In certain embodiments, compositions comprising such oligomeric compounds and lipid particles are expected to modulate target RNA function in vivo.

The variables are defined individually in further detail herein. It is to be understood that the modified nucleosides and oligomeric compounds provided herein include all combinations of the embodiments disclosed and variables defined herein.

In one embodiment, the invention provides a composition comprising a nucleic acid lipid particle comprising a single stranded RNA, wherein the nucleic acid lipid particle comprises a lipid formulation comprising 45-65 mol % of a cationic lipid, 5 mol % to about 10 mol %, of a non-cationic lipid, 25-40 mol % of a sterol, and 0.5-5 mol % of a PEG or PEG-modified lipid.

In certain embodiments, the single stranded RNA comprising a nucleoside having Formula I:

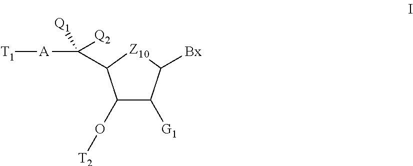

wherein:

Bx is a heterocyclic base moiety;

A is O, S or N(R₁);

Z₁₀ is O, S, N(R₁), CH₂;

R₁ is H, C₁-C₆ alkyl or substituted C₁-C₆ alkyl;

T₁ is a phosphorus moiety;

T₂ is an internucleoside linking group linking the monomer of Formula I to the remainder of the oligomeric compound;

each of Q₁ and Q₂ is independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;

G₁ is halogen, X₁—V, or O—X₂;

X₁ is O, S or CR₂R₃;

each R₂ and R₃ is, independently, H or C₁-C₆ alkyl;
V is a conjugate group, aryl, (CH₂)₂[O(CH₂)₂]ₜOCH₃, where t is from 1-3, (CH₂)₂F, CH₂COOH, CH₂CONH₂, CH₂CONR₅R₆, CH₂COOCH₂CH₃, CH₂CONH(CH₂)₁—S—R₄ where i is from 1 to 10, CH₂CONH(CH₂)ₖ₃NR₅R₆ where k₃ is from 1 to 6, CH₂CONH[(CH₂)ₖ₁—N(H)]ₖ₂—(CH₂)ₖ₁NH₂ where each k₁ is independently from 2 to 4 and k₂ is from 2 to 10;
R₄ is H, C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, C₆-C₁₄ aryl or a thio protecting group;
R₅ and R₆ are each, independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;
X₂ is [C(R₇)(R₈)]ₙ—[(C═O)ₘX]ⱼ—Z;
each R₇ and R₈ is independently, H, halogen, C₁-C₆ alkyl or substituted C₁-C₆ alkyl;

### X is O, S, or N(E₁);

Z is H, halogen, C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl or N(E₂)(E₃);
E₁, E₂, and E₃ are each independently H, C₁-C₆ alkyl, or substituted C₁-C₆ alkyl;
n is from 1 to about 6;
m is 0 or 1;
j is 0 or 1;

each substituted group comprises one or more optionally protected substituent groups independently selected from H, halogen, OJ₁, N(J₁)(J₂), ═NJ₁, SJ₁, N₃, CN, OC(=L)J₁, OC(=L)N(J₁)(J₂), C(=L)N(J₁)(J₂), C(=L)N(H)—(CH₂)₂N(J₁)(J₂) or a mono or polycyclic ring system;

L is O, S or NJ₃;

each J₁, J₂ and J₃ is, independently, H or C₁-C₆ alkyl; when j is 1 then Z is other than halogen or N(E₂)(E₃).

In certain embodiments, In certain embodiments, the single stranded RNA comprising a nucleoside having Formula II:

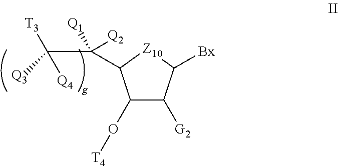

wherein:

Bx is a heterocyclic base moiety;

T₃ is a phosphorus moiety;

Z₁₀ is O, S, N(R₁), CH₂;

T₄ is an internucleoside linking group linking the monomer of Formula II to the remainder of the oligomeric compound;

Q₁, Q₂, Q₃ and Q₄ are each, independently, H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl, hydroxyl, substituted oxy, O—C₁-C₆ alkyl, substituted O—C₁-C₆ alkyl, S—C₁-C₆ alkyl, substituted S—C₁-C₆ alkyl, N(R₁)—C₁-C₆ alkyl or substituted N(R₁)—C₁-C₆ alkyl

R₁ is H, C₁-C₆ alkyl or substituted C₁-C₆ alkyl;

G₂ is H, OH, halogen, O-aryl or O—[C(R₄)(R₅)]ₙ—[(C═O), —X]ⱼ—Z;

each R₄ and R₅ is, independently, H, halogen, C₁-C₆ alkyl or substituted C₁-C₆ alkyl;

X is O, S or N(E₁);

Z is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);

E₁, E₂ and E₃ are each, independently, H, C₁-C₆ alkyl or substituted C₁-C₆ alkyl;

n is from 1 to about 6;

m is 0 or 1;

j is 0 or 1;

g is 0 or 1;

each substituted group comprises one or more optionally protected substituent groups independently selected from H, halogen, OJ₁, N(J₁)(J₂), ═NJ₁, SJ₁, N₃, CN, OC(=L)J₁, OC(=L)N(J₁)(J₂), C(=L)N(J₁)(J₂), C(=L)N(H)—(CH₂)₂N(J₁)(J₂), a mono or poly cyclic ring system, a phosphate group or a phosphorus moiety;

L is O, S or NJ₃;

each J₁, J₂ and J₃ is, independently, H or C₁-C₆ alkyl;

when j is 1 then Z is other than halogen or N(E₂)(E₃); and

when Q₁, Q₂, Q₃ and Q₄ are each H or when Q₁ and Q₂ are H and Q₃ and Q₄ are each F or when Q₁ and Q₂ are each H and one of Q₃ and Q₄ is H and the other of Q₃ and Q₄ is R₉ then G₂ is other than H, hydroxyl, OR₉, halogen, CF₃, CCl₃, CHCl₂ or CH₂OH wherein R₉ is alkyl, alkenyl, alkynyl, aryl or alkaryl.

In certain embodiments, In certain embodiments, the single stranded RNA comprising a nucleoside having Formula III:

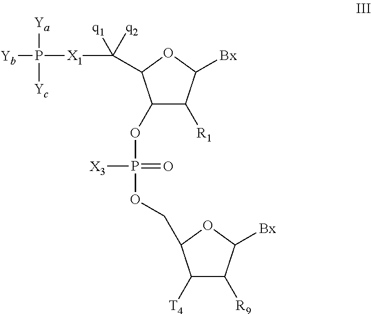

wherein:

each Bx is independently a heterocyclic base moiety;

T₄ is an internucleoside linking group attaching the nucleoside of Formula IV to the remainder of the oligonucleotide;
each of q₁ and q₂ is, independently selected from H, C₁-C₆ alkyl, C₂-C₆ alkenyl, C₂-C₆ alkynyl, substituted C₁-C₆ alkyl, substituted C₁-C₆ alkenyl and substituted C₂-C₆ alkynyl;
X₁ is S, NR₁₆, or CR₁₀R₁₁ wherein each R₁₀ and R₁₁ is, independently, H, F, C₁-C₆ haloalkyl, or C₁-C₆ alkyl; and
R₁ is selected from a halogen, X₂—V, and O—X₄;
or
each of q₁ and q₂ is, independently, selected from H, C₁-C₆ alkyl, C₂-C₆ alkenyl, C₂-C₆ alkynyl, substituted C₁-C₆ alkyl, substituted C₁-C₆ alkenyl and substituted C₂-C₆ alkynyl;
X₁ is O, S, NR₁₆R₁₇, or CR₁₀R₁₁ wherein each R₁₀ and R₁₁ is, independently, H, F, C₁-C₆ haloalkyl, or C₁-C₆ alkyl; and
R₁ is X₂—V;
or
each of q₁ and q₂ is, independently, selected from C₁-C₆ alkyl, C₂-C₆ alkenyl, C₂-C₆ alkynyl, substituted C₁-C₆ alkyl, substituted C₁-C₆ alkenyl and substituted C₂-C₆ alkynyl;
X₁ is O, S, NR₁₆R₁₇, or CR₁₀R₁₁ wherein each R₁₀ and R₁₁ is, independently, H, F, C₁-C₆ haloalkyl, or C₁-C₆ alkyl; and
R₁ is selected from halogen, X₂—V, and O—X₄;

wherein:

X₂ is O, S or CR₇R₈ wherein each R₇ and R₈ is, independently, H or C₁-C₆ alkyl;

V is selected from cholesterol, (CH₂)₂[O(CH₂)₂]ₜOCH₃, where t is from 1-3, (CH₂)₂F, CH₂COOH, CH₂CONH₂, CH₂CONR₅R₆, CH₂COOCH₂CH₃, CH₂CONH(CH₂)ᵢ—S—R₄ where i is from 1 to 10, CH₂CONH(CH₂)ⱼNR₅R₆ where j is from 1 to 6, and CH₂CONH[(CH₂)ₖ₁—N(H)]ₖ₂—(CH₂)ₖ₁NH₂ where each k₁ is independently from 2 to 4 and k₂ is from 2 to 10;

R₄ is selected from H, C₁-C₆ alkyl, C₂-C₆ alkenyl, C₂-C₆ alkynyl, substituted C₁-C₆ alkyl, substituted C₁-C₆ alkenyl, substituted C₂-C₆ alkynyl, C₆-C₁₄ aryl and a thio protecting group;

R₅ and R₆ are each, independently, selected from H, C₁-C₆ alkyl, substituted C₁-C₆ alkyl, C₂-C₆ alkenyl, substituted C₂-C₆ alkenyl, C₂-C₆ alkynyl, and substituted C₂-C₆ alkynyl;

R₁₆ is selected from H, C₁-C₆ alkyl, or substituted C₁-C₆ alkyl;

X₄ is [C(Rₐ)(R_{b})]ₙ—[(C═O)ₘX_{c}]ₖ—R_{d} wherein
each Rₐ and R_{b} is independently H or halogen;
X_{c} is O, S, or N(E₁);
R_{d} is H, C₁-C₆ alkyl, C₂-C₆ alkenyl, C₂-C₆ alkynyl, substituted C₁-C₆ alkyl, substituted C₁-C₆ alkenyl and substituted C₂-C₆ alkynyl or NE₂E₃;
each E₁, E₂, and E₃ is independently H, C₁-C₆ alkyl, or substituted C₁-C₆ alkyl;
n is 1 to 6;
m is 0 or 1; and
k is 0 or 1; and wherein

X₃ is OH or SH;

Yₐ is O or S;

each Y_{b} and Y_{c} is, independently, selected from OH, SH, alkyl, alkoxy, substituted C₁-C₆ alkyl and substituted C₁-C₆ alkoxy;

R₉ is selected from is selected from a halogen, X₂—V, and O—X₄;

wherein each substituted group is, independently, mono or poly substituted with optionally protected substituent groups independently selected from halogen, oxo, OJ₁, NJ₁J₂, SJ₁, N₃, OC(═O)J₁ and CN, wherein each J₁ and J₂ is, independently, H or C₁-C₆ alkyl; and J₄ is hydrogen, or a protecting group.

In certain of the above embodiments, R₁ is F. In certain embodiments, R₁ is OCH₃. In certain embodiments, R₁ is O—C₂-C₄ alkyl or haloalkyl. In certain embodiments, R₁ is O(CH₂)₂OCH₃. In certain embodiments, R₁ is FCH₂CH₃. In certain embodiments, R₁ is (CH₂)₂[O(CH₂)₂]₁₀CH₃, where t is from 1-3. In certain embodiments, R₁ is selected from, trifluoroalkoxy, azido, aminooxy, S-alkyl, N(J₄)-alkyl, O-alkenyl, S-alkenyl, N(J₄)-alkenyl, O-alkynyl, S-alkynyl, N(J₄)-alkynyl, and X₂—V. In certain embodiments, R_{I} is X₂—V. In certain embodiments, V is (CH₂)₂F. In certain embodiments, V is CH₂CONH(CH₂)ᵢ—S—R₄. In certain embodiments, V is CH₂CONH[(CH₂)ₖ₁—N(H)]ₖ₂—(CH₂)ₖ₁NH₂. In certain embodiments, V is CH₂CONH—(CH₂)₃—N(H)—(CH₂)₄—N(H)—(CH₂)₃NH₂. In certain embodiments, V is CH₂CONH(CH₂)ⱼNR₅R₆. In certain such embodiments, j is 2. In certain embodiments, at least one of R₅ and R₆ is other than H. In certain embodiments, at least one of R₅ and R₆ is methyl. In certain embodiments, R₅ is methyl and R₆ is methyl. In certain embodiments, X₂ is O. In certain embodiments, X₂ is S. In certain embodiments, X₂ is CR₇R₈. In certain embodiments, R₇ and R₈ are both H. In certain embodiments, at least one of q₁ and q₂ is C₁-C₆ alkyl or substituted C₁-C₆ alkyl. In certain embodiments, at least one of q₁ and q₂ is C₁-C₆ alkyl. In certain embodiments, at least one of q₁ and q₂ is methyl. In certain embodiments, at least one of q₁ and q₂ is H. In certain embodiments, one of q₁ and q₂ is methyl and the other of q₁ and q₂ is H. In certain embodiments, q₁ and q₂ are each C₁-C₆ alkyl or substituted C₁-C₆ alkyl. In certain embodiments, X₁ is O. In certain embodiments, X₁ is S. In certain embodiments, X₁ is CR₁₀R₁₁. In certain embodiments, R₁₀ and R₁₁ are both H. In certain embodiments, R₉ is selected from F, OCH₃ and O(CH₂)₂OCH₃. In certain embodiments, R₉ is OCH₃. In certain embodiments, R₉ is F. In certain embodiments, R₉ is O(CH₂)₂OCH₃.

In certain embodiments, the invention provides compositions comprising a lipid particle and an oligomeric compound wherein the oligomeric compound comprises an oligonucleotide comprising a phosphate stabilizing nucleoside at the 5′-end, wherein the phosphate stabilizing nucleoside comprises:

a 5′-terminal modified or unmodified phosphate;

a modified sugar moiety comprising:
a 5′-modification; or a 2′-modification; or both a 5′-modification and a 2′-modification; and a linking group linking the phosphate stabilizing nucleoside to the remainder of the oligonucleotide.

In certain such embodiments, the 5′-terminal modified phosphate is selected from: phosphonate, alkylphosphonate, substituted alkylphosphonate, aminoalkyl phosphonate, substituted aminoalkyl phosphonate, phosphorothioate, phosphoramidate, alkylphosphonothioate, substituted alkylphosphonothioate, phosphorodithioate, thiophosphoramidate, and phosphotriester;
the 5′-modification of the sugar moiety of the phosphate stabilizing nucleoside is selected from 5′-alkyl and 5′-halogen;

the 2′-modification of the sugar moiety of the phosphate stabilizing nucleoside is selected from: halogen, allyl, amino, azido, thio, O-allyl, —O—C₁-C₁₀ alkyl, —O—C₁-C₁₀ substituted alkyl, —OCF₃, —O—(CH₂)₂—O—CH₃, —O(CH₂)₂SCH₃, —O—(CH₂)₂—O—N(Rₘ)(Rₙ), —O—CH2-(C═O)—N(Rₘ)(Rₙ), where each Rₘ and Rₙ is, independently, H or substituted or unsubstituted C₁-C₁₀ alkyl, —O[(CH₂)ₙO]ₘCH₃, —O(CH₂)ₙNH₂, —O(CH₂)ₙCH₃, —O(CH₂)ₙONH₂, —OCH₂C(═O)N(H)CH₃, —O(CH₂)ₙON[(CH₂)ₙCH₃]₂, where n and m are from 1 to about 10; C₁ to C₁₀ alkyl, substituted alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl.

In certain embodiments, the modified phosphate is selected from: phosphonate, alkylphosphonate, substituted alkylphosphonate, aminoalkyl phosphonate, substituted aminoalkyl phosphonate, phosphotriester, phosphorothioate, phosphorodithioate, thiophosphoramidate, and phosphoramidate.

In certain embodiments, the modified phosphate is selected from phosphonate, alkylphosphonate, and substituted alkylphosphonate. In certain embodiments, the 5′-phosphate is selected from 5′-deoxy-5′-thio phosphate, phosphoramidate, methylene phosphonate, mono-fluoro methylene phosphonate and di-fluoro methylene phosphonate.

In certain embodiments, the sugar moiety of the phosphate stabilizing nucleoside comprises a 5′-modification and a 2′-modification.

In certain of any of the above embodiments, the remainder of the oligonucleotide comprises at least one modified nucleoside. In certain embodiments, the oligomeric compound comprises a modified base. In certain embodiments, the oligomeric compound comprises a sugar surrogate. In certain embodiments, the sugar surrogate is a tetrahydropyran. In certain embodiments, the tetrahydropyran is F—HNA.

In certain embodiments, the remainder of the oligonucleotide comprises at least one nucleoside comprising a modified sugar. In certain embodiments, the at least one modified nucleoside comprising a modified sugar is selected from a bicyclic nucleoside and a 2′-modified nucleoside. In certain embodiments, the at least one modified nucleoside is a bicyclic nucleoside. In certain embodiments, the bicyclic nucleoside is a (4′-CH₂—O-2′) BNA nucleoside. In certain embodiments, the bicyclic nucleoside is a (4′-(CH₂)₂—O-2′) BNA nucleoside. In certain embodiments, the bicyclic nucleoside is a (4′-C(CH₃)H—O-2′) BNA nucleoside. In certain embodiments, the at least one modified nucleoside is a 2′-modified nucleoside. In certain embodiments, the at least one 2′-modified nucleoside is selected from a 2′-F nucleoside, a 2′-OCH₃ nucleoside, and a 2′-O(CH₂)₂OCH₃ nucleoside. In certain embodiments, the at least one 2′-modified nucleoside is a 2′-F nucleoside. In certain embodiments, the at least one 2′-modified nucleoside is a 2′-OCH₃ nucleoside. In certain embodiments, the at least one 2′-modified nucleoside is a 2′-O(CH₂)₂OCH₃ nucleoside.

In certain embodiments, the remainder of the oligonucleotide comprises at least one unmodified nucleoside. In certain embodiments, the unmodified nucleoside is a ribonucleoside. In certain embodiments, the unmodified nucleoside is a deoxyribonucleoside.

In certain embodiments, the remainder of the oligomeric oligonucleotide comprises at least two modified nucleosides. In certain embodiments, the at least two modified nucleosides comprise the same modification. In certain embodiments, the at least two modified nucleosides comprise different modifications. In certain embodiments, at least one of the at least two modified nucleosides comprises a sugar surrogate. In certain embodiments, at least one of the at least two modified nucleosides comprises a 2′-modification. In certain embodiments, each of the at least two modified nucleosides is independently selected from 2′-F nucleosides, 2′-OCH₃ nucleosides and 2′-O(CH₂)₂OCH₃ nucleosides. In certain embodiments, each of the at least two modified nucleosides is a 2′-F nucleoside. In certain embodiments, each of the at least two modified nucleosides is a 2′-OCH₃ nucleosides. In certain embodiments, each of the at least two modified nucleosides is a 2′-O(CH₂)₂OCH₃ nucleoside. In certain embodiments, essentially every nucleoside of the oligomeric compound is a modified nucleoside. In certain embodiments, every nucleoside of the oligomeric compound is a modified nucleoside.

In certain embodiments, the remainder of the oligonucleotide comprises:

1-20 first-type regions, each first-type region independently comprising 1-20 contiguous nucleosides wherein each nucleoside of each first-type region comprises a first-type modification;

0-20 second-type regions, each second-type region independently comprising 1-20 contiguous nucleosides wherein each nucleoside of each second-type region comprises a second-type modification; and

0-20 third-type regions, each third-type region independently comprising 1-20 contiguous nucleosides wherein each nucleoside of each third-type region comprises a third-type modification; wherein

the first-type modification, the second-type modification, and the third-type modification are each independently selected from 2′-F, 2′-OCH₃, 2′-O(CH₂)₂OCH₃, BNA, F—HNA, 2′-H and 2′-OH;

provided that the first-type modification, the second-type modification, and the third-type modification are each different from one another.

In certain embodiments, the oligonucleotide comprises 2-20 first-type regions; 3-20 first-type regions; 4-20 first-type regions; 5-20 first-type regions; or 6-20 first-type regions. In certain embodiments, the oligonucleotide comprises 1-20 second-type regions; 2-20 second-type regions; 3-20 second-type regions; 4-20 second-type regions; or 5-20 second-type regions. In certain embodiments, the oligonucleotide comprises 1-20 third-type regions; 2-20 third-type regions; 3-20 third-type regions; 4-20 third-type regions; or 5-20 third-type regions.

In certain embodiments, the oligomeric compound comprises a third-type region at the 3′-end of the oligomeric compound. the oligomeric compound comprises a third-type region at the 3′-end of the oligomeric compound the third-type region contains from 1 to 3 modified nucleosides and the third-type modification is 2′-O(CH₂)₂OCH₃. In certain embodiments, the third same type region contains two modified nucleosides and the third-type modification is 2′-O(CH₂)₂OCH₃.

In certain embodiments, each first-type region contains from 1 to 5 modified nucleosides. In certain embodiments, each first-type region contains from 6 to 10 modified nucleosides. In certain embodiments, each first-type region contains from 11 to 15 modified nucleosides. In certain embodiments, each first-type region contains from 16 to 20 modified nucleosides.

In certain embodiments, the first-type modification is 2′-F. In certain embodiments, the first-type modification is 2′-OMe. In certain embodiments, the first-type modification is DNA. In certain embodiments, the first-type modification is 2′-O(CH₂)₂OCH₃. In certain embodiments, the first-type modification is 4′-CH₂—O-2′. In certain embodiments, the first-type modification is 4′-(CH₂)₂—O-2′. In certain embodiments, the first-type modification is 4′-C(CH₃)H—O-2′. In certain embodiments, each second-type region contains from 1 to 5 modified nucleosides. In certain embodiments, each second-type region contains from 6 to 10 modified nucleosides. In certain embodiments, each second-type region contains from 11 to 15 modified nucleosides. In certain embodiments, each second-type region contains from 16 to 20 modified nucleosides. In certain embodiments, the second-type modification is 2′-F. In certain embodiments, the second-type modification is 2′-OMe. In certain embodiments, the second-type modification is DNA. In certain embodiments, the second-type modification is 2′-O(CH₂)₂OCH₃. In certain embodiments, the second -type modification is 4′-CH₂—O-2′. In certain embodiments, the second-type modification is 4′-(CH₂)₂—O-2′. In certain embodiments, the second-type modification is 4′-C(CH₃)H—O-2′. In certain embodiments, the oligomeric compound has an alternating motif wherein the first-type regions alternate with the second-type regions.

In certain embodiments, the invention provides a composition comprising a lipid particle and an oligomeric compound wherein the oligonucleotide comprises at least one region of nucleosides having a nucleoside motif:

(A)ₙ-(B)ₙ-(A)ₙ-(B)ₙ, wherein:

A an B are differently modified nucleosides; and

each n is independently selected from 1, 2, 3, 4, and 5.

In certain embodiments, A and B are each independently selected from a bicyclic and a 2′-modified nucleoside. In certain embodiments, at least one of A and B is a bicyclic nucleoside. In certain embodiments, at least one of A and B is a (4′-CH₂—O-2′) BNA nucleoside. In certain embodiments, at least one of A and B is a (4′-(CH₂)₂—O-2′) BNA nucleoside. In certain embodiments, at least one of A and B is a (4′-C(CH₃)H—O-2′) BNA nucleoside. In certain embodiments, at least one of A and B is a 2′-modified nucleoside. In certain embodiments, the 2′-modified nucleoside is selected from: a 2′-F nucleoside, a 2′-OCH₃ nucleoside, and a 2′-O(CH₂)₂OCH₃ nucleoside. In certain embodiments, A and B are each independently selected from: a 2′-F nucleoside, a 2′-OCH₃ nucleoside, a 2′-O(CH₂)₂OCH₃ nucleoside, a (4′-CH₂—O-2′) BNA nucleoside, a (4% (CH₂)₂—O-2′) BNA nucleoside, a (4′-C(CH₃)H—O-2′) BNA nucleoside, a DNA nucleoside, an RNA nucleoside, and an F—HNA nucleoside. In certain embodiments, A and B are each independently selected from: a 2′-F nucleoside, a 2′-OCH₃ nucleoside, a (4′-CH₂—O-2′) BNA nucleoside, a (4′-(CH₂)₂—O-2′) BNA nucleoside, a (4′-C(CH₃)H—O-2′) BNA nucleoside, and a DNA nucleoside. In certain embodiments, one of A and B is a 2′-F nucleoside. In certain embodiments, one of A and B is a 2′-OCH₃ nucleoside. In certain embodiments, one of A and B is a 2′-O(CH₂)₂OCH₃ nucleoside. In certain embodiments, A is a 2′-F nucleoside and B is a 2′-OCH₃ nucleoside. In certain embodiments, A is a 2′-OCH₃ nucleoside and B is a 2′-F nucleoside. In certain embodiments, one of A and B is selected from a (4′-CH₂—O-2′) BNA nucleoside, a (4′-(CH₂)₂—O-2′) BNA nucleoside, and a (4′-C(CH₃)H—O-2′) BNA nucleoside and the other of A and B is a DNA nucleoside.

In certain embodiments, the invention provides compositions comprising oligomeric compounds wherein the remainder of the oligonucleotide comprises a nucleoside motif: (A)ₓ-(B)₂-(A)_{Y}-(B)₂-(A)_{Z}-(B)₃ wherein
A is a nucleoside of a first type;
B is a nucleoside of a second type;
X is 0-10;
Y is 1-10; and
Z is 1-10.

In certain embodiments, X is selected from 0, 1, 2 and 3. In certain embodiments, X is selected from 4, 5, 6 and 7. In certain embodiments, Y is selected from 1, 2 and 3. In certain embodiments, Y is selected from 4, 5, 6 and 7. In certain embodiments, Z is selected from 1, 2 and 3. In certain embodiments, Z is selected from 4, 5, 6 and 7. In certain embodiments, A is a 2′-F nucleoside. In certain embodiments, B is a 2′-OCH₃ nucleoside.

In certain embodiments, the invention provides compositions comprising oligomeric compounds comprising a 3′-region consisting of from 1 to 5 nucleosides at the 3′-end of the oligomeric compound wherein:

the nucleosides of the 3′-region each comprises the same modification as one another; and

the nucleosides of the 3′-region are modified differently than the last nucleoside adjacent to the 3′-region.

In certain embodiments, the modification of the 3′-region is different from any of the modifications of any of the other nucleosides of the oligomeric compound. In certain embodiments, the nucleosides of the 3′-region are 2′-O(CH₂)₂OCH₃ nucleosides. In certain embodiments, the 3′-region consists of 2 nucleosides. In certain embodiments, the 3′-region consists of 3 nucleosides. In certain embodiments, each nucleoside of the 3′-region comprises a uracil base. In certain embodiments, each nucleoside of the 3′-region comprises an adenine base. In certain embodiments, each nucleoside of the 3′-region comprises a thymine base.

In certain embodiments, the remainder of the oligonucleotide comprises a region of uniformly modified nucleosides. In certain embodiments, the region of uniformly modified nucleosides comprises 2-20 contiguous uniformly modified nucleosides. In certain embodiments, the region of uniformly modified nucleosides comprises 3-20 contiguous uniformly modified nucleosides. In certain embodiments, the region of uniformly modified nucleosides comprises 4-20 contiguous uniformly modified nucleosides. In certain embodiments, the region of uniformly modified nucleosides comprises 5-20 contiguous uniformly modified nucleosides. In certain embodiments, the region of uniformly modified nucleosides comprises 6-20 contiguous uniformly modified nucleosides. In certain embodiments, the region of uniformly modified nucleosides comprises 5-15 contiguous uniformly modified nucleosides. In certain embodiments, the region of uniformly modified nucleosides comprises 6-15 contiguous uniformly modified nucleosides. In certain embodiments, the region of uniformly modified nucleosides comprises 5-10 contiguous uniformly modified nucleosides. In certain embodiments, the region of uniformly modified nucleosides comprises 6-10 contiguous uniformly modified nucleosides.

In certain embodiments, the remainder of the oligonucleotide comprises a region of alternating modified nucleosides and a region of uniformly modified nucleosides. In certain embodiments, the region of alternating nucleotides is 5′ of the region of fully modified nucleosides. In certain embodiments, the region of alternating nucleotides is 3′ of the region of fully modified nucleosides. In certain embodiments, the alternating region and the fully modified region are immediately adjacent to one another. In certain embodiments, the oligomeric compound has additional nucleosides between the alternating region and the fully modified region.

In certain embodiments, the remainder of the oligonucleotide comprises at least one region of nucleosides having a motif I:

N_{f}(PS)Nₘ(PO), wherein:

N_{f} is a 2′-F nucleoside,

Nₘ is a 2′-OCH₃ nucleoside

PS is a phosphorothioate linking group; and

PO is a phosphodiester linking group.

In certain embodiments, the oligomeric compound comprises at least 2, or 3, or 4, or 6, or 7, or 8, or 9, or 10 separate regions of nucleosides having the motif I.

In certain embodiments, the invention provides compositions comprising a lipid particle and an oligomeric compound comprising at least one region having a nucleoside motif selected from:

AABBAA;

ABBABB;

AABAAB;

ABBABAABB;

ABABAA;

AABABAB;

ABABAA;

ABBAABBABABAA;

BABBAABBABABAA; or

ABABBAABBABABAA;

wherein A is a nucleoside of a first type and B is a nucleoside of a second type.

In certain embodiments, oligomeric compounds for use in the compositions of the invention comprise one or more conjugate groups. In certain embodiments, oligomeric compounds consist of the oligonucleotide.

In certain embodiments, the invention provides compositions comprising a lipid particle and an oligomeric compound wherein the oligomeric compound comprises an oligonucleotide comprising a contiguous sequence of linked nucleosides wherein the sequence has the formula:

5′-(Z)_{w}-(L-Q₁-L-Q₂)ₜ-(L-Q₁)ᵤ-(L-Q₃)ᵥ-(G)ₐ-3′

wherein:

each L is an internucleoside linking group;

G is a conjugate or a linking group;

a is 0 or 1;

each of Q₁, Q₂ and Q₃ is, independently, a 2′-modified nucleoside having a 2′-substituent group selected from halogen, allyl, amino, azido, O-allyl, O—C₁-C₆ alkyl, OCF₃, O—(CH₂)₂—O—CH₃, O(CH₂)₂SCH₃, O—(CH₂)₂—O—N(J₅)(J₆) and O—CH₂—C(═O)—N(J₅)(J₆), where each J₅ and J₆ is, independently, H, an amino protecting group or substituted or unsubstituted C₁-C₆ alkyl; provided that Q₁, Q₂ and Q₃ are different from one another;

t is from 4 to 8;

u is 0 or 1;

v is from 1 to 3;

w is 0 or 1; and

Z is a 5′ stabilizing nucleoside.

In certain embodiments, w is 1. In certain embodiments, w is 0. In certain embodiments, Q₁ and Q₂ is, independently, a 2′-modified nucleoside having a 2′-substituent group selected from halogen and O—C₁-C₆ alkyl. In certain embodiments, each Q₁ and Q₂ is, independently, a 2′-modified nucleoside having a 2′-substituent group selected from F and O-methyl. In certain embodiments, each Q₃ is a 2′-modified nucleoside having a 2′-substituent group of O—(CH₂)₂—OCH₃. In certain embodiments, a is 0. In certain embodiments, v is 2. In certain embodiments, u is 0. In certain embodiments, u is 1.

In certain of any of the above embodiments, the oligonucleotide consists of 8-80 linked nucleoside; 8-26 linked nucleosides; 10-24 linked nucleosides; 16-22 linked nucleosides; 16-18 linked nucleosides; 19-22 linked nucleosides.

In certain of any of the above embodiments, the second nucleoside from the 5′-end comprises a sugar moiety comprising a 2′-substituent selected from OH and a halogen. In certain embodiments, the second nucleoside from the 5′-end is a 2′-F modified nucleoside.

In certain of any of the above embodiments, the oligomeric compound comprises at least one modified linking group. In certain embodiments, each internucleoside linking group is, independently, phosphodiester or phosphorothioate. In certain embodiments, the 5′-most internucleoside linking group is a phosphorothioate linking group. In certain embodiments, at least one phosphorothioate region comprising at least two contiguous phosphorothioate linking groups. In certain embodiments, the at least one phosphorothioate region comprises from 3 to 12 contiguous phosphorothioate linking groups. In certain embodiments, the at least one phosphorothioate region comprises from 6 to 8 phosphorothioate linking groups. In certain embodiments, the at least one phosphorothioate region is located at the 3′-end of the oligomeric compound. In certain embodiments, the at least one phosphorothioate region is located within 3 nucleosides of the 3′-end of the oligomeric compound. In certain embodiments, the 7-9 internucleoside linkages at the 3′ end of the oligonucleotide are phosphorothioate linkages and the internucleoside linkage at the 5′-end is a phosphorothioate linkage.

In certain embodiments, the invention provides compositions comprising a lipid particle and an oligomeric compound wherein the oligomeric compound comprises an oligonucleotide consisting of 10 to 30 linked nucleosides wherein:
(a) the nucleoside at the 5′ end is a phosphate stabilizing nucleoside comprising:

a 5′-terminal modified or unmodified phosphate; and

a modified sugar moiety comprising:
a 5′-modification; or a 2′-modification; or both a 5′-modification and a 2′-modification;
(b) the sugar moiety of the second nucleoside from the 5′-end is selected from an unmodified 2′-OH sugar, and a modified sugar comprising a modification selected from: 2′-halogen, 2′O-alkyl, and 2′-O -substituted alkyl; and
(c) the first internucleoside linkage at the 5′-end and the last seven internucleoside linkages at the 3′-end are phosphorothioate linkages; and
(d) at least one internucleoside linkage is other than a phosphorothioate linkage.

In certain embodiments, the 5′-terminal modified phosphate is selected from: phosphonate, alkylphosphonate, substituted alkylphosphonate, aminoalkyl phosphonate, substituted aminoalkyl phosphonate, phosphorothioate, phosphoramidate, alkylphosphonothioate, substituted alkylphosphonothioate, phosphorodithioate, thiophosphoramidate, and phosphotriester;
the 5′-modification of the sugar moiety of the phosphate stabilizing nucleoside is selected from 5′-alkyl and 5′-halogen; and

the 2′-modification of the sugar moiety of the phosphate stabilizing nucleoside is selected from: halogen, allyl, amino, azido, thio, O-allyl, —O—C₁-C₁₀ alkyl, —O—C₁-C₁₀ substituted alkyl, —OCF₃, —O—(CH₂)₂—O—CH₃, —O(CH₂)₂SCH₃, —O—(CH₂)₂—O—N(Rₘ)(Rₙ), —O—CH2-C(═O)—N(Rₘ)(Rₙ), where each Rₘ and Rₙ is, independently, H or substituted or unsubstituted C₁-C₁₀ alkyl, —O[(CH₂)ₙO]ₘCH₃, —O(CH₂)ₙNH₂, —O(CH₂)ₙCH₃, —O(CH₂)ₙONH₂, —OCH₂C(═O)N(H)CH₃, —O(CH₂)ₙON[(CH₂)ₙCH₃]₂, where n and m are from 1 to about 10; C₁ to C₁₀ alkyl, substituted alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl.

In certain embodiments, the modified phosphate is selected from: phosphonate, alkylphosphonate, substituted alkylphosphonate, aminoalkyl phosphonate, substituted aminoalkyl phosphonate, phosphotriester, phosphorothioate, phosphorodithioate, thiophosphoramidate, and phosphoramidate.

In certain embodiments, the modified phosphate is selected from: phosphonate, alkylphosphonate, and substituted alkylphosphonate.

In certain embodiments, the modified phosphate is selected from 5′-deoxy-5′-thio phosphate, phosphoramidate, methylene phosphonate, mono-fluoro methylene phosphonate and di-fluoro methylene phosphonate. In certain embodiments, the sugar moiety of the phosphate stabilizing nucleoside comprises a 5′-modification and a 2′-modification.

In certain embodiments, the oligomeric compound is an antisense compound. In certain embodiments, the antisense compound is an RNAi compound. In certain embodiments, the antisense compound is an siRNAi compound. In certain embodiments, the antisense compound is a microRNA mimic. In certain embodiments, the antisense compound is an RNase H antisense compound. In certain embodiments, the antisense compound modulates splicing.

In certain embodiments, at least a portion of the nucleobase sequence of the oligonucleotide is complementary to a portion of a target nucleic acid, wherein the target nucleic acid is selected from: a target mRNA, a target pre-mRNA, a target microRNA, and a target non-coding RNA. In certain embodiments, the nucleobase sequence of the oligonucleotide a region of 100% complementarity to the target nucleic acid and wherein the region of 100% complementarity is at least 10 nucleobases. In certain embodiments, the region of 100% complementarity is at least 15 nucleobases. In certain embodiments, the region of 100% complementarity is at least 20 nucleobases. In certain embodiments, the oligonucleotide is at least 85% complementary to the target nucleic acid. In certain embodiments, the oligonucleotide is at least 90% complementary to the target nucleic acid. In certain embodiments, the oligonucleotide is at least 95% complementary to the target nucleic acid. In certain embodiments, the oligonucleotide is at least 98% complementary to the target nucleic acid. In certain embodiments, the oligonucleotide is 100% complementary to the target nucleic acid.

In certain embodiments, the antisense compound is a microRNA mimic having a nucleobase sequence comprising a portion that is at least 80% identical to the seed region of a microRNA and that has overall identity with the microRNA of at least 70%. In certain embodiments, the nucleobase sequence of the microRNA mimic has a portion that is at least 80% identical to the sequence of the seed region of a microRNA and has overall identity with the microRNA of at least 75%. In certain embodiments, the nucleobase sequence of the microRNA mimic has a portion that is at least 80% identical to the sequence of the seed region of a microRNA and has overall identity with the microRNA of at least 80%. In certain embodiments, the nucleobase sequence of the microRNA mimic has a portion that is at least 100% identical to the sequence of the seed region of a microRNA and has overall identity with the microRNA of at least 80%. In certain embodiments, the nucleobase sequence of the microRNA mimic has a portion that is at least 100% identical to the sequence of the seed region of a microRNA and has overall identity with the microRNA of at least 85%. In certain embodiments, the nucleobase sequence of the microRNA mimic has a portion that is 100% identical to the sequence of the microRNA. In certain embodiments, nucleobase sequence of the oligonucleotide comprises a region of 100% complementarity to a seed match segment of a target nucleic acid. In certain embodiments, the antisense compound is a microRNA mimic having a nucleobase sequence comprising a portion that is at least 80% identical to the seed region of a microRNA and that has overall identity with the microRNA of at least 50%. In certain embodiments, the antisense compound is a microRNA mimic having a nucleobase sequence comprising a portion that is at least 80% identical to the seed region of a microRNA and that has overall identity with the microRNA of at least 55%. In certain embodiments, the antisense compound is a microRNA mimic having a nucleobase sequence comprising a portion that is at least 80% identical to the seed region of a microRNA and that has overall identity with the microRNA of at least 60%. In certain embodiments, the antisense compound is a microRNA mimic having a nucleobase sequence comprising a portion that is at least 80% identical to the seed region of a microRNA and that has overall identity with the microRNA of at least 65%. In certain embodiments, the oligomeric compound comprises a nucleobase sequence selected from a microRNA sequence found in miRBase. In certain embodiments, the oligomeric compound consists of a nucleobase sequence selected from a microRNA sequence found in miRBase.

In certain embodiments, the target nucleic acid is a target mRNA. In certain embodiments, the target nucleic acid is a target pre-mRNA. In certain embodiments, the target nucleic acid is a non-coding RNA. In certain embodiments, the target nucleic acid is a microRNA. In certain embodiments, the target nucleic acid is a pre-mir. In certain embodiments, the target nucleic acid is a pri-mir.

In certain embodiments, the nucleobase sequence of the oligonucleotide comprises a region of 100% complementarity to the target nucleic acid and wherein the region of 100% complementarity is at least 10 nucleobases. In certain embodiments, the nucleobase sequence of the oligonucleotide comprises a region of 100% complementarity to the target nucleic acid and wherein the region of 100% complementarity is at least 6 nucleobases. In certain embodiments, the nucleobase sequence of the oligonucleotide comprises a region of 100% complementarity to the target nucleic acid and wherein the region of 100% complementarity is at least 7 nucleobases. In certain embodiments, the target nucleic acid is a mammalian target nucleic acid. In certain embodiments, the mammalian target nucleic acid is a human target nucleic acid.

In certain embodiments, oligomeric compounds comprise from 1 to 3 terminal group nucleosides on at least one end of the oligonucleotide. In certain embodiments, oligomeric compound comprise from 1 to 3 terminal group nucleosides at the 3′-end of the oligonucleotide. In certain embodiments, oligomeric compound comprise from 1 to 3 terminal group nucleosides at the 5′-end of the oligonucleotide.

In certain embodiments, oligomeric compounds for use in the compositions of the invention are single stranded.

In certain embodiments, oligomeric compounds for use in the compositions of the invention are double stranded.

In certain embodiments, the invention provides methods comprising contacting a cell with a composition described herein. In certain embodiments, such methods comprise detecting antisense activity. In certain embodiments, the detecting antisense activity comprises detecting a phenotypic change in the cell. In certain embodiments, the detecting antisense activity comprises detecting a change in the amount of target nucleic acid in the cell. In certain embodiments, the detecting antisense activity comprises detecting a change in the amount of a target protein. In certain embodiments, the cell is in vitro. In certain embodiments, the cell is in an animal. In certain embodiments, animal is a mammal. In certain embodiments, the mammal is a human.

In certain embodiments, the invention provides methods of modulating a target mRNA in a cell comprising contacting the cell with a composition of the invention and thereby modulating the mRNA in a cell. In certain embodiments, such methods comprise detecting a phenotypic change in the cell. In certain embodiments, methods comprise detecting a decrease in mRNA levels in the cell. In certain embodiments, methods comprise detecting a change in the amount of a target protein. In certain embodiments, the cell is in vitro. In certain embodiments, the cell is in an animal. In certain embodiments, the animal is a mammal. In certain embodiments, the mammal is a human.

In certain embodiments, the invention provides methods of administering to an animal a pharmaceutical composition of the invention. In certain embodiments, the animal is a mammal. In certain embodiments, the mammal is a human. In certain embodiments, the methods comprise detecting antisense activity in the animal. In certain embodiments, the methods comprise detecting a change in the amount of target nucleic acid in the animal. In certain embodiments, the methods comprise detecting a change in the amount of a target protein in the animal. In certain embodiments, the methods comprise detecting a phenotypic change in the animal. In certain embodiments, the phenotypic change is a change in the amount or quality of a biological marker of activity.

In certain embodiments, the invention provides use of a composition of the invention for the manufacture of a medicament for the treatment of a disease characterized by undesired gene expression.

In certain embodiments, the invention provides use of a composition of the invention for the manufacture of a medicament for treating a disease by inhibiting gene expression.

In certain embodiments, the invention provides methods of comprising detecting antisense activity wherein the antisense activity is microRNA mimic activity. In certain embodiments, the detecting microRNA mimic activity comprises detecting a change in the amount of a target nucleic acid in a cell. In certain embodiments, the detecting microRNA mimic activity comprises detecting a change in the amount of a target protein in cell.

In certain embodiments the invention provides compositions comprising oligomeric compounds having a nucleobase sequence selected from among SEQ ID NOs 20, 21, 23, 24, 25, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, and 91.

In certain embodiments, the present invention provides compositions comprising oligomeric compounds having a nucleobase sequence selected from the table below.

| | | SEQ ID |
|---|---|---|
| miR ID | SEQUENCE | NO |
| hsa-let-7a-1 | UGAGGUAGUAGGUUGUAUAGUU | 38 |
| hsa-let-7b | UGAGGUAGUAGGUUGUGUGGUU | 39 |
| hsa-let-7c | UGAGGUAGUAGGUUGUAUGGUU | 40 |
| hsa-let-7i | UGAGGUAGUAGUUUGUGCUGUU | 41 |
| hsa-miR-1-1 | UGGAAUGUAAAGAAGUAUGUAU | 42 |
| hsa-miR-10a | UACCCUGUAGAUCCGAAUUUGUG | 43 |
| hsa-miR-15a | UAGCAGCACAUAAUGGUUUGUG | 44 |
| hsa-miR-16-1 | UAGCAGCACGUAAAUAUUGGCG | 45 |
| hsa-miR-29a | UAGCACCAUCUGAAAUCGGUUA | 46 |
| hsa-miR-29b-1 | UAGCACCAUUUGAAAUCAGUGUU | 47 |
| hsa-miR-29c | UAGCACCAUUUGAAAUCGGUUA | 48 |
| hsa-miR-34a | UGGCAGUGUCUUAGCUGGUUGU | 49 |
| hsa-miR-34b | CAAUCACUAACUCCACUGCCAU | 50 |
| hsa-miR-34c-5p | AGGCAGUGUAGUUAGCUGAUUGC | 51 |
| hsa-miR-93 | CAAAGUGCUGUUCGUGCAGGUAG | 52 |
| hsa-miR-101-1 | UACAGUACUGUGAUAACUGAA | 53 |
| hsa-miR-122 | UGGAGUGUGACAAUGGUGUUUG | 54 |
| hsa-miR-124-1 | UAAGGCACGCGGUGAAUGCC | 55 |
| hsa-miR-125a-5p | UCCCUGAGACCCUUUAACCUGUGA | 56 |
| hsa-miR-125b-1 | UCCCUGAGACCCUAACUUGUGA | 57 |
| hsa-miR-126 | UCGUACCGUGAGUAAUAAUGCG | 58 |
| hsa-miR-132 | UAACAGUCUACAGCCAUGGUCG | 59 |
| hsa-miR-133a-1 | UUUGGUCCCCUUCAACCAGCUG | 60 |
| hsa-miR-133b | UUUGGUCCCCUUCAACCAGCUA | 61 |
| hsa-miR-146a | UGAGAACUGAAUUCCAUGGGUU | 62 |
| hsa-miR-150 | UCUCCCAACCCUUGUACCAGUG | 63 |
| hsa-miR-155 | UUAAUGCUAAUCGUGAUAGGGGU | 64 |
| hsa-miR-181a-1 | AACAUUCAACGCUGUCGGUGAGU | 65 |
| hsa-miR-181b-1 | AACAUUCAUUGCUGUCGGUGGGU | 66 |
| hsa-miR-193a-5p | UGGGUCUUUGCGGGCGAGAUGA | 67 |
| hsa-miR-196a-1 | UAGGUAGUUUCAUGUUGUUGGG | 68 |
| hsa-miR-203 | GUGAAAUGUUUAGGACCACUAG | 69 |
| hsa-miR-206 | UGGAAUGUAAGGAAGUGUGUGG | 70 |
| hsa-miR-210 | CUGUGCGUGUGACAGCGGCUGA | 71 |
| hsa-miR-296-5p | AGGGCCCCCCCUCAAUCCUGU | 72 |
| hsa-miR-335 | UCAAGAGCAAUAACGAAAAAUGU | 73 |
| hsa-miR-7 | UGGAAGACUAGUGAUUUUGUUGU | 74 |
| hsa-miR-21 | UAGCUUAUCAGACUGAUGUUGA | 75 |
| hsa-miR-22 | AAGCUGCCAGUUGAAGAACUGU | 76 |
| hsa-miR-26a | UUCAAGUAAUCCAGGAUAGGCU | 77 |
| hsa-miR-26b | UUCAAGUAAUUCAGGAUAGGU | 78 |
| hsa-miR-141 | UAACACUGUCUGGUAAAGAUGG | 79 |
| hsa-miR-143 | UGAGAUGAAGCACUGUAGCUC | 80 |
| hsa-miR-145 | GUCCAGUUUUCCCAGGAAUCCCU | 81 |
| hsa-miR-195 | UAGCAGCACAGAAAUAUUGGC | 82 |
| hsa-miR-200a | UAACACUGUCUGGUAACGAUGU | 83 |
| hsa-miR-200b | UAAUACUGCCUGGUAAUGAUGA | 84 |
| hsa-miR-200c | UAAUACUGCCGGGUAAUGAUGGA | 85 |
| hsa-miR-205 | UCCUUCAUUCCACCGGAGUCUG | 86 |
| hsa-miR-208a | AUAAGACGAGCAAAAAGCUUGU | 87 |
| hsa-miR-208b | AUAAGACGAACAAAAGGUUUGU | 88 |
| hsa-miR-221 | AGCUACAUUGUCUGCUGGGUUUC | 89 |
| hsa-miR-222 | AGCUACAUCUGGCUACUGGGU | 90 |
| hsa-miR-223 | UGUCAGUUUGUCAAAUACCCCA | 91 |

### BRIEF DESCRIPTION OF THE FIGURES

FIG. 1 is a graph illustrating the reduction of PTEN mRNA with various LNP06 formulated ssRNA.

### DETAILED DESCRIPTION OF THE INVENTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference in their entirety for any purpose. Each of the following patent applications is hereby incorporated by reference in its entirety: U.S. Provisional Applications 61/108,457, filed 2008 Oct. 24; 61/108,464, filed 2008 Oct. 24; 61/149,297, filed 2009 Feb. 2; 61/150,492, filed 2009 Feb. 6; 61/163,217, filed 2009 Mar. 25; 61/174,137, filed 2009 Apr. 30; 61/239,672, filed 2009 Sep. 3; and PCT/US2009/061913 and PCT/US2009/061916 each filed 2009 Oct. 23 (the same day as the present application).

### I. DEFINITIONS

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Certain such techniques and procedures may be found for example in “Carbohydrate Modifications in Antisense Research” Edited by Sangvi and Cook, American Chemical Society, Washington D.C., 1994; “Remington's Pharmaceutical Sciences,” Mack Publishing Co., Easton, Pa., 18th edition, 1990; and “Antisense Drug Technology, Principles, Strategies, and Applications” Edited by Stanley T. Crooke, CRC Press, Boca Raton, Fla.; and Sambrook et al., “Molecular Cloning, A laboratory Manual,” 2^{nd} Edition, Cold Spring Harbor Laboratory Press, 1989, which are hereby incorporated by reference for any purpose. Where permitted, all patents, applications, published applications and other publications and other data referred to throughout in the disclosure herein are incorporated by reference in their entirety.

Unless otherwise indicated, the following terms have the following meanings:

As used herein, “nucleoside” refers to a compound comprising a heterocyclic base moiety and a sugar moiety. Nucleosides include, but are not limited to, naturally occurring nucleosides (as found in DNA and RNA), abasic nucleosides, modified nucleosides, and nucleosides having mimetic bases and/or sugar groups. Nucleosides may be modified with any of a variety of substituents. Nucleosides may include a phosphate moiety.

As used herein, “sugar moiety” means a natural or modified sugar ring or sugar surrogate.

As used herein the term “sugar surrogate” refers to a structure that is capable of replacing the furanose ring of a naturally occurring nucleoside. In certain embodiments, sugar surrogates are non-furanose (or 4′-substituted furanose) rings or ring systems or open systems. Such structures include simple changes relative to the natural furanose ring, such as a six membered ring or may be more complicated as is the case with the non-ring system used in peptide nucleic acid. Sugar surrogates includes without limitation morpholinos, cyclohexenyls and cyclohexitols. In most nucleosides having a sugar surrogate group the heterocyclic base moiety is generally maintained to permit hybridization.

As used herein, “nucleotide” refers to a nucleoside further comprising a phosphate linking group. As used herein, “linked nucleosides” may or may not be linked by phosphate linkages and thus includes “linked nucleotides.”

As used herein, “nucleobase” refers to the heterocyclic base portion of a nucleoside. Nucleobases may be naturally occurring or may be modified. In certain embodiments, a nucleobase may comprise any atom or group of atoms capable of hydrogen bonding to a base of another nucleic acid.

As used herein, “modified nucleoside” refers to a nucleoside comprising at least one modification compared to naturally occurring RNA or DNA nucleosides. Such modification may be at the sugar moiety and/or at the nucleobases.

As used herein, “bicyclic nucleoside” or “BNA” refers to a nucleoside having a sugar moiety comprising a sugar-ring (including, but not limited to, furanose) comprising a bridge connecting two carbon atoms of the sugar ring to form a second ring. In certain embodiments, the bridge connects the 4′ carbon to the 2′ carbon of a 5-membered sugar ring.

As used herein, “4′-2′ bicyclic nucleoside” refers to a bicyclic nucleoside comprising a furanose ring comprising a bridge connecting two carbon atoms of the furanose ring connects the 2′ carbon atom and the 4′ carbon atom of the sugar ring.

As used herein, “2′-modified” or “2′-substituted” refers to a nucleoside comprising a sugar comprising a substituent at the 2′ position other than H or OH. 2′-modified nucleosides, include, but are not limited to, bicyclic nucleosides wherein the bridge connecting two carbon atoms of the sugar ring connects the 2′ carbon and another carbon of the sugar ring; and nucleosides with non-bridging 2′ substituents, such as allyl, amino, azido, thio, O-allyl, O—C₁-C₁₀ alkyl, —OCF₃, O—(CH₂)₂—O—CH₃, 2′-O(CH₂)₂SCH₃, O—(CH₂)₂—O—N(Rₘ)(Rₙ), or O—CH₂—C(═O)—N(Rₘ)(Rₙ), where each Rₘ and Rₙ is, independently, H or substituted or unsubstituted C₁-C₁₀ alkyl. 2′-modified nucleosides may further comprise other modifications, for example at other positions of the sugar and/or at the nucleobase.

As used herein, “2′-F” refers to a nucleoside comprising a sugar comprising a fluoro group at the 2′ position.

As used herein, “2′-OMe” or “2′-OCH₃” or “2′-O-methyl” each refers to a nucleoside comprising a sugar comprising an —OCH₃ group at the 2′ position of the sugar ring.

As used herein, “MOE” or “2′-MOE” or “2′-OCH₂CH₂OCH₃” or “2′-O-methoxyethyl” each refers to a nucleoside comprising a sugar comprising a —OCH₂CH₂OCH₃ group at the 2′ position of the sugar ring.

As used herein, “oligonucleotide” refers to a compound comprising a plurality of linked nucleosides. In certain embodiments, one or more of the plurality of nucleosides is modified. In certain embodiments, an oligonucleotide comprises one or more ribonucleosides (RNA) and/or deoxyribonucleosides (DNA).

As used herein “oligonucleoside” refers to an oligonucleotide in which none of the internucleoside linkages contains a phosphorus atom. As used herein, oligonucleotides include oligonucleosides.

As used herein, “modified oligonucleotide” refers to an oligonucleotide comprising at least one modified nucleoside and/or at least one modified internucleoside linkage.

As used herein “internucleoside linkage” refers to a covalent linkage between adjacent nucleosides.

As used herein “naturally occurring internucleoside linkage” refers to a 3′ to 5′ phosphodiester linkage.

As used herein, “modified internucleoside linkage” refers to any internucleoside linkage other than a naturally occurring internucleoside linkage.

As used herein, “oligomeric compound” refers to a polymeric structure comprising two or more sub-structures. In certain embodiments, an oligomeric compound is an oligonucleotide. In certain embodiments, an oligomeric compound comprises one or more conjugate groups and/or terminal groups.

As used herein, unless otherwise indicated or modified, the term “double-stranded” or refers to two separate oligomeric compounds that are hybridized to one another. Such double stranded compounds my have one or more or non-hybridizing nucleosides at one or both ends of one or both strands (overhangs) and/or one or more internal non-hybridizing nucleosides (mismatches) provided there is sufficient complementarity to maintain hybridization under physiologically relevant conditions.

As used herein, the term “self-complementary” or “hair-pin” refers to a single oligomeric compound that comprises a duplex region formed by the oligomeric compound hybridizing to itself.

As used herein, the term “single-stranded” refers to an oligomeric compound that is not hybridized to its complement and that does not have sufficient self-complementarity to form a hair-pin structure under physiologically relevant conditions. A single-stranded compound may be capable of binding to its complement to become a double-stranded or partially double-stranded compound.

As used herein, “terminal group” refers to one or more atom attached to either, or both, the 3′ end or the 5′ end of an oligonucleotide. In certain embodiments a terminal group is a conjugate group. In certain embodiments, a terminal group comprises one or more additional nucleosides.

As used herein, “conjugate” refers to an atom or group of atoms bound to an oligonucleotide or oligomeric compound. In general, conjugate groups modify one or more properties of the compound to which they are attached, including, but not limited to pharmacodynamic, pharmacokinetic, binding, absorption, cellular distribution, cellular uptake, charge and clearance. Conjugate groups are routinely used in the chemical arts and are linked directly or via an optional linking moiety or linking group to the parent compound such as an oligomeric compound. In certain embodiments, conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes. In certain embodiments, conjugates are terminal groups. In certain embodiments, conjugates are attached to a 3′ or 5′ terminal nucleoside or to an internal nucleosides of an oligonucleotide.

As used herein, “conjugate linking group” refers to any atom or group of atoms used to attach a conjugate to an oligonucleotide or oligomeric compound. Linking groups or bifunctional linking moieties such as those known in the art are amenable to the present invention.

As used herein, “antisense compound” refers to an oligomeric compound, at least a portion of which is at least partially complementary to a target nucleic acid to which it hybridizes. In certain embodiments, an antisense compound modulates (increases or decreases) expression or amount of a target nucleic acid. In certain embodiments, an antisense compound alters splicing of a target pre-mRNA resulting in a different splice variant. In certain embodiments, an antisense compound modulates expression of one or more different target proteins. Antisense mechanisms contemplated herein include, but are not limited to an RNase H mechanism, RNAi mechanisms, splicing modulation, translational arrest, altering RNA processing, inhibiting microRNA function, or mimicking microRNA function.

As used herein, “expression” refers to the process by which a gene ultimately results in a protein. Expression includes, but is not limited to, transcription, splicing, post-transcriptional modification, and translation.

As used herein, “RNAi” refers to a mechanism by which certain antisense compounds effect expression or amount of a target nucleic acid. RNAi mechanisms involve the RISC pathway.

As used herein, “RNAi compound” refers to an oligomeric compound that acts, at least in part, through an RNAi mechanism to modulate a target nucleic acid and/or protein encoded by a target nucleic acid. RNAi compounds include, but are not limited to double-stranded short interfering RNA (siRNA), single-stranded RNA (ssRNA), and microRNA, including microRNA mimics.

As used herein, “antisense oligonucleotide” refers to an antisense compound that is an oligonucleotide.

As used herein, “antisense activity” refers to any detectable and/or measurable activity attributable to the hybridization of an antisense compound to its target nucleic acid. In certain embodiments, such activity may be an increase or decrease in an amount of a nucleic acid or protein. In certain embodiments, such activity may be a change in the ratio of splice variants of a nucleic acid or protein. Detection and/or measuring of antisense activity may be direct or indirect. For example, in certain embodiments, antisense activity is assessed by detecting and/or measuring the amount of target protein or the relative amounts of splice variants of a target protein. In certain embodiments, antisense activity is assessed by detecting and/or measuring the amount of target nucleic acids and/or cleaved target nucleic acids and/or alternatively spliced target nucleic acids. In certain embodiments, antisense activity is assessed by observing a phenotypic change in a cell or animal.

As used herein “detecting” or “measuring” in connection with an activity, response, or effect indicate that a test for detecting or measuring such activity, response, or effect is performed. Such detection and/or measuring may include values of zero. Thus, if a test for detection or measuring results in a finding of no activity (activity of zero), the step of detecting or measuring the activity has nevertheless been performed. For example, in certain embodiments, the present invention provides methods that comprise steps of detecting antisense activity, detecting toxicity, and/or measuring a marker of toxicity. Any such step may include values of zero.

As used herein, “target nucleic acid” refers to any nucleic acid molecule the expression, amount, or activity of which is capable of being modulated by an antisense compound. In certain embodiments, the target nucleic acid is DNA or RNA. In certain embodiments, the target RNA is mRNA, pre-mRNA, non-coding RNA, pri-microRNA, pre-microRNA, mature microRNA, promoter-directed RNA, or natural antisense transcripts. For example, the target nucleic acid can be a cellular gene (or mRNA transcribed from the gene) whose expression is associated with a particular disorder or disease state, or a nucleic acid molecule from an infectious agent. In certain embodiments, target nucleic acid is a viral or bacterial nucleic acid.

As used herein, “target mRNA” refers to a pre-selected RNA molecule that encodes a protein.

As used herein, “target pre-mRNA” refers to a pre-selected RNA transcript that has not been fully processed into mRNA. Notably, pre-RNA includes one or more intron.

As used herein, “target microRNA” refers to a pre-selected non-coding RNA molecule about 18-30 nucleobases in length that modulates expression of one or more proteins or to a precursor of such a non-coding molecule.

As used herein, “target pdRNA” refers to refers to a pre-selected RNA molecule that interacts with one or more promoter to modulate transcription.

As used herein, “microRNA” refers to a naturally occurring, small, non-coding RNA that represses gene expression at the level of translation. In certain embodiments, a microRNA represses gene expression by binding to a target site within a 3′ untranslated region of a target nucleic acid. In certain embodiments, a microRNA has a nucleobase sequence as set forth in miRBase, a database of published microRNA sequences found at http://microrna.sanger.ac.uk/sequences/. In certain embodiments, a microRNA has a nucleobase sequence as set forth in miRBase version 10.1 released December 2007, which is herein incorporated by reference in its entirety. In certain embodiments, a microRNA has a nucleobase sequence as set forth in miRBase version 12.0 released September 2008, which is herein incorporated by reference in its entirety. As used herein, “microRNA mimic” refers to an oligomeric compound having a sequence that is at least partially identical to that of a microRNA. In certain embodiments, a microRNA mimic comprises the microRNA seed region of a microRNA. In certain embodiments, a microRNA mimic modulates translation of more than one target nucleic acids.

As used herein, “seed region” refers to a region at or near the 5′ end of an antisense compound having a nucleobase sequence that is import for target nucleic acid recognition by the antisense compound. In certain embodiments, a seed region comprises nucleobases 2-8 of an antisense compound. In certain embodiments, a seed region comprises nucleobases 2-7 of an antisense compound. In certain embodiments, a seed region comprises nucleobases 1-7 of an antisense compound. In certain embodiments, a seed region comprises nucleobases 1-6 of an antisense compound. In certain embodiments, a seed region comprises nucleobases 1-8 of an antisense compound.

As used herein, “microRNA seed region” refers to a seed region of a microRNA or microRNA mimic. In certain embodiments, a microRNA seed region comprises nucleobases 2-8 of a microRNA or microRNA mimic. In certain embodiments, a microRNA seed region comprises nucleobases 2-7 of a microRNA or microRNA mimic. In certain embodiments, a microRNA seed region comprises nucleobases 1-7 of a microRNA or microRNA mimic. In certain embodiments, a microRNA seed region comprises nucleobases 1-6 of a microRNA or microRNA mimic. In certain embodiments, a microRNA seed region comprises nucleobases 1-8 of a microRNA or microRNA mimic.

As used herein, “seed match segment” refers to a portion of a target nucleic acid having nucleobase complementarity to a seed region. In certain embodiments, a seed match segment has nucleobase complementarity to nucleobases 2-8 of an siRNA, ssRNA, natural microRNA or microRNA mimic. In certain embodiments, a seed match segment has nucleobase complementarity to nucleobases 2-7 of an siRNA, ssRNA, microRNA or microRNA mimic. In certain embodiments, a seed match segment has nucleobase complementarity to nucleobases 1-6 of an siRNA, ssRNA, microRNA or microRNA mimic. In certain embodiments, a seed match segment has nucleobase complementarity to nucleobases 1-7 of an siRNA, ssRNA, microRNA or microRNA mimic. In certain embodiments, a seed match segment has nucleobase complementarity to nucleobases 1-8 of an siRNA, ssRNA, microRNA or microRNA mimic.

As used herein, “seed match target nucleic acid” refers to a target nucleic acid comprising a seed match segment.

As used herein, “microRNA family” refers to a group of microRNAs that share a microRNA seed sequence. In certain embodiments, microRNA family members regulate a common set of target nucleic acids. In certain embodiments, the shared microRNA seed sequence is found at the same nucleobase positions in each member of a microRNA family. In certain embodiments, the shared microRNA seed sequence is not found at the same nucleobase positions in each member of a microRNA family. For example, a microRNA seed sequence found at nucleobases 1-7 of one member of a microRNA family may be found at nucleobases 2-8 of another member of a microRNA family.

As used herein, “target non-coding RNA” refers to a pre-selected RNA molecule that is not translated to generate a protein. Certain non-coding RNA are involved in regulation of expression.

As used herein, “target viral nucleic acid” refers to a pre-selected nucleic acid (RNA or DNA) associated with a virus. Such viral nucleic acid includes nucleic acids that constitute the viral genome, as well as transcripts (including reverse-transcripts and RNA transcribed from RNA) of those nucleic acids, whether or not produced by the host cellular machinery. In certain instances, viral nucleic acids also include host nucleic acids that are recruited by a virus upon viral infection.

As used herein, “targeting” or “targeted to” refers to the association of an antisense compound to a particular target nucleic acid molecule or a particular region of nucleotides within a target nucleic acid molecule. An antisense compound targets a target nucleic acid if it is sufficiently complementary to the target nucleic acid to allow hybridization under physiological conditions.

As used herein, “target protein” refers to a protein, the expression of which is modulated by an antisense compound. In certain embodiments, a target protein is encoded by a target nucleic acid. In certain embodiments, expression of a target protein is otherwise influenced by a target nucleic acid.

In certain embodiments, compositions of the invention reduce the target RNA by at least 25%, at least 30%, at least 35%, at least 40%, at least 45%, at least 50%, at least 55%, at least 60%, at least 65%, at least 70%, at least 75%, at least 80%, at least 90%, or at least 95%. The percentage of reduction are define as percentage of KnockDown (% KD).

As used herein, “nucleobase complementarity” or “complementarity” when in reference to nucleobases refers to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair. Nucleobases comprising certain modifications may maintain the ability to pair with a counterpart nucleobase and thus, are still capable of nucleobase complementarity.

As used herein, “non-complementary” in reference to nucleobases refers to a pair of nucleobases that do not form hydrogen bonds with one another or otherwise support hybridization.

As used herein, “complementary” in reference to linked nucleosides, oligonucleotides, or nucleic acids, refers to the capacity of an oligomeric compound to hybridize to another oligomeric compound or nucleic acid through nucleobase complementarity. In certain embodiments, an antisense compound and its target are complementary to each other when a sufficient number of corresponding positions in each molecule are occupied by nucleobases that can bond with each other to allow stable association between the antisense compound and the target. One skilled in the art recognizes that the inclusion of mismatches is possible without eliminating the ability of the oligomeric compounds to remain in association. Therefore, described herein are antisense compounds that may comprise up to about 20% nucleotides that are mismatched (i.e., are not nucleobase complementary to the corresponding nucleotides of the target). Preferably the antisense compounds contain no more than about 15%, more preferably not more than about 10%, most preferably not more than 5% or no mismatches. The remaining nucleotides are nucleobase complementary or otherwise do not disrupt hybridization (e.g., universal bases). One of ordinary skill in the art would recognize the compounds provided herein are at least 80%, at least 85%, at least 90%, at least 95%, at least 96%, at least 97%, at least 98%, at least 99% or 100% complementary to a target nucleic acid.

As used herein, “hybridization” refers to the pairing of complementary oligomeric compounds (e.g., an antisense compound and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleoside or nucleotide bases (nucleobases). For example, the natural base adenine is nucleobase complementary to the natural nucleobases thymidine and uracil which pair through the formation of hydrogen bonds. The natural base guanine is nucleobase complementary to the natural bases cytosine and 5-methyl cytosine. Hybridization can occur under varying circumstances.

As used herein, “specifically hybridizes” refers to the ability of an oligomeric compound to hybridize to one nucleic acid site with greater affinity than it hybridizes to another nucleic acid site. In certain embodiments, an antisense oligonucleotide specifically hybridizes to more than one target site.

As used herein, “modulation” refers to a perturbation of amount or quality of a function or activity when compared to the function or activity prior to modulation. For example, modulation includes the change, either an increase (stimulation or induction) or a decrease (inhibition or reduction) in gene expression. As a further example, modulation of expression can include perturbing splice site selection of pre-mRNA processing, resulting in a change in the amount of a particular splice-variant present compared to conditions that were not perturbed. As a further example, modulation includes perturbing translation of a protein.

As used herein, “motif” refers to a pattern of modifications in an oligomeric compound or a region thereof. Motifs may be defined by modifications at certain nucleosides and/or at certain linking groups of an oligomeric compound.

As used herein, “nucleoside motif” refers to a pattern of nucleoside modifications in an oligomeric compound or a region thereof. The linkages of such an oligomeric compound may be modified or unmodified. Unless otherwise indicated, motifs herein describing only nucleosides are intended to be nucleoside motifs. Thus, in such instances, the linkages are not limited.

As used herein, “linkage motif” refers to a pattern of linkage modifications in an oligomeric compound or region thereof. The nucleosides of such an oligomeric compound may be modified or unmodified. Unless otherwise indicated, motifs herein describing only linkages are intended to be linkage motifs. Thus, in such instances, the nucleosides are not limited.

As used herein, “different modifications” or “differently modified” refer to modifications relative to naturally occurring molecules that are different from one another, including absence of modifications. Thus, for example, a MOE nucleoside and an unmodified DNA nucleoside are “differently modified,” even though the DNA nucleoside is unmodified. Likewise, DNA and RNA are “differently modified,” even though both are naturally-occurring unmodified nucleosides. Nucleosides that are the same but for comprising different nucleobases are not differently modified, unless otherwise indicated. For example, a nucleoside comprising a 2′-OMe modified sugar and an adenine nucleobase and a nucleoside comprising a 2′-OMe modified sugar and a thymine nucleobase are not differently modified.

As used herein, “the same modifications” refer to modifications relative to naturally occurring molecules that are the same as one another, including absence of modifications. Thus, for example, two unmodified DNA nucleoside have “the same modification,” even though the DNA nucleoside is unmodified.

As used herein, “type of modification” in reference to a nucleoside or a nucleoside of a “type” refers to the modification of a nucleoside and includes modified and unmodified nucleosides. Accordingly, unless otherwise indicated, a “nucleoside having a modification of a first type” may be an unmodified nucleoside.

As used herein, “separate regions” refers to a portion of an oligomeric compound wherein the nucleosides and internucleoside linkages within the region all comprise the same modifications; and the nucleosides and/or the internucleoside linkages of any neighboring portions include at least one different modification.

As used herein, “alternating motif” refers to an oligomeric compound or a portion thereof, having at least four separate regions of modified nucleosides in a pattern (AB)ₙAₘ where A represents a region of nucleosides having a first type of modification; B represent a region of nucleosides having a different type of modification; n is 2-15; and m is 0 or 1. Thus, in certain embodiments, alternating motifs include 4, 5, 6, 7, 8, 9, 10, 11, 12, 13, 14, 15, 16, 17, 18, 19, or 20 or more alternating regions. In certain embodiments, each A region and each B region independently comprises 1-4 nucleosides.

As used herein, “fully modified” refers to an oligomeric compound or portion thereon wherein each nucleoside is a modified nucleoside. The modifications of the nucleosides of a fully modified oligomeric compound may all be the same or one or more may be different from one another.

As used herein, “uniform modified” or “uniformly modified” refer to oligomeric compounds or portions thereof that comprise the same modifications. The nucleosides of a region of uniformly modified nucleosides all comprise the same modification.

As used herein the term “gapmer” or “gapped oligomeric compound” refers to an oligomeric compound having two external regions or wings and an internal region or gap. The three regions form a contiguous sequence of monomer subunits; with the sugar groups of the external regions being different than the sugar groups of the internal region and wherein the sugar group of each monomer subunit within a particular region is essentially the same.

As used herein, “pharmaceutically acceptable carrier or diluent” refers to any substance suitable for use in administering to an animal. In certain embodiments, a pharmaceutically acceptable carrier or diluent is sterile saline. In certain embodiments, such sterile saline is pharmaceutical grade saline.

The terms “substituent” and “substituent group,” as used herein, are meant to include groups that are typically added to other groups or parent compounds to enhance desired properties or provide other desired effects. Substituent groups can be protected or unprotected and can be added to one available site or to many available sites in a parent compound. Substituent groups may also be further substituted with other substituent groups and may be attached directly or via a linking group such as an alkyl or hydrocarbyl group to a parent compound.

Substituent groups amenable herein include without limitation, halogen, hydroxyl, alkyl, alkenyl, alkynyl, acyl (—C(O)Rₐₐ), carboxyl (—C(O)O—Rₐₐ), aliphatic groups, alicyclic groups, alkoxy, substituted oxy (—O—Rₐₐ), aryl, aralkyl, heterocyclic radical, heteroaryl, heteroarylalkyl, amino (—N(R_{bb})(R_{cc})), imino(═NR_{bb}), amido (—C(O)N(R_{bb})(R_{cc}) or —N(R_{bb})C(O)Rₐₐ), azido (—N₃), nitro (—NO₂), cyano (—CN), carbamido (—OC(O)N(R_{bb})(R_{cc}) or —N(R_{bb})C(O)ORₐₐ), ureido (—N(R_{bb})C(O)N(R_{bb})(R_{cc})), thioureido (—N(R_{bb})C(S)N(R_{bb})—(R_{cc})), guanidinyl (—N(R_{bb})C(═NR)N(R_{bb})(R_{cc})), amidinyl (—C(═NR_{bb})N(R_{bb})(R_{cc}) or —N(R_{bb})C(═NR_{bb})(Rₐₐ)), thiol (—SR_{bb}), sulfinyl (—S(O)R_{bb}), sulfonyl (—S(O)₂R_{bb}) and sulfonamidyl (—S(O)₂N(R_{bb})(R_{cc}) or —N(R_{bb})S—(O)₂R_{bb}). Wherein each Rₐₐ, R_{bb} and R_{cc} is, independently, H, an optionally linked chemical functional group or a further substituent group with a preferred list including without limitation, H, alkyl, alkenyl, alkynyl, aliphatic, alkoxy, acyl, aryl, aralkyl, heteroaryl, alicyclic, heterocyclic and heteroarylalkyl. Selected substituents within the compounds described herein are present to a recursive degree.

In this context, “recursive substituent” means that a substituent may recite another instance of itself. Because of the recursive nature of such substituents, theoretically, a large number may be present in any given claim. One of ordinary skill in the art of medicinal chemistry and organic chemistry understands that the total number of such substituents is reasonably limited by the desired properties of the compound intended. Such properties include, by way of example and not limitation, physical properties such as molecular weight, solubility or log P, application properties such as activity against the intended target and practical properties such as ease of synthesis.

Recursive substituents are an intended aspect of the invention. One of ordinary skill in the art of medicinal and organic chemistry understands the versatility of such substituents. To the degree that recursive substituents are present in a claim of the invention, the total number will be determined as set forth above.

The terms “stable compound” and “stable structure” as used herein are meant to indicate a compound that is sufficiently robust to survive isolation to a useful degree of purity from a reaction mixture, and formulation into an efficacious therapeutic agent. Only stable compounds are contemplated herein.

The term “alkyl,” as used herein, refers to a saturated straight or branched hydrocarbon radical containing up to twenty four carbon atoms. Examples of alkyl groups include without limitation, methyl, ethyl, propyl, butyl, isopropyl, n-hexyl, octyl, decyl, dodecyl and the like. Alkyl groups typically include from 1 to about 24 carbon atoms, more typically from 1 to about 12 carbon atoms (C₁-C₁₂ alkyl) with from 1 to about 6 carbon atoms being more preferred. The term “lower alkyl” as used herein includes from 1 to about 6 carbon atoms. Alkyl groups as used herein may optionally include one or more further substituent groups.

The term “alkenyl,” as used herein, refers to a straight or branched hydrocarbon chain radical containing up to twenty four carbon atoms and having at least one carbon-carbon double bond. Examples of alkenyl groups include without limitation, ethenyl, propenyl, butenyl, 1-methyl-2-buten-1-yl, dienes such as 1,3-butadiene and the like. Alkenyl groups typically include from 2 to about 24 carbon atoms, more typically from 2 to about 12 carbon atoms with from 2 to about 6 carbon atoms being more preferred. Alkenyl groups as used herein may optionally include one or more further substituent groups.

The term “alkynyl,” as used herein, refers to a straight or branched hydrocarbon radical containing up to twenty four carbon atoms and having at least one carbon-carbon triple bond. Examples of alkynyl groups include, without limitation, ethynyl, 1-propynyl, 1-butynyl, and the like. Alkynyl groups typically include from 2 to about 24 carbon atoms, more typically from 2 to about 12 carbon atoms with from 2 to about 6 carbon atoms being more preferred. Alkynyl groups as used herein may optionally include one or more further substituent groups.

The term “acyl,” as used herein, refers to a radical formed by removal of a hydroxyl group from an organic acid and has the general Formula —C(O)—X where X is typically aliphatic, alicyclic or aromatic. Examples include aliphatic carbonyls, aromatic carbonyls, aliphatic sulfonyls, aromatic sulfinyls, aliphatic sulfinyls, aromatic phosphates, aliphatic phosphates and the like. Acyl groups as used herein may optionally include further substituent groups.

The term “alicyclic” refers to a cyclic ring system wherein the ring is aliphatic. The ring system can comprise one or more rings wherein at least one ring is aliphatic. Preferred alicyclics include rings having from about 5 to about 9 carbon atoms in the ring. Alicyclic as used herein may optionally include further substituent groups.

The term “aliphatic,” as used herein, refers to a straight or branched hydrocarbon radical containing up to twenty four carbon atoms wherein the saturation between any two carbon atoms is a single, double or triple bond. An aliphatic group preferably contains from 1 to about 24 carbon atoms, more typically from 1 to about 12 carbon atoms with from 1 to about 6 carbon atoms being more preferred. The straight or branched chain of an aliphatic group may be interrupted with one or more heteroatoms that include nitrogen, oxygen, sulfur and phosphorus. Such aliphatic groups interrupted by heteroatoms include without limitation, polyalkoxys, such as polyalkylene glycols, polyamines, and polyimines. Aliphatic groups as used herein may optionally include further substituent groups.

The term “alkoxy,” as used herein, refers to a radical formed between an alkyl group andian oxygen atom wherein the oxygen atom is used to attach the alkoxy group to a parent molecule. Examples of alkoxy groups include without limitation, methoxy, ethoxy, propoxy, isopropoxy, n-butoxy, sec-butoxy, tert-butoxy, n-pentoxy, neopentoxy, n-hexoxy and the like. Alkoxy groups as used herein may optionally include further substituent groups.

The term “aminoalkyl” as used herein, refers to an amino substituted C₁-C₁₂ alkyl radical. The alkyl portion of the radical forms a covalent bond with a parent molecule. The amino group can be located at any position and the aminoalkyl group can be substituted with a further substituent group at the alkyl and/or amino portions.

The terms “aralkyl” and “arylalkyl,” as used herein, refer to an aromatic group that is covalently linked to a C₁-C₁₂ alkyl radical. The alkyl radical portion of the resulting aralkyl (or arylalkyl) group forms a covalent bond with a parent molecule. Examples include without limitation, benzyl, phenethyl and the like. Aralkyl groups as used herein may optionally include further substituent groups attached to the alkyl, the aryl or both groups that form the radical group.

The terms “aryl” and “aromatic,” as used herein, refer to a mono- or polycyclic carbocyclic ring system radicals having one or more aromatic rings. Examples of aryl groups include without limitation, phenyl, naphthyl, tetrahydronaphthyl, indanyl, idenyl and the like. Preferred aryl ring systems have from about 5 to about 20 carbon atoms in one or more rings. Aryl groups as used herein may optionally include further substituent groups.

The terms “halo” and “halogen,” as used herein, refer to an atom selected from fluorine, chlorine, bromine and iodine.

The terms “heteroaryl,” and “heteroaromatic,” as used herein, refer to a radical comprising a mono- or poly-cyclic aromatic ring, ring system or fused ring system wherein at least one of the rings is aromatic and includes one or more heteroatoms. Heteroaryl is also meant to include fused ring systems including systems where one or more of the fused rings contain no heteroatoms. Heteroaryl groups typically include one ring atom selected from sulfur, nitrogen or oxygen. Examples of heteroaryl groups include without limitation, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl and the like. Heteroaryl radicals can be attached to a parent molecule directly or through a linking moiety such as an aliphatic group or hetero atom. Heteroaryl groups as used herein may optionally include further substituent groups.

The term “heteroarylalkyl,” as used herein, refers to a heteroaryl group as previously defined that further includes a covalently attached C₁-C₁₂ alkyl radical. The alkyl radical portion of the resulting heteroarylalkyl group is capable of forming a covalent bond with a parent molecule. Examples include without limitation, pyridinylmethyl, pyrimidinylethyl, napthyridinylpropyl and the like. Heteroarylalkyl groups as used herein may optionally include further substituent groups on one or both of the heteroaryl or alkyl portions.

The term “heterocyclic radical” as used herein, refers to a radical mono-, or poly-cyclic ring system that includes at least one heteroatom and is unsaturated, partially saturated or fully saturated, thereby including heteroaryl groups. Heterocyclic is also meant to include fused ring systems wherein one or more of the fused rings contain at least one heteroatom and the other rings can contain one or more heteroatoms or optionally contain no heteroatoms. A heterocyclic radical typically includes at least one atom selected from sulfur, nitrogen or oxygen. Examples of heterocyclic radicals include, [1,3]dioxolanyl, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, tetrahydrofuryl and the like. Heterocyclic groups as used herein may optionally include further substituent groups.

The term “hydrocarbyl” includes radical groups that comprise C, O and H. Included are straight, branched and cyclic groups having any degree of saturation. Such hydrocarbyl groups can include one or more heteroatoms selected from N, O and S and can be further mono or poly substituted with one or more substituent groups.

The term “mono or poly cyclic structure” as used herein includes all ring systems selected from single or polycyclic radical ring systems wherein the rings are fused or linked and is meant to be inclusive of single and mixed ring systems individually selected from aliphatic, alicyclic, aryl, heteroaryl, aralkyl, arylalkyl, heterocyclic, heteroaryl, heteroaromatic and heteroarylalkyl. Such mono and poly cyclic structures can contain rings that each have the same level of saturation or each, independently, have varying degrees of saturation including fully saturated, partially saturated or fully unsaturated. Each ring can comprise ring atoms selected from C, N, O and S to give rise to heterocyclic rings as well as rings comprising only C ring atoms which can be present in a mixed motif such as for example benzimidazole wherein one ring has only carbon ring atoms and the fused ring has two nitrogen atoms. The mono or poly cyclic structures can be further substituted with substituent groups such as for example phthalimide which has two ═O groups attached to one of the rings. Mono or poly cyclic structures can be attached to parent molecules using various strategies such as directly through a ring atom, through a substituent group or through a bifunctional linking moiety.

The term “oxo” refers to the group (═O).

Linking groups or bifunctional linking moieties such as those known in the art are useful for attachment of chemical functional groups, conjugate groups, reporter groups and other groups to selective sites in a parent compound such as for example an oligomeric compound. In general, a bifunctional linking moiety comprises a hydrocarbyl moiety having two functional groups. One of the functional groups is selected to bind to a parent molecule or compound of interest and the other is selected to bind to essentially any selected group such as a chemical functional group or a conjugate group. In some embodiments, the linker comprises a chain structure or a polymer of repeating units such as ethylene glycols or amino acid units. Examples of functional groups that are routinely used in bifunctional linking moieties include without limitation, electrophiles for reacting with nucleophilic groups and nucleophiles for reacting with electrophilic groups. In some embodiments, bifunctional linking moieties include amino, hydroxyl, carboxylic acid, thiol, unsaturations (e.g., double or triple bonds), and the like. Some nonlimiting examples of bifunctional linking moieties include 8-amino-3,6-dioxaoctanoic acid (ADO), succinimidyl 4-(N-maleimidomethyl)cyclohexane-1-carboxylate (SMCC) and 6-aminohexanoic acid (AHEX or AHA). Other linking groups include without limitation, substituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

The term “phosphate moiety” as used herein, refers to a terminal phosphate group that includes phosphates as well as modified phosphates. The phosphate moiety can be located at either terminus but is preferred at the 5′-terminal nucleoside. In one aspect, the terminal phosphate is unmodified having the formula —O—P(═O)(OH)OH. In another aspect, the terminal phosphate is modified such that one or more of the O and OH groups are replaced with H, O, S, N(R) or alkyl where R is H, an amino protecting group or unsubstituted or substituted alkyl. In certain embodiments, the 5′ and or 3′ terminal group can comprise from 1 to 3 phosphate moieties that are each, independently, unmodified (di or tri-phosphates) or modified.

As used herein, the term “phosphorus moiety” refers to a group having the formula:

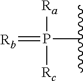

wherein:

Rₐ and R_{c} are each, independently, OH, SH, C₁-C₆ alkyl, substituted C₁-C₆ alkyl, C₁-C₆ alkoxy, substituted C₁-C₆ alkoxy, amino or substituted amino; and

R_{c} is O or S.

Phosphorus moieties included herein can be attached to a monomer, which can be used in the preparation of oligomeric compounds, wherein the monomer may be attached using O, S, NR_{d} or CRₑR_{f}, wherein R_{d} includes without limitation H, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl, substituted C₂-C₆ alkynyl or substituted acyl, and Rₑ and R_{f} each, independently, include without limitation H, halogen, C₁-C₆ alkyl, substituted C₁-C₆ alkyl, C₁-C₆ alkoxy or substituted C₁-C₆ alkoxy. Such linked phosphorus moieties include without limitation, phosphates, modified phosphates, thiophosphates, modified thiophosphates, phosphonates, modified phosphonates, phosphoramidates and modified phosphoramidates.

As used herein, “phosphate stabilizing modification” refers to a nucleoside modification that results in stabilization of a 5′-phosphate group of nucleoside, relative to the stability of a 5′-phosphate of an unmodified nucleoside under biologic conditions. Such stabilization of a 5′-phosphate group includes but is not limit to resistance to removal by phosphatases.

As used herein, “phosphate stabilizing nucleoside” refers to a nucleoside comprising at least one phosphate stabilizing modification. In certain embodiments the phosphate stabilizing modification is a 2′-modification. In certain embodiments, the phosphate stabilizing modification is at the 5′ position of the nucleoside. In certain embodiments, a phosphate stabilizing modification is at the 5′ position of the nucleoside and at the 2′ position of the nucleoside.

As used herein, “5′-stabilizing nucleoside” refers to a nucleoside that, when placed at the 5′-end of an oligonucleotide, results in an oligonucleotide that is more resistant to exonuclease digestion, and/or has a stabilized phosphate group.

The term “protecting group,” as used herein, refers to a labile chemical moiety which is known in the art to protect reactive groups including without limitation, hydroxyl, amino and thiol groups, against undesired reactions during synthetic procedures. Protecting groups are typically used selectively and/or orthogonally to protect sites during reactions at other reactive sites and can then be removed to leave the unprotected group as is or available for further reactions. Protecting groups as known in the art are described generally in Greene's Protective Groups in Organic Synthesis, 4th edition, John Wiley & Sons, New York, 2007.

Groups can be selectively incorporated into oligomeric compounds as provided herein as precursors. For example an amino group can be placed into a compound as provided herein as an azido group that can be chemically converted to the amino group at a desired point in the synthesis. Generally, groups are protected or present as precursors that will be inert to reactions that modify other areas of the parent molecule for conversion into their final groups at an appropriate time. Further representative protecting or precursor groups are discussed in Agrawal et al., Protocols for Oligonucleotide Conjugates, Humana Press; New Jersey, 1994, 26, 1-72.

The term “orthogonally protected” refers to functional groups which are protected with different classes of protecting groups, wherein each class of protecting group can be removed in any order and in the presence of all other classes (see, Barany et al., J. Am. Chem. Soc., 1977, 99, 7363-7365; Barany et al., J. Am. Chem. Soc., 1980, 102, 3084-3095). Orthogonal protection is widely used in for example automated oligonucleotide synthesis. A functional group is deblocked in the presence of one or more other protected functional groups which is not affected by the deblocking procedure. This deblocked functional group is reacted in some manner and at some point a further orthogonal protecting group is removed under a different set of reaction conditions. This allows for selective chemistry to arrive at a desired compound or oligomeric compound.

Examples of hydroxyl protecting groups include without limitation, acetyl, t-butyl, t-butoxymethyl, methoxymethyl, tetrahydropyranyl, 1-ethoxyethyl, 1-(2-chloroethoxy)ethyl, p-chlorophenyl, 2,4-dinitrophenyl, benzyl, 2,6-dichlorobenzyl, diphenylmethyl, p-nitrobenzyl, bis(2-acetoxyethoxy)methyl (ACE), 2-trimethylsilylethyl, trimethylsilyl, triethylsilyl, t-butyldimethylsilyl, t-butyldiphenylsilyl, triphenylsilyl, [(triisopropylsilyl)oxy]methyl (TOM), benzoylformate, chloroacetyl, trichloroacetyl, trifluoro-acetyl, pivaloyl, benzoyl, p-phenylbenzoyl, 9-fluorenylmethyl carbonate, mesylate, tosylate, triphenylmethyl (trityl), monomethoxytrityl, dimethoxytrityl (DMT), trimethoxytrityl, 1(2-fluorophenyl)-4-methoxypiperidin-4-yl (FPMP), 9-phenylxanthine-9-yl (Pixyl) and 9-(p-methoxyphenyl)xanthine-9-yl (MOX). Wherein more commonly used hydroxyl protecting groups include without limitation, benzyl, 2,6-dichlorobenzyl, t-butyldimethylsilyl, t-butyldiphenylsilyl, benzoyl, mesylate, tosylate, dimethoxytrityl (DMT), 9-phenylxanthine-9-yl (Pixyl) and 9-(p-methoxyphenyl)xanthine-9-yl (MOX).

Examples of protecting groups commonly used to protect phosphate and phosphorus hydroxyl groups include without limitation, methyl, ethyl, benzyl (Bn), phenyl, isopropyl, tert-butyl, allyl, cyclohexyl (cHex), 4-methoxybenzyl, 4-chlorobenzyl, 4-nitrobenzyl, 4-acyloxybenzyl, 2-methylphenyl, 2,6-dimethylphenyl, 2-chlorophenyl, diphenylmethyl, 4-methylthio-1-butyl, 2-(S-Acetylthio)ethyl (SATE), 2-cyanoethyl, 2-cyano-1,1-dimethylethyl (CDM), 4-cyano-2-butenyl, 2-(trimethylsilyl)ethyl (TSE), 2-(phenylthio)ethyl, 2-(triphenylsilyl)ethyl, 2-(benzylsulfonyl)ethyl, 2,2,2-trichloroethyl, 2,2,2-tribromoethyl, 2,3-dibromopropyl, 2,2,2-trifluoroethyl, thiophenyl, 2-chloro-4-tritylphenyl, 2-bromophenyl, 2-[N-isopropyl-N-(4-methoxybenzoyl)amino]ethyl, 4-(N-trifluoroacetylamino)butyl, 4-oxopentyl, 4-tritylaminophenyl, 4-benzylaminophenyl and morpholino. Wherein more commonly used phosphate and phosphorus protecting groups include without limitation, methyl, ethyl, benzyl (Bn), phenyl, isopropyl, tert-butyl, 4-methoxybenzyl, 4-chlorobenzyl, 2-chlorophenyl and 2-cyanoethyl.

Examples of amino protecting groups include without limitation, carbamate-protecting groups, such as 2-trimethylsilylethoxycarbonyl (Teoc), 1-methyl-1-(4-biphenylyl)ethoxycarbonyl (Bpoc), t-butoxycarbonyl (BOC), allyloxycarbonyl (Alloc), 9-fluorenylmethyloxycarbonyl (Fmoc), and benzyl-oxycarbonyl (Cbz); amide-protecting groups, such as formyl, acetyl, trihaloacetyl, benzoyl, and nitrophenylacetyl; sulfonamide-protecting groups, such as 2-nitrobenzenesulfonyl; and imine- and cyclic imide-protecting groups, such as phthalimido and dithiasuccinoyl.

Examples of thiol protecting groups include without limitation, triphenylmethyl (trityl), benzyl (Bn), and the like.

In certain embodiments, oligomeric compounds as provided herein can be prepared having one or more optionally protected phosphorus containing internucleoside linkages. Representative protecting groups for phosphorus containing internucleoside linkages such as phosphodiester and phosphorothioate linkages include β-cyanoethyl, diphenylsilylethyl, δ-cyanobutenyl, cyano p-xylyl (CPX), N-methyl-N-trifluoroacetyl ethyl (META), acetoxy phenoxy ethyl (APE) and butene-4-yl groups. See for example U.S. Pat. No. 4,725,677 and Re. 34,069 (β-cyanoethyl); Beaucage et al., Tetrahedron, 1993, 49(10), 1925-1963; Beaucage et al., Tetrahedron, 1993, 49(46), 10441-10488; Beaucage et al., Tetrahedron, 1992, 48(12), 2223-2311.

In certain embodiments, compounds having reactive phosphorus groups are provided that are useful for forming internucleoside linkages including for example phosphodiester and phosphorothioate internucleoside linkages. Such reactive phosphorus groups are known in the art and contain phosphorus atoms in P^{III} or P^{V} valence state including, but not limited to, phosphoramidite, H-phosphonate, phosphate triesters and phosphorus containing chiral auxiliaries. In certain embodiments, reactive phosphorus groups are selected from diisopropylcyanoethoxy phosphoramidite (—O*—P[N[(CH(CH₃)₂]₂]O(CH₂)₂CN) and H-phosphonate (—O*—P(═O)(H)OH), wherein the O* is provided from the Markush group for the monomer. A preferred synthetic solid phase synthesis utilizes phosphoramidites (P^{III} chemistry) as reactive phosphites. The intermediate phosphite compounds are subsequently oxidized to the phosphate or thiophosphate (P^{V} chemistry) using known methods to yield, phosphodiester or phosphorothioate internucleoside linkages. Additional reactive phosphates and phosphites are disclosed in Tetrahedron Report Number 309 (Beaucage and Iyer, Tetrahedron, 1992, 48, 2223-2311).

### Certain Oligomeric Compounds

In certain embodiments, the invention provides compositions comprising a liped molecule and an oligomeric compound comprising a 5′ modified nucleoside having Formula I:

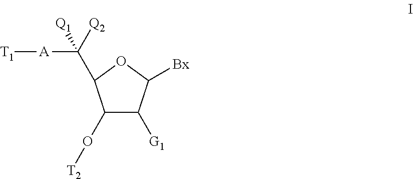

wherein:

Bx is a heterocyclic base moiety;

A is O, S or N(R₁);

R₁ is H, C₁-C₆ alkyl or substituted C₁-C₆ alkyl;

T₁ is a phosphorus moiety;

T₂ is an internucleoside linking group linking the monomer of Formula I to the remainder of the oligomeric compound;

each of Q₁ and Q₂ is independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;

G₁ is halogen, X₁—V, or O—X₂;

X_{I} is O, S or CR₂R₃;

each R₂ and R₃ is, independently, H or C₁-C₆ alkyl;
V is a conjugate group, aryl, (CH₂)₂[O(CH₂)₂]ₜOCH₃, where t is from 1-3, (CH₂)₂F, CH₂COOH, CH₂CONH₂, CH₂CONR₅R₆, CH₂COOCH₂CH₃, CH₂CONH(CH₂)ᵢ—S—R₄ where i is from 1 to 10, CH₂CONH(CH₂)ₖ₃NR₅R₆ where k₃ is from 1 to 6, CH₂CONH[(CH₂)ₖ₁—N(H)]ₖ₂—(CH₂)ₖ₁NH₂ where each k₁ is independently from 2 to 4 and k₂ is from 2 to 10;
R₄ is H, C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, C₆-C₁₄ aryl or a thio protecting group;
R₅ and R₆ are each, independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;
X₂ is [C(R₇)(R₈)]ₙ—[(C═O)ₘX]ⱼ—Z;
each R₇ and R₈ is independently, H, halogen, C₁-C₆ alkyl or substituted C₁-C₆ alkyl;

### X is O, S, or N(E₁);

Z is H, halogen, C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl or N(E₂)(E₃);
E₁, E₂, and E₃ are each independently H, C₁-C₆ alkyl, or substituted C₁-C₆ alkyl;
n is from 1 to about 6;
m is 0 or 1;
j is 0 or 1;

each substituted group comprises one or more optionally protected substituent groups independently selected from H, halogen, OJ₁, N(J₁)(J₂), ═NJ_{I}, SJ_{I}, N₃, CN, OC(=L)J₁, OC(=L)N(J₁)(J₂), C(═ON(J₁)(J₂), C(=L)N(H)—(CH₂)₂N(J₁)(J₂) or a mono or polycyclic ring system;

L is O, S or NJ₃;

each J₁, J₂ and J₃ is, independently, H or C₁-C₆ alkyl;

when j is 1 then Z is other than halogen or N(E₂)(E₃);

and a lipid particle.

In certain embodiments, the invention provides compositions comprising a lipid particle and an oligomeric compound wherein the oligomeric compound comprises an oligonucleotide comprising a nucleoside having Formula II:

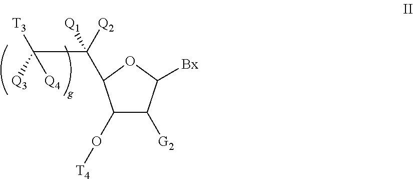

wherein:

Bx is a heterocyclic base moiety;

T₃ is a phosphorus moiety;

T₄ is an internucleoside linking group linking the monomer of Formula II to the remainder of the oligomeric compound;

Q₁, Q₂, Q₃ and Q₄ are each, independently, H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl, hydroxyl, substituted oxy, O—C₁-C₆ alkyl, substituted O—C₁-C₆ alkyl, S—C₁-C₆ alkyl, substituted S—C₁-C₆ alkyl, N(R₁)—C₁-C₆ alkyl or substituted N(R₁)—C₁-C₆ alkyl

R₁ is H, C₁-C₆ alkyl or substituted C₁-C₆ alkyl;

G₂ is H, OH, halogen, O-aryl or O—-[C(R₄)(R₅)]ₙ—[(C═O)ₘ—X]ⱼ—Z;

each R₄ and R₅ is, independently, H, halogen, C₁-C₆ alkyl or substituted C₁-C₆ alkyl;

X is O, S or N(E₁);

Z is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);

E₁, E₂ and E₃ are each, independently, H, C₁-C₆ alkyl or substituted C₁-C₆ alkyl;

n is from 1 to about 6;

m is 0 or 1;

j is 0 or 1;

g is 0 or 1;

each substituted group comprises one or more optionally protected substituent groups independently selected from H, halogen, OJ₁, N(J₁)(J₂), ═NJ₁, SJ₁, N₃, CN, OC(=L)J₁, OC(=L)N(J₁)(J₂), C(═ON(J₁)(J₂), C(=L)N(H)—(CH₂)₂N(h)(J₂), a mono or poly cyclic ring system, a phosphate group or a phosphorus moiety;

L is O, S or NJ₃;

each J₁, J₂ and J₃ is, independently, H or C₁-C₆ alkyl;

when j is 1 then Z is other than halogen or N(E₂)(E₃); and

when Q₁, Q₂, Q₃ and Q₄ are each H or when Q₁ and Q₂ are H and Q₃ and Q₄ are each F or when Q₁ and Q₂ are each H and one of Q₃ and Q₄ is H and the other of Q₃ and Q₄ is R₉ then G₂ is other than H, hydroxyl, OR₉, halogen, CF₃, CCl₃, CHCl₂ or CH₂OH wherein R₉ is alkyl, alkenyl, alkynyl, aryl or alkaryl; and a lipid particle.

A. Modified Sugar and Phosphorous Moieties

In certain embodiments the invention provides compositions comprising an oligomeric compounds wherein the 5′-terminal nucleoside comprises a modified phosphate or phosphorus moiety at the 5′-end. In certain embodiments, the invention provides compositions comprising oligomeric compounds comprising nucleosides comprising a modification at the 5′-position of the sugar. Herein, modifications at the 5′-position of the sugar or its substituents are typically referred to as modified sugars and modifications distal to that position are referred to as modified phosphates. One of skill in the art will appreciate that the boundary between these terms, particularly once modifications are introduced, becomes arbitrary. The example below shows a modified nucleoside comprising a sulfur atom in place of the oxygen that links the phosphorus moiety and the sugar of a natural nucleoside. Herein, such modifications are typically referred to as modified phosphates, however, one of skill the art will recognize that such a modification could also be referred to as a modified sugar comprising a sulfer linked to the 5′-position of the sugar.

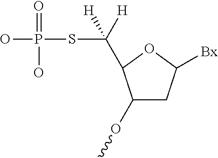

In certain embodiments, compostions of the present invention comprise oligomeric compounds comprising nucleosides having modified phosphates. In certain embodiments, comprise 5′-sugar modifications. In certain embodiments, nucleosides comprise both modified phosphates and 5′-sugar modifications. Examples of nucleosides having such modified phosphorus moieties and/or 5′-modifications include, but are not limited to:

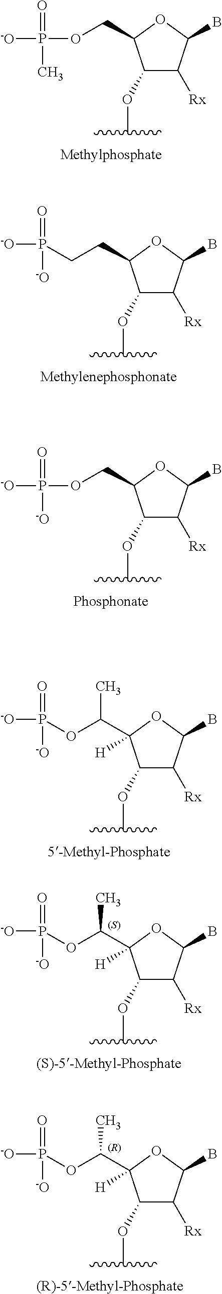

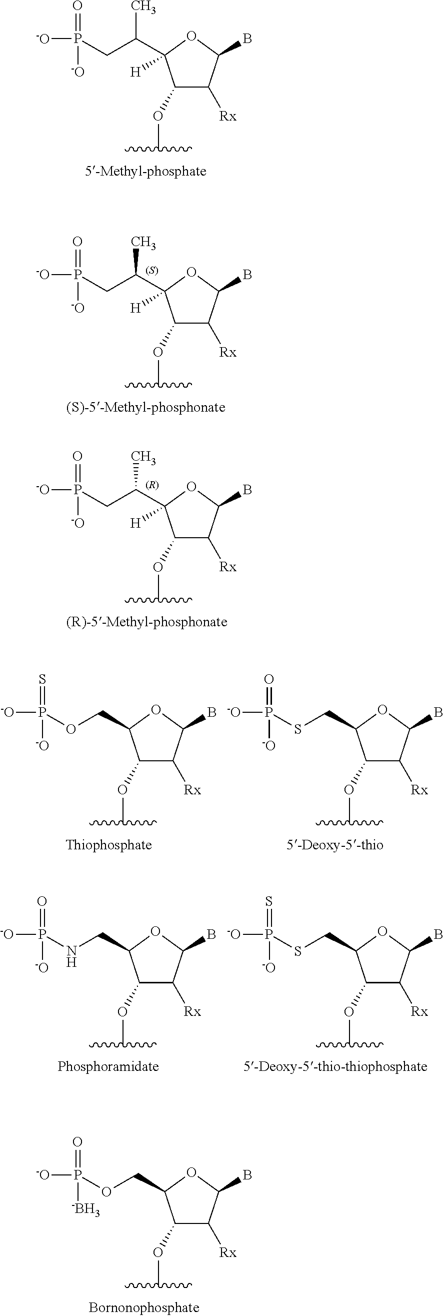

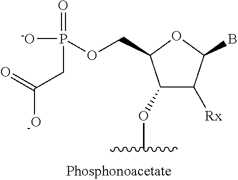

The above examples are intended to illustrate and not to limit the invention as regards modifications at the 5′-phosphate and the 5′-position of the sugar. In the above illustrative examples, the 2′-position of the sugar is labeled Rx. However, in certain embodiments, nucleosides comprising modified phosphate and/or 5′-modified sugar groups may further comprise a modification at the 2′-position of the sugar. Many such 2′-modifications are known in the art. In certain embodiments, Rx in any of the above examples may be selected from: a halogen (including, but not limited to F), allyl, amino, azido, thio, O-allyl, —O—C₁-C₁₀ alkyl, —O—C₁-C₁₀ substituted alkyl, —OCF₃, —O—(CH₂)₂—O—CH₃, —O(CH₂)₂SCH₃, —O—(CH₂)₂—O—N(Rₘ)(Rₙ), —O—CH2-C(═O)—N(Rₘ)(Rₙ), where each Rₘ and Rₙ is, independently, H or substituted or unsubstituted C₁-C₁₀ alkyl, —O[(CH₂)ₙO]ₘCH₃, —O(CH₂)ₙNH₂, —O(CH₂)ₙCH₃, —O(CH₂)ₙONH₂, —OCH₂C(═O)N(H)CH₃, —O(CH₂)ₙON[(CH₂)ₙCH₃]₂, where n and m are from 1 to about 10; C₁ to C₁₀ alkyl, substituted alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl. In certain embodiments, Rx is selected from: —O-Methyl, —O-Ethyl, —O-Propyl, —O-Phenyl, O-methoxyethyl, S-Methyl, NMA, DMAEAc, DMAEOE, —O—CH₂CH₂F. In certain embodiments, Rx is any substituents described herein or known in the art. In certain embodiments, the nucleoside is not modified at the 2′-position (i.e., Rx is H (DNA) or Rx is OH(RNA)). In certain embodiments, such nucleosides are at the 5′ end of an oligonucleotide.

In certain embodiments, nucleosides incorporated in oligomeric compounds include, but are not limited to any of the following:

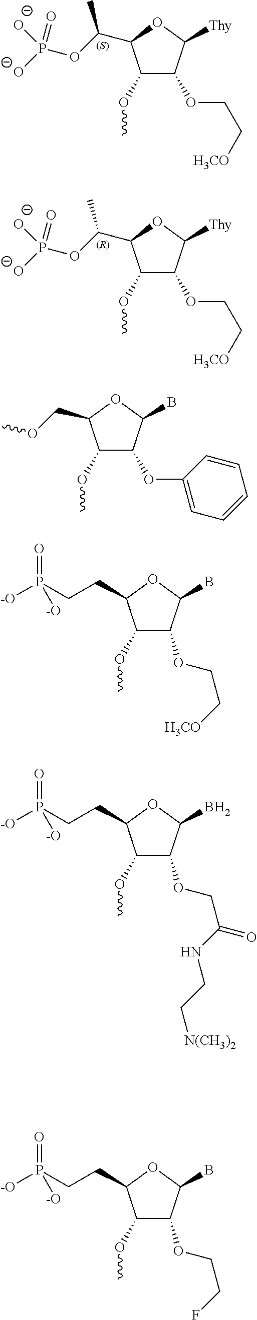

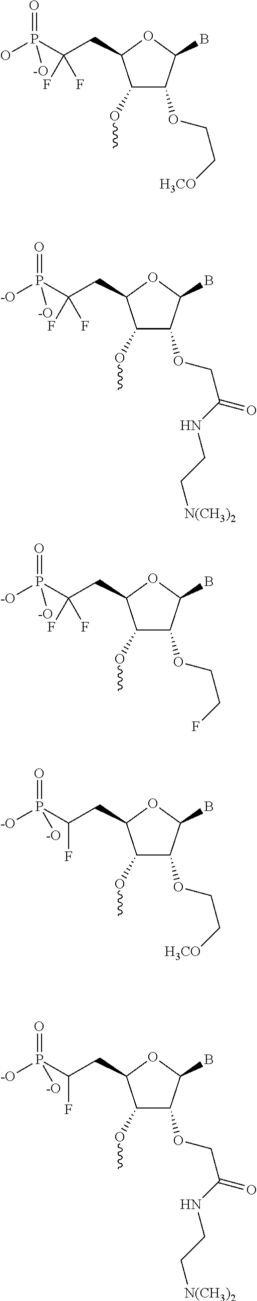

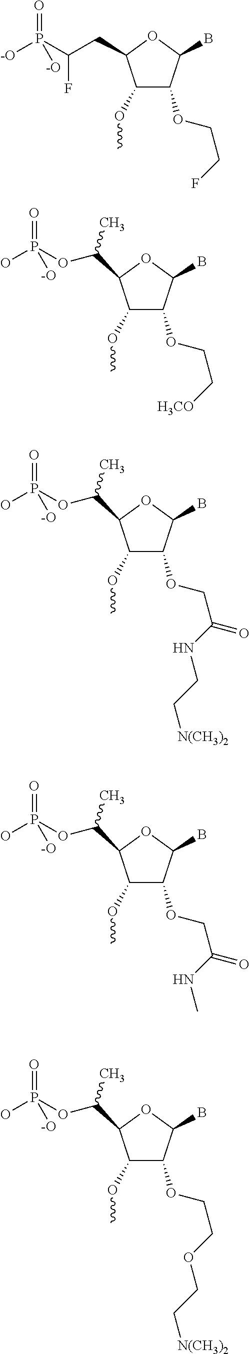

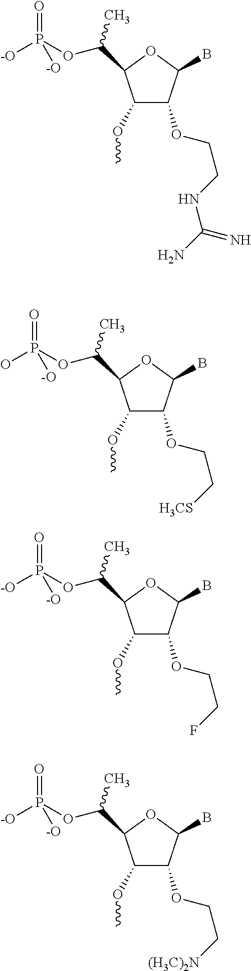

In certain embodiments, such nucleosides are incorporated into oligomeric compounds, which are paired with a lipid particle to form a composition. In certain embodiments, such nucleosides are incorporated at the 5′-terminal end of an oligonucleotide or oligomeric compound.

In certain embodiments, oligomeric compounds comprise a nucleoside of Formula I or II or a di-nucleoside of Formula III. In certain such embodiments, the remainder of the oligomeric compound comprises one or more modifications. Such modifications may include modified sugar moieties, modified nucleobases and/or modified internucleoside linkages. Certain such modifications which may be incorporated in an oligomeric compound comprising a nucleoside of Formula I or II or a di-nucleoside of Formula III is at the 5′-terminus are known in the art.

Certain Modified Sugar Moieties

Oligomeric compounds for use in the compositions of the invention can optionally contain one or more nucleosides wherein the sugar group has been modified. Such sugar modified nucleosides may impart enhanced nuclease stability, increased binding affinity, or some other beneficial biological property to the antisense compounds. In certain embodiments, nucleosides comprise a chemically modified ribofuranose ring moiety. Examples of chemically modified ribofuranose rings include, without limitation, addition of substitutent groups (including 5′ and/or 2′ substituent groups; bridging of two ring atoms to form bicyclic nucleic acids (BNA); replacement of the ribosyl ring oxygen atom with S, N(R), or C(R1)(R)2 (R═H, C₁-C₁₂ alkyl or a protecting group); and combinations thereof. Examples of chemically modified sugars include, 2′-F-5′-methyl substituted nucleoside (see, PCT International Application WO 2008/101157, published on Aug. 21, 2008 for other disclosed 5′,2′-bis substituted nucleosides), replacement of the ribosyl ring oxygen atom with S with further substitution at the 2′-position (see, published U.S. Patent Application US2005/0130923, published on Jun. 16, 2005), or, alternatively, 5′-substitution of a BNA (see, PCT International Application WO 2007/134181, published on Nov. 22, 2007, wherein LNA is substituted with, for example, a 5′-methyl or a 5′-vinyl group).

Examples of nucleosides having modified sugar moieties include, without limitation, nucleosides comprising 5′-vinyl, 5′-methyl (R or S), 4′-S, 2′-F, 2′-OCH₃, and 2′-O(CH₂)2OCH₃ substituent groups. The substituent at the 2′ position can also be selected from allyl, amino, azido, thio, O-allyl, O—C₁-C₁₀ alkyl, OCF₃, O(CH₂)2SCH₃, O(CH₂)2-O—N(Rm)(Rn), and O—CH₂—C(═O)—N(Rm)(Rn), where each Rm and Rn is, independently, H or substituted or unsubstituted C1-C10 alkyl.

In certain embodiments, oligomeric compounds for use in the compositions of the present invention include one or more bicyclic nucleoside. In certain such embodiments, the bicyclic nucleoside comprises a bridge between the 4′ and the 2′ ribosyl ring atoms. In certain embodiments, oligomeric compounds provided herein include one or more bicyclic nucleosides wherein the bridge comprises a 4′ to 2′ bicyclic nucleoside. Examples of such 4′ to 2′ bicyclic nucleosides, include, but are not limited to, one of the formulae: 4′-(CH₂)—O-2′ (LNA); 4′-(CH₂)—S-2; 4′-(CH₂)₂—O-2′ (ENA); 4′-CH(CH₃)—O-2′ and 4′-CH(CH₂OCH₃)—O-2′, and analogs thereof (see, U.S. Pat. No. 7,399,845, issued on Jul. 15, 2008); 4′-C(CH₃)(CH₃)—O-2′ and analogs thereof, (see, published International Application WO2009/006478, published Jan. 8, 2009); 4′-CH₂—N(OCH₃)-2′ and analogs thereof (see, published PCT International Application WO2008/150729, published Dec. 11, 2008); 4′-CH₂—O—N(CH₃)-2′ (see published U.S. Patent Application US2004/0171570, published Sep. 2, 2004); 4′-CH₂—N(R)—O-2′, wherein R is H, C₁-C₁₂ alkyl, or a protecting group (see, U.S. Pat. No. 7,427,672, issued on Sep. 23, 2008); 4′-CH₂—C(H)(CH₃)-2′ (see Chattopadhyaya, et al., J. Org. Chem., 2009, 74, 118-134); and 4′-CH₂—C(═CH₂)-2′ and analogs thereof (see, published PCT International Application WO 2008/154401, published on Dec. 8, 2008). Also see, for example: Singh et al., Chem. Commun., 1998, 4, 455-456; Koshkin et al., Tetrahedron, 1998, 54, 3607-3630; Wahlestedt et al., Proc. Natl. Acad. Sci. U.S. A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 129(26) 8362-8379 (Jul. 4, 2007); Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; U.S. Pat. Nos. 7,053,207, 6,268,490, 6,770,748, 6,794,499, 7,034,133, 6,525,191, 6,670,461, and 7,399,845; International applications WO 2004/106356, WO 1994/14226, WO 2005/021570, and WO 2007/134181; U.S. Patent Publication Nos. US2004/0171570, US2007/0287831, and US2008/0039618; U.S. patent Ser. Nos. 12/129,154, 60/989,574, 61/026,995, 61/026,998, 61/056,564, 61/086,231, 61/097,787, and 61/099,844; and PCT International Applications Nos. PCT/US2008/064591, PCT/US2008/066154, and PCT/US2008/068922. Each of the foregoing bicyclic nucleosides can be prepared having one or more stereochemical sugar configurations including for example α-L-ribofuranose and β-D-ribofuranose (see PCT international application PCT/DK98/00393, published on Mar. 25, 1999 as WO 99/14226).

In certain embodiments, bicyclic sugar moieties of BNA nucleosides include, but are not limited to, compounds having at least one bridge between the 4′ and the 2′ position of the pentofuranosyl sugar moiety wherein such bridges independently comprises 1 or from 2 to 4 linked groups independently selected from —[C(Rₐ)(R_{b})]ₙ—, —C(Rₐ)═C(R_{b})—, —C(Rₐ)═N—, —C(═NRₐ)—, —C(═O)—, —C(═S)—, —O—, —Si(Rₐ)₂—, —S(═O)_{X}—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

In certain embodiments, the bridge of a bicyclic sugar moiety is, —[C(Rₐ)(R_{b})]ₙ—, —[C(Rₐ)(R_{b})]ₙ—O—, —C(RₐR_{b})—N(R)—O— or, —C(RₐR_{b})—O—N(R)—. In certain embodiments, the bridge is 4′-CH₂-2′,4′-(CH₂)₂-2′,4′-(CH₂)₃-2′,4′-CH₂—O-2′,4′-(CH₂)₂—O-2′,4′-CH₂—O—N(R)-2′, and 4′-CH₂—N(R)—O-2′-, wherein each R is, independently, H, a protecting group, or C₁-C₁₂ alkyl.

In certain embodiments, bicyclic nucleosides are further defined by isomeric configuration. For example, a nucleoside comprising a 4′-2′ methylene-oxy bridge, may be in the α-L configuration or in the β-D configuration. Previously, α-L-methyleneoxy (4′-CH₂—O-2′) BNA's have been incorporated into antisense oligonucleotides that showed antisense activity (Frieden et al., Nucleic Acids Research, 2003, 21, 6365-6372).

In certain embodiments, bicyclic nucleosides include, but are not limited to, (A) α-L-Methyleneoxy (4′-CH₂—O-2′) BNA, (B) β-D-Methyleneoxy (4′-CH₂—O-2′) BNA, (C) Ethyleneoxy (4′-(CH₂)₂—O-2′) BNA, (D) Aminooxy (4′-CH₂—O—N(R)-2′) BNA, (E) Oxyamino (4′-CH₂—N(R)—O-2′) BNA, (F) Methyl(methyleneoxy) (4′-CH(CH₃)—O-2′) BNA (also referred to as constrained ethyl or cEt), (G) methylene-thio (4′-CH₂—S-2′) BNA, (H) methylene-amino (4′-CH2-N(R)-2′) BNA, (I) methyl carbocyclic (4′-CH₂—CH(CH₃)-2′) BNA, and (J) propylene carbocyclic (4′-(CH₂)₃-2′) BNA as depicted below.

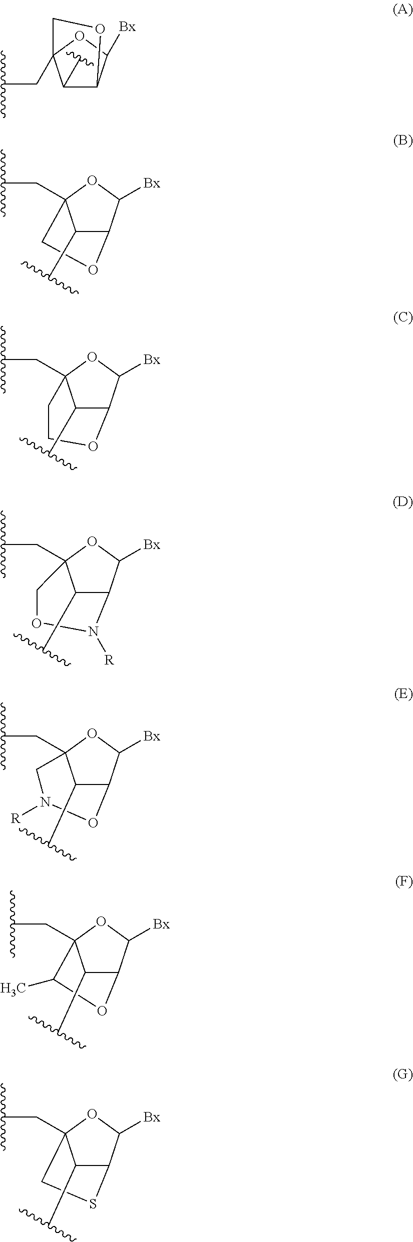

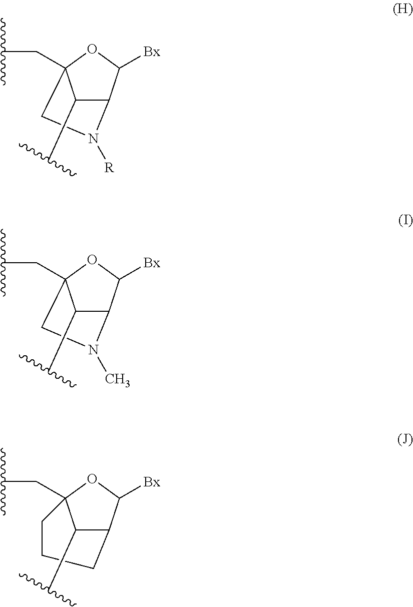

wherein Bx is the base moiety and R is, independently, H, a protecting group, or C₁-C₁₂ alkyl.

In certain embodiments, bicyclic nucleoside having Formula I:

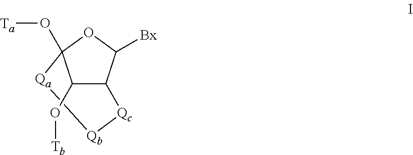

wherein:

Bx is a heterocyclic base moiety;

-Qₐ-Q_{b}-Q_{c}- is —CH₂—N(R_{c}—CH₂—, —C(═O)—N(R_{c})—CH₂—, —CH₂—N(R_{c})—O—, or —N(R_{c})—O—CH₂;

R_{c} is C₁-C₁₂ alkyl or an amino protecting group; and

Tₐ and T_{b} are each, independently, H, a hydroxyl protecting group, a conjugate group, a reactive phosphorus group, a phosphorus moiety, or a covalent attachment to a support medium.

In certain embodiments, bicyclic nucleoside having Formula II:

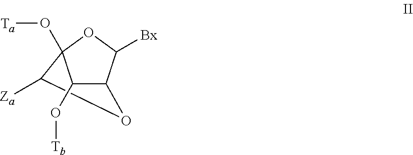

wherein:

Bx is a heterocyclic base moiety;

Tₐ and T_{b} are each, independently, H, a hydroxyl protecting group, a conjugate group, a reactive phosphorus group, a phosphorus moiety, or a covalent attachment to a support medium;

Zₐ is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, substituted amide, thiol, or substituted thio.

In certain embodiments, each of the substituted groups is, independently, mono or poly substituted with substituent groups independently selected from halogen, oxo, hydroxyl, OJ_{c}, NJ_{c}J_{d}, SJ_{c}, N₃, OC(═X)J_{c}, and NJₑC(═X)NJ_{c}J_{d}, wherein each J_{c}, J_{d}, and Jₑ is, independently, H, C₁-C₆ alkyl, or substituted C₁-C₆ alkyl and X is O or NJ_{c}.

In certain embodiments, bicyclic nucleoside having Formula III:

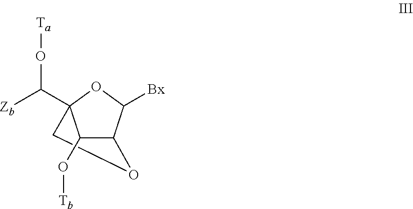

wherein:

Bx is a heterocyclic base moiety;

Tₐ and T_{b} are each, independently, H, a hydroxyl protecting group, a conjugate group, a reactive phosphorus group, a phosphorus moiety, or a covalent attachment to a support medium;

Z_{b} is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, or substituted acyl

In certain embodiments, bicyclic nucleoside having Formula IV:

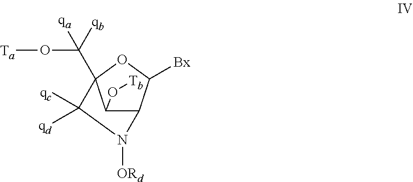

wherein:

Bx is a heterocyclic base moiety;

Tₐ and T_{b} are each, independently H, a hydroxyl protecting group, a conjugate group, a reactive phosphorus group, a phosphorus moiety, or a covalent attachment to a support medium;

R_{d} is C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl;

each qₐ, q_{b}, q_{c} and q_{d} is, independently, H, halogen, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl, C₁-C₆ alkoxyl, substituted C₁-C₆ alkoxyl, acyl, substituted acyl, C₁-C₆ aminoalkyl, or substituted C₁-C₆ aminoalkyl;

In certain embodiments, bicyclic nucleoside having Formula V:

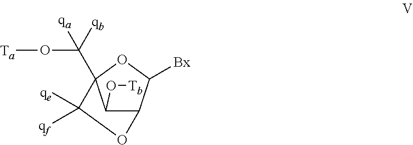

wherein:

Bx is a heterocyclic base moiety;

Tₐ and T_{b} are each, independently, H, a hydroxyl protecting group, a conjugate group, a reactive phosphorus group, a phosphorus moiety, or a covalent attachment to a support medium;

qₐ, q_{b}, qₑ and q_{f} are each, independently, hydrogen, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJⱼ, SJᵢ, SOJⱼ, NJⱼJₖ, N₃, CN, C(═O)OJⱼ, C(═O)NJⱼJₖ, C(═O)Jⱼ, O—C(═O)—NJⱼJₖ, N(H)C(═NH)NJⱼJₖ, N(H)C(═O)NJⱼJₖ or N(H)C(═S)NJⱼJₖ;

or qₑ and q_{f} together are ═C(q_{g})(q_{b});

q_{g} and q₁, are each, independently, H, halogen, C₁-C₁₂ alkyl, or substituted C₁-C₁₂ alkyl.

The synthesis and preparation of the methyleneoxy (4′-CH₂—O-2′) BNA monomers adenine, cytosine, guanine, 5-methyl-cytosine, thymine, and uracil, along with their oligomerization, and nucleic acid recognition properties have been described (see, e.g., Koshkin et al., Tetrahedron, 1998, 54, 3607-3630). BNAs and preparation thereof are also described in WO 98/39352 and WO 99/14226.

Analogs of methyleneoxy (4′-CH₂—O-2′) BNA, methyleneoxy (4′-CH₂—O-2′) BNA, and 2′-thio-BNAs, have also been prepared (see, e.g., Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (see, e.g., Wengel et al., WO 99/14226). Furthermore, synthesis of 2′-amino-BNA, a novel comformationally restricted high-affinity oligonucleotide analog, has been described in the art (see, e.g., Singh et al., J. Org. Chem., 1998, 63, 10035-10039). In addition, 2′-amino- and 2′-methylamino-BNA's have been prepared and the thermal stability of their duplexes with complementary RNA and DNA strands has been previously reported.

In certain embodiments, bicyclic nucleoside having Formula VI:

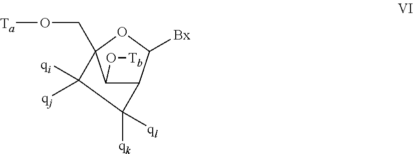

wherein:

Bx is a heterocyclic base moiety;

Tₐ and T_{b} are each, independently, H, a hydroxyl protecting group, a conjugate group, a reactive phosphorus group, a phosphorus moiety, or a covalent attachment to a support medium;

each qᵢ, qⱼ, qₖ and qₗ is, independently, H, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxyl, substituted C₁-C₁₂ alkoxyl, OJⱼ, SJⱼ, SOJⱼ, SO₂Jⱼ, NJⱼJₖ, N₃, CN, C(═O)OJⱼ, C(═O)NJⱼJₖ, C(═O)NJⱼ, O—C(═)NJⱼJₖ, N(H)C(═NH)NJⱼJₖ, N(H)C(═O)NJⱼJₖ, or N(H)C(═S)NJⱼJₖ; and

qᵢ and qⱼ or qₗ and qₖ together are ═C(q_{g}(qₕ), wherein q_{g} and qₕ are each, independently, H, halogen, C₁-C₁₂ alkyl, or substituted C₁-C₁₂ alkyl.

One carbocyclic bicyclic nucleoside having a 4′-(CH₂)₃-2′ bridge and the alkenyl analog, bridge 4′-CH═CH—CH₂-2′, have been described (see, e.g., Freier et al., Nucleic Acids Research, 1997, 25(22), 4429-4443 and Albaek et al., J. Org. Chem., 2006, 71, 7731-7740). The synthesis and preparation of carbocyclic bicyclic nucleosides along with their oligomerization and biochemical studies have also been described (see, e.g., Srivastava et al., J. Am. Chem. Soc. 2007, 129(26), 8362-8379).

In certain embodiments, oligomeric compounds comprise one or more modified tetrahydropyran nucleoside, which is a nucleoside having a six-membered tetrahydropyran in place of the pentofuranosyl residue in naturally occurring nucleosides. Modified tetrahydropyran nucleosides include, but are not limited to, what is referred to in the art as hexitol nucleic acid (HNA), anitol nucleic acid (ANA), manitol nucleic acid (MNA) (see Leumann, C J. Bioorg. & Med. Chem. (2002) 10:841-854), fluoro HNA (F—HNA), or those compounds having Formula X:

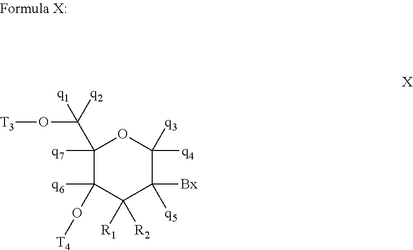

wherein independently for each of said at least one tetrahydropyran nucleoside analog of Formula X:

Bx is a heterocyclic base moiety;

T₃ and T₄ are each, independently, an internucleoside linking group linking the tetrahydropyran nucleoside analog to the antisense compound or one of T₃ and T₄ is an internucleoside linking group linking the tetrahydropyran nucleoside analog to the antisense compound and the other of T₃ and T₄ is H, a hydroxyl protecting group, a linked conjugate group, or a 5′ or 3′-terminal group;

q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and

one of R₁ and R₂ is hydrogen and the other is selected from halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, the modified THP nucleosides of Formula X are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, THP nucleosides of Formula X are provided wherein one of R₁ and R₂ is F. In certain embodiments, R₁ is fluoro and R₂ is H, R₁ is methoxy and R₂ is H, and R₁ is methoxyethoxy and R₂ is H.

Many other bicyclo and tricyclo sugar surrogate ring systems are also known in the art that can be used to modify nucleosides for incorporation into antisense compounds (see, e.g., review article: Leumann, J. C, Bioorganic & Medicinal Chemistry, 2002, 10, 841-854). Combinations of these modifications are also provided for herein without limitation, such as 2′-F-5′-methyl substituted nucleosides (see PCT International Application WO 2008/101157 Published on Aug. 21, 2008 for other disclosed 5′,2′-bis substituted nucleosides) and replacement of the ribosyl ring oxygen atom with S and further substitution at the 2′-position (see published U.S. Patent Application US2005-0130923, published on Jun. 16, 2005) or alternatively 5′-substitution of a bicyclic nucleic acid (see PCT International Application WO 2007/134181, published on Nov. 22, 2007 wherein a 4′-CH₂—O-2′ bicyclic nucleoside is further substituted at the 5′ position with a 5′-methyl or a 5′-vinyl group). Such ring systems can undergo various additional substitutions to enhance activity.

Methods for the preparations of modified sugars are well known to those skilled in the art.

In nucleotides having modified sugar moieties, the nucleobase moieties (natural, modified, or a combination thereof) are maintained for hybridization with an appropriate nucleic acid target.

In certain embodiments, antisense compounds comprise one or more nucleotides having modified sugar moieties. In certain embodiments, the modified sugar moiety is 2′-MOE. In certain embodiments, the 2′-MOE modified nucleotides are arranged in a gapmer motif. In certain embodiments, the modified sugar moiety is a cEt. In certain embodiments, the cEt modified nucleotides are arranged throughout the wings of a gapmer motif

### Certain Modified Nucleobases

In certain embodiments, nucleosides for use in the compositions of the present invention comprise one or more unmodified nucleobases. In certain embodiments, nucleosides for use in the compositions of the present invention comprise one or more modified nucleobases.

As used herein the terms, “unmodified nucleobase” and “naturally occurring nucleobase” include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine, universal bases, hydrophobic bases, promiscuous bases, size-expanded bases, and fluorinated bases as defined herein. Further modified nucleobases include tricyclic pyrimidines such as phenoxazine cytidine([5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, Kroschwitz, J. I., Ed., John Wiley & Sons, 1990, 858-859; those disclosed by Englisch et al., Angewandte Chemie, International Edition, 1991, 30, 613; and those disclosed by Sanghvi, Y. S., Chapter 15, Antisense Research and Applications, Crooke, S. T. and Lebleu, B., Eds., CRC Press, 1993, 273-288.

The heterocyclic base moiety of each of the nucleosides can be modified with one or more substituent groups to enhance one or more properties such as affinity for a target strand or affect some other property in an advantageous manner. Modified nucleobases include without limitation, universal bases, hydrophobic bases, promiscuous bases, size-expanded bases, and fluorinated bases as defined herein. Certain of these nucleobases are particularly useful for increasing the binding affinity of the oligomeric compounds as provided herein. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Antisense Research and Applications, Sanghvi, Y. S., Crooke, S. T. and Lebleu, B., Eds., CRC Press, Boca Raton, 1993, 276-278).

Representative United States patents that teach the preparation of certain of the above noted modified nucleobases as well as other modified nucleobases include without limitation, U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096, certain of which are commonly owned with the instant application, and each of which is herein incorporated by reference in its entirety.

Certain Internucleoside Linkages

In certain embodiments, the present invention provides compositions comprising oligomeric compounds comprising linked nucleosides. In such embodiments, nucleosides may be linked together using any internucleoside linkage. The two main classes of internucleoside linking groups are defined by the presence or absence of a phosphorus atom. Representative phosphorus containing internucleoside linkages include, but are not limited to, phosphodiesters (P═O), phosphotriesters, methylphosphonates, phosphoramidate, and phosphorothioates (P═S). Representative non-phosphorus containing internucleoside linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Oligonucleotides having non-phosphorus internucleoside linking groups may be referred to as oligonucleotides. Modified linkages, compared to natural phosphodiester linkages, can be used to alter, typically increase, nuclease resistance of the oligomeric compound. In certain embodiments, internucleoside linkages having a chiral atom can be prepared a racemic mixture, as separate enantomers. Representative chiral linkages include, but are not limited to, alkylphosphonates and phosphorothioates. Methods of preparation of phosphorous-containing and non-phosphorous-containing internucleoside linkages are well known to those skilled in the art.

The oligonucleotides described herein contain one or more asymmetric centers and thus give rise to enantomers, diastereomers, and other stereoisomeric configurations that may be defined, in terms of absolute stereochemistry, as (R) or (S), a or β such as for sugar anomers, or as (D) or (L) such as for amino acids et al. Included in the antisense compounds provided herein are all such possible isomers, as well as their racemic and optically pure forms.

As used herein the phrase “neutral internucleoside linkage” is intended to include internucleoside linkages that are non-ionic. Neutral internucleoside linkages include without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

Certain Lengths

In certain embodiments, the present invention provides compositions comprising oligomeric compounds including oligonucleotides of any of a variety of ranges of lengths. In certain embodiments, the invention provides oligomeric compounds or oligonucleotides consisting of X to Y linked nucleosides, where X represents the fewest number of nucleosides in the range and Y represents the largest number of nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≦Y. For example, in certain embodiments, the invention provides oligomeric compounds which comprise oligonucleotides consisting of 8 to 9, 8 to 10, 8 to 11, 8 to 12, 8 to 13, 8 to 14, 8 to 15, 8 to 16, 8 to 17, 8 to 18, 8 to 19, 8 to 20, 8 to 21, 8 to 22, 8 to 23, 8 to 24, 8 to 25, 8 to 26, 8 to 27, 8 to 28, 8 to 29, 8 to 30, 9 to 10, 9 to 11, 9 to 12, 9 to 13, 9 to 14, 9 to 15, 9 to 16, 9 to 17, 9 to 18, 9 to 19, 9 to 20, 9 to 21, 9 to 22, 9 to 23, 9 to 24, 9 to 25, 9 to 26, 9 to 27, 9 to 28, 9 to 29, 9 to 30, 10 to 11, 10 to 12, 10 to 13, 10 to 14, 10 to 15, 10 to 16, 10 to 17, 10 to 18, 10 to 19, 10 to 20, 10 to 21, 10 to 22, 10 to 23, 10 to 24, 10 to 25, 10 to 26, 10 to 27, 10 to 28, 10 to 29, 10 to 30, 11 to 12, 11 to 13, 11 to 14, 11 to 15, 11 to 16, 11 to 17, 11 to 18, 11 to 19, 11 to 20, 11 to 21, 11 to 22, 11 to 23, 11 to 24, 11 to 25, 11 to 26, 11 to 27, 11 to 28, 11 to 29, 11 to 30, 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides. In embodiments where the number of nucleosides of an oligomeric compound or oligonucleotide is limited, whether to a range or to a specific number, the oligomeric compound or oligonucleotide may, nonetheless further comprise additional other substituents. For example, an oligonucleotide comprising 8-30 nucleosides excludes oligonucleotides having 31 nucleosides, but, unless otherwise indicated, such an oligonucleotide may further comprise, for example one or more conjugates, terminal groups, or other substituents.

### Certain Motifs

In certain embodiments, the present invention provides compositions comprising oligonucleotides comprising one or more regions having a particular nucleoside motif.

1. Certain 5′-Terminal Nucleosides

In certain embodiments, the 5′-terminal nucleoside of a modified oligonucleotide for use in the compositions of the present invention comprises a phosphorous moiety at the 5′-end. In certain embodiments the 5′-terminal nucleoside comprises a 2′-modification. In certain such embodiments, the 2′-modification of the 5′-terminal nucleoside is a cationic modification. In certain embodiments, the 5′-terminal nucleoside comprises a 5′-modification. In certain embodiments, the 5′-terminal nucleoside comprises a 2′-modification and a 5′-modification.

In certain embodiments, the 5′-terminal nucleoside is a 5′-stabilizing nucleoside. In certain embodiments, the modifications of the 5′-terminal nucleoside stabilize the 5′-phosphate. In certain embodiments, oligonucleotides comprising modifications of the 5′-terminal nucleoside are resistant to exonucleases. In certain embodiments, oligonucleotides comprising modifications of the 5′-terminal nucleoside have improved antisense properties. In certain such embodiments, oligonucleotides comprising modifications of the 5′-terminal nucleoside have improved association with members of the RISC pathway. In certain embodiments, oligonucleotides comprising modifications of the 5′-terminal nucleoside have improved affinity for Ago2.

In certain embodiments, the 5′ terminal nucleoside is attached to a plurality of nucleosides by a modified linkage. In certain such embodiments, the 5′ terminal nucleoside is a plurality of nucleosides by a phosphorothioate linkage.

2. Certain Alternating Regions

In certain embodiments, oligonucleotides for use in the compositions of the present invention comprise one or more regions of alternating modifications. In certain embodiments, oligonucleotides comprise one or more regions of alternating nucleoside modifications. In certain embodiments, oligonucleotides comprise one or more regions of alternating linkage modifications. In certain embodiments, oligonucleotides comprise one or more regions of alternating nucleoside and linkage modifications.

In certain embodiments, oligonucleotides for use in the compositions of the present invention comprise one or more regions of alternating 2′-F modified nucleosides and 2′-OMe modified nucleosides. In certain such embodiments, such regions of alternating 2′F modified and 2′OMe modified nucleosides also comprise alternating linkages. In certain such embodiments, the linkages at the 3′ end of the 2′-F modified nucleosides are phosphorothioate linkages. In certain such embodiments, the linkages at the 3′ end of the 2′OMe nucleosides are phosphodiester linkages. In certain embodiments, such alternating regions are:

(2′-F)-(PS)-(2′-OMe)-(PO)

In certain embodiments, oligomeric compounds comprise 2, 3, 4, 5, 6, 7, 8, 9, 10, or 11 such alternating regions. Such regions may be contiguous or may be interrupted by differently modified nucleosides or linkages.

In certain embodiments, one or more alternating regions in an alternating motif include more than a single nucleoside of a type. For example, oligomeric compounds of the present invention may include one or more regions of any of the following nucleoside motifs:

AABBAA;

ABBABB;

AABAAB;

ABBABAABB;

ABABAA;

AABABAB;

ABABAA;

ABBAABBABABAA;

BABBAABBABABAA; or

ABABBAABBABABAA;

wherein A is a nucleoside of a first type and B is a nucleoside of a second type. In certain embodiments, A and B are each selected from 2′-F, 2′-OMe, BNA, DNA, and MOE.

In certain embodiments, A is DNA. In certain embodiments, B is 4′-CH₂O-2′-BNA. In certain embodiments, A is DNA and B is 4′-CH₂O-2′-BNA. In certain embodiments A is 4′-CH₂O-2′-BNA. In certain embodiments, B is DNA. In certain embodiments A is 4′-CH₂O-2′-BNA and B is DNA. In certain embodiments, A is 2′-F. In certain embodiments, B is 2′-OMe. In certain embodiments, A is 2′-F and B is 2′-OMe. In certain embodiments, A is 2′-OMe. In certain embodiments, B is 2′-F. In certain embodiments, A is 2′-OMe and B is 2′-F. In certain embodiments, A is DNA and B is 2′-OMe. In certain embodiments, A is 2′-OMe and B is DNA.

In certain embodiments, oligomeric compounds having such an alternating motif also comprise a 5′ terminal nucleoside comprising a phosphate stabilizing modification. In certain embodiments, oligomeric compounds having such an alternating motif also comprise a 5′ terminal nucleoside comprising a 2′-cationic modification. In certain embodiments, oligomeric compounds having such an alternating motif also comprise a 5′ terminal nucleoside of formula II, IV, VI, VII, VIII, XIII, or XIV. In certain embodiments, oligomeric compounds having such an alternating motif comprise a 5′ terminal di-nucleoside of formula IX or X.

3. Two-Two-Three Motifs

In certain embodiments, oligonucleotides for use in the compositions of the present invention comprise a region having a 2-2-3 motif. Such regions comprises the following motif:

5′-(E)_{w}-(A)₂-(B)ₓ-(A)₂-(C)_{y}-(A)₃-(D)_{z}

wherein: A is a first type of modified nucleosde;

B, C, D, and E are nucleosides that are differently modified than A, however, B, C, D, and E may have the same or different modifications as one another;

w and z are from 0 to 15;

x and y are from 1 to 15.

In certain embodiments, A is a 2′-OMe modified nucleoside. In certain embodiments, B, C, D, and E are all 2′-F modified nucleosides. In certain embodiments, A is a 2′-OMe modified nucleoside and B, C, D, and E are all 2′-F modified nucleosides.

In certain embodiments, the linkages of a 2-2-3 motif are all modified linkages. In certain embodiments, the linkages are all phosphorothioate linkages. In certain embodiments, the linkages at the 3′-end of each modification of the first type are phosphodiester.

In certain embodiments, Z is 0. In such embodiments, the region of three nucleosides of the first type are at the 3′-end of the oligonucleotide. In certain embodiments, such region is at the 3′-end of the oligomeric compound, with no additional groups attached to the 3′ end of the region of three nucleosides of the first type. In certain embodiments, an oligomeric compound comprising an oligonucleotide where Z is 0, may comprise a terminal group attached to the 3′-terminal nucleoside. Such terminal groups may include additional nucleosides. Such additional nucleosides are typically non-hybridizing nucleosides.

In certain embodiments, Z is 1-3. In certain embodiments, Z is 2. In certain embodiments, the nucleosides of Z are 2′-MOE nucleosides. In certain embodiments, Z represents non-hybridizing nucleosides. To avoid confusion, it is noted that such non-hybridizing nucleosides might also be described as a 3′-terminal group with Z=0.

B. Combinations of Motifs

It is to be understood, that certain of the above described motifs and modifications may be combined. Since a motif may comprises only a few nucleosides, a particular oligonucleotide may comprise two or more motifs. By way of non-limiting example, in certain embodiments, oligomeric compounds may have nucleoside motifs as described in the table below. In the table below, the term “None” indicates that a particular feature is not present in the oligonucleotide. For example, “None” in the column labeled “5′ motif/modification” indicates that the 5′ end of the oligonucleotide comprises the first nucleoside of the central motif.

| 5′ motif/modification | Central Motif | 3′-motif |
|---|---|---|
| Formula I or II | Alternating | 2 MOE nucleosides |
| Formula I or II | 2-2-3 motif | 2 MOE nucleosides |
| Formula I or II | Uniform | 2 MOE nucleosides |
| Formula I or II | Alternating | 2 MOE nucleosides |
| Formula I or II | Alternating | 2 MOE A's |
| Formula I or II | 2-2-3 motif | 2 MOE A's |
| Formula I or II | Uniform | 2 MOE A's |
| Formula I or II | Alternating | 2 MOE U's |
| Formula I or II | 2-2-3 motif | 2 MOE U's |
| Formula I or II | Uniform | 2 MOE U's |
| None | Alternating | 2 MOE nucleosides |
| None | 2-2-3 motif | 2 MOE nucleosides |
| None | Uniform | 2 MOE nucleosides |

Oligomeric compounds having any of the various nucleoside motifs described herein, may have any linkage motif. For example, the oligomeric compounds, including but not limited to those described in the above table, may have a linkage motif selected from non-limiting the table below:

| 5′ most linkage | Central region | 3′-region |
|---|---|---|
| PS | Alternating PO/PS | 6 PS |
| PS | Alternating PO/PS | 7 PS |
| PS | Alternating PO/PS | 8 PS |

As is apparent from the above, non-limiting tables, the lengths of the regions defined by a nucleoside motif and that of a linkage motif need not be the same. For example, the 3′ region in the nucleoside motif table above is 2 nucleosides, while the 3′-region of the linkage motif table above is 6-8 nucleosides. Combining the tables results in an oligonucleotide having two 3′-terminal MOE nucleosides and six to eight 3′-terminal phosphorothioate linkages (so some of the linkages in the central region of the nucleoside motif are phosphorothioate as well). To further illustrate, and not to limit in any way, nucleoside motifs and sequence motifs are combined to show five non-limiting examples in the table below. The first column of the table lists nucleosides and linkages by position from N1 (the first nucleoside at the 5′-end) to N20 (the 20^{th} position from the 5′-end). In certain embodiments, oligonucleotides for use in the compositions of the present invention are longer than 20 nucleosides (the table is merely exemplary). Certain positions in the table recite the nucleoside or linkage “none” indicating that the oligonucleotide has no nucleoside at that position.

| Pos | A | B | C | D | E | F |
|---|---|---|---|---|---|---|
| N1 | Formula I or II | Formula I or II | Formula I or II | Formula I or II | Formula I or II | 2′-F |
| L1 | PS | PS | PS | PS | PO | PO |
| N2 | 2′-F | 2′-F | 2′-F | 2′-OMe | MOE | 2′-OMe |
| L2 | PS | PS | PS | PO | PS | PO |
| N3 | 2′-OMe | 2′-F | 2′-F | 2′-F | 2′-F | 2′-F |
| L3 | PO | PS | PS | PS | PS | PS |
| N4 | 2′-F | 2′-F | 2′-F | 2′-OMe | 2′-F | 2′-OMe |
| L4 | PS | PS | PS | PO | PS | PO |
| N5 | 2′-OMe | 2′-F | 2′-F | 2′-F | 2′-OMe | 2′-F |
| L5 | PO | PS | PS | PS | PO | PS |
| N6 | 2′-F | 2′-OMe | 2′-F | 2′-OMe | 2′-OMe | 2′-OMe |
| L6 | PS | PO | PS | PO | PO | PO |
| N7 | 2′-OMe | 2′-OMe | 2′-F | 2′-F | 2′-OMe | 2′-F |
| L7 | PO | PO | PS | PS | PO | PS |
| N8 | 2′-F | 2′-F | 2′-F | 2′-OMe | 2′-F | 2′-OMe |
| L8 | PS | PS | PS | PO | PS | PO |
| N9 | 2′-OMe | 2′-F | 2′-F | 2′-F | 2′-F | 2′-F |
| L9 | PO | PS | PS | PS | PS | PS |
| N10 | 2′-F | 2′-OMe | 2′-F | 2′-OMe | 2′-OMe | 2′-OMe |
| L10 | PS | PO | PS | PO | PO | PO |
| N11 | 2′-OMe | 2′-OMe | 2′-F | 2′-F | 2′OMe | 2′-F |
| L11 | PO | PO | PS | PS | PO | PS |
| N12 | 2′-F | 2′-F | 2′-F | 2′-F | 2′-F | 2′-OMe |
| L12 | PS | PS | PS | PO | PS | PO |
| N13 | 2′-OMe | 2′-F | 2′-F | 2′-F | 2′-F | 2′-F |
| L13 | PO | PS | PS | PS | PS | PS |
| N14 | 2′-F | 2′-OMe | 2′-F | 2′-F | 2′-F | 2′-F |
| L14 | PS | PS | PS | PS | PS | PS |
| N15 | 2′-OMe | 2′OMe | 2′-F | 2′-F | 2′-MOE | 2′-F |
| L15 | PS | PS | PS | PS | PS | PS |
| N16 | 2′-F | 2′OMe | 2′-F | 2′-F | 2′-MOE | 2′-F |
| L16 | PS | PS | PS | PS | PS | PS |
| N17 | 2′-OMe | 2′-MOE U | 2′-F | 2′-F | 2′-MOE | 2′-F |
| L17 | PS | PS | PS | PS | None | PS |
| N18 | 2′-F | 2′-MOE U | 2′-F | 2′-OMe | None | MOE A |
| L18 | PS | None | PS | PS | None | PS |
| N19 | 2′-MOE U | None | 2′-MOE U | 2′-MOE A | None | MOE U |
| L19 | PS | None | PS | PS | None | None |
| N20 | 2′-MOE U | None | 2′-MOE U | 2′-MOE A | None | None |

In the above, non-limiting examples:

Column A represent an oligomeric compound consisting of 20 linked nucleosides, wherein the oligomeric compound comprises: a modified 5′-terminal nucleoside of Formula I or II; a region of alternating nucleosides; a region of alternating linkages; two 3′-terminal MOE nucleosides, each of which comprises a uracil base; and a region of six phosphorothioate linkages at the 3′-end.

Column B represents an oligomeric compound consisting of 18 linked nucleosides, wherein the oligomeric compound comprises: a modified 5′-terminal nucleoside of Formula I or II; a 2-2-3 motif wherein the modified nucleoside of the 2-2-3 motif are 2′O-Me and the remaining nucleosides are all 2′-F; two 3′-terminal MOE nucleosides, each of which comprises a uracil base; and a region of six phosphorothioate linkages at the 3′-end.

Column C represents an oligomeric compound consisting of 20 linked nucleosides, wherein the oligomeric compound comprises: a modified 5′-terminal nucleoside of Formula I or II; a region of uniformly modified 2′-F nucleosides; two 3′-terminal MOE nucleosides, each of which comprises a uracil base; and wherein each internucleoside linkage is a phosphorothioate linkage.

Column D represents an oligomeric compound consisting of 20 linked nucleosides, wherein the oligomeric compound comprises: a modified 5′-terminal nucleoside of Formula I or II; a region of alternating 2′-OMe/2′-F nucleosides; a region of uniform 2′F nucleosides; a region of alternating phosphorothioate/phosphodiester linkages; two 3′-terminal MOE nucleosides, each of which comprises an adenine base; and a region of six phosphorothioate linkages at the 3′-end.

Column E represents an oligomeric compound consisting of 17 linked nucleosides, wherein the oligomeric compound comprises: a modified 5′-terminal nucleoside of Formula I or II; a 2-2-3 motif wherein the modified nucleoside of the 2-2-3 motif are 2′F and the remaining nucleosides are all 2′-OMe; three 3′-terminal MOE nucleosides.

Column F represents an oligomeric compound consisting of 18 linked nucleosides, wherein the oligomeric compound comprises: a region of alternating 2′-OMe/2′-F nucleosides; a region of uniform 2′F nucleosides; a region of alternating phosphorothioate/phosphodiester linkages; two 3′-terminal MOE nucleosides, one of which comprises a uracil base and the other of which comprises an adenine base; and a region of six phosphorothioate linkages at the 3′-end.

The above examples are provided solely to illustrate how the described motifs may be used in combination and are not intended to limit the invention to the particular combinations or the particular modifications used in illustrating the combinations. Further, specific examples herein, including, but not limited to those in the above table are intended to encompass more generic embodiments. For example, column A in the above table exemplifies a region of alternating 2′-OMe and 2′-F nucleosides. Thus, that same disclosure also exemplifies a region of alternating different 2′-modifications. It also exemplifies a region of alternating 2′-O-alkyl and 2′-halogen nucleosides. It also exemplifies a region of alternating differently modified nucleosides. All of the examples throughout this specification contemplate such generic interpretation.

It is also noted that the lengths of oligomeric compounds, such as those exemplified in the above tables, can be easily manipulated by lengthening or shortening one or more of the described regions, without disrupting the motif.

### IV. OLIGOMERIC COMPOUNDS

In certain embodiments, the compositions of the present invention comprises oligomeric compounds. In certain embodiments, oligomeric compounds comprise an oligonucleotide. In certain embodiments, an oligomeric compound comprises an oligonucleotide and one or more conjugate and/or terminal groups. Such conjugate and/or terminal groups may be added to oligonucleotides having any of the chemical motifs discussed above. Thus, for example, an oligomeric compound comprising an oligonucleotide having region of alternating nucleosides may comprise a terminal group.

A. Certain Conjugate Groups

In certain embodiments, oligomeric compounds are modified by attachment of one or more conjugate groups. In general, conjugate groups modify one or more properties of the attached oligomeric compound including but not limited to pharmacodynamics, pharmacokinetics, stability, binding, absorption, cellular distribution, cellular uptake, charge and clearance. Conjugate groups are routinely used in the chemical arts and are linked directly or via an optional conjugate linking moiety or conjugate linking group to a parent compound such as an oligomeric compound, such as an oligonucleotide. Conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes. Certain conjugate groups have been described previously, for example: cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-5-tritylthiol (Manoharan et al., Aim. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937).

In certain embodiments, a conjugate group comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130.

Representative U.S. patents that teach the preparation of oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

In certain embodiments, conjugate groups are directly attached to oligonucleotides in oligomeric compounds. In certain embodiments, conjugate groups are attached to oligonucleotides by a conjugate linking group. In certain such embodiments, conjugate linking groups, including, but not limited to, bifunctional linking moieties such as those known in the art are amenable to the compounds provided herein. Conjugate linking groups are useful for attachment of conjugate groups, such as chemical stabilizing groups, functional groups, reporter groups and other groups to selective sites in a parent compound such as for example an oligomeric compound. In general a bifunctional linking moiety comprises a hydrocarbyl moiety having two functional groups. One of the functional groups is selected to bind to a parent molecule or compound of interest and the other is selected to bind essentially any selected group such as chemical functional group or a conjugate group. In some embodiments, the conjugate linker comprises a chain structure or an oligomer of repeating units such as ethylene glycol or amino acid units. Examples of functional groups that are routinely used in a bifunctional linking moiety include, but are not limited to, electrophiles for reacting with nucleophilic groups and nucleophiles for reacting with electrophilic groups. In some embodiments, bifunctional linking moieties include amino, hydroxyl, carboxylic acid, thiol, unsaturations (e.g., double or triple bonds), and the like.

Some nonlimiting examples of conjugate linking moieties include pyrrolidine, 8-amino-3,6-dioxaoctanoic acid (ADO), succinimidyl 4-(N-maleimidomethyl)cyclohexane-1-carboxylate (SMCC) and 6-aminohexanoic acid (AHEX or AHA). Other linking groups include, but are not limited to, substituted C1-C10 alkyl, substituted or unsubstituted C2-C10 alkenyl or substituted or unsubstituted C2-C10 alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

Conjugate groups may be attached to either or both ends of an oligonucleotide (terminal conjugate groups) and/or at any internal position.

In certain embodiments, conjugate groups are at the 3′-end of an oligonucleotide of an oligomeric compound. In certain embodiments, conjugate groups are near the 3′-end. In certain embodiments, conjugates are attached at the 3′ end of an oligomeric compound, but before one or more terminal group nucleosides. In certain embodiments, conjugate groups are placed within a terminal group. Solely to illustrate such groups at a 3′-end, and not to limit such groups, the following examples are provided.

| | SEQ ID |
|---|---|
| Exemplified oligomeric compounds | NO: |
| Po-U_{fo}U_{fo}G_{fo}U_{fo}C_{fo}U_{fo}C_{fo}U_{fo}G_{fo}G_{fo}U_{fo}C_{fo}C_{fo}U_{fs}U_{fs}A_{fs}C_{fs}U_{fs}U_{fs}AₑₛAₑ | 6 |
| Po-U_{fo}U_{fo}G_{fo}U_{fo}C_{fo}U_{fo}C_{fo}U_{fo}G_{fo}G_{fo}U_{fo}C_{fo}C_{fo}U_{fs}U_{fs}A_{fs}C_{fs}U_{fs}U_{fs}AₑₛAₑₛpy-acetyl | 6 |
| Po-U_{fo}U_{fo}G_{fo}U_{fo}C_{fo}U_{fo}C_{fo}U_{fo}G_{fo}G_{fo}U_{fo}C_{fo}C_{fo}U_{fs}U_{fs}A_{fs}C_{fs}U_{fs}U_{fs}AₑₛAₑₛpy-ibuprofin | 6 |
| Po-U_{fo}U_{fo}G_{fo}U_{fo}C_{fo}U_{fo}C_{fo}U_{fo}G_{fo}G_{fo}U_{fo}C_{fo}C_{fo}U_{fs}U_{fs}A_{fs}C_{fs}U_{fs}U_{fs}AₑₛAₑₛpy-C₁₆ | 26 |
| Po-U_{fo}U_{fo}G_{fo}U_{fo}C_{fo}U_{fo}C_{fo}U_{fo}G_{fo}G_{fo}U_{fo}C_{fo}C_{fo}U_{fs}U_{fs}A_{fs}C_{fs}U_{fs}U_{fs}Aₑₛpy-acetyl | 27 |
| Po-U_{fo}U_{fo}G_{fo}U_{fo}C_{fo}U_{fo}C_{fo}U_{fo}G_{fo}G_{fo}U_{fo}C_{fo}C_{fo}U_{fs}U_{fs}A_{fs}C_{fs}U_{fs}U_{fs}Aₑₛpy-ibuprofin | 27 |
| Po-U_{fo}U_{fo}G_{fo}U_{fo}C_{fo}U_{fo}C_{fo}U_{fo}G_{fo}G_{fo}U_{fo}C_{fo}C_{fo}U_{fs}U_{fs}A_{fs}C_{fs}U_{fs}U_{fs}Aₑₛpy-C₁₆ | 26 |
| Po-U_{fo}U_{fo}G_{fo}U_{fo}C_{fo}U_{fo}C_{fo}U_{fo}G_{fo}G_{fo}U_{fo}C_{fo}C_{fo}U_{fs}U_{fs}A_{fs}C_{fs}U_{fs}U_{fs}Aₑₛpy-acetyl-Aₑₛ | 6 |
| Po-U_{fo}U_{fo}G_{fo}U_{fo}C_{fo}U_{fo}C_{fo}U_{fo}G_{fo}G_{fo}U_{fo}C_{fo}C_{fo}U_{fs}U_{fs}A_{fs}C_{fs}U_{fs}U_{fs}Aₑₛpy-ibuprofin-Aₑₛ | 6 |
| Po-U_{fo}U_{fo}G_{fo}U_{fo}C_{fo}U_{fo}C_{fo}U_{fo}G_{fo}G_{fo}U_{fo}C_{fo}C_{fo}U_{fs}U_{fs}A_{fs}C_{fs}U_{fs}U_{fs}Aₑₛpy-C_{16-Aes} | 28 |

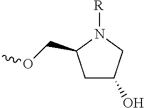

Py = pyrrolidine 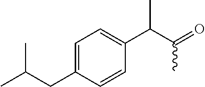
R = Ac, Ibuprofen, C₁₆

In certain embodiments, conjugate groups are attached to a nucleoside. Such a nucleoside may be incorporated into an oligomeric compound or oligonucleotide. In certain embodiments conjugated nucleotides may be incorporated into an oligonucleotide at the 5′ terminal end. In certain embodiments conjugated nucleotides may be incorporated into an oligonucleotide at the 3′ terminal end. In certain embodiments conjugated nucleotides may be incorporated into an oligonucleotide internally. Solely for illustration, and not to limit the conjugate or its placement, the following example shows oligonucleotides where each uracil nucleoside is, separately replaced with a conjugated thymidine nucleoside:

| | SEQ |
|---|---|
| | ID |
| | NO: |
| Po-U_{fo}U_{fo}G_{fo}U_{fo}C_{fo}U_{fo}C_{fo}U_{fo}G_{fo}G_{fo}U_{fo}C_{fo}C_{fo}U_{fs}U_{fs}A_{fs}C_{fs}U_{fs}U_{fs}AₑₛAₑ | 6 |
| Po-U_{fo}U_{fo}G_{fo}U_{fo}C_{fo}U_{fo}C_{fo}U_{fo}G_{fo}G_{fo}U_{fo}C_{fo}C_{fo}U_{fs}U_{fs}A_{fs}C_{fs}U_{fs}T_{Xs}AₑₛAₑ | 29 |
| Po-U_{fo}U_{fo}G_{fo}U_{fo}C_{fo}U_{fo}C_{fo}U_{fo}G_{fo}G_{fo}U_{fo}C_{fo}C_{fo}U_{fs}U_{fs}A_{fs}C_{fs}T_{Xs}U_{fs}AₑₛAₑ | 30 |
| Po-U_{fo}U_{fo}G_{fo}U_{fo}C_{fo}U_{fo}C_{fo}U_{fo}G_{fo}G_{fo}U_{fo}C_{fo}C_{fo}U_{fs}T_{Xs}A_{fs}C_{fs}U_{fs}U_{fs}AₑₛAₑ | 31 |
| Po-U_{fo}U_{fo}G_{fo}U_{fo}C_{fo}U_{fo}C_{fo}U_{fo}G_{fo}G_{fo}U_{fo}C_{fo}C_{fo}T_{Xs}U_{fs}A_{fs}C_{fs}U_{fs}U_{fs}AₑₛAₑ | 32 |
| Po-U_{fo}U_{fo}G_{fo}U_{fo}C_{fo}U_{fo}C_{fo}U_{fo}G_{fo}G_{fo}T_{Xo}C_{fo}C_{fo}U_{fs}U_{fs}A_{fs}C_{fs}U_{fs}U_{fs}AₑₛAₑ | 33 |
| Po-U_{fo}U_{fo}G_{fo}U_{fo}C_{fo}U_{fo}C_{fo}T_{Xo}G_{fo}G_{fo}U_{fo}C_{fo}C_{fo}U_{fs}U_{fs}A_{fs}C_{fs}U_{fs}U_{fs}AₑₛAₑ | 34 |
| Po-U_{fo}U_{fo}G_{fo}U_{fo}C_{fo}T_{Xo}C_{fo}U_{fo}G_{fo}G_{fo}U_{fo}C_{fo}C_{fo}U_{fs}U_{fs}A_{fs}C_{fs}U_{fs}U_{fs}AₑₛAₑ | 35 |
| Po-U_{fo}U_{fo}G_{fo}T_{Xo}C_{fo}U_{fo}C_{fo}U_{fo}G_{fo}G_{fo}U_{fo}C_{fo}C_{fo}U_{fs}U_{fs}A_{fs}C_{fs}U_{fs}U_{fs}AₑₛAₑ | 36 |
| Po-U_{fo}T_{Xo}G_{fo}U_{fo}C_{fo}U_{fo}C_{fo}U_{fo}G_{fo}G_{fo}U_{fo}C_{fo}C_{fo}U_{fs}U_{fs}A_{fs}C_{fs}U_{fs}U_{fs}AₑₛAₑ | 37 |
| Po-T_{Xo}U_{fo}G_{fo}U_{fo}C_{fo}U_{fo}C_{fo}U_{fo}G_{fo}G_{fo}U_{fo}C_{fo}C_{fo}U_{fs}U_{fs}A_{fs}C_{fs}U_{fs}U_{fs}AₑₛAₑ | 5 |

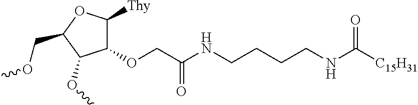

x = aba-C₁₆

B. Terminal Groups

In certain embodiments, oligomeric compounds comprise terminal groups at one or both ends. In certain embodiments, a terminal group may comprise any of the conjugate groups discussed above. In certain embodiments, terminal groups may comprise additional nucleosides and/or inverted abasic nucleosides. In certain embodiments, a terminal group is a stabilizing group.

In certain embodiments, oligomeric compounds comprise one or more terminal stabilizing group that enhances properties such as for example nuclease stability. Included in stabilizing groups are cap structures. The terms “cap structure” or “terminal cap moiety,” as used herein, refer to chemical modifications, which can be attached to one or both of the termini of an oligomeric compound. These terminal modifications protect the oligomeric compounds having terminal nucleic acid moieties from exonuclease degradation, and can help in delivery and/or localization within a cell. The cap can be present at the 5′-terminus (5′-cap) or at the 3′-terminus (3′-cap) or can be present on both termini. In non-limiting examples, the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl ribonucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270).

Particularly suitable 3′-cap structures include, for example 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxy-pentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Tyer, 1993, Tetrahedron 49, 1925 and Published U.S. Patent Application Publication No. US 2005/0020525 published on Jan. 27, 2005). Further 3′ and 5′-stabilizing groups that can be used to cap one or both ends of an oligomeric compound to impart nuclease stability include those disclosed in WO 03/004602.

1. Terminal-Group Nucleosides

In certain embodiments, one or more additional nucleosides is added to one or both terminal ends of an oligonucleotide of an oligomeric compound. Such additional terminal nucleosides are referred to herein as terminal-group nucleosides. In a double-stranded compound, such terminal-group nucleosides are terminal (3′ and/or 5′) overhangs. In the setting of double-stranded antisense compounds, such terminal-group nucleosides may or may not be complementary to a target nucleic acid.

In a single-stranded antisense oligomeric compound, terminal-group nucleosides are typically non-hybridizing. The terminal-group nucleosides are typically added to provide a desired property other than hybridization with target nucleic acid. Nonetheless, the target may have complementary bases at the positions corresponding with the terminal-group nucleosides. Whether by design or accident, such complementarity of one or more terminal-group nucleosides does not alter their designation as terminal-group nucleosides. In certain embodiments, the bases of terminal-group nucleosides are each selected from adenine (A), uracil (U), guanine (G), cytosine (C), thymine (T), and analogs thereof. In certain embodiments, the bases of terminal-group nucleosides are each selected from adenine (A), uracil (U), guanine (G), cytosine (C), and thymine (T). In certain embodiments, the bases of terminal-group nucleosides are each selected from adenine (A), uracil (U), and thymine (T). In certain embodiments, the bases of terminal-group nucleosides are each selected from adenine (A) and thymine (T). In certain embodiments, the bases of terminal-group nucleosides are each adenine (A). In certain embodiments, the bases of terminal-group nucleosides are each thymine (T). In certain embodiments, the bases of terminal-group nucleosides are each uracil (U). In certain embodiments, the bases of terminal-group nucleosides are each cytosine (C). In certain embodiments, the bases of terminal-group nucleosides are each guanine (G).

In certain embodiments, terminal-group nucleosides are sugar modified. In certain such embodiments, such additional nucleosides are 2′-modified. In certain embodiments, the 2′-modification of terminal-group nucleosides are selected from 2′-F, 2′-OMe, and 2′-MOE. In certain embodiments, terminal-group nucleosides are 2′-MOE modified. In certain embodiments, terminal-group nucleosides comprise 2′-MOE sugar moieties and adenine nucleobases (2′-MOE A nucleosides). In certain embodiments, terminal-group nucleosides comprise 2′-MOE sugar moieties and uracil nucleobases (2′-MOE U nucleosides). In certain embodiments, terminal-group nucleosides comprises 2′-MOE sugar moieties and guanine nucleobases (2′-MOE G nucleosides). In certain embodiments, terminal-group nucleosides comprises 2′-MOE sugar moieties and thymine nucleobases (2′-MOE T nucleosides). In certain embodiments, terminal-group nucleosides comprises 2′-MOE sugar moieties and cytosine nucleobases (2′-MOE C nucleosides).

In certain embodiments, terminal-group nucleosides comprise bicyclic sugar moieties. In certain such embodiments, terminal-group nucleosides comprise LNA sugar moieties. In certain embodiments, terminal-group nucleosides comprise LNA sugar moieties and adenine nucleobases (LNA A nucleosides). In certain embodiments, terminal-group nucleosides comprise LNA sugar moieties and uracil nucleobases (LNA nucleosides). In certain embodiments, terminal-group nucleosides comprise LNA sugar moieties and guanine nucleobases (LNA G nucleosides). In certain embodiments, terminal-group nucleosides comprise LNA sugar moieties and thymine nucleobases (LNA T nucleosides). In certain embodiments, terminal-group nucleosides comprise LNA sugar moieties and cytosine nucleobases (LNA C nucleosides).

In certain embodiments, oligomeric compounds comprise 1-4 terminal-group nucleosides at the 3′ end of the oligomeric compound. In certain embodiments, oligomeric compounds comprise 1-3 terminal-group nucleosides at the 3′ end of the oligomeric compound. In certain embodiments, oligomeric compounds comprise 1-2 terminal-group nucleosides at the 3′ end of the oligomeric compound. In certain embodiments, oligomeric compounds comprise 2 terminal-group nucleosides at the 3′ end of the oligomeric compound. In certain embodiments, oligomeric compounds comprise 1 terminal-group nucleoside at the 3′ end of the oligomeric compound. In certain embodiments having two or more terminal-group nucleosides, the two or more terminal-group nucleosides all have the same modification type and the same base. In certain embodiments having two or more terminal-group nucleosides, the terminal-group nucleosides differ from one another by modification and/or base.

In certain embodiments, oligomeric compounds comprise a 3′-terminal group comprising 2 terminal-group nucleosides, wherein each terminal group nucleoside is a 2′-MOE T. In certain embodiments, oligomeric compounds comprise a 3′-terminal group comprising 2 terminal-group nucleosides, wherein each terminal group nucleoside is a 2′-MOE A. In certain embodiments, oligomeric compounds comprise a 3′-terminal group comprising 2 terminal-group nucleosides, wherein each terminal group nucleoside is a 2′-MOE U. In certain embodiments, oligomeric compounds comprise a 3′-terminal group comprising 2 terminal-group nucleosides, wherein each terminal group nucleoside is a 2′-MOE C. In certain embodiments, oligomeric compounds comprise a 3′-terminal group comprising 2 terminal-group nucleosides, wherein each terminal group nucleoside is a 2′-MOE G.

In certain embodiments, oligomeric compounds comprise a 3′-terminal group comprising 2 terminal-group nucleosides, wherein each terminal group nucleoside is a LNA T. In certain embodiments, oligomeric compounds comprise a 3′-terminal group comprising 2 terminal-group nucleosides, wherein each terminal group nucleoside is a LNA A. In certain embodiments, oligomeric compounds comprise a 3′-terminal group comprising 2 terminal-group nucleosides, wherein each terminal group nucleoside is a LNA U. In certain embodiments, oligomeric compounds comprise a 3′-terminal group comprising 2 terminal-group nucleosides, wherein each terminal group nucleoside is a LNA C. In certain embodiments, oligomeric compounds comprise a 3′-terminal group comprising 2 terminal-group nucleosides, wherein each terminal group nucleoside is a LNA G.

### V. ANTISENSE COMPOUNDS

In certain embodiments, oligomeric compounds for use in the compositions of the present invention are antisense compounds. In such embodiments, the oligomeric compound is complementary to a target nucleic acid. In certain embodiments, a target nucleic acid is an RNA. In certain embodiments, a target nucleic acid is a non-coding RNA. In certain embodiments, a target nucleic acid encodes a protein. In certain embodiments, a target nucleic acid is selected from a mRNA, a pre-mRNA, a microRNA, a non-coding RNA, including small non-coding RNA, and a promoter-directed RNA. In certain embodiments, oligomeric compounds are at least partially complementary to more than one target nucleic acid. For example, oligomeric compounds of the present invention may be microRNA mimics, which typically bind to multiple targets.

Antisense mechanisms include any mechanism involving the hybridization of an oligomeric compound with target nucleic acid, wherein the hybridization results in a biological effect. In certain embodiments, such hybridization results in either target nucleic acid degradation or occupancy with concomitant inhibition or stimulation of the cellular machinery involving, for example, translation, transcription, or splicing of the target nucleic acid.

One type of antisense mechanism involving degradation of target RNA is RNase H mediated antisense. RNase H is a cellular endonuclease which cleaves the RNA strand of an RNA:DNA duplex. It is known in the art that single-stranded antisense compounds which are “DNA-like” elicit RNase H activity in mammalian cells. Activation of RNase H, therefore, results in cleavage of the RNA target, thereby greatly enhancing the efficiency of DNA-like oligonucleotide-mediated inhibition of gene expression.

Antisense mechanisms also include, without limitation RNAi mechanisms, which utilize the RISC pathway. Such RNAi mechanisms include, without limitation siRNA, ssRNA and microRNA mechanisms. Such mechanism include creation of a microRNA mimic and/or an anti-microRNA.

Antisense mechanisms also include, without limitation, mechanisms that hybridize or mimic non-coding RNA other than microRNA or mRNA. Such non-coding RNA includes, but is not limited to promoter-directed RNA and short and long RNA that effects transcription or translation of one or more nucleic acids.

In certain embodiments, antisense compounds specifically hybridize when there is a sufficient degree of complementarity to avoid non-specific binding of the antisense compound to non-target nucleic acid sequences under conditions in which specific binding is desired, i.e., under physiological conditions in the case of in vivo assays or therapeutic treatment, and under conditions in which assays are performed in the case of in vitro assays.

As used herein, “stringent hybridization conditions” or “stringent conditions” refers to conditions under which an antisense compound will hybridize to its target sequence, but to a minimal number of other sequences. Stringent conditions are sequence-dependent and will be different in different circumstances, and “stringent conditions” under which antisense compounds hybridize to a target sequence are determined by the nature and composition of the antisense compounds and the assays in which they are being investigated.

It is understood in the art that incorporation of nucleotide affinity modifications may allow for a greater number of mismatches compared to an unmodified compound. Similarly, certain oligonucleotide sequences may be more tolerant to mismatches than other oligonucleotide sequences. One of ordinary skill in the art is capable of determining an appropriate number of mismatches between oligonucleotides, or between an oligonucleotide and a target nucleic acid, such as by determining melting temperature (Tₘ). Tₘ or ΔTₘ can be calculated by techniques that are familiar to one of ordinary skill in the art. For example, techniques described in Freier et al. (Nucleic Acids Research, 1997, 25, 22: 4429-4443) allow one of ordinary skill in the art to evaluate nucleotide modifications for their ability to increase the melting temperature of an RNA:DNA duplex.

In certain embodiments, oligomeric compounds are RNAi compounds. In certain embodiments, oligomeric compounds are ssRNA compounds. In certain embodiments, oligomeric compounds are paired with a second oligomeric compound to form an siRNA. In certain such embodiments, the second oligomeric compound is also an oligomeric compound as described herein. In certain embodiments, the second oligomeric compound is any modified or unmodified nucleic acid. In certain embodiments, the oligomeric compound is the antisense strand in an siRNA compound. In certain embodiments, the oligomeric compound is the sense strand in an siRNA compound.

1. Single-Stranded Antisense Compounds

In certain embodiments, oligomeric compounds for use in the compositions of the present invention are particularly suited for use as single-stranded antisense compounds. In certain such embodiments, such oligomeric compounds are single-stranded RNAi compounds. In certain embodiments, such oligomeric compounds are ssRNA compounds or microRNA mimics. Certain 5′-terminal nucleosides described herein are suited for use in such single-stranded oligomeric compounds. In certain embodiments, such 5′-terminal nucleosides stabilize the 5′-phosphorous moiety. In certain embodiments, 5′-terminal nucleosides are resistant to nucleases. In certain embodiments, the motifs for use in the compositions of the present invention are particularly suited for use in single-stranded oligomeric compounds.

Use of single-stranded RNAi compounds has been limited. In certain instances, single stranded RNAi compounds are quickly degraded and/or do not load efficiently into RISC. In certain embodiments, the 5′-terminal phosphorous moiety of an oligomeric compound for use in the compositions of the present invention is stabilized. In certain such embodiments, the 5′-nucleoside is resistant to nuclease cleavage. In certain embodiments, the 5′-terminal end loads efficiently into RISC. In certain embodiments, the motif stabilizes the oligomeric compound. In certain embodiments the 3′-terminal end of the oligomeric compound is stabilized.

Design of single-stranded RNAi compounds for use in cells and/or for use in vivo presents several challenges. For example, the compound must be chemically stable, resistant to nuclease degradation, capable of entering cells, capable of loading into RISC (e.g., binding Ago1 or Ago2), capable of hybridizing with a target nucleic acid, and not toxic to cells or animals. In certain instances, a modification or motif that improves one such feature may worsen another feature, rendering a compound having such modification or motif unsuitable for use as an RNAi compound. For example, certain modifications, particularly if placed at or near the 5′-end of an oligomeric compound, may make the compound more stable and more resistant to nuclease degradation, but may also inhibit or prevent loading into RISC by blocking the interaction with RISC components, such as Ago1 or Ago2. Despite its improved stability properties, such a compound would be unsuitable for use in RNAi. Thus, the challenge is to identify modifications and combinations and placement of modifications that satisfy each parameter at least sufficient to provide a functional single-stranded RNAi compound. In certain embodiments, oligomeric compounds combine modifications to provide single-stranded RNAi compounds that are active as single-stranded RNAi compounds.

In certain instances, a single-stranded oligomeric compound comprising a 5′-phosphorous moiety is desired. For example, in certain embodiments, such 5′-phosphorous moiety is necessary or useful for RNAi compounds, particularly, single-stranded RNAi compounds. In such instances, it is further desirable to stabilize the phosphorous moiety against degradation or de-phosphorylation, which may inactivate the compound. Further, it is desirable to stabilize the entire 5′-nucleoside from degradation, which could also inactivate the compound. Thus, in certain embodiments, oligonucleotides in which the 5′-phosphorous moiety and the 5′-nucleoside have been stabilized are desired. In certain embodiments, the present invention incorporates modified nucleosides that may be placed at the 5′-end of an oligomeric compound, resulting in stabilized phosphorous and stabilized nucleoside. In certain such embodiments, the phosphorous moiety is resistant to removal in biological systems, relative to unmodified nucleosides and/or the 5′-nucleoside is resistant to cleavage by nucleases. In certain embodiments, such nucleosides are modified at one, at two or at all three of: the 2′-position, the 5′-position, and at the phosphorous moiety. Such modified nucleosides may be incorporated at the 5′-end of an oligomeric compound.

Although certain oligomeric compounds for use in the compositions of the present invention have particular use as single-stranded compounds, such compounds may also be paired with a second strand to create a double-stranded oligomeric compound. In such embodiments, the second strand of the double-stranded duplex may or may not also be an oligomeric compound as described herein.

In certain embodiments, oligomeric compounds for use in the compositions of the present invention bind and/or activate one or more nucleases. In certain embodiments, such binding and/or activation ultimately results in antisense activity. In certain embodiments, an oligomeric compound for use in the compositions of the invention interacts with a target nucleic acid and with a nuclease, resulting in activation of the nuclease and cleavage of the target nucleic acid. In certain embodiments, an oligomeric compound interacts with a target nucleic acid and with a nuclease, resulting in activation of the nuclease and inactivation of the target nucleic acid. In certain embodiments, an oligomeric compound forms a duplex with a target nucleic acid and that duplex activates a nuclease, resulting in cleavage and/or inactivation of one or both of the oligomeric compound and the target nucleic acid. In certain embodiments, an oligomeric compound binds and/or activates a nuclease and the bound and/or activated nuclease cleaves or inactivates a target nucleic acid. Nucleases include, but are not limited to, ribonucleases (nucleases that specifically cleave ribonucleotides), double-strand nucleases (nucleases that specifically cleave one or both strands of a double-stranded duplex), and double-strand ribonucleases. For example, nucleases include, but are not limited to RNase H, an argonaute protein (including, but not limited to Ago2), and dicer.

In certain embodiments, oligomeric compounds for use in the compositions of the present invention activate RNase H. RNase H is a cellular nuclease that cleaves the RNA strand of a duplex comprising an RNA strand and a DNA or DNA-like strand. In certain embodiments, an oligomeric compound for use in the compositions of the present invention is sufficiently DNA-like to activate RNase H, resulting in cleavage of an RNA nucleic acid target. In certain such embodiments, the oligomeric compound comprises at least one region comprised of DNA or DNA-like nucleosides and one or more regions comprised of nucleosides that are otherwise modified. In certain embodiments, such otherwise modified nucleosides increase stability of the oligomeric compound and/or its affinity for the target nucleic acid. Certain such oligomeric compounds posses a desirable combination of properties. For example, certain such compounds, by virtue of the DNA or DNA-like region, are able to support RNase H activity to cleave a target nucleic acid; and by virtue of the otherwise modified nucleosides, have enhanced affinity for the target nucleic acid and/or enhanced stability (including resistance to single-strand-specific nucleases). In certain embodiments, such otherwise modified nucleosides result in oligomeric compounds having desired properties, such as metabolic profile and/or pharmacologic profile.

In certain embodiments, oligomeric compounds for use in the compositions of the present invention interact with an argonaute protein (Ago). In certain embodiments, such oligomeric compounds first enter the RISC pathway by interacting with another member of the pathway (e.g., dicer). In certain embodiments, oligomeric compounds first enter the RISC pathway by interacting with Ago. In certain embodiments, such interaction ultimately results in antisense activity. In certain embodiments, the invention provides methods of activating Ago comprising contacting a cell with a composition of the present invention. In certain embodiments, such composition comprises an oligomeric compound comprising a modified 5′-phosphate group. In certain embodiments, the invention provides methods of modulating the expression or amount of a target nucleic acid in a cell comprising contacting the cell with a composition comprising an oligomeric compound capable of activating Ago, ultimately resulting in cleavage of the target nucleic acid. In certain embodiments, the cell is in an animal. In certain embodiments, the cell is in vitro. In certain embodiments, the methods are performed in the presence of manganese. In certain embodiments, the manganese is endogenous. In certain embodiment the methods are performed in the absence of magnesium. In certain embodiments, the Ago is endogenous to the cell. In certain such embodiments, the cell is in an animal. In certain embodiments, the Ago is human Ago. In certain embodiments, the Ago is Ago2. In certain embodiments, the Ago is human Ago2.

In certain embodiments, oligomeric compounds for use in the compositions of the present invention interact with the enzyme dicer. In certain such embodiments, oligomeric compounds bind to dicer and/or are cleaved by dicer. In certain such embodiments, such interaction with dicer ultimately results in antisense activity. In certain embodiments, the dicer is human dicer. In certain embodiments, oligomeric compounds that interact with dicer are double-stranded oligomeric compounds. In certain embodiments, oligomeric compounds that interact with dicer are single-stranded oligomeric compounds.

In embodiments in which a double-stranded oligomeric compound interacts with dicer, such double-stranded oligomeric compound forms a dicer duplex. In certain embodiments, any oligomeric compound described herein may be suitable as one or both strands of a dicer duplex. In certain embodiments, each strand of the dicer duplex is an oligomeric compound as described herein. In certain embodiments, one strand of the dicer duplex is an oligomeric compound as described herein and the other strand is any modified or unmodified oligomeric compound. In certain embodiments, one or both strands of a dicer duplex comprises a nucleoside of Formula I or II at the 5′ end. In certain embodiments, one strand of a dicer duplex is an antisense oligomeric compound and the other strand is its sense complement.

In certain embodiments, the dicer duplex comprises a 3′-overhang at one or both ends. In certain embodiments, such overhangs are additional nucleosides. In certain embodiments, the dicer duplex comprises a 3′ overhang on the sense oligonucleotide and not on the antisense oligonucleotide. In certain embodiments, the dicer duplex comprises a 3′ overhang on the antisense oligonucleotide and not on the sense oligonucleotide. In certain embodiments, 3′ overhangs of a dicer duplex comprise 1-4 nucleosides. In certain embodiments, such overhangs comprise two nucleosides. In certain embodiments, the nucleosides in the 3′-overhangs comprise purine nucleobases. In certain embodiments, the nucleosides in the 3′ overhangs comprise adenine nucleobases. In certain embodiments, the nucleosides in the 3′ overhangs comprise pyrimidines. In certain embodiments, dicer duplexes comprising 3′-purine overhangs are more active as antisense compounds than dicer duplexes comprising 3′ pyrimidine overhangs. In certain embodiments, oligomeric compounds of a dicer duplex comprise one or more 3′ deoxy nucleosides. In certain such embodiments, the 3′ deoxy nucleosides are dT nucleosides.

In certain embodiments, the 5′ end of each strand of a dicer duplex comprises a phosphate moiety. In certain embodiments the antisense strand of a dicer duplex comprises a phosphate moiety and the sense strand of the dicer duplex does not comprise a phosphate moiety. In certain embodiments the sense strand of a dicer duplex comprises a phosphate moiety and the antisense strand of the dicer duplex does not comprise a phosphate moiety. In certain embodiments, a dicer duplex does not comprise a phosphate moiety at the 3′ end. In certain embodiments, a dicer duplex is cleaved by dicer. In such embodiments, dicer duplexes do not comprise 2′-OMe modifications on the nucleosides at the cleavage site. In certain embodiments, such cleavage site nucleosides are RNA.

In certain embodiments, interaction of an oligomeric compound with dicer ultimately results in antisense activity. In certain embodiments, dicer cleaves one or both strands of a double-stranded oligomeric compound and the resulting product enters the RISC pathway, ultimately resulting in antisense activity. In certain embodiments, dicer does not cleave either strand of a double-stranded oligomeric compound, but nevertheless facilitates entry into the RISC pathway and ultimately results in antisense activity. In certain embodiments, dicer cleaves a single-stranded oligomeric compound and the resulting product enters the RISC pathway, ultimately resulting in antisense activity. In certain embodiments, dicer does not cleave the single-stranded oligomeric compound, but nevertheless facilitates entry into the RISC pathway and ultimately results in antisense activity.

In certain embodiments, the invention provides methods of activating dicer comprising contacting a cell with a composition of the present invention. In certain such embodiments, the cell is in an animal.

Dicer

In certain embodiments, oligomeric compounds for use in the compositions of the present invention interact with the enzyme dicer. In certain such embodiments, oligomeric compounds bind to dicer and/or are cleaved by dicer. In certain such embodiments, such interaction with dicer ultimately results in antisense activity. In certain embodiments, the dicer is human dicer. In certain embodiments, oligomeric compounds that interact with dicer are double-stranded oligomeric compounds. In certain embodiments, oligomeric compounds that interact with dicer are single-stranded oligomeric compounds.

In embodiments in which a double-stranded oligomeric compound interacts with dicer, such double-stranded oligomeric compound forms a dicer duplex. In certain embodiments, any oligomeric compound described herein may be suitable as one or both strands of a dicer duplex. In certain embodiments, each strand of the dicer duplex is an oligomeric compound as described herein. In certain embodiments, one strand of the dicer duplex is an oligomeric compound as described herein and the other strand is any modified or unmodified oligomeric compound. In certain embodiments, one or both strands of a dicer duplex comprises a nucleoside of Formula I or II at the 5′. In certain embodiments, one strand of a dicer duplex is an antisense oligomeric compound and the other strand is its sense complement.

In certain embodiments, a dicer duplex comprises a first and second oligomeric compound wherein each oligomeric compound comprises an oligonucleotide consisting of 25 to 30 linked nucleosides. In certain such embodiments, each oligonucleotide of the dicer duplex consists of 27 linked nucleosides.

In certain embodiments, the dicer duplex comprises a 3′-overhang at one or both ends. In certain embodiments, such overhangs are additional nucleosides. In certain embodiments, the dicer duplex comprises a 3′ overhang on the sense oligonucleotide and not on the antisense oligonucleotide. In certain embodiments, the dicer duplex comprises a 3′ overhang on the antisense oligonucleotide and not on the sense oligonucleotide. In certain embodiments, 3′ overhangs of a dicer duplex comprise 1-4 nucleosides. In certain embodiments, such overhangs comprise two nucleosides. In certain embodiments, 3′-overhangs comprise purine nucleobases. In certain embodiments, 3′-overhangs comprise adenine overhangs. In certain embodiments, 3′-overhangs are pyrimidines. In certain embodiments, dicer duplexes comprising 3′-purine overhangs are more active as antisense compounds than dicer duplexes comprising 3′-pyrimidine overhangs. In certain embodiments, oligomeric compounds of a dicer duplex comprise 3′-deoxy nucleosides. In certain such embodiments, the 3′-deoxy nucleosides are dT nucleosides.

In certain embodiments, the 5′ end of each strand of a dicer duplex comprises phosphate moiety. In certain embodiments the antisense strand of a dicer duplex comprises a phosphate moiety and the sense strand of the dicer duplex does not comprises a phosphate moiety. In certain embodiments the sense strand of a dicer duplex comprises a phosphate moiety and the antisense strand of the dicer duplex does not comprises a phosphate moiety. In certain embodiments, a dicer duplex does not comprise a phosphate moiety at the 3′-end. In certain embodiments, a dicer duplex is cleaved by dicer. In such embodiments, dicer duplexes do not comprise 2′-OMe modifications at the nucleosides at the cleavage site. In certain embodiments, such cleavage site nucleosides are RNA.

One of skill will appreciate that the above described features of dicer duplexes may be combined. For example, in certain embodiments, a dicer duplex comprises a first oligomeric compound comprising an antisense oligonucleotide and a second oligomeric compound comprising a sense oligonucleotide; wherein the sense oligonucleotide comprises a 3′ overhang consisting of two purine nucleosides and the antisense oligonucleotide comprises a 3′ overhang consisting of two adenosine or modified adenosine nucleosides; each of the sense and antisense oligonucleotides consists of 25 to 30 linked nucleosides, the 5′ end of the antisense oligonucleotide comprises a phosphorous moiety, and wherein the dicer cleavage sites of the dicer duplex are not O-Me modified nucleosides.

In certain embodiments, the invention provides compositions comprising single-stranded oligomeric compounds that interact with dicer. In certain embodiments, such single-stranded dicer compounds comprise a nucleoside of Formula I or II. In certain embodiments, single-stranded dicer compounds do not comprise a phosphorous moiety at the 3′-end. In certain embodiments, such single-stranded dicer compounds may comprise a 3′-overhangs. In certain embodiments, such 3′-overhangs are additional nucleosides. In certain embodiments, such 3′-overhangs comprise 1-4 additional nucleosides that are not complementary to a target nucleic acid and/or are differently modified from the adjacent 3′ nucleoside of the oligomeric compound. In certain embodiments, a single-stranded oligomeric compound comprises an antisense oligonucleotide having two 3′-end overhang nucleosides wherein the overhang nucleosides are adenine or modified adenine nucleosides. In certain embodiments, single stranded oligomeric compounds that interact with dicer comprise a nucleoside of Formula I or II.

In certain embodiments, interaction of an oligomeric compound with dicer ultimately results in antisense activity. In certain embodiments, dicer cleaves one or both strands of a double-stranded oligomeric compound and the resulting product enters the RISC pathway, ultimately resulting in antisense activity. In certain embodiments, dicer does not cleave either strand of a double-stranded oligomeric compound, but nevertheless facilitates entry into the RISC pathway and ultimately results in antisense activity. In certain embodiments, dicer cleaves a single-stranded oligomeric compound and the resulting product enters the RISC pathway, ultimately resulting in antisense activity. In certain embodiments, dicer does not cleave the single-stranded oligomeric compound, but nevertheless facilitates entry into the RISC pathway and ultimately results in antisense activity.

In certain embodiments, the invention provides methods of activating dicer comprising contacting a cell with a composition of the present invention. In certain such embodiments, the cell is in an animal.

Ago

In certain embodiments, oligomeric compounds for use in the compositions of the present invention interact with Ago. In certain embodiments, such oligomeric compounds first enter the RISC pathway by interacting with another member of the pathway (e.g., dicer). In certain embodiments, oligomeric compounds first enter the RISC pathway by interacting with Ago. In certain embodiments, such interaction ultimately results in antisense activity. In certain embodiments, the invention provides methods of activating Ago comprising contacting a cell with a composition of the present invention. In certain such embodiments, the cell is in an animal.

2. Oligomeric Compound Identity

In certain embodiments, a portion of an oligomeric compound is 100% identical to the nucleobase sequence of a microRNA, but the entire oligomeric compound is not fully identical to the microRNA. In certain such embodiments, the length of an oligomeric compound having a 100% identical portion is greater than the length of the microRNA. For example, a microRNA mimic consisting of 24 linked nucleosides, where the nucleobases at positions 1 through 23 are each identical to corresponding positions of a microRNA that is 23 nucleobases in length, has a 23 nucleoside portion that is 100% identical to the nucleobase sequence of the microRNA and has approximately 96% overall identity to the nucleobase sequence of the microRNA.

In certain embodiments, the nucleobase sequence of oligomeric compound is fully identical to the nucleobase sequence of a portion of a microRNA. For example, a single-stranded microRNA mimic consisting of 22 linked nucleosides, where the nucleobases of positions 1 through 22 are each identical to a corresponding position of a microRNA that is 23 nucleobases in length, is fully identical to a 22 nucleobase portion of the nucleobase sequence of the microRNA. Such a single-stranded microRNA mimic has approximately 96% overall identity to the nucleobase sequence of the entire microRNA, and has 100% identity to a 22 nucleobase portion of the microRNA.

E. Synthesis, Purification and Analysis

Oligomerization of modified and unmodified nucleosides and nucleotides can be routinely performed according to literature procedures for DNA (Protocols for Oligonucleotides and Analogs, Ed. Agrawal (1993), Humana Press) and/or RNA (Scaringe, Methods (2001), 23, 206-217. Gait et al., Applications of Chemically synthesized RNA in RNA: Protein Interactions, Ed. Smith (1998), 1-36. Gallo et al., Tetrahedron (2001), 57, 5707-5713).

Oligomeric compounds provided herein can be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives. The invention is not limited by the method of antisense compound synthesis.

Methods of purification and analysis of oligomeric compounds are known to those skilled in the art. Analysis methods include capillary electrophoresis (CE) and electrospray-mass spectroscopy. Such synthesis and analysis methods can be performed in multi-well plates. The method of the invention is not limited by the method of oligomer purification.

F. Nucleic Acid Lipid Particle

In one embodiment, an ssRNA featured in the invention is fully encapsulated in the lipid formulation, e.g., to form a nucleic acid-lipid particle, e.g., Nucleic acid-lipid particles typically contain a cationic lipid, a non-cationic lipid, a sterol, and a lipid that prevents aggregation of the particle (e.g., a PEG-lipid conjugate). Nucleic acid-lipid particles are extremely useful for systemic applications, as they exhibit extended circulation lifetimes following intravenous (i.v.) injection and accumulate at distal sites (e.g., sites physically separated from the administration site). In addition, the nucleic acids when present in the nucleic acid-lipid particles of the present invention are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; and PCT Publication No. WO 96/40964.

Nucleic acid-lipid particles can further include one or more additional lipids and/or other components such as cholesterol. Other lipids may be included in the liposome compositions for a variety of purposes, such as to prevent lipid oxidation or to attach ligands onto the liposome surface. Any of a number of lipids may be present, including amphipathic, neutral, cationic, and anionic lipids. Such lipids can be used alone or in combination. Specific examples of additional lipid components that may be present are described herein.

Additional components that may be present in a nucleic acid-lipid particle include bilayer stabilizing components such as polyamide oligomers (see, e.g., U.S. Pat. No. 6,320,017), peptides, proteins, detergents, lipid-derivatives, such as PEG coupled to phosphatidylethanolamine and PEG conjugated to ceramides (see, U.S. Pat. No. 5,885,613).

A nucleic acid-lipid particle can include one or more of a second amino lipid or cationic lipid, a neutral lipid, a sterol, and a lipid selected to reduce aggregation of lipid particles during formation, which may result from steric stabilization of particles which prevents charge-induced aggregation during formation.

Nucleic acid-lipid particles include, e.g., a SPLP, pSPLP, and SNALP. The term“SNALP” refers to a stable nucleic acid-lipid particle, including SPLP. The term “SPLP” refers to a nucleic acid-lipid particle comprising plasmid DNA encapsulated within a lipid vesicle. SPLPs include “pSPLP,” which include an encapsulated condensing agent-nucleic acid complex as set forth in PCT Publication No. WO 00/03683.

The particles of the present invention typically have a mean diameter of about 50 nm to about 150 nm, more typically about 60 nm to about 130 nm, more typically about 70 nm to about 110 nm, most typically about 70 nm to about 90 nm, and are substantially nontoxic

In one embodiment, the lipid to drug ratio (mass/mass ratio) (e.g., lipid to ssRNA ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1, or about 6:1, 7:1, 8:1, 9:1, 10:1, 11:1, 12:1, or 33:1.

Cationic Lipids

The nucleic acid-lipid particles of the invention typically include a cationic lipid. The cationic lipid may be, for example, N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.C1), 1,2-Dilinoleoyl-3-trimethylaminopropane chloride salt (DLin-TAP.C1), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl-4-(dimethylamino)butanoate, or a mixture thereof. Synthesis of these lipids are known in the art or are described, e.g., in U.S. Provisional Ser. No. 61/244,834, filed Sep. 22, 2009, and U.S. Provisional Ser. No. 61/185,800, filed Jun. 10, 2009, application number PCT/US09/63933 filed on Nov. 10, 2009, which is herein incorporated by reference.

Other cationic lipids, which carry a net positive charge at about physiological pH, in addition to those specifically described above, may also be included in lipid particles of the invention. Such cationic lipids include, but are not limited to, N,N-dioleyl-N,N-dimethylammonium chloride (“DODAC”); N-(2,3-dioleyloxy)propyl-N,N-N-triethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 1,2-Dioleyloxy-3-trimethylaminopropane chloride salt (“DOTAP.Cl”); 3β-(N—(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethylammonium trifluoracetate (“DOSPA”), dioctadecylamidoglycyl carboxyspermine (“DOGS”), 1,2-dileoyl-sn-3-phosphoethanolamine (“DOPE”), 1,2-dioleoyl-3-dimethylammonium propane (“DODAP”), N,N-dimethyl-2,3-dioleyloxy)propylamine (“DODMA”), and N-(1,2-dimyristyloxyprop-3-yl)-N,N-dimethyl-N-hydroxyethyl ammonium bromide (“DMRIE”). Additionally, a number of commercial preparations of cationic lipids can be used, such as, e.g., LIPOFECTIN (including DOTMA and DOPE, available from GIBCO/BRL), and LIPOFECTAMINE (comprising DOSPA and DOPE, available from GIBCO/BRL). In particular embodiments, a cationic lipid is an amino lipid.

As used herein, the term “amino lipid” is meant to include those lipids having one or two fatty acid or fatty alkyl chains and an amino head group (including an alkylamino or dialkylamino group) that may be protonated to form a cationic lipid at physiological pH.

Other amino lipids would include those having alternative fatty acid groups and other dialkylamino groups, including those in which the alkyl substituents are different (e.g., N-ethyl-N-methylamino-, N-propyl-N-ethylamino- and the like). In general, amino lipids having less saturated acyl chains are more easily sized, particularly when the complexes must be sized below about 0.3 microns, for purposes of filter sterilization. Amino lipids containing unsaturated fatty acids with carbon chain lengths in the range of C₁₄ to C₂₂ are preferred. Other scaffolds can also be used to separate the amino group and the fatty acid or fatty alkyl portion of the amino lipid. Suitable scaffolds are known to those of skill in the art.

In certain embodiments, the cationic lipid of the invention cationic lipid comprises formula A, wherein formula A is

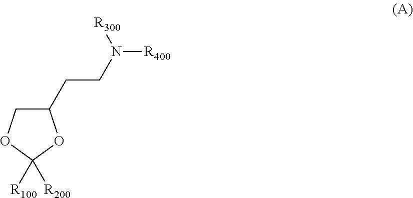

where R₁₀₀ and R₂₀₀ are independently alkyl, alkenyl or alkynyl, each can be optionally substituted, and R₃₀₀ and R₄₀₀ are independently lower alkyl or R₃₀₀ and R₄₀₀ can be taken together to form an optionally substituted heterocyclic ring.

In one embodiment, the cationic lipid comprises 2,2-Dilinoleyl-4-dimethylaminoethyl-[1,3]-dioxolane, the non-cationic lipid comprises DSPC, the sterol comprises cholesterol and the PEG lipid comprises PEG-DMG.

In one embodiment, representative nucleic acid lipid particles include, but not limited to,

| | |
|---|---|
| LNP05 | Cationic lipid/DSPC/Cholesterol/PEG-DMG |
| | 57.5/7.5/31.5/3.5 |
| | lipid:siRNA ~6:1 |
| LNP06 | Cationic lipid/DSPC/Cholesterol/PEG-DMG |
| | 57.5/7.5/31.5/3.5 |
| | lipid:siRNA ~11:1 |
| LNP07 | Cationic lipid/DSPC/Cholesterol/PEG-DMG |
| | 60/7.5/31/1.5, |
| | lipid:siRNA ~6:1 |
| LNP08 | Cationic lipid/DSPC/Cholesterol/PEG-DMG |
| | 60/7.5/31/1.5, |
| | lipid:siRNA ~11:1 |
| LNP09 | Cationic lipid/DSPC/Cholesterol/PEG-DMG |
| | 50/10/38.5/1.5 |
| | lipid:siRNA ~10:1 |
| LNP13 | Cationic lipid/DSPC/Cholesterol/PEG-DMG |
| | 50/10/38.5/1.5 |
| | lipid:siRNA ~33:1 |
| LNP22 | Cationic lipid/DSPC/Cholesterol/PEG-DSG |
| | 50/10/38.5/1.5 |
| | lipid:siRNA ~10. |

wherein the cationic lipid comprises 2,2-Dilinoleyl-4-dimethylaminoethyl-[1,3]-dioxolane.

In certain embodiments, amino or cationic lipids of the invention have at least one protonatable or deprotonatable group, such that the lipid is positively charged at a pH at or below physiological pH (e.g. pH 7.4), and neutral at a second pH, preferably at or above physiological pH. It will, of course, be understood that the addition or removal of protons as a function of pH is an equilibrium process, and that the reference to a charged or a neutral lipid refers to the nature of the predominant species and does not require that all of the lipid be present in the charged or neutral form. Lipids that have more than one protonatable or deprotonatable group, or which are zwiterrionic, are not excluded from use in the invention.

In certain embodiments, protonatable lipids according to the invention have a pKa of the protonatable group in the range of about 4 to about 11. Most preferred is pKa of about 4 to about 7, because these lipids will be cationic at a lower pH formulation stage, while particles will be largely (though not completely) surface neutralized at physiological pH around pH 7.4. One of the benefits of this pKa is that at least some nucleic acid associated with the outside surface of the particle will lose its electrostatic interaction at physiological pH and be removed by simple dialysis; thus greatly reducing the particle's susceptibility to clearance.

One example of a cationic lipid is 1,2-Dilinolenyloxy-N,N-dimethylaminopropane (DLinDMA). Synthesis and preparation of nucleic acid-lipid particles including DlinDMA is described in International application number PCT/CA2009/00496, filed Apr. 15, 2009.

In one embodiment, the cationic lipid is 2,2-Dilinoleyl-4-dimethylaminoethyl-[1,3]-dioxolane is used to prepare nucleic acid-lipid particles. Synthesis of 2,2-Dilinoleyl-4-dimethylaminoethyl-[1,3]-dioxolane is described in U.S. provisional patent application No. 61/107,998 filed on Oct. 23, 2008, which is herein incorporated by reference.

The cationic lipid may comprise from about 20 mol % to about 70 mol % or about 45-65 mol % or about 40 mol % of the total lipid present in the particle.

Non-Cationic Lipids

The nucleic acid-lipid particles of the invention can include a non-cationic lipid. The non-cationic lipid may be an anionic lipid or a neutral lipid. Examples include but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof.

Anionic lipids suitable for use in lipid particles of the invention include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids.

Neutral lipids, when present in the lipid particle, can be any of a number of lipid species which exist either in an uncharged or neutral zwitterionic form at physiological pH. Such lipids include, for example diacylphosphatidylcholine, diacylphosphatidylethanolamine, ceramide, sphingomyelin, dihydrosphingomyelin, cephalin, and cerebrosides. The selection of neutral lipids for use in the particles described herein is generally guided by consideration of, e.g., liposome size and stability of the liposomes in the bloodstream. Preferably, the neutral lipid component is a lipid having two acyl groups, (i.e., diacylphosphatidylcholine and diacylphosphatidylethanolamine). Lipids having a variety of acyl chain groups of varying chain length and degree of saturation are available or may be isolated or synthesized by well-known techniques. In one group of embodiments, lipids containing saturated fatty acids with carbon chain lengths in the range of C₁₄ to C₂₂ are preferred. In another group of embodiments, lipids with mono- or di-unsaturated fatty acids with carbon chain lengths in the range of C₁₄ to C₂₂ are used. Additionally, lipids having mixtures of saturated and unsaturated fatty acid chains can be used. Preferably, the neutral lipids used in the invention are DOPE, DSPC, POPC, or any related phosphatidylcholine. The neutral lipids useful in the invention may also be composed of sphingomyelin, dihydrosphingomyeline, or phospholipids with other head groups, such as serine and inositol.

In one embodiment the non-cationic lipid is distearoylphosphatidylcholine (DSPC). In another embodiment the non-cationic lipid is dipalmitoylphosphatidylcholine (DPPC).

The non-cationic lipid may be from about 5 mol % to about 90 mol %, about 5 mol % to about 10 mol %, about 10 mol %, or about 58 mol % if cholesterol is included, of the total lipid present in the particle.

Conjugated Lipids

Conjugated lipids can be used in nucleic acid-lipid particle to prevent aggregation, including polyethylene glycol (PEG)-modified lipids, monosialoganglioside Gm1, and polyamide oligomers (“PAO”) such as (described in U.S. Pat. No. 6,320,017). Other compounds with uncharged, hydrophilic, steric-barrier moieties, which prevent aggregation during formulation, like PEG, Gm1 or ATTA, can also be coupled to lipids for use as in the methods and compositions of the invention. ATTA-lipids are described, e.g., in U.S. Pat. No. 6,320,017, and PEG-lipid conjugates are described, e.g., in U.S. Pat. Nos. 5,820,873, 5,534,499 and 5,885,613. Typically, the concentration of the lipid component selected to reduce aggregation is about 1 to 15% (by mole percent of lipids).

Specific examples of PEG-modified lipids (or lipid-polyoxyethylene conjugates) that are useful in the invention can have a variety of “anchoring” lipid portions to secure the PEG portion to the surface of the lipid vesicle. Examples of suitable PEG-modified lipids include PEG-modified phosphatidylethanolamine and phosphatidic acid, PEG-ceramide conjugates (e.g., PEG-CerC14 or PEG-CerC20) which are described in co-pending U.S. Ser. No. 08/486,214, incorporated herein by reference, PEG-modified dialkylamines and PEG-modified 1,2-diacyloxypropan-3-amines. Particularly preferred are PEG-modified diacylglycerols and dialkylglycerols.

In embodiments where a sterically-large moiety such as PEG or ATTA are conjugated to a lipid anchor, the selection of the lipid anchor depends on what type of association the conjugate is to have with the lipid particle. It is well known that mePEG (mw2000)-diastearoylphosphatidylethanolamine (PEG-DSPE) will remain associated with a liposome until the particle is cleared from the circulation, possibly a matter of days. Other conjugates, such as PEG-CerC20 have similar staying capacity. PEG-CerC14, however, rapidly exchanges out of the formulation upon exposure to serum, with a T_{1/2} less than 60 mins. in some assays. As illustrated in U.S. patent application Ser. No. 08/486,214, at least three characteristics influence the rate of exchange: length of acyl chain, saturation of acyl chain, and size of the steric-barrier head group. Compounds having suitable variations of these features may be useful for the invention. For some therapeutic applications, it may be preferable for the PEG-modified lipid to be rapidly lost from the nucleic acid-lipid particle in vivo and hence the PEG-modified lipid will possess relatively short lipid anchors. In other therapeutic applications, it may be preferable for the nucleic acid-lipid particle to exhibit a longer plasma circulation lifetime and hence the PEG-modified lipid will possess relatively longer lipid anchors. Exemplary lipid anchors include those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons.

It should be noted that aggregation preventing compounds do not necessarily require lipid conjugation to function properly. Free PEG or free ATTA in solution may be sufficient to prevent aggregation. If the particles are stable after formulation, the PEG or ATTA can be dialyzed away before administration to a subject.

The conjugated lipid that inhibits aggregation of particles may be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate may be, for example, a PEG-dilauryloxypropyl (Ci₂), a PEG-dimyristyloxypropyl (Ci₄), a PEG-dipalmityloxypropyl (Ci₆), or a PEG-distearyloxypropyl (Ci₈). Additional conjugated lipids include polyethylene glycol-didimyristoyl glycerol (C14-PEG or PEG-C14, where PEG has an average molecular weight of 2000 Da) (PEG-DMG); (R)-2,3-bis(octadecyloxy)propyl1-(methoxy poly(ethylene glycol)2000)propylcarbamate) (PEG-DSG); PEG-carbamoyl-1,2-dimyristyloxypropylamine, in which PEG has an average molecular weight of 2000 Da (PEG-cDMA); N-Acetylgalactosamine-((R)-2,3-bis(octadecyloxy)propyl1-(methoxy poly(ethylene glycol)2000)propylcarbamate)) (GalNAc-PEG-DSG); and polyethylene glycol-dipalmitoylglycerol (PEG-DPG).

In one embodiment the conjugated lipid is PEG-DMG. In another embodiment the conjugated lipid is PEG-cDMA. In still another embodiment the conjugated lipid is PEG-DPG. Alternatively the conjugated lipid is GalNAc-PEG-DSG.

The conjugated lipid that prevents aggregation of particles may be from 0 mol % to about 20 mol % or about 0.5 to about 5.0 mol % or about 2 mol % of the total lipid present in the particle.

The sterol component of the lipid mixture, when present, can be any of those sterols conventionally used in the field of liposome, lipid vesicle or lipid particle preparation. A preferred sterol is cholesterol.

In some embodiments, the nucleic acid-lipid particle further includes a sterol, e.g., a cholesterol at, e.g., about 10 mol % to about 60 mol % or about 25 to about 40 mol % or about 48 mol % of the total lipid present in the particle.

Lipoproteins

In one embodiment, the formulations of the invention further comprise an apolipoprotein. As used herein, the term “apolipoprotein” or “lipoprotein” refers to apolipoproteins known to those of skill in the art and variants and fragments thereof and to apolipoprotein agonists, analogues or fragments thereof described below.

Suitable apolipoproteins include, but are not limited to, ApoA-I, ApoA-II, ApoA-IV, ApoA-V and ApoE, and active polymorphic forms, isoforms, variants and mutants as well as fragments or truncated forms thereof. In certain embodiments, the apolipoprotein is a thiol containing apolipoprotein. “Thiol containing apolipoprotein” refers to an apolipoprotein, variant, fragment or isoform that contains at least one cysteine residue. The most common thiol containing apolipoproteins are ApoA-I Milano (ApoA-I_{M}) and ApoA-I Paris (ApoA-I_{P}) which contain one cysteine residue (Jia et al., 2002, Biochem. Biophys. Res. Comm. 297: 206-13; Bielicki and Oda, 2002, Biochemistry 41: 2089-96). ApoA-II, ApoE2 and ApoE3 are also thiol containing apolipoproteins. Isolated ApoE and/or active fragments and polypeptide analogues thereof, including recombinantly produced forms thereof, are described in U.S. Pat. Nos. 5,672,685; 5,525,472; 5,473,039; 5,182,364; 5,177,189; 5,168,045; 5,116,739; the disclosures of which are herein incorporated by reference. ApoE3 is disclosed in Weisgraber, et al., “Human E apoprotein heterogeneity: cysteine-arginine interchanges in the amino acid sequence of the apo-E isoforms,” J. Biol. Chem. (1981) 256: 9077-9083; and Rall, et al., “Structural basis for receptor binding heterogeneity of apolipoprotein E from type III hyperlipoproteinemic subjects,” Proc. Nat. Acad. Sci. (1982) 79: 4696-4700. (See also GenBank accession number K00396.)

In certain embodiments, the apolipoprotein can be in its mature form, in its preproapolipoprotein form or in its proapolipoprotein form. Homo- and heterodimers (where feasible) of pro- and mature ApoA-I (Duverger et al., 1996, Arterioscler. Thromb. Vasc. Biol. 16(12):1424-29), ApoA-I Milano (Kion et al., 2000, Biophys. J. 79:(3)1679-87; Franceschini et al., 1985, J. Biol. Chem. 260: 1632-35), ApoA-I Paris (Daum et al., 1999, J. Mol. Med. 77:614-22), ApoA-II (Shelness et al., 1985, J. Biol. Chem. 260(14):8637-46; Shelness et al., 1984, J. Biol. Chem. 259(15):9929-35), ApoA-IV (Duverger et al., 1991, Euro. J. Biochem. 201(2):373-83), and ApoE (McLean et al., 1983, J. Biol. Chem. 258(14):8993-9000) can also be utilized within the scope of the invention.

In certain embodiments, the apolipoprotein can be a fragment, variant or isoform of the apolipoprotein. The term “fragment” refers to any apolipoprotein having an amino acid sequence shorter than that of a native apolipoprotein and which fragment retains the activity of native apolipoprotein, including lipid binding properties. By “variant” is meant substitutions or alterations in the amino acid sequences of the apolipoprotein, which substitutions or alterations, e.g., additions and deletions of amino acid residues, do not abolish the activity of native apolipoprotein, including lipid binding properties. Thus, a variant can comprise a protein or peptide having a substantially identical amino acid sequence to a native apolipoprotein provided herein in which one or more amino acid residues have been conservatively substituted with chemically similar amino acids. Examples of conservative substitutions include the substitution of at least one hydrophobic residue such as isoleucine, valine, leucine or methionine for another. Likewise, the present invention contemplates, for example, the substitution of at least one hydrophilic residue such as, for example, between arginine and lysine, between glutamine and asparagine, and between glycine and serine (see U.S. Pat. Nos. 6,004,925, 6,037,323 and 6,046,166). The term “isoform” refers to a protein having the same, greater or partial function and similar, identical or partial sequence, and may or may not be the product of the same gene and usually tissue specific (see Weisgraber 1990, J. Lipid Res. 31(8):1503-11; Hixson and Powers 1991, J. Lipid Res. 32(9):1529-35; Lackner et al., 1985, J. Biol. Chem. 260(2):703-6; Hoeg et al., 1986, J. Biol. Chem. 261(9):3911-4; Gordon et al., 1984, J. Biol. Chem. 259(1):468-74; Powell et al., 1987, Cell 50(6):831-40; Aviram et al., 1998, Arterioscler. Thromb. Vase. Biol. 18(10):1617-24; Aviram et al., 1998, J. Clin. Invest. 101(8):1581-90; Billecke et al., 2000, Drug Metab. Dispos. 28(11):1335-42; Draganov et al., 2000, J. Biol. Chem. 275(43):33435-42; Steinmetz and Utermann 1985, J. Biol. Chem. 260(4):2258-64; Widler et al., 1980, J. Biol. Chem. 255(21):10464-71; Dyer et al., 1995, J. Lipid Res. 36(1):80-8; Sacre et al., 2003, FEBS Lett. 540(1-3):181-7; Weers, et al., 2003, Biophys. Chem. 100(1-3):481-92; Gong et al., 2002, J. Biol. Chem. 277(33):29919-26; Ohta et al., 1984, J. Biol. Chem. 259(23):14888-93 and U.S. Pat. No. 6,372,886).

In certain embodiments, the methods and compositions of the present invention include the use of a chimeric construction of an apolipoprotein. For example, a chimeric construction of an apolipoprotein can be comprised of an apolipoprotein domain with high lipid binding capacity associated with an apolipoprotein domain containing ischemia reperfusion protective properties. A chimeric construction of an apolipoprotein can be a construction that includes separate regions within an apolipoprotein (i.e., homologous construction) or a chimeric construction can be a construction that includes separate regions between different apolipoproteins (i.e., heterologous constructions). Compositions comprising a chimeric construction can also include segments that are apolipoprotein variants or segments designed to have a specific character (e.g., lipid binding, receptor binding, enzymatic, enzyme activating, antioxidant or reduction-oxidation property) (see Weisgraber 1990, J. Lipid Res. 31(8):1503-11; Hixson and Powers 1991, J. Lipid Res. 32(9):1529-35; Lackner et al., 1985, J. Biol. Chem. 260(2):703-6; Hoeg et al., 1986, J. Biol. Chem. 261(9):3911-4; Gordon et al., 1984, J. Biol. Chem. 259(1):468-74; Powell et al., 1987, Cell 50(6):831-40; Aviram et al., 1998, Arterioscler. Thromb. Vasc. Biol. 18(10):1617-24; Aviram et al., 1998, J. Clin. Invest. 101(8):1581-90; Billecke et al., 2000, Drug Metab. Dispos. 28(11):1335-42; Draganov et al., 2000, J. Biol. Chem. 275(43):33435-42; Steinmetz and Utermann 1985, J. Biol. Chem. 260(4):2258-64; Widler et al., 1980, J. Biol. Chem. 255(21):10464-71; Dyer et al., 1995, J. Lipid Res. 36(1):80-8; Sorenson et al., 1999, Arterioscler. Thromb. Vasc. Biol. 19(9):2214-25; Palgunachari 1996, Arterioscler. Throb. Vasc. Biol. 16(2):328-38: Thurberg et al., J. Biol. Chem. 271(11):6062-70; Dyer 1991, J. Biol. Chem. 266(23):150009-15; Hill 1998, J. Biol. Chem. 273(47):30979-84).

Apolipoproteins utilized in the invention also include recombinant, synthetic, semi-synthetic or purified apolipoproteins. Methods for obtaining apolipoproteins or equivalents thereof, utilized by the invention are well-known in the art. For example, apolipoproteins can be separated from plasma or natural products by, for example, density gradient centrifugation or immunoaffinity chromatography, or produced synthetically, semi-synthetically or using recombinant DNA techniques known to those of the art (see, e.g., Mulugeta et al., 1998, J. Chromatogr. 798(1-2): 83-90; Chung et al., 1980, J. Lipid Res. 21(3):284-91; Cheung et al., 1987, J. Lipid Res. 28(8):913-29; Persson, et al., 1998, J. Chromatogr. 711:97-109; U.S. Pat. Nos. 5,059,528, 5,834,596, 5,876,968 and 5,721,114; and PCT Publications WO 86/04920 and WO 87/02062).

Apolipoproteins utilized in the invention further include apolipoprotein agonists such as peptides and peptide analogues that mimic the activity of ApoA-I, ApoA-I Milano (ApoA-I_{M}), ApoA-I Paris (ApoA-I_{P}), ApoA-II, ApoA-IV, and ApoE. For example, the apolipoprotein can be any of those described in U.S. Pat. Nos. 6,004,925, 6,037,323, 6,046,166, and 5,840,688, the contents of which are incorporated herein by reference in their entireties.

Apolipoprotein agonist peptides or peptide analogues can be synthesized or manufactured using any technique for peptide synthesis known in the art including, e.g., the techniques described in U.S. Pat. Nos. 6,004,925, 6,037,323 and 6,046,166. For example, the peptides may be prepared using the solid-phase synthetic technique initially described by Merrifield (1963, J. Am. Chem. Soc. 85:2149-2154). Other peptide synthesis techniques may be found in Bodanszky et al., Peptide Synthesis, John Wiley & Sons, 2d Ed., (1976) and other references readily available to those skilled in the art. A summary of polypeptide synthesis techniques can be found in Stuart and Young, Solid Phase Peptide. Synthesis, Pierce Chemical Company, Rockford, Ill., (1984). Peptides may also be synthesized by solution methods as described in The Proteins, Vol. II, 3d Ed., Neurath et al., Eds., p. 105-237, Academic Press, New York, N.Y. (1976). Appropriate protective groups for use in different peptide syntheses are described in the above-mentioned texts as well as in McOmie, Protective Groups in Organic Chemistry, Plenum Press, New York, N.Y. (1973). The peptides of the present invention might also be prepared by chemical or enzymatic cleavage from larger portions of, for example, apolipoprotein A-I.

In certain embodiments, the apolipoprotein can be a mixture of apolipoproteins. In one embodiment, the apolipoprotein can be a homogeneous mixture, that is, a single type of apolipoprotein. In another embodiment, the apolipoprotein can be a heterogeneous mixture of apolipoproteins, that is, a mixture of two or more different apolipoproteins. Embodiments of heterogenous mixtures of apolipoproteins can comprise, for example, a mixture of an apolipoprotein from an animal source and an apolipoprotein from a semi-synthetic source. In certain embodiments, a heterogenous mixture can comprise, for example, a mixture of ApoA-I and ApoA-I Milano. In certain embodiments, a heterogeneous mixture can comprise, for example, a mixture of ApoA-I Milano and ApoA-I Paris. Suitable mixtures for use in the methods and compositions of the invention will be apparent to one of skill in the art.

If the apolipoprotein is obtained from natural sources, it can be obtained from a plant or animal source. If the apolipoprotein is obtained from an animal source, the apolipoprotein can be from any species. In certain embodiments, the apolipoprotien can be obtained from an animal source. In certain embodiments, the apolipoprotein can be obtained from a human source. In preferred embodiments of the invention, the apolipoprotein is derived from the same species as the individual to which the apolipoprotein is administered.

Other Components

In numerous embodiments, amphipathic lipids are included in lipid particles of the invention.

“Amphipathic lipids” refer to any suitable material, wherein the hydrophobic portion of the lipid material orients into a hydrophobic phase, while the hydrophilic portion orients toward the aqueous phase. Such compounds include, but are not limited to, phospholipids, aminolipids, and sphingolipids. Representative phospholipids include sphingomyelin, phosphatidylcholine, phosphatidylethanolamine, phosphatidylserine, phosphatidylinositol, phosphatidic acid, palmitoyloleoyl phosphatdylcholine, lysophosphatidylcholine, lysophosphatidylethanolamine, dipalmitoylphosphatidylcholine, dioleoylphosphatidylcholine, distearoylphosphatidylcholine, or dilinoleylphosphatidylcholine. Other phosphorus-lacking compounds, such as sphingolipids, glycosphingolipid families, diacylglycerols, and β-acyloxyacids, can also be used. Additionally, such amphipathic lipids can be readily mixed with other lipids, such as triglycerides and sterols.

Also suitable for inclusion in the lipid particles of the invention are programmable fusion lipids. Such lipid particles have little tendency to fuse with cell membranes and deliver their payload until a given signal event occurs. This allows the lipid particle to distribute more evenly after injection into an organism or disease site before it starts fusing with cells. The signal event can be, for example, a change in pH, temperature, ionic environment, or time. In the latter case, a fusion delaying or “cloaking” component, such as an ATTA-lipid conjugate or a PEG-lipid conjugate, can simply exchange out of the lipid particle membrane over time. Exemplary lipid anchors include those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons.

A lipid particle conjugated to a nucleic acid agent can also include a targeting moiety, e.g., a targeting moiety that is specific to a cell type or tissue. Targeting of lipid particles using a variety of targeting moieties, such as ligands, cell surface receptors, glycoproteins, vitamins (e.g., riboflavin) and monoclonal antibodies, has been previously described (see, e.g., U.S. Pat. Nos. 4,957,773 and 4,603,044). The targeting moieties can include the entire protein or fragments thereof. Targeting mechanisms generally require that the targeting agents be positioned on the surface of the lipid particle in such a manner that the targeting moiety is available for interaction with the target, for example, a cell surface receptor. A variety of different targeting agents and methods are known and available in the art, including those described, e.g., in Sapra, P. and Allen, T M, Prog. Lipid Res. 42(5):439-62 (2003); and Abra, R M et al., J. Liposome Res. 12:1-3, (2002).

The use of lipid particles, i.e., liposomes, with a surface coating of hydrophilic polymer chains, such as polyethylene glycol (PEG) chains, for targeting has been proposed (Allen, et al., Biochimica et Biophysica Acta 1237: 99-108 (1995); DeFrees, et al., Journal of the American Chemistry Society 118: 6101-6104 (1996); Blume, et al., Biochimica et Biophysica Acta 1149: 180-184 (1993); Klibanov, et al., Journal of Liposome Research 2: 321-334 (1992); U.S. Pat. No. 5,013,556; Zalipsky, Bioconjugate Chemistry 4: 296-299 (1993); Zalipsky, FEBS Letters 353: 71-74 (1994); Zalipsky, in Stealth Liposomes Chapter 9 (Lasic and Martin, Eds) CRC Press, Boca Raton Fl (1995). In one approach, a ligand, such as an antibody, for targeting the lipid particle is linked to the polar head group of lipids forming the lipid particle. In another approach, the targeting ligand is attached to the distal ends of the PEG chains forming the hydrophilic polymer coating (Klibanov, et al., Journal of Liposome Research 2: 321-334 (1992); Kirpotin et al., FEBS Letters 388: 115-118 (1996)).

Standard methods for coupling the target agents can be used. For example, phosphatidylethanolamine, which can be activated for attachment of target agents, or derivatized lipophilic compounds, such as lipid-derivatized bleomycin, can be used. Antibody-targeted liposomes can be constructed using, for instance, liposomes that incorporate protein A (see, Renneisen, et al., J. Bio. Chem., 265:16337-16342 (1990) and Leonetti, et al., Proc. Natl. Acad. Sci. (USA), 87:2448-2451 (1990). Other examples of antibody conjugation are disclosed in U.S. Pat. No. 6,027,726, the teachings of which are incorporated herein by reference. Examples of targeting moieties can also include other proteins, specific to cellular components, including antigens associated with neoplasms or tumors. Proteins used as targeting moieties can be attached to the liposomes via covalent bonds (see, Heath, Covalent Attachment of Proteins to Liposomes, 149 Methods in Enzymology 111-119 (Academic Press, Inc. 1987)). Other targeting methods include the biotin-avidin system.

Production of Nucleic Acid-Lipid Particles

In one embodiment, the nucleic acid-lipid particle formulations of the invention are produced via an extrusion method or an in-line mixing method.

The extrusion method (also refer to as preformed method or batch process) is a method where the empty liposomes (i.e. no nucleic acid) are prepared first, followed by the addition of nucleic acid to the empty liposome. Extrusion of liposome compositions through a small-pore polycarbonate membrane or an asymmetric ceramic membrane results in a relatively well-defined size distribution. Typically, the suspension is cycled through the membrane one or more times until the desired liposome complex size distribution is achieved. The liposomes may be extruded through successively smaller-pore membranes, to achieve a gradual reduction in liposome size. In some instances, the lipid-nucleic acid compositions which are formed can be used without any sizing. These methods are disclosed in the U.S. Pat. No. 5,008,050; U.S. Pat. No. 4,927,637; U.S. Pat. No. 4,737,323; Biochim Biophys Acta. 1979 Oct. 19; 557(1):9-23; Biochim Biophys Acta. 1980 Oct. 2; 601(3):559-7; Biochim Biophys Acta. 1986 Jun. 13; 858(1):161-8; and Biochim. Biophys. Acta 1985 812, 55-65, which are hereby incorporated by reference in their entirety.

The in-line mixing method is a method wherein both the lipids and the nucleic acid are added in parallel into a mixing chamber. The mixing chamber can be a simple T-connector or any other mixing chamber that is known to one skill in the art. These methods are disclosed in U.S. Pat. No. 6,534,018 and U.S. Pat. No. 6,855,277; US publication 2007/0042031 and Pharmaceuticals Research, Vol. 22, No. 3, March 2005, p. 362-372, which are hereby incorporated by reference in their entirety.

It is further understood that the formulations of the invention can be prepared by any methods known to one of ordinary skill in the art.

Characterization of Nucleic Acid-Lipid Particles

Formulations prepared by either the standard or extrusion-free method can be characterized in similar manners. For example, formulations are typically characterized by visual inspection. They should be whitish translucent solutions free from aggregates or sediment. Particle size and particle size distribution of lipid-nanoparticles can be measured by light scattering using, for example, a Malvern Zetasizer Nano ZS (Malvern, USA). Particles should be about 20-300 nm, such as 40-100 nm in size. The particle size distribution should be unimodal. The total siRNA concentration in the formulation, as well as the entrapped fraction, is estimated using a dye exclusion assay. A sample of the formulated siRNA can be incubated with an RNA-binding dye, such as Ribogreen (Molecular Probes) in the presence or absence of a formulation disrupting surfactant, e.g., 0.5% Triton-X100. The total siRNA in the formulation can be determined by the signal from the sample containing the surfactant, relative to a standard curve. The entrapped fraction is determined by subtracting the “free” siRNA content (as measured by the signal in the absence of surfactant) from the total siRNA content. Percent entrapped siRNA is typically >85%. In one embodiment, the formulations of the invention are entrapped by at least 75%, at least 80% or at least 90%.

For nucleic acid-lipid particle formulations, the particle size is at least 30 nm, at least 40 nm, at least 50 nm, at least 60 nm, at least 70 nm, at least 80 nm, at least 90 nm, at least 100 nm, at least 110 nm, and at least 120 nm. The suitable range is typically about at least 50 nm to about at least 110 mm, about at least 60 nm to about at least 100 nm, or about at least 80 nm to about at least 90 nm.

Certain Antisense Oligomeric Compounds

In certain embodiments, the invention provides compositions comprising one or more lipid particle and one or more oligomeric compound comprising or consisting of antisense oligonucleotides. In certain embodiments, an antisense oligonucleotide comprises a phosphate stabilizing nucleoside. In certain embodiments, an antisense oligonucleotide comprises a phosphate stabilizing nucleoside at the 5′-end. In certain embodiments, a phosphate stabilizing nucleoside comprises a modified phosphate group and/or a modified sugar moiety.

In certain embodiments, an antisense oligonucleotide comprises a 5′-stabilizing nucleotide. In certain embodiments, the 5′-stabilizing nucleoside comprises a modified sugar moiety.

In certain embodiments, the 5′-end of an antigens compound comprises a phosphate stabilizing modification and a 5′-stabilizing nucleoside. In certain embodiments, a single modification results in both phosphate stabilization and nucleoside stabilization. In certain embodiments, the phosphate stabilizing modification and the nucleoside stabilizing modification are different modifications. In certain embodiments, tow or more modifications at the 5′-end of an oligomeric compound together provide phosphate stabilization and nucleoside stabilization.

In certain embodiments, an antisense oligomeric compound comprises the following features selected from: a 5′-phosphate or 5′-modified phosphate; a 5′-most nucleoside (position 1 nucleoside); a nucleoside second from the 5′-end (position 2 nucleoside); a nucleoside third from the 5′-end (position 3 nucleoside); a region having a nucleoside motif; a region having a linkage motif; a terminal group.

In certain embodiments, the 5′-phosphate is selected from: unmodified phosphate, modified phosphate, phosphonate, alkylphosphonate, substituted alkylphosphonate, aminoalkyl phosphonate, substituted aminoalkyl phosphonate, phosphorothioate, phosphoramidate, alkylphosphonothioate, substituted alkylphosphonothioate, phosphorodithioate, thiophosphoramidate, and phosphotriester.

In certain embodiments, the 5′-phosphate is selected from: modified phosphate, phosphonate, alkylphosphonate, substituted alkylphosphonate, aminoalkyl phosphonate, substituted aminoalkyl phosphonate, phosphotriester, phosphorothioate, phosphorodithioate, thiophosphoramidate, and phosphoramidate.

In certain embodiments, the 5′-phosphate is selected from: modified phosphate, phosphonate, alkylphosphonate, and substituted alkylphosphonate. In certain embodiments, the 5′-phosphate is selected from 5′-deoxy-5′-thio phosphate, phosphoramidate, methylene phosphonate, mono-fluoro methylene phosphonate and di-fluoro methylene phosphonate.

In certain embodiments, the position 1 nucleoside comprises a modified sugar. In certain such embodiments, the sugar comprises a 5′-modification. In certain embodiments, the sugar of the position 1 nucleoside comprises a 2′-modification. In certain embodiments, the sugar of the position 1 nucleoside comprises a 5′-modification and a 2′-modification. In certain embodiments, the 5′-modification of the sugar of the position 1 nucleoside is selected from 5′-alkyl, 5′-substituted alkyl, 5′-alkoxy, 5′-substituted alkoxy, and 5′-halogen. In certain embodiments, the 5′ modification of the sugar at position 1 is selected from 5′-alkyl and 5′-substituted alkyl. In certain such embodiments, the modification is selected from methyl and ethyl. In certain embodiments, the 2′ modification is selected from: halogen (including, but not limited to F), allyl, amino, azido, thio, O-allyl, —O—C₁-C₁₀ alkyl, —O—C₁-C₁₀ substituted alkyl, —OCF₃, —O—(CH₂)₂—O—CH₃, —O(CH₂)₂SCH₃, —O—(CH₂)₂—O—N(Rₘ)(Rₙ), —O—CH2-C(═O)—N(Rₘ)(Rₙ), where each Rₘ and Rₙ is, independently, H or substituted or unsubstituted C₁-C₁₀ alkyl, —O[(CH₂)ₙO]ₘCH₃, —O(CH₂)ₙNH₂, —O(CH₂)ₙCH₃; —O(CH₂)ₙONH₂, —OCH₂C(═O)N(H)CH₃, —O(CH₂)ₙON[(CH₂)ₙCH₃]₂, where n and m are from 1 to about 10; C₁ to C₁₀ alkyl, substituted alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl. In certain embodiments, the 2′-modification of the sugar of the position 1 nucleoside is selected from: F, —O—C₁-C₁₀ alkyl, —O—C₁-C₁₀ substituted alkyl, —OCF₃, —O—(CH₂)₂—O—CH₃, —O(CH₂)₂SCH₃, —O—(CH₂)₂—O—N(Rₘ)(Rₙ), —O—CH₂—C(═O)—N(Rₘ)(Rₙ), where each Rₘ and Rₙ is, independently, H or substituted or unsubstituted C₁-C₁₀ alkyl, —O[(CH₂)ₙO]ₘCH₃, —O(CH₂)ₙNH₂, —O(CH₂)ₙCH₃, —O(CH₂)ₙONH₂, —OCH₂C(═O)N(H)CH₃, —O(CH₂)ₙON[(CH₂)ₙCH₃]₂, where n and m are from 1 to about 10; —O-aryl, S-alkyl, NMA, DMAEAc, DMAEOE, and —O-alkyl-F. In certain embodiments, the 2′-modification of the sugar of the position 1 nucleoside is selected from: F, —O—C₁-C₁₀ alkyl, —O—C₁-C₁₀ substituted alkyl, —O—(CH₂)₂—O—CH₃, —O(CH₂)₂SCH₃, —O—(CH₂)₂—O—N(Rₘ)(Rₙ), —O—CH2-C(═O)—N(Rₘ)(Rₙ), where each Rₘ and Rₙ is, independently, H or substituted or unsubstituted C₁-C₁₀ alkyl, —O[(CH₂)ₙO]ₘCH₃, —O(CH₂)ₙNH₂, —O(CH₂)ₙCH₃, —O(CH₂)ₙONH₂, —OCH₂C(═O)N(H)CH₃, —O(CH₂)ₙON[(CH₂)ₙCH₃]₂; where n and m are from 1 to about 10; —O-aryl, S-alkyl, NMA, DMAEAc, DMAEOE, and —O-alkyl-F.

In certain embodiments, the position 2 nucleoside comprises a 2′-modification. In certain such embodiments, the 2′-modification of the position 2 nucleoside is selected from halogen, alkyl, and substituted alkyl. In certain embodiments, the 2′-modification of the position 2 nucleoside is selected from 2′-F and 2′-alkyl. In certain embodiments, the 2′-modification of the position 2 nucleoside is 2′-F. In certain embodiments, the 2′-substituted of the position 2 nucleoside is an unmodified OH (as in naturally occurring RNA).

In certain embodiments, the position 3 nucleoside is a modified nucleoside. In certain embodiments, the position 3 nucleoside is a bicyclic nucleoside. In certain embodiments, the position 3 nucleoside comprises a sugar surrogate. In certain such embodiments, the sugar surrogate is a tetrahydropyran. In certain embodiments, the sugar of the position 3 nucleoside is a F—HNA.

In certain embodiments, an antisense oligomeric compound comprises an oligonucleotide comprising 10 to 30 linked nucleosides wherein the oligonucleotide comprises:

a 5′-terminal phosphate or modified phosphate:

a position 1 modified nucleoside comprising a modified sugar moiety comprising:
a 5′-modification; or a 2′-modification; or both a 5′-modification and a 2′-modification;

a position 2 nucleoside comprising a sugar moiety which is differently modified compared to the sugar moiety of the position 1 modified nucleoside; and

from 1 to 4 3′-terminal group nucleosides each comprising a 2′-modification; and

wherein at least the seven 3′-most internucleoside linkages are phosphorothioate linkages.

In certain such embodiments, the 5′-terminal modified phosphate is selected from: phosphonate, alkylphosphonate, aminoalkyl phosphonate, phosphorothioate, phosphoramidite, alkylphosphonothioate, phosphorodithioate, thiophosphoramidate, phosphotriester;

the 5′-modification of the sugar moiety of the position 1 modified nucleoside is selected from 5′-alkyl and 5′-halogen;

the 2′-modification of the sugar moiety of the position 1 modified nucleoside is selected from: halogen (including, but not limited to F), allyl, amino, azido, thio, O-allyl, —O—C₁-C₁₀ alkyl, —O—C₁-C₁₀ substituted alkyl, —OCF₃, —O—(CH₂)₂—O—CH₃, —O(CH₂)₂SCH₃, —O—(CH₂)₂—O—N(Rₘ)(Rₙ), —O—CH2-C(═O)—N(Rₘ)(Rₙ), where each Rₘ and Rₙ is, independently, H or substituted or unsubstituted C₁-C₁₀ alkyl, —O[(CH₂)ₙO]ₘCH₃, —O(CH₂)ₙNH₂, —O(CH₂)ₙCH₃, —O(CH₂)ₙONH₂, —OCH₂C(═O)N(H)CH₃, —O(CH₂)ₙON[(CH₂)ₙCH₃]₂, where n and m are from 1 to about 10; C₁ to C₁₀ alkyl, substituted alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl; and

the sugar moiety of the position 2 nucleoside is selected from unmodified 2′-OH(RNA) sugar, and a modified sugar comprising a modification selected from: 2′-halogen, 2′O-alkyl, 2′-alkyl, 2′-substituted alkyl.

In certain embodiments, the sugar moiety of the position 2 nucleoside comprises a 2′-F.

In certain embodiments, such oligonucleotides comprises 8 to 20, 10 to 15, 11 to 14, or 12 to 13 phosphorothioate internucleoside linkages overall. In certain embodiments, the remaining internucleoside linkages are phosphodiester. In certain embodiments, the eighth internucleoside linkage from the 3′ end of the oligonucleotide is a phosphodiester. In certain embodiments, the ninth internucleoside linkage from the 3′ end is a phosphodiester. In certain embodiments, each internucleoside linkage is either a phosphorothioate or a phosphodiester linkage.

In certain such embodiments, antisense oligomeric compounds have the features described in the following non-limiting table:

| | Sugar moiety of position | | | Positions 3 to | | |
|---|---|---|---|---|---|---|
| | 1 nucleoside | | Position | 3′-end motifs | 3′-terminal | |
| 5′-phophate | 5' | 2' | 2 | or features | group | Linkages |
| unmodified phosphate | methyl | MOE | 2′-F | Alternating | 1-4 MOE | At least 7 PS |
| | | | | modifications | | at 3′ end |
| thiophosphate | methyl | MOE | 2′-F | Alternating | 1-4 MOE | At least 7 PS |
| | | | | OMe/F | | at 3′ end |
| Phosphonate | methyl | DMAEAc | 2′-F | Alternating | 1-4 MOE | At least 7 PS |
| | | | | OMe/F | | at 3′ end |
| Methylphosphonate | methyl | Tri-MOE | 2′-F | 2-2-3 | None | 6-8 PS at 3′ |
| | | | | | | end and total |
| | | | | | | of 10 PS |
| | | | | | | throughout |
| alkylphosphonothioate | unmod | O-alkyl | 2′-F | any | 2 MOE | 7 PS at 3′ end |
| | | | | | adenosines | and total of ≧ |
| | | | | | | 10 PS |
| | | | | | | throughout |
| Phosphonate or | Methyl | MOE, O- | 2′-F | any | 1-4 MOE | 7-8 PS at 3′ |
| alkylphosphonate | or | alkyl; O- | | | adenosines | end; total of |
| | unmod. | subst. alkyl; | | | | 10-15 PS |
| | | F, —O-aryl, | | | | linkages |
| | | S-alkyl, | | | | throughout; |
| | | NMA, | | | | remaining |
| | | DMAEAc, | | | | linkages are |
| | | DMAEOE, | | | | PO |
| | | —O-alkyl-F | | | | |
| Posphonate or | Alkyl | MOE, O- | 2′-F | BNA at | 1-4 MOE | 7-8 PS at 3′ |
| modified phosphonate | | alkyl; O- | | position 3 | adenosines | end; total of |
| | | subst. alkyl; | | | | 10-15 PS |
| | | F, —O-aryl, | | | | linkages |
| | | S-alkyl, | | | | throughout; |
| | | NMA, | | | | remaining |
| | | DMAEAc, | | | | linkages are |
| | | DMAEOE, | | | | PO |
| | | —O-alkyl-F | | | | |

In certain embodiments, the third nucleoside from the 5′-end (position 3) is a modified nucleoside. In certain embodiments, the nucleoside at position 3 comprises a sugar modification. In certain such embodiments, the sugar moiety of the position 3 nucleoside is a bicyclic nucleoside. In certain embodiments the position 3 nucleoside is a modified non-bicyclic nucleoside. In certain embodiments, the position 3 nucleoside is selected from: F—HNA and 2′-OMe.

### Certain Methods/Uses

In certain embodiments, the present invention provides compositions and methods for reducing the amount or activity of a target nucleic acid. In certain embodiments, the invention provides compositions comprising antisense compounds and methods. In certain embodiments, the invention provides compositions comprising antisense compounds and methods based on activation of RNase H. In certain embodiments, the invention provides RNAi compounds and methods.

In certain instances it is desirable to use an antisense compound that functions at least in part through RISC. In certain such instances unmodified RNA, whether single-stranded or double stranded is not suitable. Single-stranded RNA is relatively unstable and double-stranded RNA does not easily enter cells. The challenge has been to identify modifications and motifs that provide desirable properties, such as improved stability, without interfering with (and possibly even improving upon) the antisense activity of RNA through RNAi.

In certain embodiments, the present invention provides compositions comprising oligonucleotides having motifs (nucleoside motifs and/or linkage motifs) that result in improved properties. Certain such motifs result in single-stranded oligonucleotides with improved stability and/or cellular uptake properties while retaining antisense activity. For example, oligonucleotides having an alternating nucleoside motif and seven phosphorothioate linkages at to 3′-terminal end have improved stability and activity. Similar compounds that comprise phosphorothioate linkages at each linkage have further improved stability, but are not active as RNAi compounds, presumably because the additional phosphorothioate linkages interfere with the interaction of the oligonucleotide with the RISC pathway components (e.g., with Ago). In certain embodiments, the oligonucleotides having motifs herein result in single-stranded RNAi compounds having desirable properties. In certain embodiments, such oligonucleotides may be paired with a second strand to form a double-stranded RNAi compound. In such embodiments, the second strand of such double-stranded RNAi compounds may comprise a motif as described herein, or may comprise another motif of modifications or may be unmodified.

It has been shown that in certain circumstances for single-stranded RNA comprising a 5′-phosphate group has RNAi activity if but has much less RNAi activity if it lacks such 5′-phosphate group. The present inventors have recognized that in certain circumstances unmodified 5′-phosphate groups may be unstable (either chemically or enzymatically). Accordingly, in certain circumstances, it is desirable to modify the oligonucleotide to stabilize the 5′-phosphate. In certain embodiments, this is achieved by modifying the phosphate group. In certain embodiments, this is achieved by modifying the sugar of the 5′-terminal nucleoside. In certain embodiments, this is achieved by modifying the phosphate group and the sugar. In certain embodiments, the sugar is modified at the 5′-position, the 2′-position, or both the 5′-position and the 2′-position. As with motifs, above, in embodiments in which RNAi activity is desired, a phosphate stabilizing modification must not interfere with the ability of the oligonucleotide to interact with RISC pathway components (e.g., with Ago).

In certain embodiments, the invention provides compositions comprising oligonucleotides comprising a phosphate-stabilizing modification and a motif described herein. In certain embodiments, such oligonucleotides are useful as single-stranded RNAi compounds having desirable properties. In certain embodiments, such oligonucleotides may be paired with a second strand to form a double-stranded RNAi compound. In such embodiments, the second strand may comprise a motif as described herein, may comprise another motif of modifications or may be unmodified RNA.

The target for such antisense compounds comprising a motif and/or 5′-phosphate stabilizing modification can be any naturally occurring nucleic acid. In certain embodiments, the target is selected from: pre-mRNA, mRNA, non-coding RNA, small non-coding RNA, pd-RNA, and microRNA. In embodiments, in which a target nucleic acid is a pre-RNA or a mRNA, the target may be the same as that of a naturally occurring micro-RNA (i.e., the oligonucleotide may be a microRNA mimic). In such embodiments, there may be more than one target mRNA.

In certain embodiments, the invention provides compositions and methods for antisense activity in a cell. In certain embodiments, the cell is in an animal. In certain embodiments, the animal is a human. In certain embodiments, the invention provides methods of administering a composition of the present invention to an animal to modulate the amount or activity or function of one or more target nucleic acid.

In certain embodiments compositions comprise oligonucleotides comprising one or more motifs of the present invention, but do not comprise a phosphate stabilizing modification. In certain embodiments, the motif and the lipid particle are sufficient to result in activity without phosphate stabilization.

### Nonlimiting Disclosure and Incorporation by Reference

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH for the natural 2′-H of DNA) or as an RNA having a modified base (thymine (methylated uracil) for natural uracil of RNA).

Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified bases, such as “AT^{me}CGAUCG,” wherein ^{me}C indicates a cytosine base comprising a methyl group at the 5-position.

Likewise, one of skill will appreciate that in certain circumstances using the conventions described herein, the same compound may be described in more than one way. For example, an antisense oligomeric compound having two non-hybridizing 3′-terminal 2′-MOE modified nucleosides, but otherwise fully complementary to a target nucleic acid may be described as an oligonucleotide comprising a region of 2′-MOE-modified nucleosides, wherein the oligonucleotide is less than 100% complementary to its target. Or that same compound may be described as an oligomeric compound comprising: (1) an oligonucleotide that is 100% complementary to its nucleic acid target and (2) a terminal group wherein the terminal group comprises two 2′-MOE modified terminal-group nucleosides. Such descriptions are not intended to be exclusive of one another or to exclude overlapping subject matter.

### Example 1

### Synthesis of Nucleoside Phosphoramidites

The preparation of nucleoside phosphoramidites is performed following procedures that are illustrated herein and in the art such as but not limited to U.S. Pat. No. 6,426,220 and published PCT WO 02/36743.

### Example 2

### Synthesis of Oligomeric Compounds

The oligomeric compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as alkylated derivatives and those having phosphorothioate linkages.

Oligomeric compounds: Unsubstituted and substituted phosphodiester (P═O) oligomeric compounds, including without limitation, oligonucleotides can be synthesized on an automated DNA synthesizer (Applied Biosystems model 394) using standard phosphoramidite chemistry with oxidation by iodine.

In certain embodiments, phosphorothioate internucleoside linkages (P═S) are synthesized similar to phosphodiester internucleoside linkages with the following exceptions: thiation is effected by utilizing a 10% w/v solution of 3,H-1,2-benzodithiole-3-one 1,1-dioxide in acetonitrile for the oxidation of the phosphite linkages. The thiation reaction step time is increased to 180 sec and preceded by the normal capping step. After cleavage from the CPG column and deblocking in concentrated ammonium hydroxide at 55° C. (12-16 hr), the oligomeric compounds are recovered by precipitating with greater than 3 volumes of ethanol from a 1 M NH₄OAc solution. Phosphinate internucleoside linkages can be prepared as described in U.S. Pat. No. 5,508,270.

Alkyl phosphonate internucleoside linkages can be prepared as described in U.S. Pat. No. 4,469,863.

3′-Deoxy-3′-methylene phosphonate internucleoside linkages can be prepared as described in U.S. Pat. No. 5,610,289 or 5,625,050.

Phosphoramidite internucleoside linkages can be prepared as described in U.S. Pat. No. 5,256,775 or U.S. Pat. No. 5,366,878.

Alkylphosphonothioate internucleoside linkages can be prepared as described in published PCT applications PCT/US94/00902 and PCT/US93/06976 (published as WO 94/17093 and WO 94/02499, respectively).

3′-Deoxy-3′-amino phosphoramidate internucleoside linkages can be prepared as described in U.S. Pat. No. 5,476,925.

Phosphotriester internucleoside linkages can be prepared as described in U.S. Pat. No. 5,023,243.

Borano phosphate internucleoside linkages can be prepared as described in U.S. Pat. Nos. 5,130,302 and 5,177,198.

Oligomeric compounds having one or more non-phosphorus containing internucleoside linkages including without limitation methylenemethylimino linked oligonucleosides, also identified as MMI linked oligonucleosides, methylenedimethylhydrazo linked oligonucleosides, also identified as MDH linked oligonucleosides, methylenecarbonylamino linked oligonucleosides, also identified as amide-3 linked oligonucleosides, and methyleneaminocarbonyl linked oligonucleosides, also identified as amide-4 linked oligonucleosides, as well as mixed backbone oligomeric compounds having, for instance, alternating MMI and P═O or P═S linkages can be prepared as described in U.S. Pat. Nos. 5,378,825, 5,386,023, 5,489,677, 5,602,240 and 5,610,289.

Formacetal and thioformacetal internucleoside linkages can be prepared as described in U.S. Pat. Nos. 5,264,562 and 5,264,564.

Ethylene oxide internucleoside linkages can be prepared as described in U.S. Pat. No. 5,223,618.

### Example 3

### Isolation and Purification of Oligomeric Compounds

After cleavage from the controlled pore glass solid support or other support medium and deblocking in concentrated ammonium hydroxide at 55° C. for 12-16 hours, the oligomeric compounds, including without limitation oligonucleotides and oligonucleosides, are recovered by precipitation out of 1 M NH₄OAc with >3 volumes of ethanol. Synthesized oligomeric compounds are analyzed by electrospray mass spectroscopy (molecular weight determination) and by capillary gel electrophoresis. The relative amounts of phosphorothioate and phosphodiester linkages obtained in the synthesis is determined by the ratio of correct molecular weight relative to the −16 amu product (+/−32+/−48). For some studies oligomeric compounds are purified by HPLC, as described by Chiang et al., J. Biol. Chem. 1991, 266, 18162-18171. Results obtained with HPLC-purified material are generally similar to those obtained with non-HPLC purified material.

### Example 4

### Synthesis of Oligomeric Compounds using the 96 Well Plate Format

Oligomeric compounds, including without limitation oligonucleotides, can be synthesized via solid phase P(III) phosphoramidite chemistry on an automated synthesizer capable of assembling 96 sequences simultaneously in a 96-well format. Phosphodiester internucleoside linkages are afforded by oxidation with aqueous iodine. Phosphorothioate internucleoside linkages are generated by sulfurization utilizing 3,H-1,2 benzodithiole-3-one 1,1 dioxide (Beaucage Reagent) in anhydrous acetonitrile. Standard base-protected beta-cyanoethyl-diiso-propyl phosphoramidites can be purchased from commercial vendors (e.g. PE-Applied Biosystems, Foster City, Calif., or Pharmacia, Piscataway, N.J.). Non-standard nucleosides are synthesized as per standard or patented methods and can be functionalized as base protected beta-cyanoethyldiisopropyl phosphoramidites.

Oligomeric compounds can be cleaved from support and deprotected with concentrated NH₄OH at elevated temperature (55-60° C.) for 12-16 hours and the released product then dried in vacuo. The dried product is then re-suspended in sterile water to afford a master plate from which all analytical and test plate samples are then diluted utilizing robotic pipettors.

### Example 5

### Analysis of Oligomeric Compounds Using the 96-Well Plate Format

The concentration of oligomeric compounds in each well can be assessed by dilution of samples and UV absorption spectroscopy. The full-length integrity of the individual products can be evaluated by capillary electrophoresis (CE) in either the 96-well format (Beckman P/ACE™ MDQ) or, for individually prepared samples, on a commercial CE apparatus (e.g., Beckman P/ACE™ 5000, ABI 270). Base and backbone composition is confirmed by mass analysis of the oligomeric compounds utilizing electrospray-mass spectroscopy. All assay test plates are diluted from the master plate using single and multi-channel robotic pipettors. Plates are judged to be acceptable if at least 85% of the oligomeric compounds on the plate are at least 85% full length.

### Example 6

### In Vitro Treatment of Cells with Oligomeric Compounds

The effect of oligomeric compounds on target nucleic acid expression is tested in any of a variety of cell types provided that the target nucleic acid is present at measurable levels. This can be routinely determined using, for example, PCR or Northern blot analysis. Cell lines derived from multiple tissues and species can be obtained from American Type Culture Collection (ATCC, Manassas, Va.).

The following cell type is provided for illustrative purposes, but other cell types can be routinely used, provided that the target is expressed in the cell type chosen. This can be readily determined by methods routine in the art, for example Northern blot analysis, ribonuclease protection assays or RT-PCR.

b.END cells: The mouse brain endothelial cell line b.END was obtained from Dr. Werner Risau at the Max Plank Institute (Bad Nauheim, Germany). b.END cells are routinely cultured in DMEM, high glucose (Invitrogen Life Technologies, Carlsbad, Calif.) supplemented with 10% fetal bovine serum (Invitrogen Life Technologies, Carlsbad, Calif.). Cells are routinely passaged by trypsinization and dilution when they reached approximately 90% confluence. Cells are seeded into 96-well plates (Falcon-Primaria #353872, BD Biosciences, Bedford, Mass.) at a density of approximately 3000 cells/well for uses including but not limited to oligomeric compound transfection experiments.

Experiments involving treatment of cells with oligomeric compounds:

When cells reach appropriate confluency, they are treated with oligomeric compounds using a transfection method as described.

Lipofectin™

When cells reached 65-75% confluency, they are treated with one or more oligomeric compounds. The oligomeric compound is mixed with LIPOFECTIN™ Invitrogen Life Technologies, Carlsbad, Calif.) in Opti-MEM™-1 reduced serum medium (Invitrogen Life Technologies, Carlsbad, Calif.) to achieve the desired concentration of the oligomeric compound(s) and a LIPOFECTIN™ concentration of 2.5 or 3 μg/mL per 100 nM oligomeric compound(s). This transfection mixture is incubated at room temperature for approximately 0.5 hours. For cells grown in 96-well plates, wells are washed once with 100 μL OPTI-MEM™-1 and then treated with 130 μL of the transfection mixture. Cells grown in 24-well plates or other standard tissue culture plates are treated similarly, using appropriate volumes of medium and oligomeric compound(s). Cells are treated and data are obtained in duplicate or triplicate. After approximately 4-7 hours of treatment at 37° C., the medium containing the transfection mixture is replaced with fresh culture medium. Cells are harvested 16-24 hours after treatment with oligomeric compound(s).

Other suitable transfection reagents known in the art include, but are not limited to, CYTOFECTIN™, LIPOFECTAMINE™, OLIGOFECTAMINE™, and FUGENE™. Other suitable transfection methods known in the art include, but are not limited to, electroporation.

### Example 7

### Real-Time Quantitative PCR Analysis of Target mRNA Levels

Quantitation of target mRNA levels is accomplished by real-time quantitative PCR using the ABI PRISM™ 7600, 7700, or 7900 Sequence Detection System (PE-Applied Biosystems, Foster City, Calif.) according to manufacturer's instructions. This is a closed-tube, non-gel-based, fluorescence detection system which allows high-throughput quantitation of polymerase chain reaction (PCR) products in real-time. As opposed to standard PCR in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., FAM or JOE, obtained from either PE-Applied Biosystems, Foster City, Calif., Operon Technologies Inc., Alameda, Calif. or Integrated DNA Technologies Inc., Coralville, Iowa) is attached to the 5′ end of the probe and a quencher dye (e.g., TAMRA, obtained from either PE-Applied Biosystems, Foster City, Calif., Operon Technologies Inc., Alameda, Calif. or Integrated DNA Technologies Inc., Coralville, Iowa) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular intervals by laser optics built into the ABI PRISM™ Sequence Detection System. In each assay, a series of parallel reactions containing serial dilutions of mRNA from untreated control samples generates a standard curve that is used to quantitate the percent inhibition after antisense oligonucleotide treatment of test samples.

Prior to quantitative PCR analysis, primer-probe sets specific to the target gene being measured are evaluated for their ability to be “multiplexed” with a GAPDH amplification reaction. In multiplexing, both the target gene and the internal standard gene GAPDH are amplified concurrently in a single sample. In this analysis, mRNA isolated from untreated cells is serially diluted. Each dilution is amplified in the presence of primer-probe sets specific for GAPDH only, target gene only (“single-plexing”), or both (multiplexing). Following PCR amplification, standard curves of GAPDH and target mRNA signal as a function of dilution are generated from both the single-plexed and multiplexed samples. If both the slope and correlation coefficient of the GAPDH and target signals generated from the multiplexed samples fall within 10% of their corresponding values generated from the single-plexed samples, the primer-probe set specific for that target is deemed multiplexable. Other methods of PCR are also known in the art.

RT and PCR reagents are obtained from Invitrogen Life Technologies (Carlsbad, Calif.). RT, real-time PCR is carried out by adding 20 μL PCR cocktail (2.5×PCR buffer minus MgCl₂, 6.6 mM MgCl₂, 375 μM each of dATP, dCTP, dCTP and dGTP, 375 nM each of forward primer and reverse primer, 125 nM of probe, 4 Units RNAse inhibitor, 1.25 Units PLATINUM® Taq, 5 Units MuLV reverse transcriptase, and 2.5×ROX dye) to 96-well plates containing 30 μL total RNA solution (20-200 ng). The RT reaction is carried out by incubation for 30 minutes at 48° C. Following a 10 minute incubation at 95° C. to activate the PLATINUM® Taq, 40 cycles of a two-step PCR protocol are carried out: 95° C. for 15 seconds (denaturation) followed by 60° C. for 1.5 minutes (annealing/extension).

Gene target quantities obtained by RT, real-time PCR are normalized using either the expression level of GAPDH, a gene whose expression is constant, or by quantifying total RNA using RIBOGREEN™ (Molecular Probes, Inc. Eugene, Oreg.). GAPDH expression is quantified by real time RT-PCR, by being run simultaneously with the target, multiplexing, or separately. Total RNA is quantified using RiboGreen™ RNA quantification reagent (Molecular Probes, Inc. Eugene, Oreg.). Methods of RNA quantification by RIBOGREEN™ are taught in Jones, L. J., et al, (Analytical Biochemistry, 1998, 265, 368-374).

In this assay, 170 μL of RIBOGREEN™ working reagent (RIBOGREEN™ reagent diluted 1:350 in 10 mM Tris-HCl, 1 mM EDTA, pH 7.5) is pipetted into a 96-well plate containing 30 μL purified, cellular RNA. The plate is read in a CytoFluor 4000 (PE Applied Biosystems) with excitation at 485 nm and emission at 530 nm.

### Example 8

### Analysis of Oligonucleotide Inhibition of Target Expression

Antisense modulation of a target expression can be assayed in a variety of ways known in the art. For example, a target mRNA levels can be quantitated by, e.g., Northern blot analysis, competitive polymerase chain reaction (PCR), or real-time PCR. Real-time quantitative PCR is presently desired. RNA analysis can be performed on total cellular RNA or poly(A)+ mRNA. One method of RNA analysis of the present disclosure is the use of total cellular RNA as described in other examples herein. Methods of RNA isolation are well known in the art. Northern blot analysis is also routine in the art. Real-time quantitative (PCR) can be conveniently accomplished using the commercially available ABI PRISM™ 7600, 7700, or 7900 Sequence Detection System, available from PE-Applied Biosystems, Foster City, Calif. and used according to manufacturer's instructions.

Protein levels of a target can be quantitated in a variety of ways well known in the art, such as immunoprecipitation, Western blot analysis (immunoblotting), enzyme-linked immunosorbent assay (ELISA) or fluorescence-activated cell sorting (FACS). Antibodies directed to a target can be identified and obtained from a variety of sources, such as the MSRS catalog of antibodies (Aerie Corporation, Birmingham, Mich.), or can be prepared via conventional monoclonal or polyclonal antibody generation methods well known in the art. Methods for preparation of polyclonal antisera are taught in, for example, Ausubel, F. M. et al., Current Protocols in Molecular Biology, Volume 2, pp. 11.12.1-11.12.9, John Wiley & Sons, Inc., 1997. Preparation of monoclonal antibodies is taught in, for example, Ausubel, F. M. et al., Current Protocols in Molecular Biology, Volume 2, pp. 11.4.1-11.11.5, John Wiley & Sons, Inc., 1997.

Immunoprecipitation methods are standard in the art and can be found at, for example, Ausubel, F. M. et al., Current Protocols in Molecular Biology, Volume 2, pp. 10.16.1-10.16.11, John Wiley & Sons, Inc., 1998. Western blot (immunoblot) analysis is standard in the art and can be found at, for example, Ausubel, F. M. et al., Current Protocols in Molecular Biology, Volume 2, pp. 10.8.1-10.8.21, John Wiley & Sons, Inc., 1997. Enzyme-linked immunosorbent assays (ELISA) are standard in the art and can be found at, for example, Ausubel, F. M. et al., Current Protocols in Molecular Biology, Volume 2, pp. 11.2.1-11.2.22, John Wiley & Sons, Inc., 1991.

### Example 9

### Design of Phenotypic Assays and In Vivo Studies for the Use of Target Inhibitors

### Phenotypic Assays

Once target inhibitors have been identified by the methods disclosed herein, the oligomeric compounds are further investigated in one or more phenotypic assays, each having measurable endpoints predictive of efficacy in the treatment of a particular disease state or condition.

Phenotypic assays, kits and reagents for their use are well known to those skilled in the art and are herein used to investigate the role and/or association of a target in health and disease. Representative phenotypic assays, which can be purchased from any one of several commercial vendors, include those for determining cell viability, cytotoxicity, proliferation or cell survival (Molecular Probes, Eugene, Oreg.; PerkinElmer, Boston, Mass.), protein-based assays including enzymatic assays (Panvera, LLC, Madison, Wis.; BD Biosciences, Franklin Lakes, N.J.; Oncogene Research Products, San Diego, Calif.), cell regulation, signal transduction, inflammation, oxidative processes and apoptosis (Assay Designs Inc., Ann Arbor, Mich.), triglyceride accumulation (Sigma-Aldrich, St. Louis, Mo.), angiogenesis assays, tube formation assays, cytokine and hormone assays and metabolic assays (Chemicon International Inc., Temecula, Calif.; Amersham Biosciences, Piscataway, N.J.).

In one non-limiting example, cells determined to be appropriate for a particular phenotypic assay (i.e., MCF-7 cells selected for breast cancer studies; adipocytes for obesity studies) are treated with a target inhibitors identified from the in vitro studies as well as control compounds at optimal concentrations which are determined by the methods described above. At the end of the treatment period, treated and untreated cells are analyzed by one or more methods specific for the assay to determine phenotypic outcomes and endpoints.

Phenotypic endpoints include changes in cell morphology over time or treatment dose as well as changes in levels of cellular components such as proteins, lipids, nucleic acids, hormones, saccharides or metals. Measurements of cellular status which include pH, stage of the cell cycle, intake or excretion of biological indicators by the cell, are also endpoints of interest.

Measurement of the expression of one or more of the genes of the cell after treatment is also used as an indicator of the efficacy or potency of the a target inhibitors. Hallmark genes, or those genes suspected to be associated with a specific disease state, condition, or phenotype, are measured in both treated and untreated cells.

### In Vivo Studies

The individual subjects of the in vivo studies described herein are warm-blooded vertebrate animals, which includes humans.

### Example 10

### RNA Isolation

Poly(A)+ mRNA Isolation

Poly(A)+ mRNA is isolated according to Miura et al., (Clin. Chem., 1996, 42, 1758-1764). Other methods for poly(A)+ mRNA isolation are routine in the art. Briefly, for cells grown on 96-well plates, growth medium is removed from the cells and each well is washed with 200 μL cold PBS. 60 μL lysis buffer (10 mM Tris-HCl, pH 7.6, 1 mM EDTA, 0.5 M NaCl, 0.5% NP-40, 20 mM vanadyl-ribonucleoside complex) is added to each well, the plate is gently agitated and then incubated at room temperature for five minutes. 55 μL of lysate is transferred to Oligo d(T) coated 96-well plates (AGCT Inc., Irvine Calif.). Plates are incubated for 60 minutes at room temperature, washed 3 times with 200 μL of wash buffer (10 mM Tris-HCl pH 7.6, 1 mM EDTA, 0.3 M NaCl). After the final wash, the plate is blotted on paper towels to remove excess wash buffer and then air-dried for 5 minutes. 60 μL of elution buffer (5 mM Tris-HCl pH 7.6), preheated to 70° C., is added to each well, the plate is incubated on a 90° C. hot plate for 5 minutes, and the eluate is then transferred to a fresh 96-well plate.

Cells grown on 100 mm or other standard plates may be treated similarly, using appropriate volumes of all solutions.

### Total RNA Isolation

Total RNA is isolated using an RNEASY 96™ kit and buffers purchased from Qiagen Inc. (Valencia, Calif.) following the manufacturer's recommended procedures. Briefly, for cells grown on 96-well plates, growth medium is removed from the cells and each well is washed with 200 μL cold PBS. 150 μL Buffer RLT is added to each well and the plate vigorously agitated for 20 seconds. 150 μL of 70% ethanol is then added to each well and the contents mixed by pipetting three times up and down. The samples are then transferred to the RNEASY 96™ well plate attached to a QIAVAC™ manifold fitted with a waste collection tray and attached to a vacuum source. Vacuum is applied for 1 minute. 500 μL of Buffer RW1 is added to each well of the RNEASY 96™ plate and incubated for 15 minutes and the vacuum is again applied for 1 minute. An additional 5004 of Buffer RW1 is added to each well of the RNEASY 96™ plate and the vacuum is applied for 2 minutes. 1 mL of Buffer RPE is then added to each well of the RNEASY 96™ plate and the vacuum applied for a period of 90 seconds. The Buffer RPE wash is then repeated and the vacuum is applied for an additional 3 minutes. The plate is then removed from the QIAVAC™ manifold and blotted dry on paper towels. The plate is then re-attached to the QIAVAC™ manifold fitted with a collection tube rack containing 1.2 mL collection tubes. RNA is then eluted by pipetting 140 μL of RNAse free water into each well, incubating 1 minute, and then applying the vacuum for 3 minutes.

The repetitive pipetting and elution steps may be automated using a QIAGEN Bio-Robot 9604 (Qiagen, Inc., Valencia Calif.). Essentially, after lysing of the cells on the culture plate, the plate is transferred to the robot deck where the pipetting, DNase treatment and elution steps are carried out.

### Example 11

### Target-Specific Primers and Probes

Probes and primers may be designed to hybridize to a target sequence, using published sequence information.

For example, for human PTEN, the following primer-probe set was designed using published sequence information (GENBANK™ accession number U92436.1. SEQ ID NO: 1).

(SEQ ID NO: 2)
Forward primer: AATGGCTAAGTGAAGATGACAATCAT
(SEQ ID NO: 3)
Reverse primer: TGCACATATCATTACACCAGTTCGT

And the PCR probe:

(SEQ ID NO: 4)
FAM-TTGCAGCAATTCACTGTAAAGCTGGAAAGG-TAMRA,

where FAM is the fluorescent dye and TAMRA is the quencher dye.

### Example 12

### Western Blot Analysis of Target Protein Levels

Western blot analysis (immunoblot analysis) is carried out using standard methods. Cells are harvested 16-20 h after oligonucleotide treatment, washed once with PBS, suspended in Laemmli buffer (100 μl/well), boiled for 5 minutes and loaded on a 16% SDS-PAGE gel. Gels are run for 1.5 hours at 150 V, and transferred to membrane for western blotting. Appropriate primary antibody directed to a target is used, with a radiolabeled or fluorescently labeled secondary antibody directed against the primary antibody species. Bands are visualized using a PHOSPHORIMAGER™ (Molecular Dynamics, Sunnyvale Calif.).

### Example 13

### Preparation of Compound 3

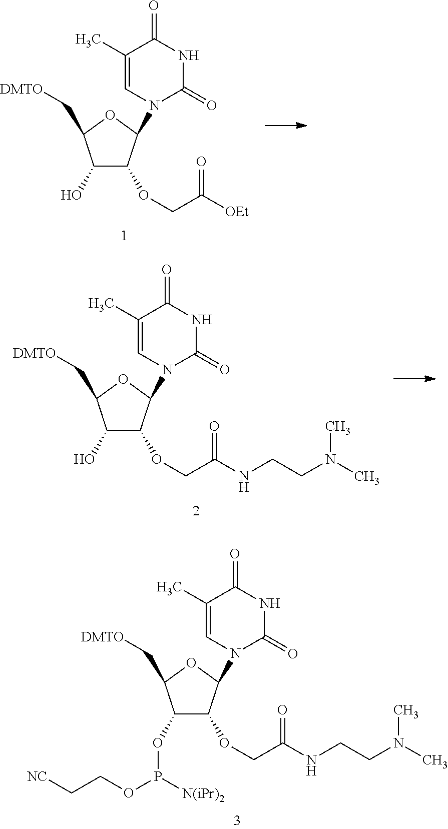

### a) Preparation of 5′-O-(4,4′-dimethoxytrityl)-2′-O-(2-N-[2-(dimethylamino)ethyl]-acetamide)-5-methyluridine (Compound 2)

Compound 1 was prepared according to published literature (Prakash et al., Org. Let. 2003, 5, 403-406) using ethyl-2-bromoacetate for alkylation. Compound 1 (5.378 g, 8.50 mmol) was dissolved in anhydrous THF (66 mL). To this was added N,N-dimethylethylenediamine (18.7 mL, 170 mmol) and the reaction mixture was stirred at ambient temperature. After 6 h, toluene (80 mL) was added and the solvent was evaporated in vacuo to give Compound 2 as a white foam (6.12 g, 95%). ¹H NMR (CDCl₃): δ 7.64 (s, 3H), 7.41-6.79 (m, 13H), 5.94 (d, 1H, Jᵣ=2.4 Hz), 4.41 (m, 1H), 4.31 (q ab, 2H), 4.19 (m, 1H), 3.95 (m, 1H), 3.75 (s, 6H), 3.52 (m, 2H), 2.75 (m, 2H), 2.48 (m, 2H), 2.24 (s, 6H), 1.36 (s, 3H). ¹³C NMR (CDCl₃): δ 170.1, 164.7, 158.7, 151.0, 144.4, 135.5, 135.3, 134.9, 130.1, 129.0, 128.1, 127.7, 127.1, 113.3, 110.9, 88.5, 86.7, 84.8, 83.3, 70.7, 68.2, 61.8, 58.4, 45.4, 36.0, 12.0. HRMS (MALDI) calcd for C₃₇H₄₄N₄O₉+Na⁺: 711.3006. Found: 711.3001. TLC: CH₂Cl₂-EtOAc-MeOH-NEt₃, 64:21:21:5, v/v/v/v; R_{f} 0.4.

### b) Preparation of 5′-O-(4,4′-dimethoxytrityl)-2′-O-(2-N-[2-(dimethylamino)ethyl]-acetamide)-5-methyluridine-3′-(2-cyanoethyl-N,N-diisopropylphosphoramidite) (Compound 3)

Compound 2 (5.754 g, 8.35 mmol) was dried by coevaporation with anhydrous pyridine (2×75 mL) and then dissolved in CH₂Cl₂ (60 mL). To this solution, diisopropylamine tetrazolide (715 mg, 4.18 mmol) and 2-cyanoethyl-N,N,N′,N′-tetraisopropylphosphordiamidite (3.18 mL, 10.02 mmol) were added. After 13 h, EtOAc (420 mL) was added and about 60 mL of solvent was evaporated in vacuo. The organic was washed with half-saturated NaHCO₃ (3×80 mL), then with brine (2×40 mL), dried over MgSO₄, filtered and evaporated in vacuo at 27° C. to give an oil. The resulting residue was coevaporated with toluene (2×300 mL) to give a foam which was then dissolved in CH₂Cl₂ (20 mL). Hexanes (1000 mL) were slowly added to the rapidly stirred solution via an addition funnel to yield a wax and the supernatant was decanted. The wax was washed with hexanes thrice and the washes were decanted. The precipitation was repeated one more time to give a white wax which was dried in vacuo at ambient temperature to give Compound 3 as a foam (6.60 g, 89%). LRMS (ES): m/z 889 (M+H⁺), 911 (M+Na⁺). ³¹P NMR (CDCl₃): δ 151.5, 151.0.

Compound 3 was incorporated into oligonucleotides according to standard solid phase synthesis procedures. Phosphorylation at the 5′ end of oligonucleotides was achieved during synthesis by using Glen Research (Sterling, Va.) chemical phosphorylation reagent.

### Example 14

### Preparation of Compound 4

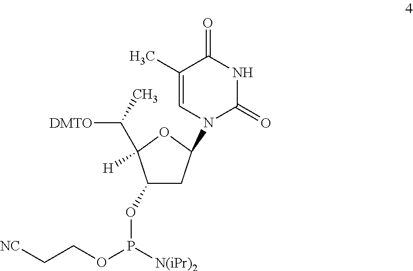

Compound 4 was prepared according to the procedures described in published patent application WO 94/22890. Compound 4 was incorporated into oligonucleotides according to standard solid phase synthesis procedures. Phosphorylation at the 5′ end of oligonucleotides was achieved during synthesis by using Glen Research (Sterling, Va.) chemical phosphorylation reagent.

### Example 15

### Preparation of Compound 13

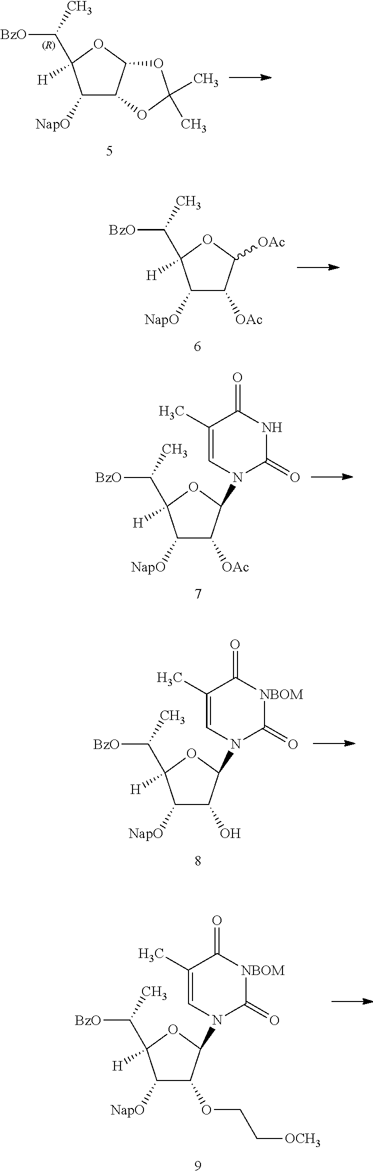

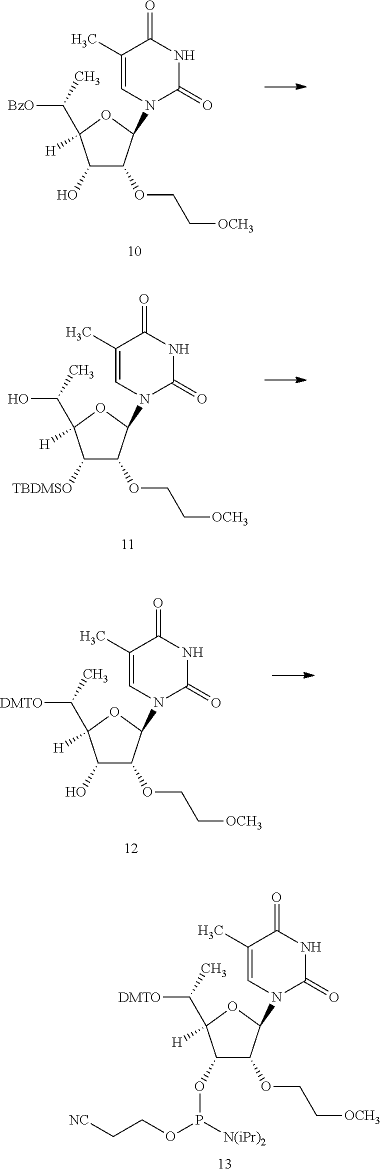

### a) Preparation of 5-O-Benzyol-3-O-(2-methylnaphthalene)-1,2-O-bis(acetyl)-5-(R)-methyl-ribose (Compound 6)

Compound 5 was prepared according to the method of De Mesmaeker wherein NapBr was used instead of BnBr (Mesmaeker et al., Synlett, 1997, 1287-1290). Dried Compound 5 (21.1 g, 47.04 mmol) was dissolved in a mixture of glacial acetic acid (104 mL) and acetic anhydride (17.2 mL). To this solution was added 14 drops of concentrated H₂SO₄. After 1.5 h, the resulting light brown solution was diluted in EtOAc (600 mL), washed with sat. NaHCO₃ (5×600 mL), dried over anhydrous Na₂SO₄, filtered, evaporated and dried under high vacuum to yield Compound 6 (22.7 g, 99%) as a pale oil. ES MS m/z 515.1 [M+Na]⁺.

### b) Preparation of 5′-O-Benzyol-3′-O-(2-methylnaphthalene)-5′-(R)-methyl-5-methyluridine (Compound 7)

A mixture of Compound 6 (23.3 g, 46.70 mmol) and thymine (10.01 g, 79.40 mmol) was suspended in anhydrous CH₃CN (233 mL). To this mixture was added N,O-bis-trimethylsilylacetamide (41.06 mL, 167.94 mmol), followed by heating at 55° C. for 1 h. The mixture was cooled to 0° C., then trimethylsilyl trifluoromethanesulfonate (19.07 mL, 105.54 mmol) was added dropwise over 15 min. The mixture was subsequently heated at 55° C. After 3 hours the mixture was cooled to 0° C. and quenched with the dropwise addition of saturated aqueous NaHCO₃ (20 mL). The mixture was poured into EtOAc, washed with brine (4×0.8 mL), dried over anhydrous Na₂SO₄, filtered, evaporated and dried under high vacuum. The residue was purified by silica gel column chromatography and eluted with 20% to 50% EtOAc in hexanes to yield Compound 7 (22.27 g, 85%) as a white foam. ES MS m/z 559.2 [M+H]⁺.

### c) Preparation of 3′-O-(2-methylnaphthalene)-5′-(R)-methyl-3-N-(benzyloxymethyl)-5-methyluridine (Compound 8)

Compound 7 (11.71 g, 20.98 mmol) was dissolved in anhydrous DMF (115 mL). To this was added 1,8-diazabicycl-[5-4-0]undec-7-ene (DBU, 9.30 mL, 62.41 mmol). The reaction mixture was cooled in an ice bath. To this was added benzyl chloromethyl ether (4.36 mL, 31.47 mmol), and stirred at 0° C. for 1 hour. The mixture was diluted with EtOAc (200 mL), washed with saturated aqueous NaHCO₃ (200 mL) and brine (200 mL) then dried (Na₂SO₄), filtered and evaporated. The residue obtained was dissolved in methanol (89 mL) and K₂CO₃ (8.76 g, 63.40 mmol). The reaction mixture was stirred at room temperature for 1 h. The mixture was poured into EtOAc (200 mL), washed with water (200 mL) and brine (200 mL), dried over anhydrous Na₂SO₄, filtered and evaporated. The residue was purified by silica gel column chromatography and eluted with 5% methanol in CH₂Cl₂ to yield Compound 8 (8.93 g, 80%) as a white foam. ES MS m/z 533.2 [M+H]⁺.

### d) Preparation of 2′-O-(2-methoxyethyl)-3′-O-(2-methylnaphthalene)-5′-(R)-methyl-3-N-(benzyloxymethyl)-5-methyluridine (Compound 9)

Compound 8 (4.30 g, 8.07 mmol) was dried over P₂O₅ under reduced pressure and dissolved in anhydrous DMF (24 mL). The mixture was cooled to −20° C. To this was added NaH (0.48 g, 12.11 mmol, 60% dispersion in mineral oil) with stirring for 30 minutes followed by addition of 1-methoxy-2-iodoethane (2.25 g, 12.11 mmol). The reaction mixture was warmed up to 0° C. After stirring for 1.5 h at 0° C. the reaction mixture was cooled to −20° C. and additional NaH (0.48 g, 12.11 mmol, 60% dispersion in mineral oil) was added. Stirring was continued at −20° C. for 30 minutes and 1-methoxy-2-iodoethane (2.25 g, 12.11 mmol) was added. The reaction mixture was warmed to 0° C. and with stirring for an additional 1.5 h. The reaction was quenched with methanol (5 mL), diluted with EtOAc (100 mL), washed with water (100 mL) and brine (100 mL), dried over Na₂SO₄, filtered and evaporated under reduced pressure. The residue was purified by silica gel column chromatography and eluted with 5% methanol in CH₂Cl₂ to yield Compound 9 (2.95 g, 62%). ES MS m/z 591.2 [M+H]⁺.

### e) Preparation of 5′-O-Benzoyl-2′-O-(2-methoxyethyl)-5′-(R)-methyl-5-methyluridine (Compound 10)

Compound 9 (2.2 g, 3.73 mmol) was dissolved in anhydrous pyridine (7 mL) and cooled in an ice bath. To this benzoyl chloride (0.88 mL, 7.61 mmol) was added and once the addition was over, reaction mixture was allowed to come to room temperature. The reaction mixture was stirred at room temperature for 4 h under an argon atmosphere and subsequently cooled the reaction mixture in an ice bath and quenched by adding saturated aqueous NaHCO₃ (5 mL). Diluted the reaction mixture with EtOAc (50 mL) and washed with saturated aqueous NaHCO₃ (2×50 mL), brine (50 mL), dried over Na₂SO₄, filtered and concentrated. The residue obtained was dissolved in CH₂Cl₂ (40 mL) and added 2,4-dichloro-5,6-dicyano-1,4-benzoquinone (DDQ, 1.93 g, 8.5 mmol) and H₂O (0.15 mL, 8.5 mmol) and stirred at room temperature. After 18 h, diluted the reaction mixture with EtOAc (60 mL), washed with saturated aqueous NaHCO₃ (2×80 mL), brine (50 mL), dried over Na₂SO₄, filtered and evaporated under reduced pressure. The residue was dissolved in MeOH (30 mL) and palladium hydroxide (1.1 g, 20 wt % Pd on carbon dry base) and stirred under H₂ atmosphere for 6 h. To this acetic acid (0.56 mL) was added and stirred for 5 min. The reaction mixture was filtered through a pad of celite 545, and washed the celite with copious amount of MeOH. The combined filtrate and washing were concentrated under reduced pressure and the residue was purified by silica gel column chromatography and eluted with 5% methanol in CH₂Cl₂ to yield Compound 10 (1.43 g, 88%). ES MS m/z 435.1 [M+H]⁺.

### f) Preparation of 2′-O-(2-methoxyethyl)-5′-(R)-methyl-3′-O-tert-butyldimethylsilyl-5-methyluridine (Compound 11)

A mixture of Compound 10 (1.33 g, 3.06 mmol) and imidazole (2.09, 30.70 mmol) was dissolved in anhydrous DMF (11.4 mL). To this solution tert-butyldimethylsilyl chloride (2.31 g, 15.33 mmol) was added with stirring at room temperature for 16 h under an atmosphere of argon. The reaction mixture was diluted with EtOAc (75 mL) and washed with saturated aqueous NaHCO₃ (2×60 mL) and brine (50 mL), dried over Na₂SO₄, filtered and concentrated. The residue obtained was dissolved in methanolic ammonia (20 mL, 7M) and stirred for 24 h at 55° C. The solvent was removed under reduced pressure and the residue was purified by silica gel column chromatography and eluted with 50% EtOAc in hexanes to yield Compound 11 (1.21 g, 89%). ES MS m/z 455.2 [M+H]⁺.

### g) Preparation of 5′-O-(4,4′-dimethoxytrityl)-2′-O-(2-methoxyethyl)-5′-(R)-methyl-5-methyluridine (Compound 12)

Compound 11 (0.42 g, 0.96 mmol) was mixed with 4,4′-dimethoxytrityl chloride (0.82 g, 2.41 mmol) and dried over P₂O₅ under reduced pressure. The mixture was dissolved in anhydrous pyridine (3 mL) and stirred at 45° C. for 18 h under an atmosphere of argon. The reaction mixture was cooled to room temperature and diluted with EtOAc (40 mL) and washed with saturated aqueous NaHCO₃ (60 mL) and brine (40 mL), dried over Na₂SO₄, filtered and concentrated. The residue obtained was purified by silica gel column chromatography and eluted first with 50% EtOAc in hexanes and then with 5% methanol in CH₂Cl₂. The product obtained was dissolved in a mixture of triethylamine trihydrofluoride (1.38 mL, 8.44 mmol) and triethylamine (0.58 mL, 4.22 mmol) in THF (8.4 mL). After 72 h the mixture was diluted with EtOAc (60 mL), washed with water (40 mL), saturated aqueous NaHCO₃ (40 mL) and brine (40 mL) then dried over Na₂SO₄, filtered and evaporated. The residue obtained was purified by silica gel column chromatography and eluted with 70% EtOAc in hexanes to yield Compound 12 (0.44 g, 73%). ES MS m/z 631.2 [M+H]⁺.

### h) Preparation of 5′-O-(4,4′-dimethoxytrityl)-2′-O-(2-methoxyethyl)-5′-(R)-methyl-5-methyluridine-3′-(2-cyanoethyl-N,N-diisopropylphosphoramidite (Compound 13)

Compound 12 (0.35 g, 0.55 mmol) was dried over P₂O₅ under reduced pressure then dissolved in anhydrous DMF (1.8 mL). To this 1-H-tetrazole (0.033 mg, 0.48 mmol), N-methylimidazole (0.012 mL, 0.15 mmol) and 2-cyanoethyl-N,N,N′,N′-tetraisopropylphosphordiamidite (0.27 mL, 0.86 mmol) were added. After 3 h, EtOAc (40 mL) was added and the mixture was washed with saturated NaHCO₃ (30 mL) and brine (40 mL), dried over anhydrous Na₂SO₄, filtered and evaporated in vacuo to give an oil. The oily residue was purified by silica gel column chromatography by eluting with EtOAc/hexane (1:1) to yield Compound 13 (0.38 g, 83%) as a white foam. MS (ES): m/z 831 [M+H]⁺; ³¹P NMR (121 MHz, CDCl₃): δ 150.2, 149.

### Example 16

### Preparation of Compound 22

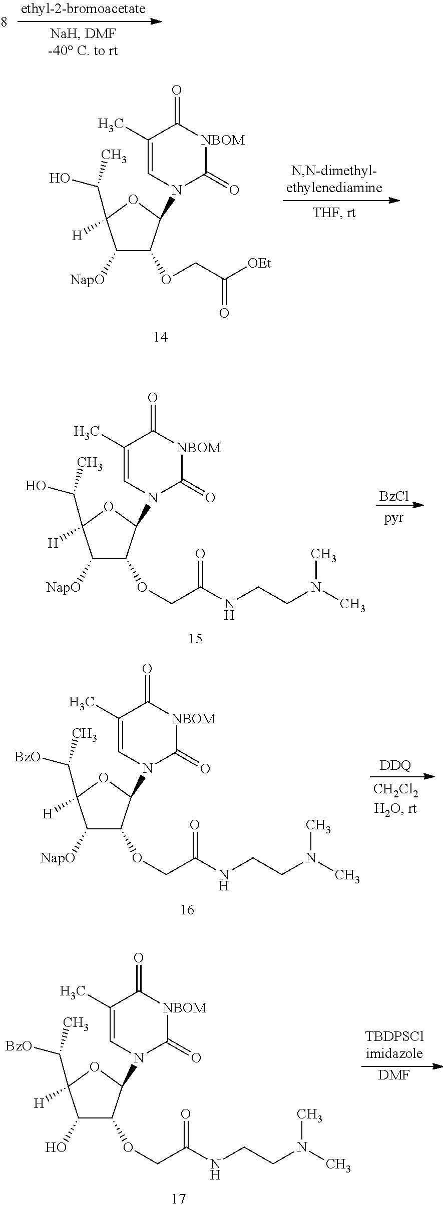

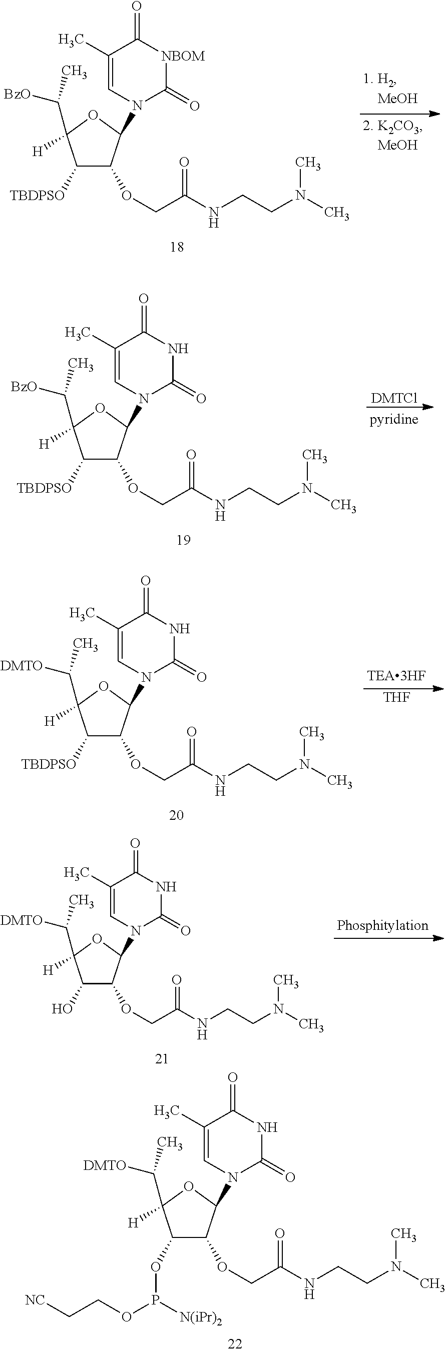

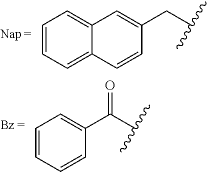

Compound 8 is prepared as per the procedures illustrated in Example 15. Compound 22 is prepared according to the scheme illustrated above. Compound 22 is incorporated into oligonucleotides according to standard solid phase synthesis procedures. Phosphorylation at the 5′ end of oligonucleotides is achieved during synthesis by using Glen Research (Sterling, Va.) chemical phosphorylation reagent.

### Example 17

### Preparation of Compound 26

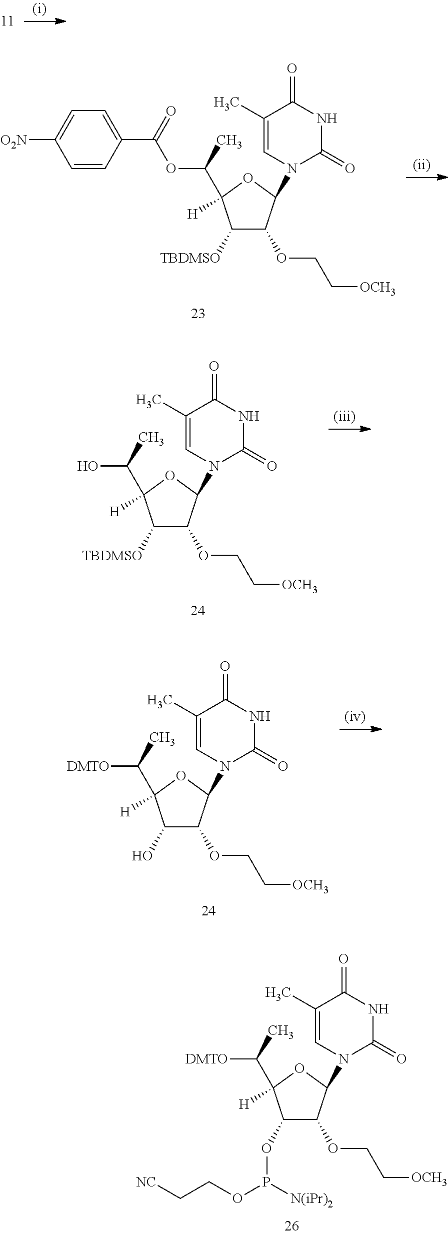

Scheme 1. (i) 4-nitrobenzoic acid, triphenylphosphine, diisopropyl azodicarboxylate, rt; (ii) NH₃, MeOH, 55° C.; (iii) a. DMTCl, pyridine, 45° C., b. THF.3HF, TEA, THF; (iv) 2-cyanoethyl-N,N,N′,N′-tetraisopropylphosphordiamidite, 1-H-tetrazole, N-methyl-imidazole, DMF.

Compound 11 is prepared as per the procedures illustrated in Example 15.

### Example 18

### Preparation of Compound 30

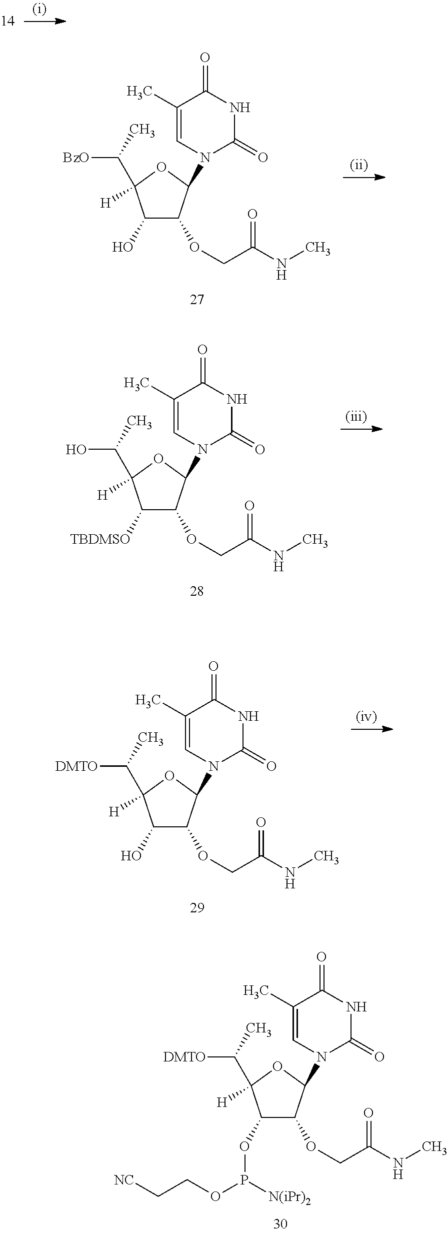

Scheme 2. Nap: 2-methylnaphthalene; Bz: benzoyl; TBDMS: tert-butyldimethylsilyl; (i) DMF, 2-bromoethyl acetate, NaH; (ii) a. aqueous CH₃NH₂, THF, b. BzCl, pyridine, rt, c. DDQ, CH₂Cl₂, H₂O, rt, c. Pd(OH)₂, MeOH, H₂, AcOH; (iii) a. TBDMSCl, Im, DMF, rt, b. NH₃, MeOH, 55° C.; (iv) a. DMTC1, Py, 45° C., b. TEA.3HF, TEA, THF; (v) 2-cyanoethyl-N,N,N′,N′-tetraisopropyl-phosphordiamidite, 1-H-tetrazole, N-methylimidazole, DMF.

Compound 14 is prepared as per the procedures illustrated in Example 16.

### Example 19

### Preparation of Compound 34

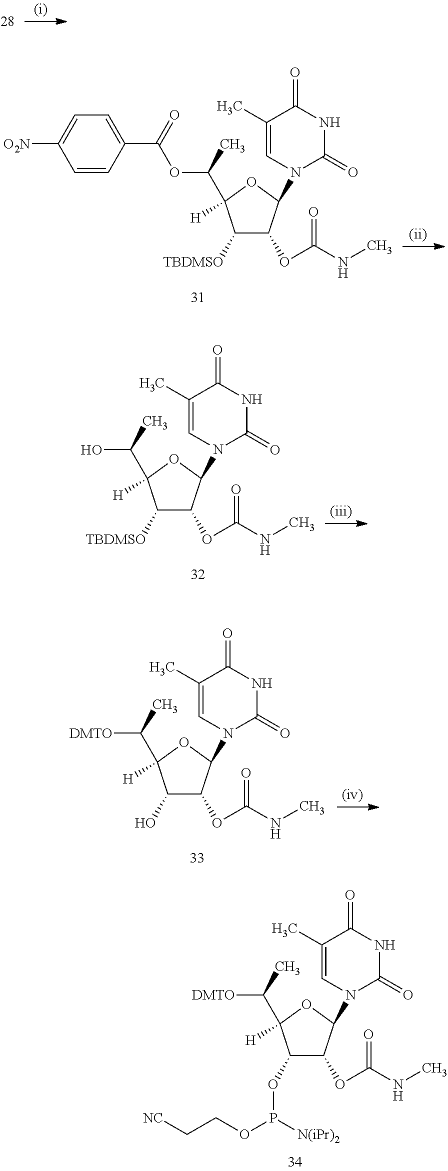

Scheme 3. (i) 4-nitrobenzoic acid, triphenylphosphine, diisopropyl azodicarboxylate, rt; (ii) NH₃, MeOH, 55° C.; (iii) a. DMTCl, pyridine, 45° C., b. TEA.3HF, TEA, THF; (iv) 2-cyanoethyl-N,N,N′N′-tetraisopropylphosphordiamidite, 1-H-tetrazole, N-methylimidazole, DMF.

Compound 28 is prepared as per the procedures illustrated in Example 18

### Example 20

### Preparation of Compound 37

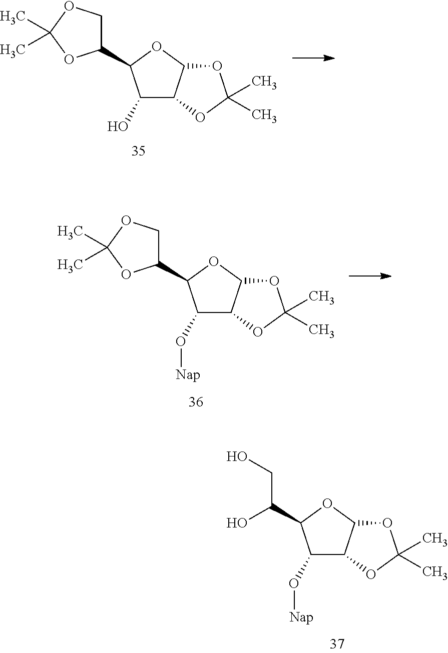

### a) Preparation of Compound 36

Commercially available 1,2;5,6-di-O-isopropylidene-α-D-allofuranose, Compound 35, (135 g, 519.0 mmol) and 2-(bromomethyl)-naphthalene (126 g, 570.0 mmol) were dissolved in DMF (500 mL) in a three-necked flask (500 mL) and the reaction was cooled in an ice bath. Sodium hydride (60% w/w, 29 g, 727.0 mmol) was carefully added (6 g portions every 10 minutes) to the reaction and the stirring was continued for another 60 minutes after the addition was complete. At this time TLC analysis showed no more sugar (Compound 35). The reaction was carefully poured onto crushed ice (ca. 500 g) and the resulting slurry was stirred vigorously until all the ice melted. The resulting off-white solid was collected by filtration and suspended in water. The suspension was stirred vigorously using a mechanical stirrer for 30 minutes after which the solid was collected by filtration and suspended in hexanes. The suspension was stirred vigorously for 30 minutes after which the solid was collected by filtration and air dried for 4-6 hours and then dried under high vacuum over P₂O₅ for 16 hours to provide Compound 36 (206.0 g, 99%) as an off-white solid. ¹H NMR (300 MHz, CDCl₃) δ: 7.85 (m, 4H), 7.48 (m, 3H), 5.74 (s, 1H), 4.92 (d, 1H, J=11.7), 4.75 (d, 1H, J=11.6), 4.58 (m, 1H), 4.36 (m, 1H), 4.15 (m, 1H), 4.03-3.86 (m, 3H), 1.61 (s, 3H), 1.36 (s, 9H).

### b) Preparation of Compound 37

Compound 36 (200.0 g, 0.5 moles) was added in small portions to a solution of acetic acid (2.2 L) and water (740 mL). The reaction was stirred at room temperature for 16 h after which, TLC analysis (30% EtOAc/hexanes) indicated complete consumption of Compound 36. The reaction was then concentrated under reduced pressure until most of the acetic acid was removed. The remaining solution was poured into a stirred mixture of EtOAc (1 L) and water (1 L). Solid KOH was then added to the above mixture until the aqueous layer was strongly basic (pH>12). The organic layer was then separated, washed with saturated sodium bicarbonate solution and brine then dried (Na₂SO₄), filtered and concentrated under reduced pressure to provide Compound 37 as a yellow foam, which was used without any further purification.

### Example 21

### Preparation of Compound 45

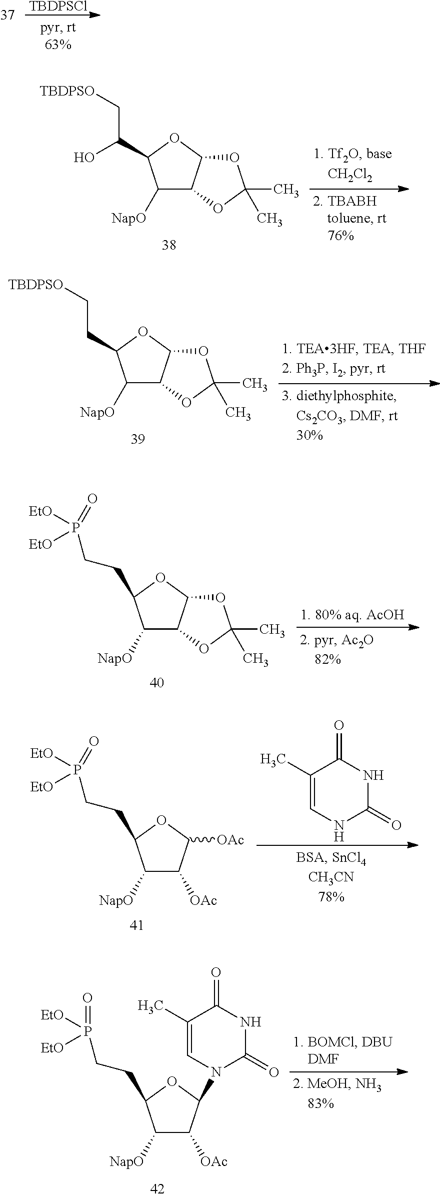

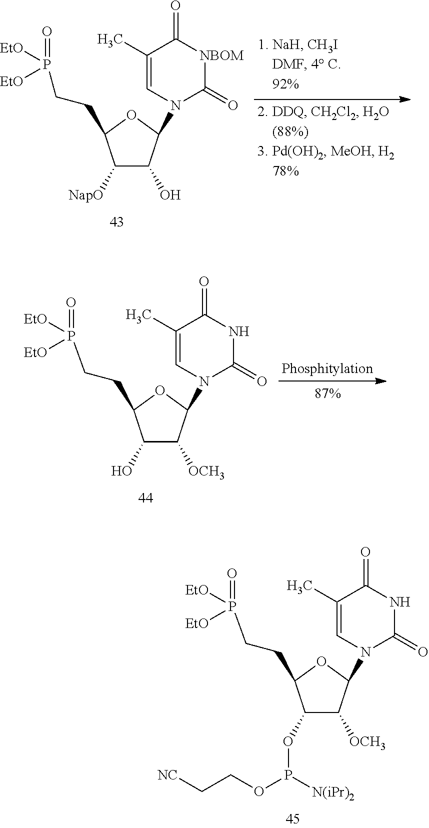

Compound 37 is prepared as per the procedures illustrated in Example 20.

### Example 22

### Preparation of Compound 47

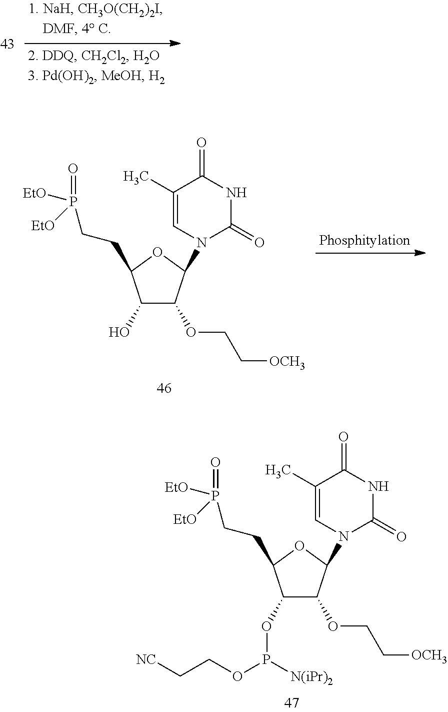

Compound 43 is prepared as per the procedures illustrated in Example 21.

### Example 23

### Preparation of Compound 50

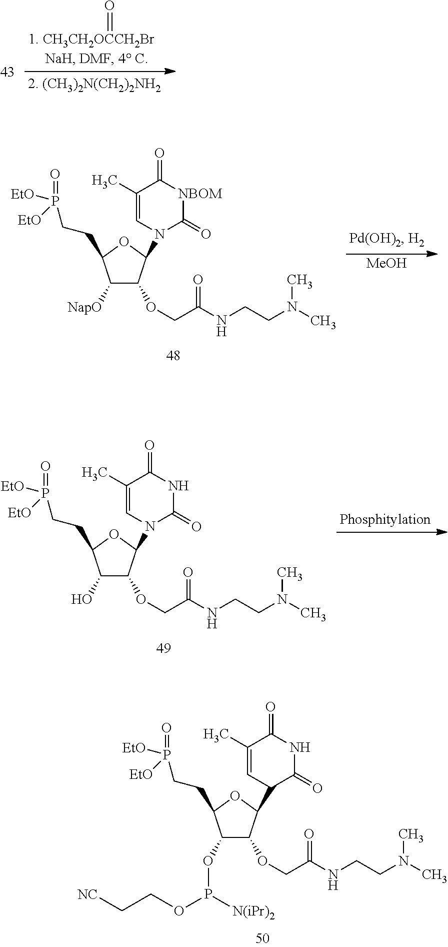

Compound 43 is prepared as per the procedures illustrated in Example 21.

### Example 24

### Preparation of Compound 53

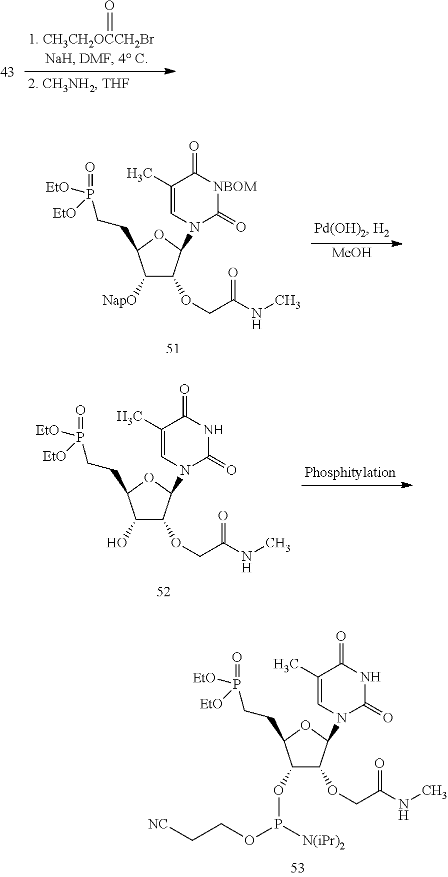

Compound 43 is prepared as per the procedures illustrated in Example 21.

### Example 25

### Preparation of Compound 57

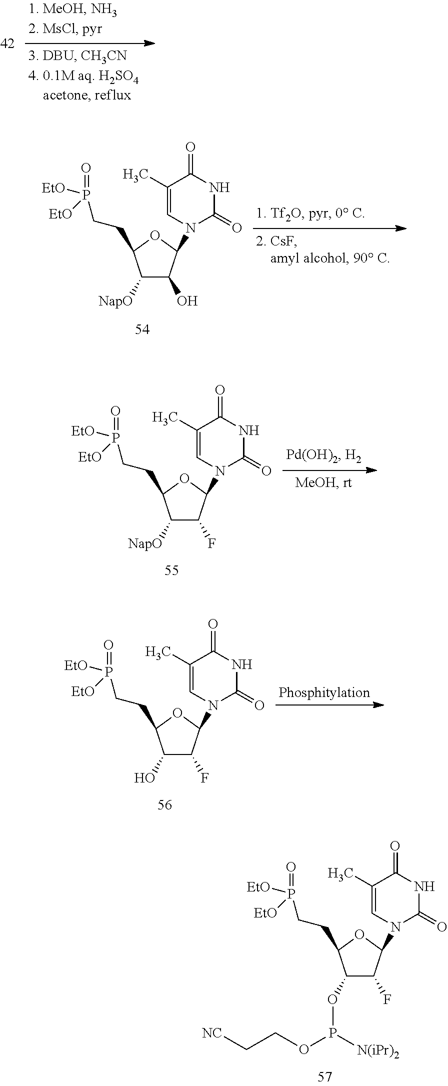

Compound 42 is prepared as per the procedures illustrated in Example 21.

### Example 26

### Preparation of Compound 58

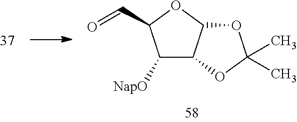

Compound 37 was prepared as per the procedures illustrated in Example 20. A solution of NaIO₄ (107.0 g) in water (3 L) was added over 40 minutes to a stirred (mechanical stirrer) solution of Compound 37 (crude from above) in dioxane (1.5 L). After 60 minutes the reaction mixture was poured into EtOAc (1.5 L) and the organic layer was separated, washed with water (1 L) and brine (1 L) then dried (Na₂SO₄) and concentrated to provide Compound 58 as a yellow oil, which was used without any further purification.

### Example 27

### Preparation of Compound 67

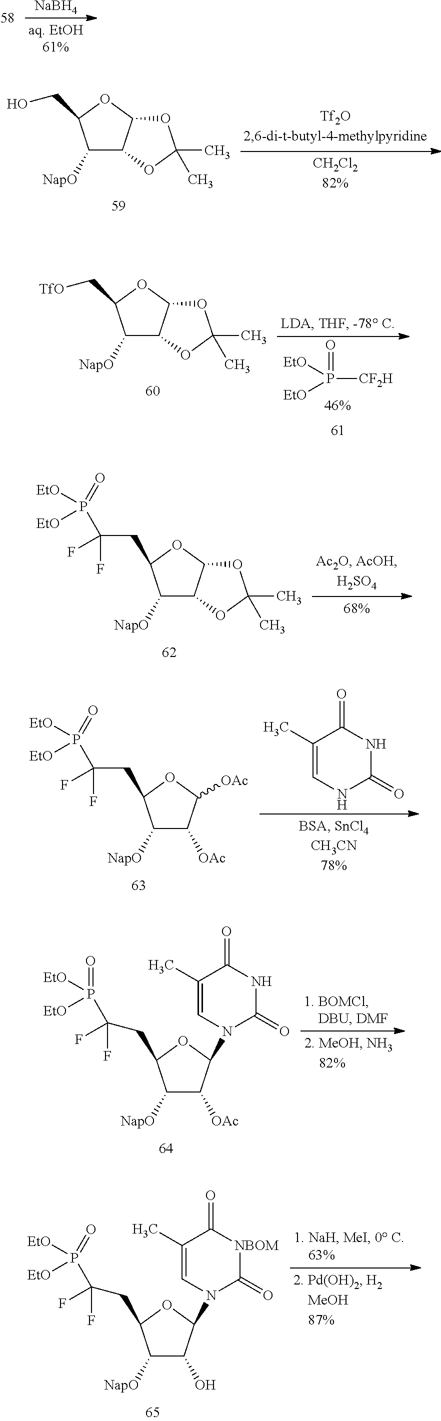

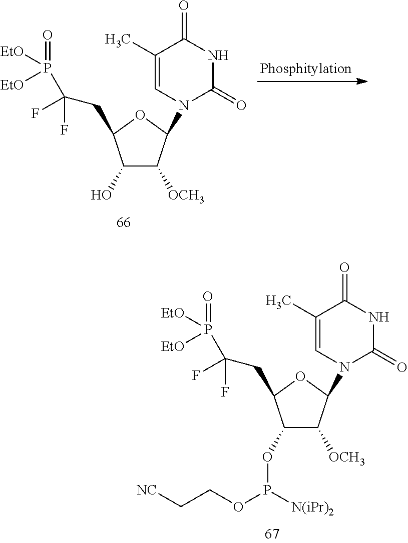

Compound 58 was prepared as per the procedures illustrated in Example 26. Compound 61, diethyl-(difluoromethane)phosphonate is commercially available. The preparation of Compound 67 was achieved as per the procedures illustrated in Example 27 and confirmed by spectral analysis, ¹HNMR and mass spectroscopy.

### Example 28

### Preparation of Compound 69

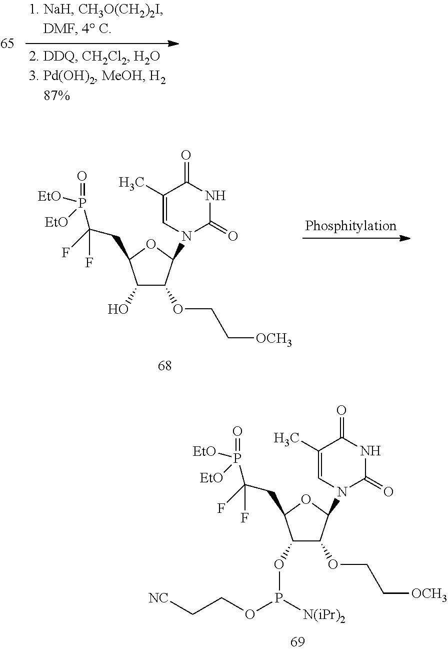

Compound 65 was prepared as per the procedures illustrated in Example 27. The preparation of Compound 69 was achieved as per illustrated in Example 28 and confirmed by spectral analysis, ¹HNMR and mass spectroscopy.

### Example 29

### Preparation of Compound 72

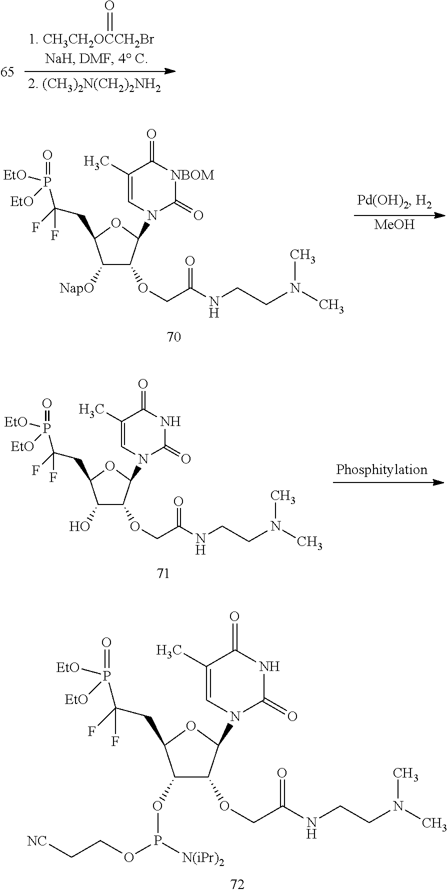

Compound 65 is prepared as per the procedures illustrated in Example 27.

### Example 30

### Preparation of Compound 75

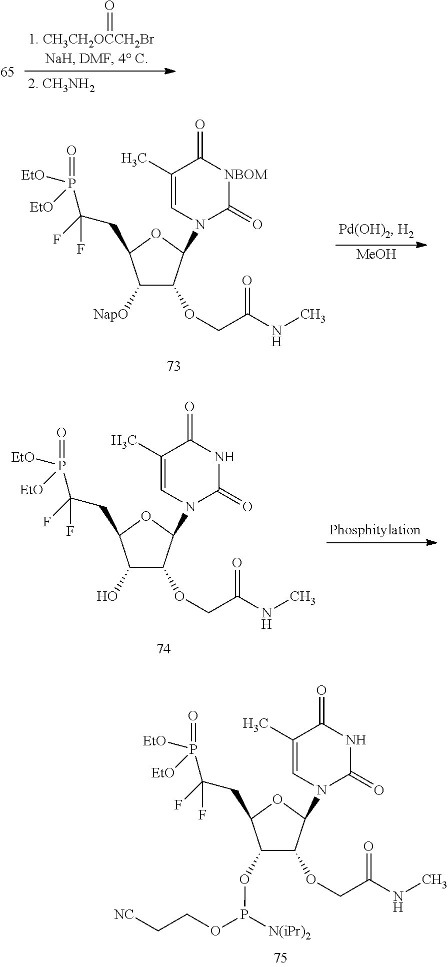

Compound 65 is prepared as per the procedures illustrated in Example 27.

### Example 31

### Preparation of Compound 79

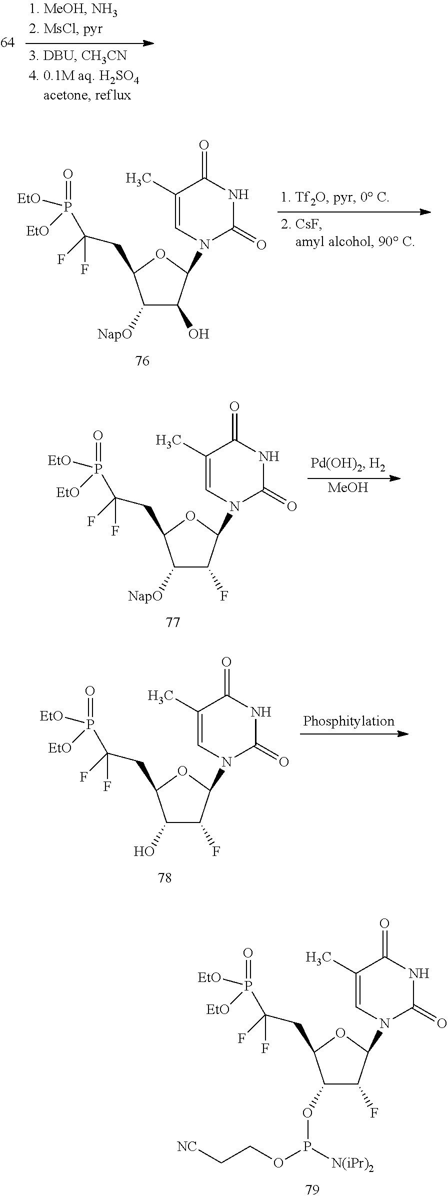

Compound 64 is prepared as per the procedures illustrated in Example 27.

### Example 32

### Preparation of Compound 86

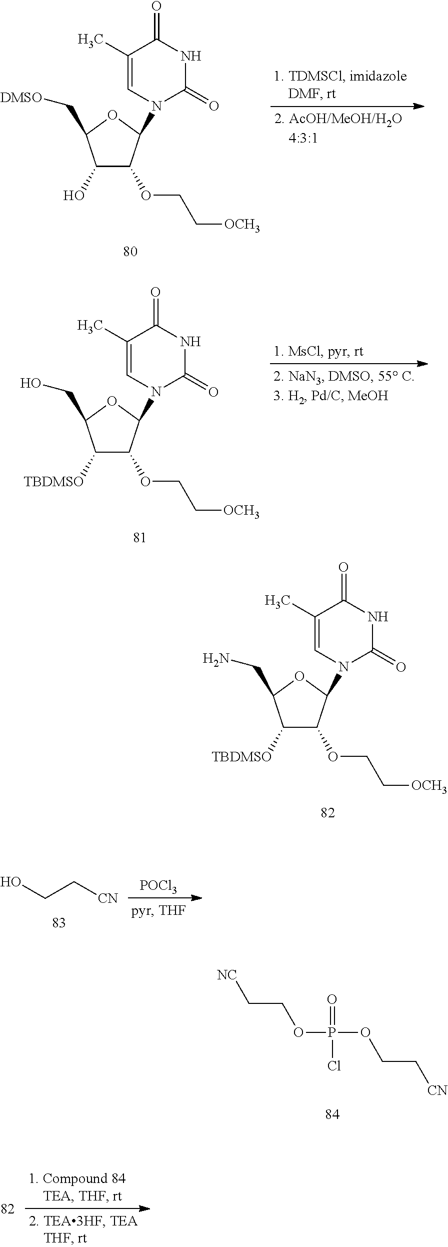

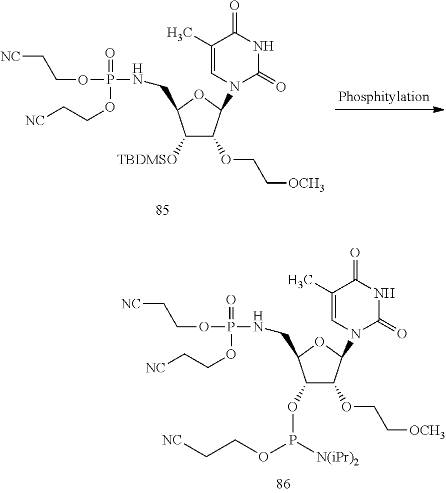

Compound 80 is prepared according to the procedures illustrated in published U.S. Pat. No. 5,969,116.

### Example 33

### Preparation of 5′-N-(4-methoxytrityl)-5′-amino-5′ deoxy-thymidine-3′-(2-cyanoethyl-N,N-diisopropylphosphoramidite) (Compound 89)

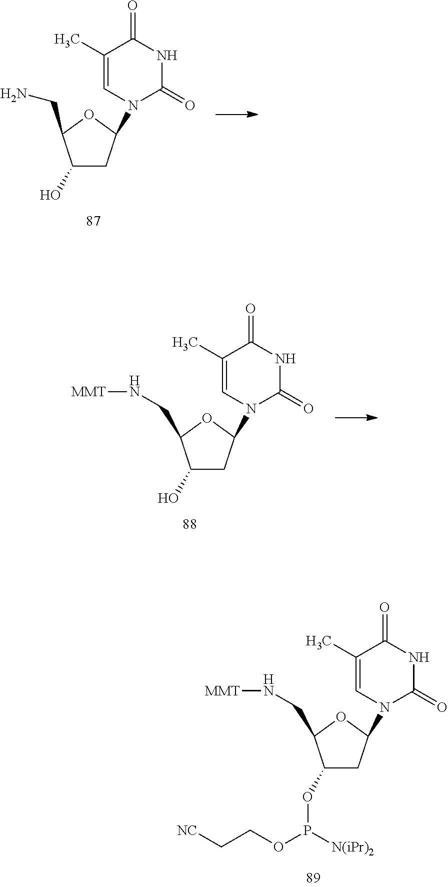

### a) Preparation of 5′-N-(4-methoxytrityl)-5′-amino-5′ deoxy-thymidine (Compound 88)

Compound 87, 5′-amino-deoxythymidine is commercially available. Compound 88 is prepared according to the method of Mag and Engels (Mag, M.; Engles, J. W. Nucleic Acids Res. 1989, 17, 5973-5988).

### b) Preparation of 5′-N-(4-methoxytrityl)-5′-amino-5′ deoxy-thymidine-3′-(2-cyanoethyl-N,N-diisopropylphosphoramidite) (Compound 89)

To the solution of Compound 88 (1.05 g, 1.88 mmol) and tetrazole (0.11 g, 1.5 mmol) in anhydrous DMF (9 mL) was added 1-methylimidazole (0.039 mL, 0.5 mmol) while stirring under a nitrogen atmosphere. The reaction mixture was cooled to 0° C. and 2-cyanoethyl-N,N,N′,N′-tetraisopropylphosphordiamidite (0.89 mL, 2.8 mmol) was added. After 3.5 h, the reaction was quenched with butanol (2 mL) and the reaction volume was reduced to 50% by volume under reduced pressure. The reaction mixture was diluted with EtOAc (50 mL), washed with saturated NaHCO₃ (35 mL), then with brine (50 mL) and dried briefly over anhydrous Na₂SO₄. The organic phase was filtered and concentrated under reduced pressure. The resulting residue was dissolved in diethyl ether:CH₂Cl₂ (1:1, 2.25 mL) and was added drop-wise into an ice cold pentane (300 mL) solution. The resulting solid was filtered to afford Compound 89 (1.24 g, 86.7%). ³¹P NMR (121 MHz, CD₃CN): δ 148.31 and 148.08.

### Example 34

### Preparation of Compound 92

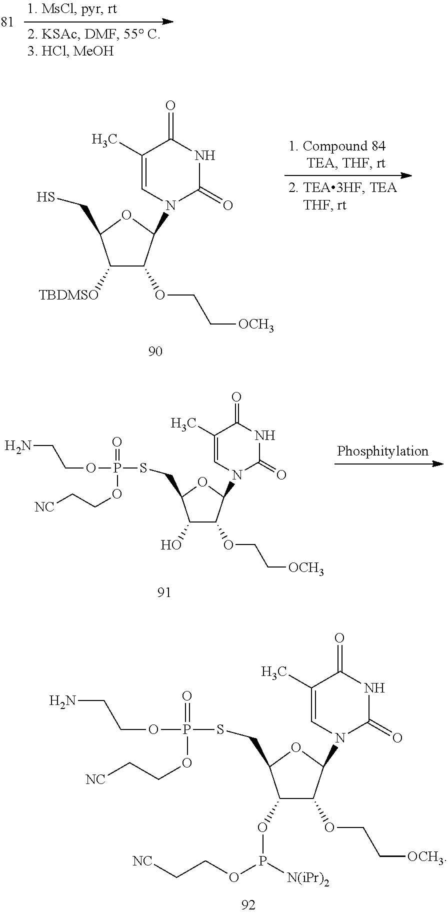

Compounds 81 and 84 are prepared as per the procedures illustrated in Example 32.

### Example 35

### Preparation of 5′-S-(4,4′-dimethoxytrityl)-5′-thiothymidine 3′-(2-cyanoethyl-diisopropylphosphoramidite) (Compound 93)

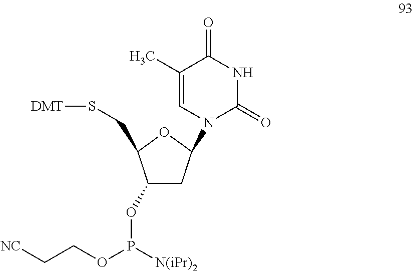

Compound 93 is prepared according to the method of Jahn-Hofmann and Engels (Jahn-Hofmann, K.; Engles, J. W. Helvetica Chimica Acta 2004, 87, 2812-2828).

### Example 36

### Preparation of Compound 102

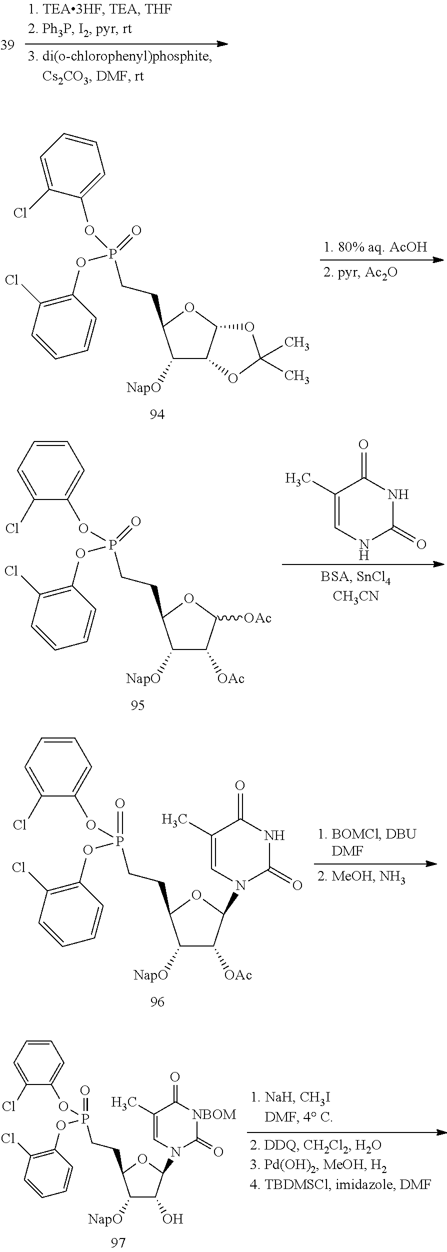

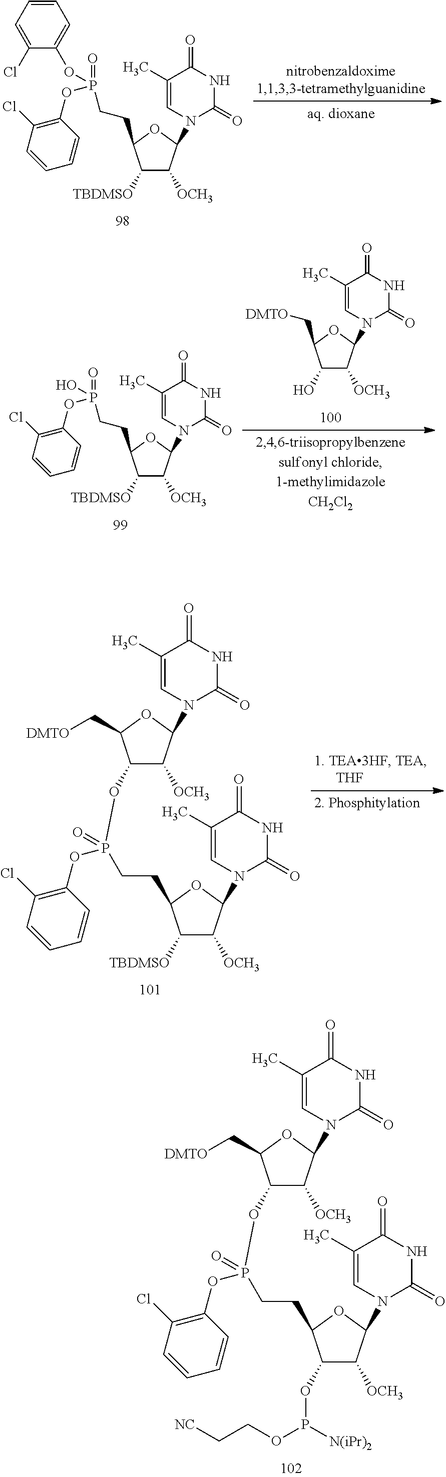

Compound 39 is prepared as per the procedures illustrated in Example 21. Compound 100 is prepared according to the method published by Inoue, H. et al. Nucleic Acids Research 1987, 15, 6131-6148.

### Example 37

### Preparation of Compound 106

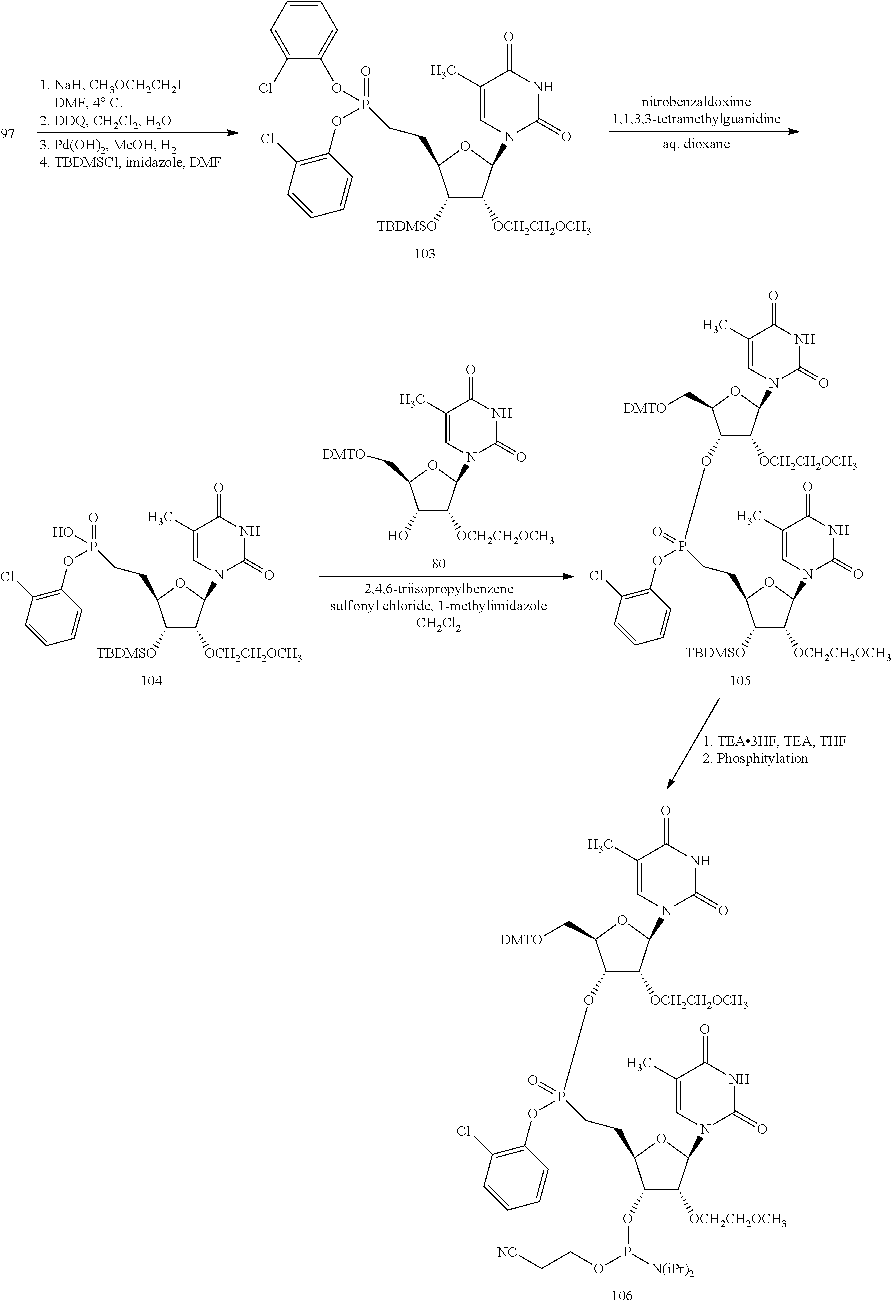

Compound 97 is prepared as per the procedures illustrated in Example 36. Compound 80 is prepared according to the procedures published in U.S. Pat. No. 5,969,116.

### Example 38

### Preparation of Compound 109

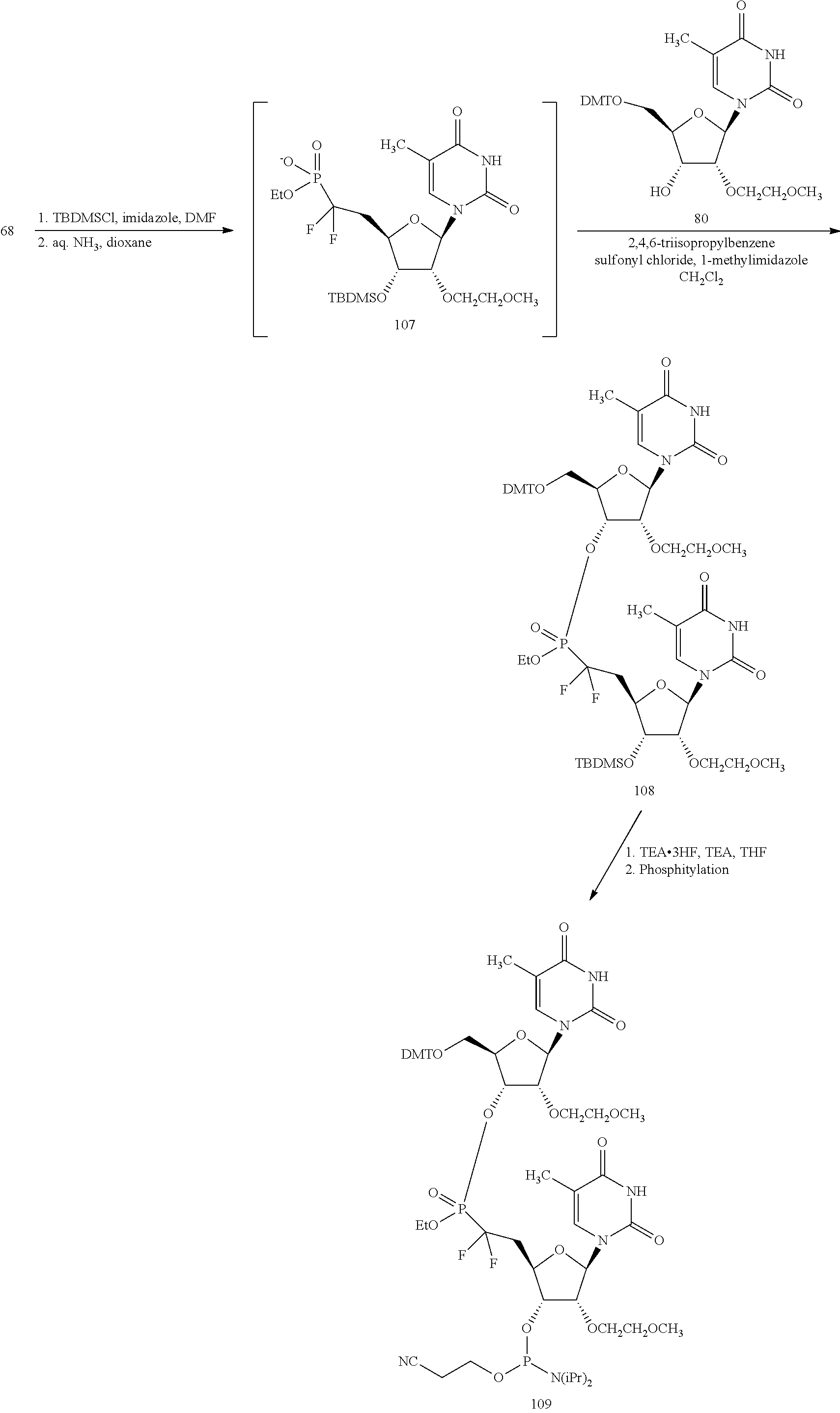

Compound 68 is prepared as per the procedures illustrated in Example 28. Compound 80 is prepared according to the procedures published in U.S. Pat. No. 5,969,116.

### Example 39

### Preparation of Compound 112

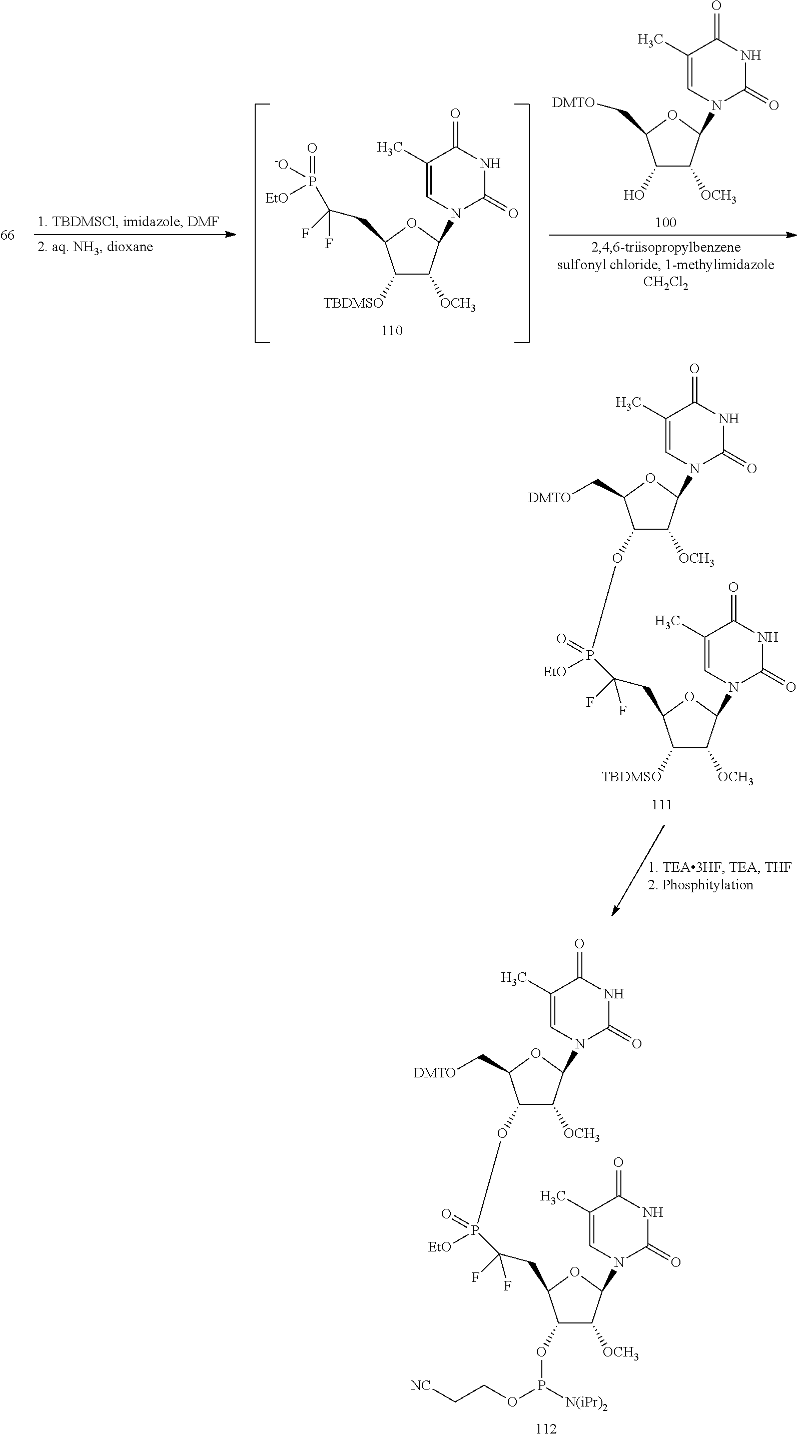

Compound 66 is prepared as per the procedures illustrated in Example 27. Compound 100 is prepared according to the method published by Inoue, H. et al. Nucleic Acids Research 1987, 15, 6131-6148.

### Example 40

### Preparation of Compound 116

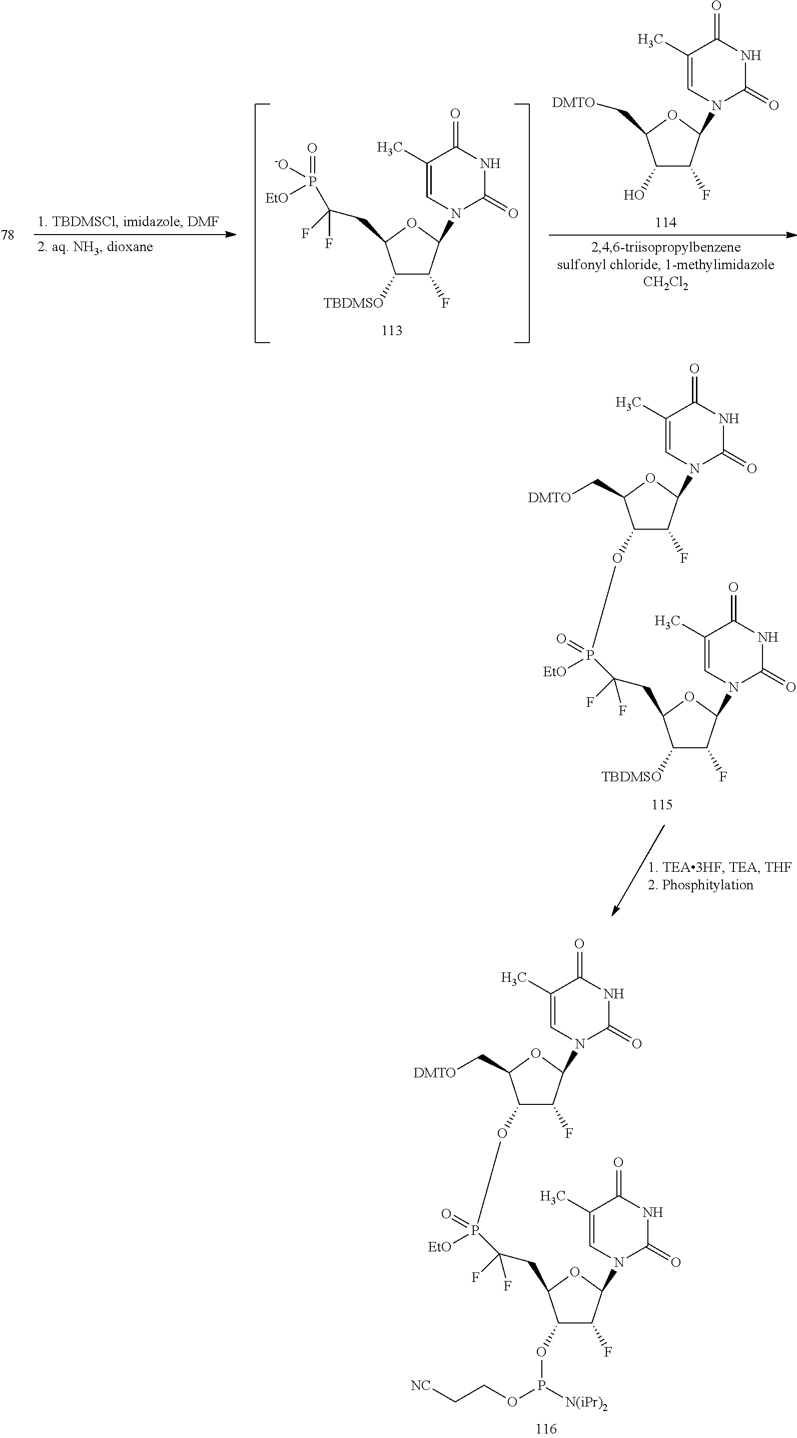

Compound 78 is prepared as per the procedures illustrated in Example 31. Compound 114 is prepared according to procedures published by Ikeda, H. et al. Nucleic Acids Research 1998, 26, 2237-2244.

### Example 41

### Preparation of Compounds 119, 120 and 121

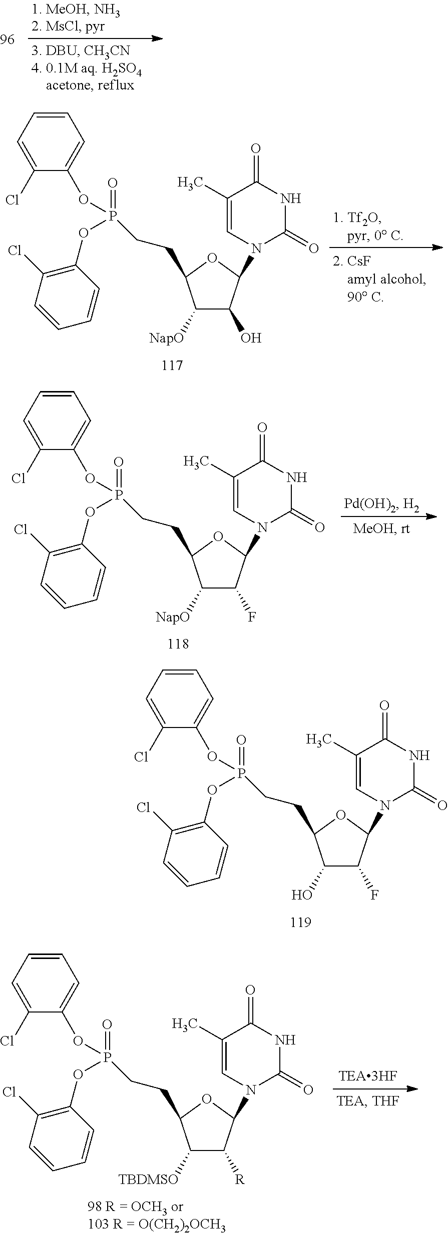

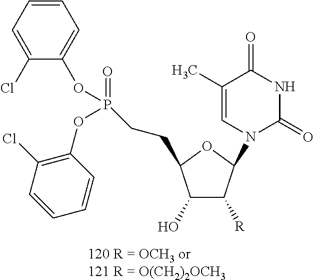

Compounds 96 and 98 are prepared as per the procedures illustrated in Example 36. Compound 103 is prepared as per the procedures illustrated in Example 37.

### Example 42

### Preparation of Compound 125

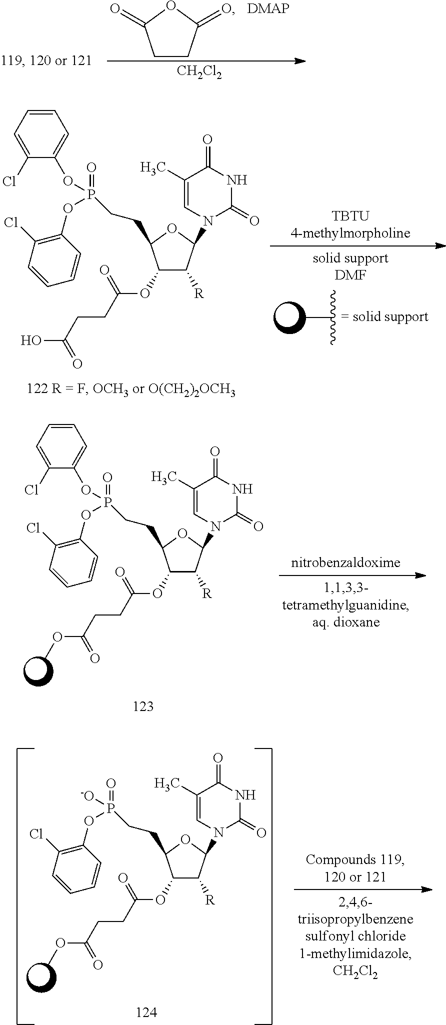

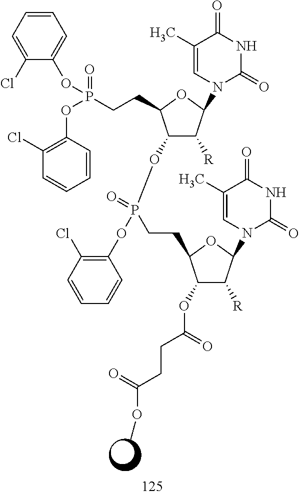

Compounds 119, 120 and 121 are prepared as per the procedures illustrated in Example 41.

### Example 43

### Preparation of Compounds 126 and 127

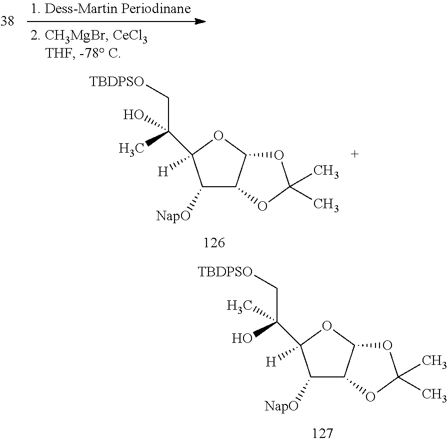

Compound 38 is prepared as per the procedures illustrated in Example 21.

### Example 44

### Preparation of Compounds 134 and 136

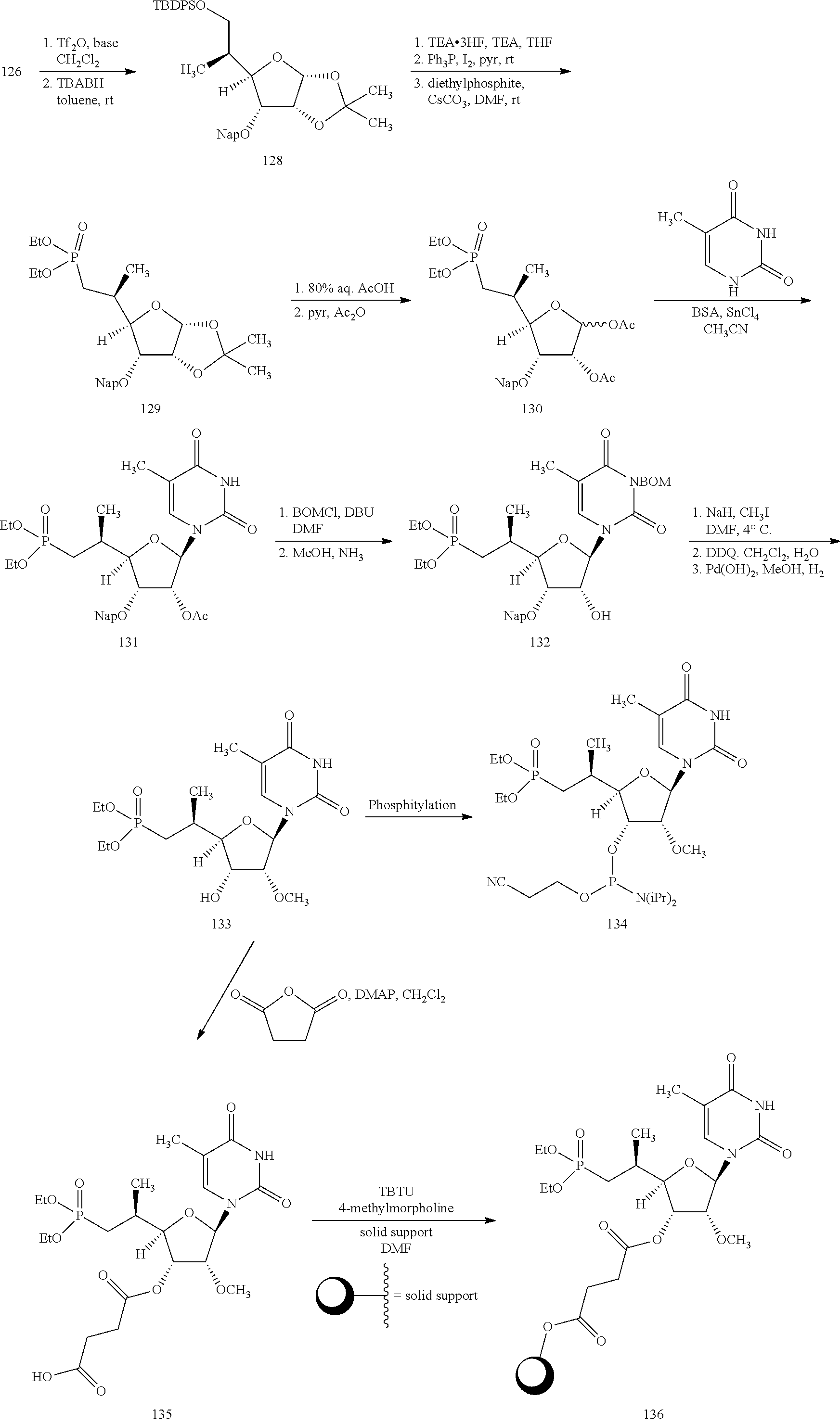

Compound 126 is prepared as per the procedures illustrated in Example 43.

### Example 45

### Preparation of Compounds 143 and 145

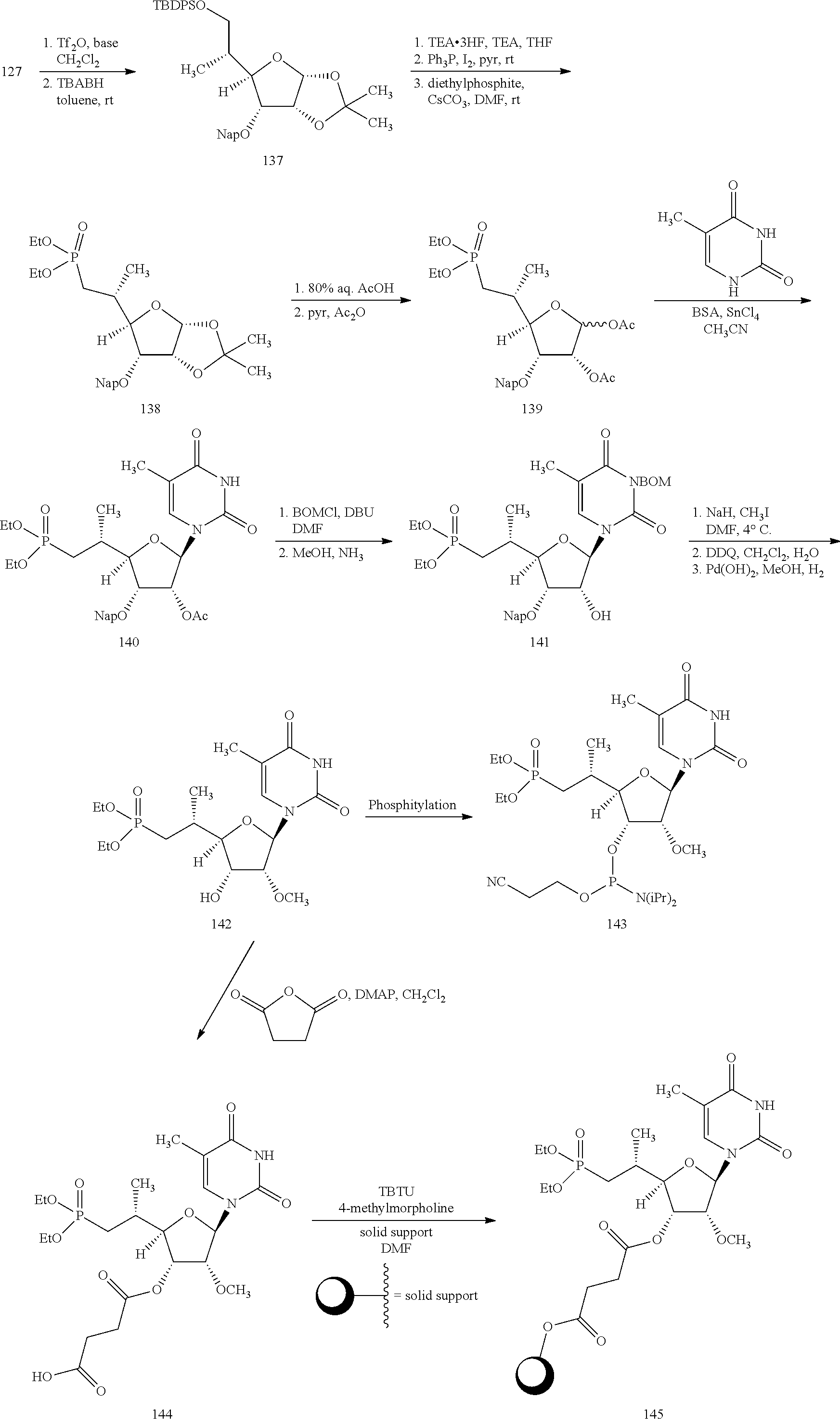

Compound 127 is prepared as per the procedures illustrated in Example 43.

### Example 46

### Preparation of Compounds 147 and 149

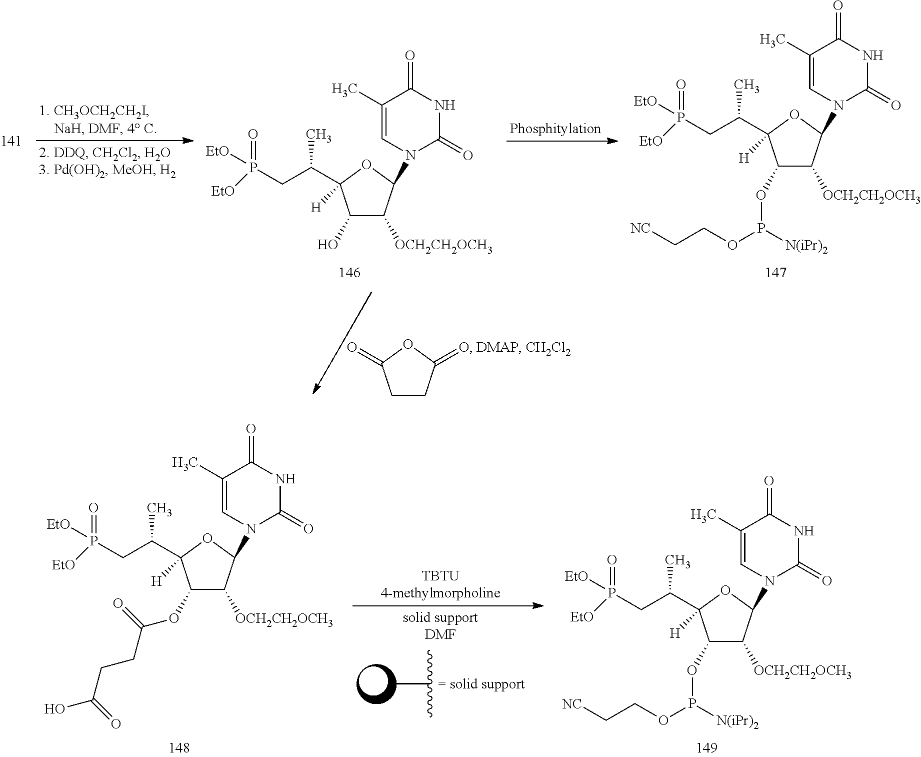

Compound 141 is prepared as per the procedures illustrated in Example 45.

### Example 47

### Preparation of Compounds 151 and 153

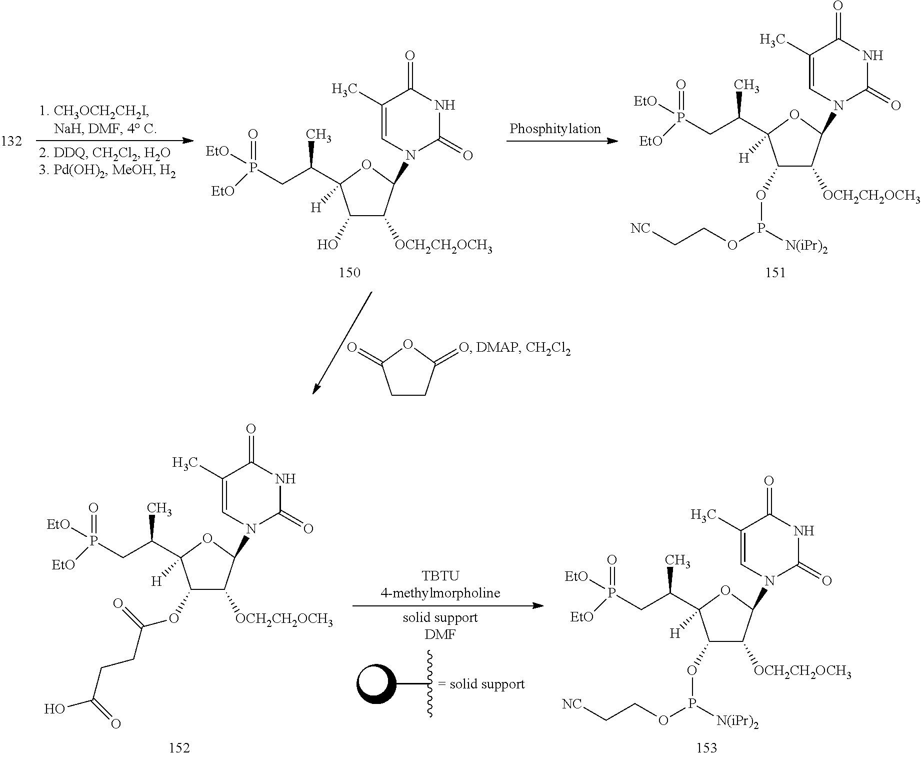

Compound 132 is prepared as per the procedures illustrated in Example 44.

### Example 48

### Preparation of Compounds 155 and 157

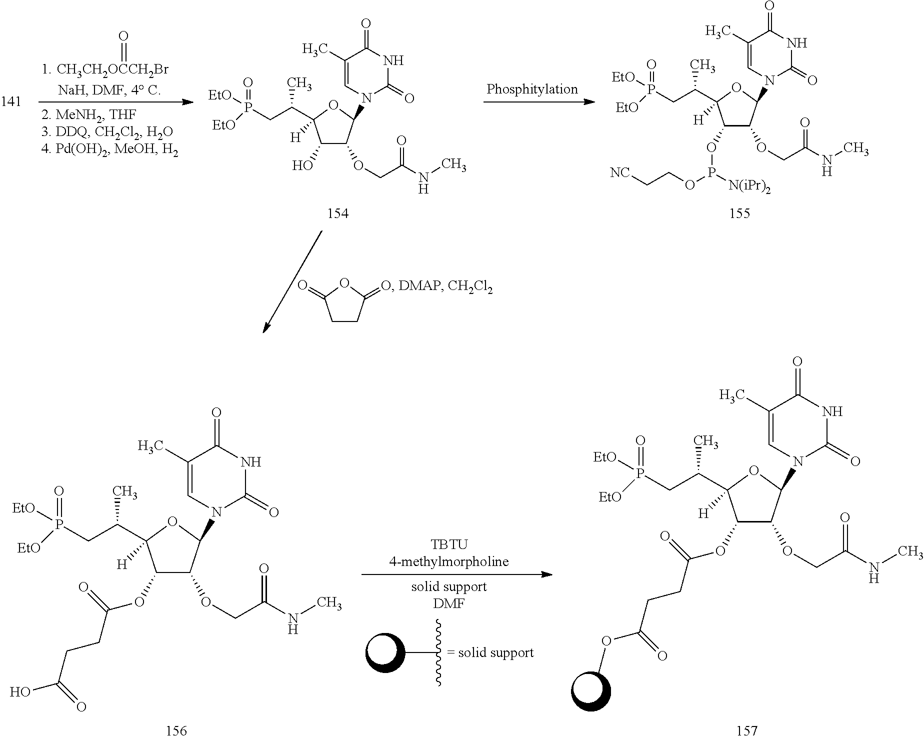

Compound 141 is prepared as per the procedures illustrated in Example 45.

### Example 49

### Preparation of Compounds 159 and 161

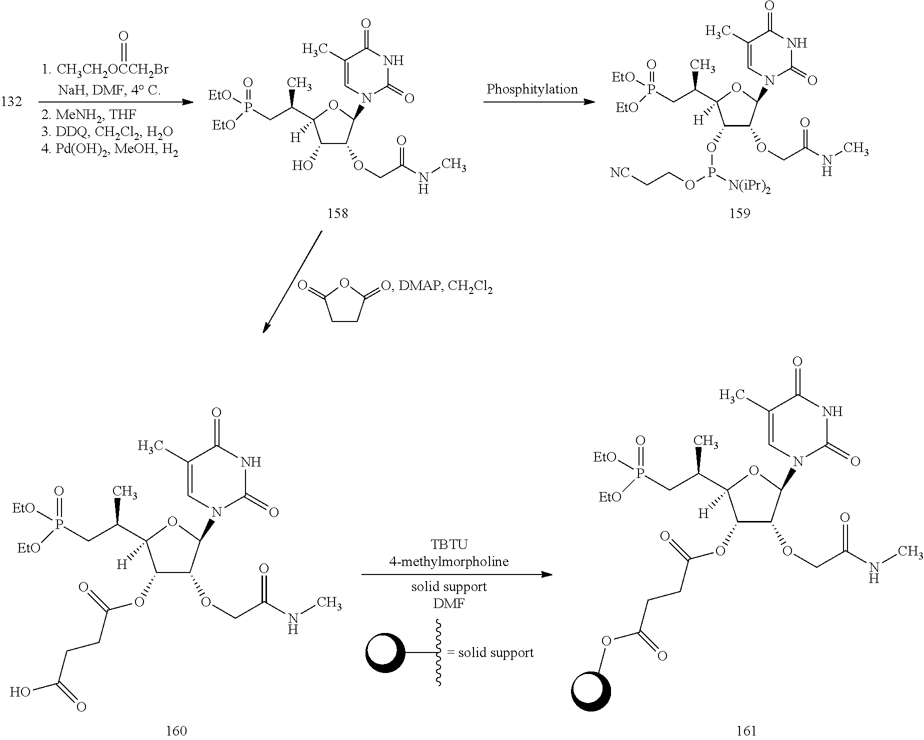

Compound 132 is prepared as per the procedures illustrated in Example 44.

### Example 50

### Preparation of Compounds 163 and 165

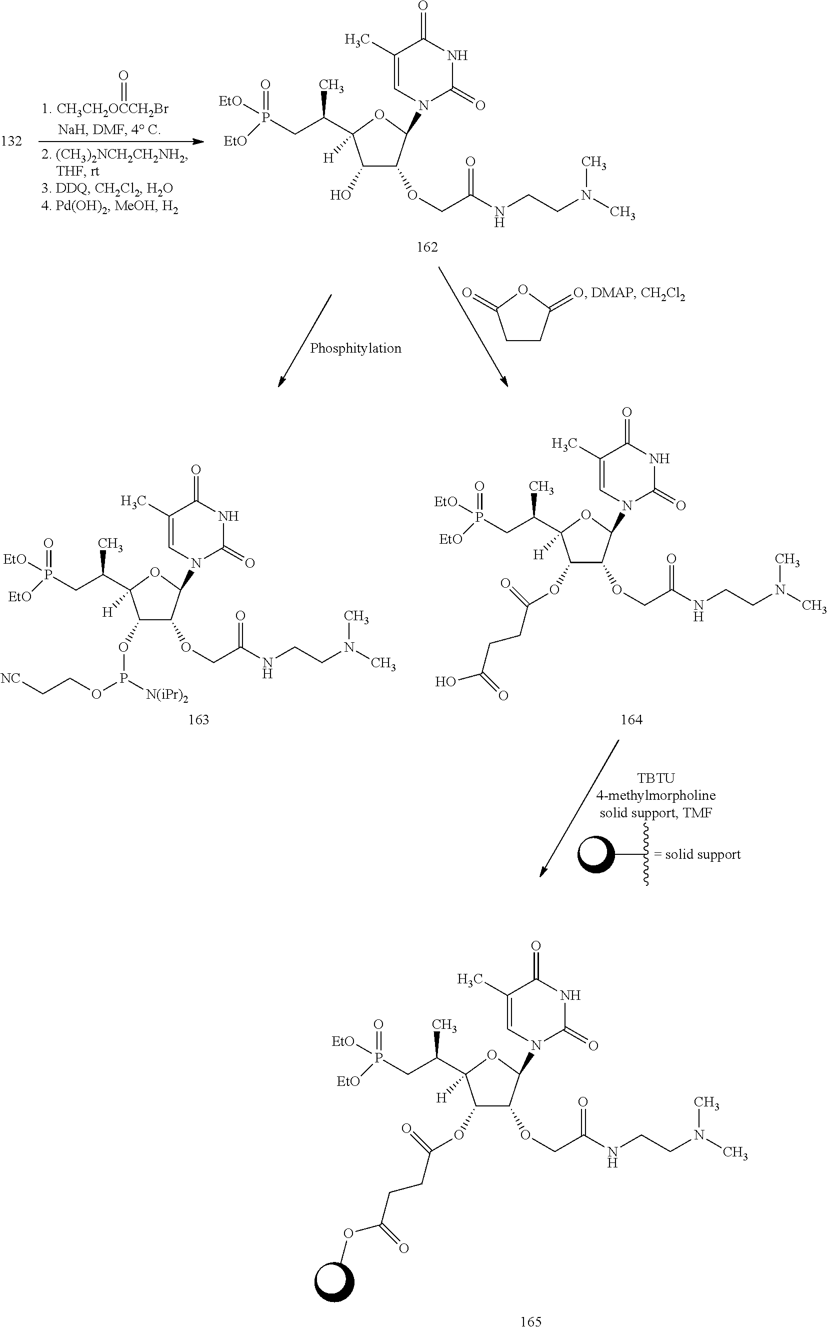

Compound 132 is prepared as per the procedures illustrated in Example 44.

### Example 51

### Preparation of Compounds 167 and 169

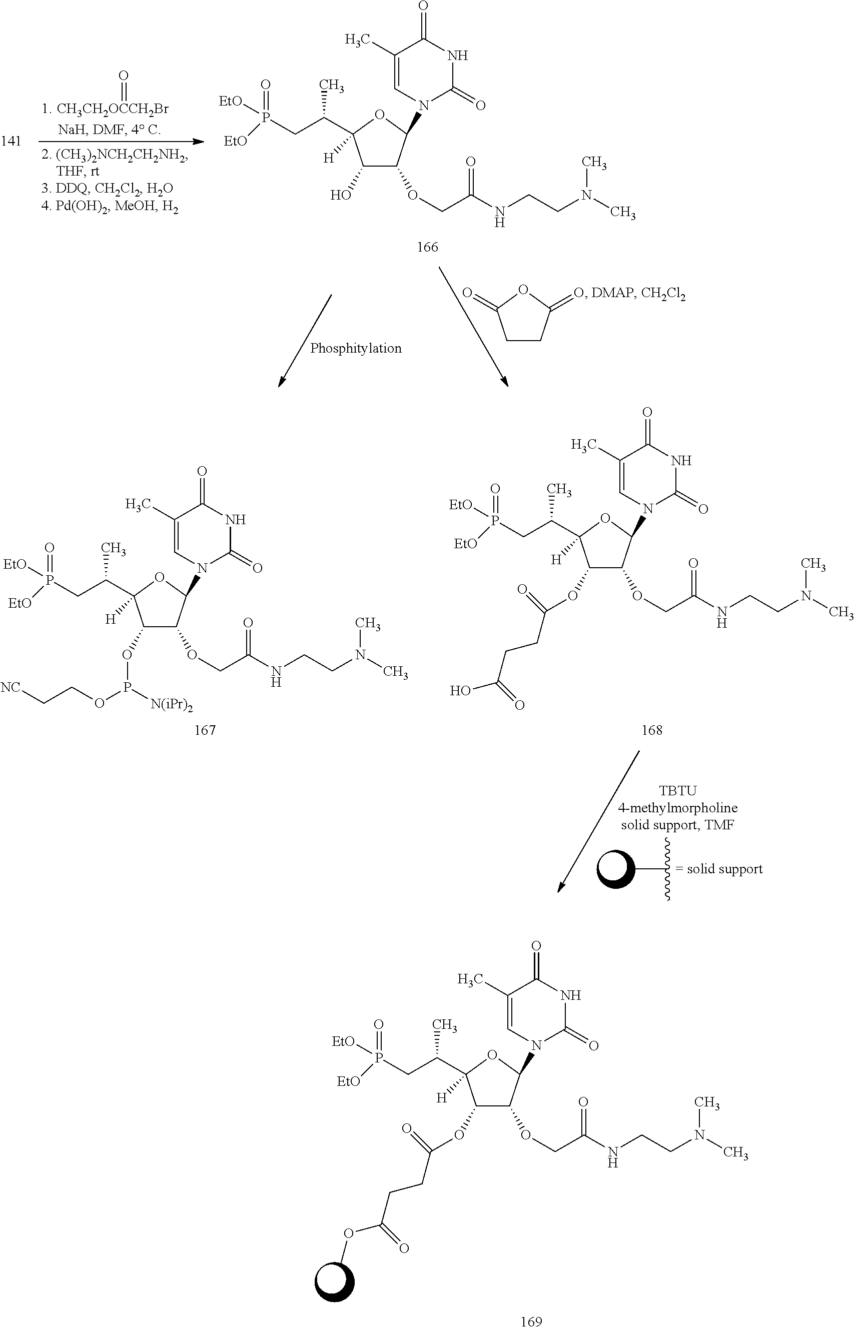

Compound 141 is prepared as per the procedures illustrated in Example 45.

### Example 52

### Preparation of Compounds 172 and 174

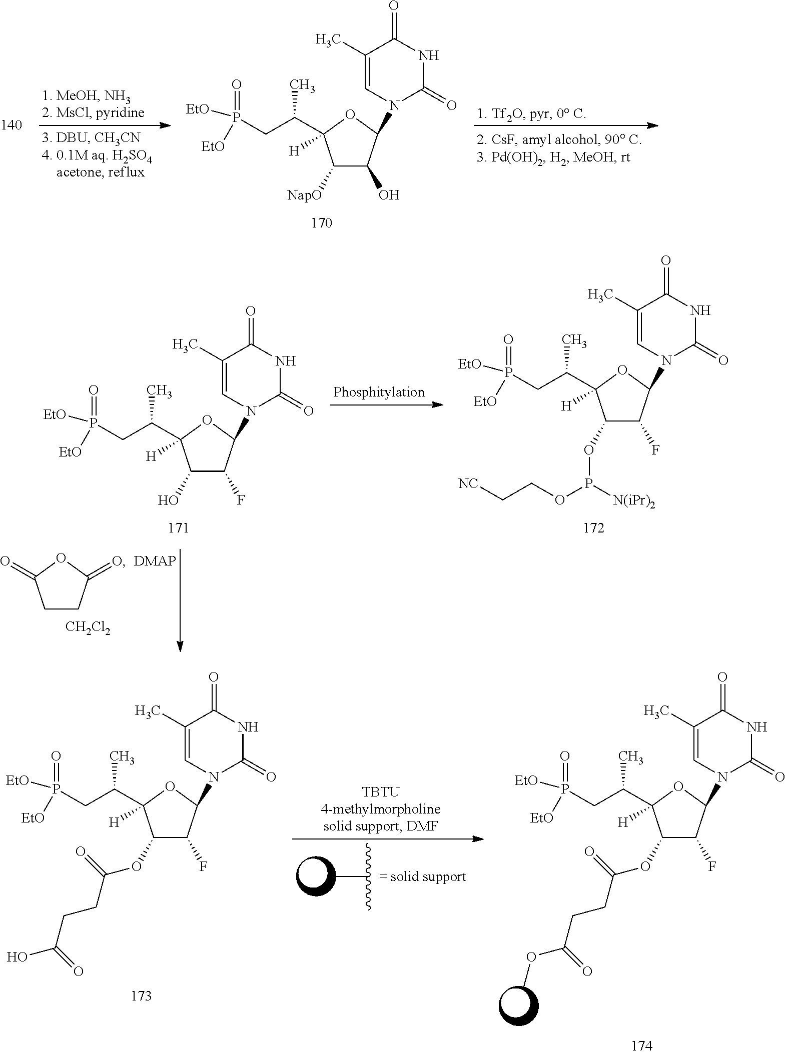

Compound 140 is prepared as per the procedures illustrated in Example 45.

### Example 53

### Preparation of Compounds 177 and 179

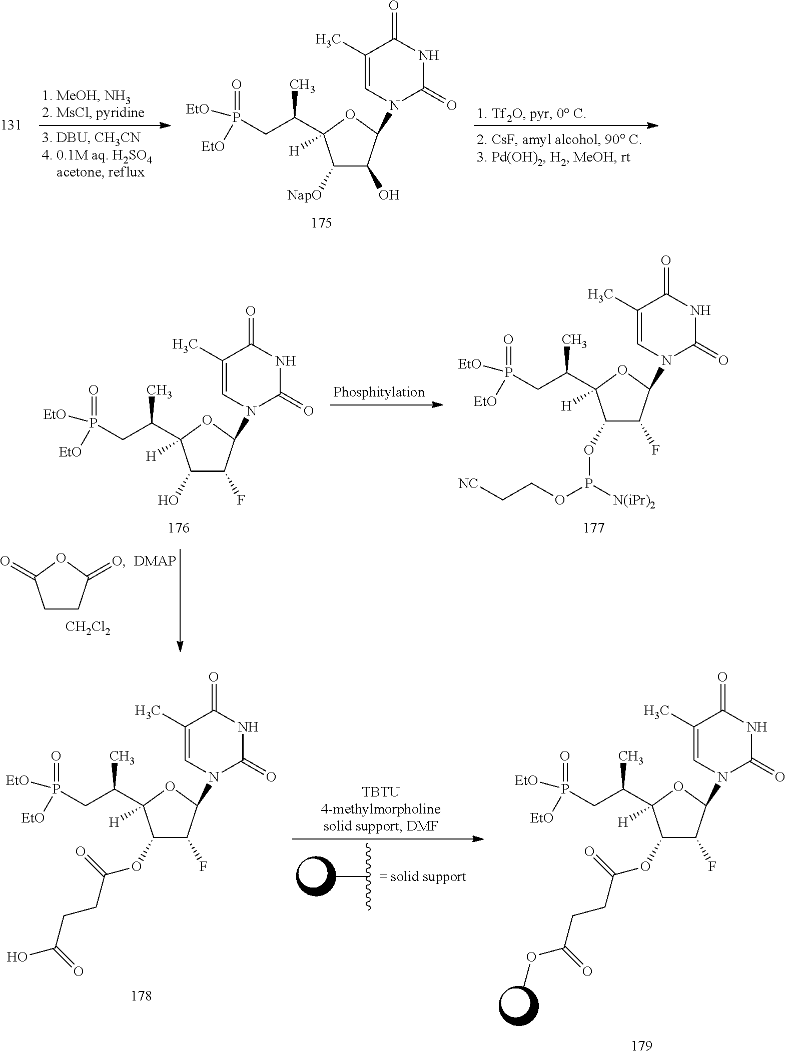

Compound 131 is prepared as per the procedures illustrated in Example 44.

### Example 54

### General Procedure for the Preparation of Compounds of Formula IIa and IIb

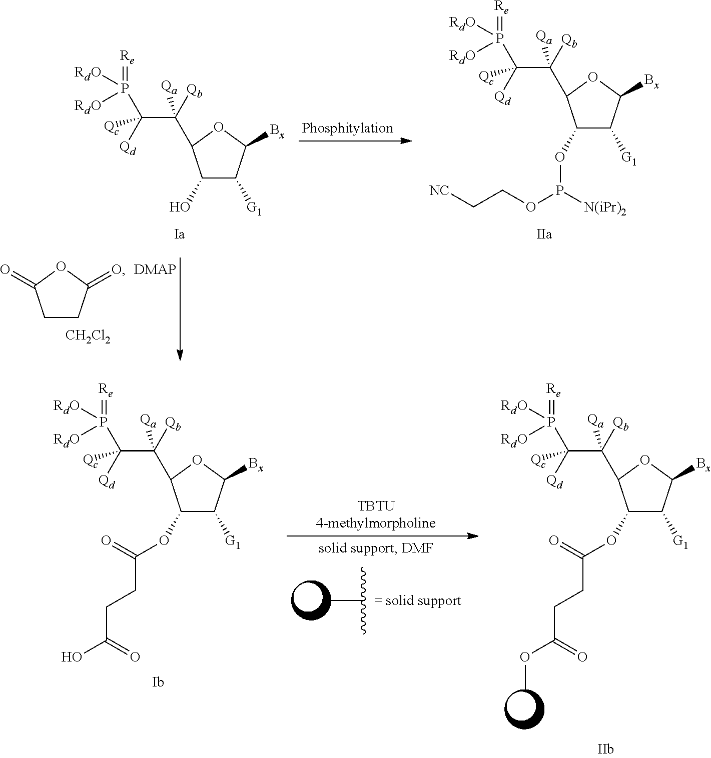 [0753] Bₓ is a heterocylic base moiety;
Qₐ, Q_{b}, Q_{c} and Q_{d} are each independently H or a substituent group;
each R_{d} is, independently, H, C₁-C₆ alkyl, substituted C₁-C₆ alkyl, aryl, substituted aryl or an internucleoside linkage to an oligomeric compound;
Rₑ is O or S; and
G₁ is a sugar substituent group.

The preparation of compounds of Formula Ia, Ib, IIa and IIb are illustrated in Examples 21-25, 27-35 and 44-53.

### Example 55

### General Procedure for the Preparation of Compounds of Formula IIIa

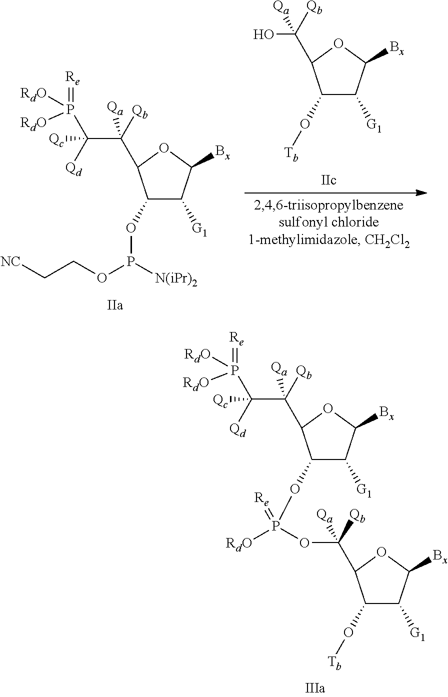 [0760] Bₓ is a heterocylic base moiety;
Qₐ, Q_{b}, Q_{c} and Q_{d} are each independently H or a substituent group;
each R_{d} is, independently, H, C₁-C₆ alkyl, substituted C₁-C₆ alkyl, aryl, substituted aryl or a linkage to an oligomeric compound;
T_{b} is a protecting group, a 3′-terminal group or a linkage to an oligomeric compound;
Rₑ is O or S; and
G₁ is a sugar substituent group.

The preparation of compounds of Formula IIa, IIc, and IIIa are illustrated in Examples 13, 15-19, 21-25 and 27-53.

### Example 56

### General Procedure for the Preparation of Compounds of Formula IIIb and IIIc

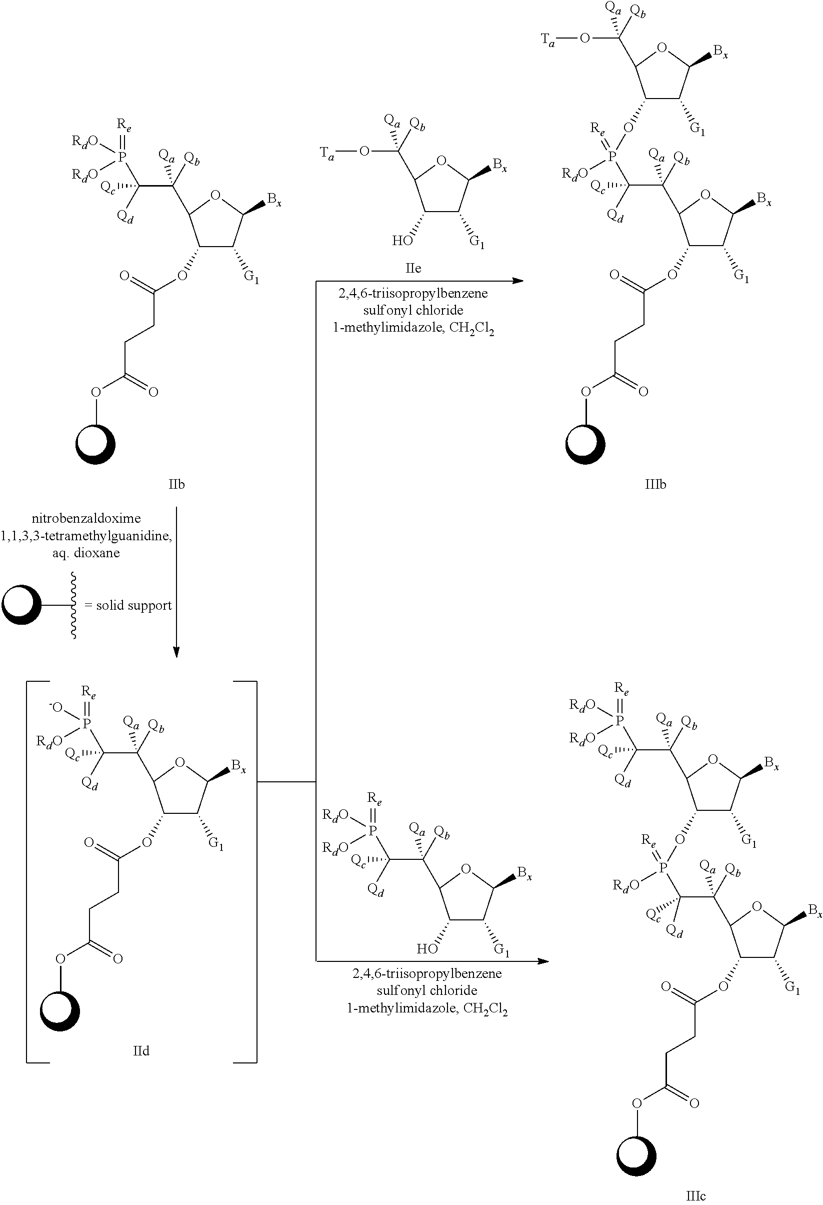 [0768] Bₓ is a heterocylic base moiety;
Qₐ, Q_{b}, Q_{c} and Q_{d} are each independently H or a substituent group;
each R_{d} is, independently, H, C₁-C₆ alkyl, substituted C₁-C₆ alkyl, aryl, substituted aryl or a linkage to an oligomeric compound;
Tₐ is H, a protecting group or a 5′-terminal group;
Rₑ is O or S; and
G₁ is a sugar substituent group.

The preparation of compounds of Formula IIb, IId, IIe, IIf, IIIb and IIIc are illustrated in Examples 13, 15-19, 21-25 and 27-53.

### Example 57

### Chemically Modified ssRNAs Targeting PTEN—In Vivo Study

The antisense activity of oligomeric compounds can be tested in vivo. Five- to six-week old Balb/c mice (Jackson Laboratory, Bar Harbor, Me.) are injected with modified ssRNA targeted to PTEN at doses of 80 mg/kg daily, 60 mg/kg daily, or 40 mg/kg twice daily for several days. The mice are sacrificed 72 hours following the last administration. Liver tissues are homogenized and mRNA levels are quantitated using real-time PCR using procedures illustrated herein for comparison to untreated control levels (% UTC). Other modifications and motifs as disclosed herein are also amenable to in vivo testing. Liver transaminase levels, alanine aminotranferease (ALT) and aspartate aminotransferase (AST), in serum are also measured relative to saline injected mice. At the end of the study, liver and spleen tissues are harvested from animals treated with the modified ssRNAs, the tissues are weighed to assess gross organ alterations.

| SEQ ID NO./ | |
|---|---|
| ISIS NO. | Composition (5′ to 3′) |
| 05/422391 | P-T_{d}U_{f}G_{f}U_{f}C_{f}U_{f}C_{f}U_{f}G_{f}G_{f}U_{f}C_{f}C_{f}U_{f}U_{f}A_{f}C_{f}U_{f}U_{f}AₑAₑ |
| 05/435394 | P-T_{d}U_{f}G_{f}U_{f}C_{f}U_{f}C_{f}U_{f}G_{f}G_{f}U_{f}C_{f}C_{f}U_{f}U_{f}A_{f}C_{f}U_{f}U_{f}AₑAₑ |
| 05/435395 | Pₛ-T_{d}U_{f}G_{f}U_{f}C_{f}U_{f}C_{f}U_{f}G_{f}G_{f}U_{f}C_{f}C_{f}U_{f}U_{f}A_{f}C_{f}U_{f}U_{f}AₑAₑ |
| 05/435397 | P-T_{d}U_{f}GₘU_{f}CₘU_{f}CₘU_{f}GₘG_{f}UₘC_{f}CₘU_{f}UₘA_{f}CₘU_{f}UₘAₑAₑ |
| 05/435402 | P-T_{d}U_{f}GₘU_{f}CₘU_{f}CₘU_{f}GₘG_{f}UₘC_{f}CₘU_{f}UₘA_{f}CₘU_{f}UₘAₑAₑ |
| 05/435401 | P-T_{d}U_{f}GₘU_{f}CₘU_{f}CₘU_{f}GₘG_{f}UₘC_{f}CₘU_{f}UₘA_{f}CₘU_{f}UₘAₑAₑ |
| 05/435400 | P-T_{d}U_{f}GₘU_{f}CₘU_{f}CₘU_{f}GₘG_{f}UₘC_{f}C_{f}U_{f}UₘA_{f}CₘU_{f}UₘAₑAₑ |
| 05/435399 | P-T_{d}U_{f}GₘU_{f}CₘU_{f}CₘU_{f}GₘG_{f}UₘC_{f}C_{f}U_{f}UₘA_{f}CₘU_{f}UₘAₑAₑ |
| 05/435404 | P-T_{d}U_{f}GₘU_{f}CₘU_{f}CₘU_{f}GₘG_{f}U_{f}C_{f}C_{f}U_{f}U_{f}A_{f}C_{f}U_{f}U_{f}AₑAₑ |
| 05/xxxxx | P-T_{R}U_{f}GₘU_{f}CₘU_{f}CₘU_{f}GₘG_{f}UₘC_{f}CₘU_{f}UₘA_{f}CₘU_{f}UₘAₑAₑ |
| 05/xxxxx | P-T_{R}U_{f}GₘU_{f}CₘU_{f}CₘU_{f}GₘG_{f}UₘC_{f}CₘU_{f}UₘA_{f}CₘU_{f}UₘAₑAₑ |
| 05/xxxxx | P-T_{R}U_{f}GₘU_{f}CₘU_{f}CₘU_{f}GₘG_{f}UₘC_{f}CₘU_{f}U_{f}A_{f}C_{f}U_{f}U_{f}AₑAₑ |
| 05/xxxxx | P-T_{R}U_{f}GₘU_{f}CₘU_{f}CₘU_{f}GₘG_{f}UₘC_{f}C_{f}U_{f}U_{f}A_{f}C_{f}U_{f}U_{f}AₑAₑ |
| 05/xxxxx | P-T_{R}U_{f}GₘU_{f}CₘU_{f}CₘU_{f}GₘG_{f}UₘC_{f}C_{f}U_{f}U_{f}A_{f}C_{f}U_{f}U_{f}AₑAₑ |
| 05/xxxxx | P-T_{R}U_{f}GₘU_{f}CₘU_{f}CₘU_{f}GₘG_{f}U_{f}C_{f}C_{f}U_{f}U_{f}A_{f}C_{f}U_{f}U_{f}AₑAₑ |
| 05/xxxxx | P-T_{S}U_{f}GₘU_{f}CₘU_{f}CₘU_{f}GₘG_{f}UₘC_{f}CₘU_{f}UₘA_{f}CₘU_{f}UₘAₑAₑ |
| 05/xxxxx | P-T_{S}U_{f}GₘU_{f}CₘU_{f}CₘU_{f}GₘG_{f}UₘC_{f}CₘU_{f}UₘA_{f}CₘU_{f}UₘAₑAₑ |
| 05/xxxxx | P-T_{S}U_{f}GₘU_{f}CₘU_{f}CₘU_{f}GₘG_{f}UₘC_{f}CₘU_{f}U_{f}A_{f}C_{f}U_{f}U_{f}AₑAₑ |
| 05/xxxxx | P-T_{S}U_{f}GₘU_{f}CₘU_{f}CₘU_{f}GₘG_{f}UₘC_{f}C_{f}U_{f}U_{f}A_{f}C_{f}U_{f}U_{f}AₑAₑ |
| 05/xxxxx | P-T_{S}U_{f}GₘU_{f}CₘU_{f}CₘU_{f}GₘG_{f}UₘC_{f}C_{f}U_{f}U_{f}A_{f}C_{f}U_{f}U_{f}AₑAₑ |
| 05/xxxxx | P-T_{S}U_{f}GₘU_{f}CₘU_{f}CₘU_{f}GₘG_{f}U_{f}C_{f}C_{f}U_{f}U_{f}A_{f}C_{f}U_{f}U_{f}AₑAₑ |
| 05/xxxxx | P-T_{d}U_{f}GₘU_{f}CₘU_{f}CₘU_{f}GₘG_{f}UₘC_{f}CₘU_{f}UₘA_{f}CₘU_{f}UₘAₑAₑ |
| 05/xxxxx | P-T_{d}U_{f}GₘU_{f}CₘU_{f}CₘU_{f}GₘG_{f}UₘC_{f}CₘU_{f}UₘA_{f}CₘU_{f}UₘAₑAₑ |
| 05/xxxxx | P-T_{d}U_{f}GₘU_{f}CₘU_{f}CₘU_{f}GₘG_{f}UₘC_{f}CₘU_{f}U_{f}A_{f}C_{f}U_{f}U_{f}AₑAₑ |
| 05/xxxxx | P-T_{d}U_{f}GₘU_{f}CₘU_{f}CₘU_{f}GₘG_{f}UₘC_{f}C_{f}U_{f}U_{f}A_{f}C_{f}U_{f}U_{f}AₑAₑ |
| 05/xxxxx | P-T_{d}U_{f}GₘU_{f}CₘU_{f}CₘU_{f}GₘG_{f}UₘC_{f}C_{f}U_{f}U_{f}A_{f}C_{f}U_{f}U_{f}AₑAₑ |
| 05/xxxxx | P-T_{d}U_{f}GₘU_{f}CₘU_{f}CₘU_{f}GₘG_{f}U_{f}C_{f}C_{f}U_{f}U_{f}A_{f}C_{f}U_{f}U_{f}AₑAₑ |
| 05/xxxxx | P-T_{Rd}U_{f}GₘU_{f}CₘU_{f}CₘU_{f}GₘG_{f}UₘC_{f}CₘU_{f}UₘA_{f}CₘU_{f}UₘAₑAₑ |
| 05/xxxxx | P-T_{R}_{d}U_{f}GₘU_{f}CₘU_{f}CₘU_{f}GₘG_{f}UₘC_{f}CₘU_{f}UₘA_{f}CₘU_{f}UₘAₑAₑ |
| 05/xxxxx | P-T_{R}_{d}U_{f}GₘU_{f}CₘU_{f}CₘU_{f}GₘG_{f}UₘC_{f}CₘU_{f}U_{f}A_{f}C_{f}U_{f}U_{f}AₑAₑ |
| 05/xxxxx | P-T_{Rd}U_{f}GₘU_{f}CₘU_{f}CₘU_{f}GₘG_{f}UₘC_{f}C_{f}U_{f}U_{f}A_{f}C_{f}U_{f}U_{f}AₑAₑ |
| 05/xxxxx | P-T_{Rd}U_{f}GₘU_{f}CₘU_{f}CₘU_{f}GₘG_{f}UₘC_{f}C_{f}U_{f}U_{f}A_{f}C_{f}U_{f}U_{f}AₑAₑ |
| 05/xxxxx | P-T_{R}_{d}U_{f}GₘU_{f}CₘU_{f}CₘU_{f}GₘG_{f}U_{f}C_{f}C_{f}U_{f}U_{f}A_{f}C_{f}U_{f}U_{f}AₑAₑ |
| 05/xxxxx | P-T_{Sd}U_{f}GₘU_{f}CₘU_{f}CₘU_{f}GₘG_{f}UₘC_{f}CₘU_{f}UₘA_{f}CₘU_{f}UₘAₑAₑ |
| 05/xxxxx | P-T_{S}_{d}U_{f}GₘU_{f}CₘU_{f}CₘU_{f}GₘG_{f}UₘC_{f}CₘU_{f}UₘA_{f}CₘU_{f}UₘAₑAₑ |
| 05/xxxxx | P-T_{S}_{d}U_{f}GₘU_{f}CₘU_{f}CₘU_{f}GₘG_{f}UₘC_{f}CₘU_{f}U_{f}A_{f}C_{f}U_{f}U_{f}AₑAₑ |
| 05/xxxxx | P-T_{S}_{d}U_{f}GₘU_{f}CₘU_{f}CₘU_{f}GₘG_{f}UₘC_{f}C_{f}U_{f}U_{f}A_{f}C_{f}U_{f}U_{f}AₑAₑ |
| 05/xxxxx | P-T_{Sd}U_{f}GₘU_{f}CₘU_{f}CₘU_{f}GₘG_{f}UₘC_{f}C_{f}U_{f}U_{f}A_{f}C_{f}U_{f}U_{f}AₑAₑ |
| 05/xxxxx | P-T_{S}_{d}U_{f}GₘU_{f}CₘU_{f}CₘU_{f}GₘG_{f}U_{f}C_{f}C_{f}U_{f}U_{f}A_{f}C_{f}U_{f}U_{f}AₑAₑ |
| 06/409044 | P-UₘU_{f}GₘU_{f}CₘU_{f}CₘU_{f}GₘG_{f}UₘC_{f}CₘU_{f}UₘA_{f}CₘU_{f}UₘAₑAₑ |
| 06/418042 | P-UₘU_{f}GₘU_{f}CₘU_{f}CₘU_{f}GₘG_{f}UₘC_{f}CₘU_{f}UₘA_{f}CₘU_{f}UₘAₑAₑ |
| 06/414291 | P-UₘU_{f}GₘU_{f}CₘU_{f}CₘU_{f}GₘG_{f}UₘC_{f}CₘU_{f}UₘA_{f}CₘU_{f}UₘAₑAₑ |
| 06/416598 | P-UₘU_{f}GₘU_{f}CₘU_{f}CₘU_{f}GₘG_{f}UₘC_{f}CₘU_{f}UₘA_{f}CₘU_{f}UₘAₑAₑ |
| 06/418043 | P-UₘU_{f}GₘU_{f}CₘU_{f}CₘU_{f}GₘG_{f}UₘC_{f}CₘU_{f}UₘA_{f}CₘU_{f}UₘAₑAₑ |
| 06/418044 | P-UₘU_{f}GₘU_{f}CₘU_{f}CₘU_{f}GₘG_{f}UₘC_{f}CₘU_{f}UₘA_{f}CₘU_{f}UₘAₑAₑ |
| 06/418045 | P-UₘU_{f}GₘU_{f}CₘU_{f}CₘU_{f}GₘG_{f}UₘC_{f}CₘU_{f}UₘA_{f}CₘU_{f}UₘAₑAₑ |
| 06/418046 | P-UₘU_{f}GₘU_{f}CₘU_{f}CₘU_{f}GₘG_{f}UₘC_{f}CₘU_{f}UₘA_{f}CₘU_{f}UₘAₑAₑ |
| 06/418127 | P-U_{R}U_{f}G_{f}U_{f}C_{f}U_{f}C_{f}U_{f}G_{f}G_{f}U_{f}C_{f}C_{f}U_{f}U_{f}A_{f}C_{f}U_{f}U_{f}AₑAₑ |
| 06/xxxxx | P-UₓU_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-UₓU_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{Sd}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{Sd}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{Sd}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{Sd}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{Sd}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{Sd}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{Sd}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{Sd}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{Rd}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{Rd}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{Rd}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{Rd}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{Rd}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{Rd}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{Rd}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{Rd}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{d}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{d}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{d}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{d}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{d}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{d}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{d}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{d}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{R}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{R}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{R}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{R}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{R}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{R}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{R}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{R}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{S}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{S}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{S}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{S}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{S}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{S}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{S}U_{f}GₘU_{f}CₘU_{f}CₘU_{f}GₘG_{f}UₘC_{f}CₘU_{f}UₘA_{f}CₘU_{f}UₘAₑAₑ |
| 06/xxxxx | P-U_{S}U_{f}GₘU_{f}CₘU_{f}CₘU_{f}GₘG_{f}UₘC_{f}CₘU_{f}UₘA_{f}CₘU_{f}UₘAₑAₑ |
| 07/410146 | P-Aₑ^{Me}C_{ef}AₑAₑAₑ^{Me}C_{ef}A^{Me}C_{ef}^{Me}C_{ef}AₑT_{ef}T_{ef}GₑT^{Me}_{ef}CAₑ^{Me}C_{ef}Aₑ^{Me}C_{ef}Aₑ^{Me}C_{ef}^{Me}C_{ef}Aₑ |
| 07/327895 | P-A^{Me}CₑAₑAₑAₑ^{Me}CₑA^{Me}Cₑ^{Me}CₑAₑTₑTₑGₑT^{Me}ₑCA^{Me}CₑAₑ^{Me}CₑAₑ^{Me}Cₑ^{Me}CₑAₑ |

Each nucleoside is connected to the following nucleoside by a phosphodiester internucleoside linkage except underlined nucleosides which are connected to the following nucleoside by a phosphorothioate internucleoside linkage (going 5′ to 3′). A “P” at the 5′-end indicates a 5′-phosphate group. A “Pₛ” at the 5′-end indicates a 5′-thiophosphate group. Nucleosides followed by a subscript d, ef, f, m, e or x are sugar modified nucleosides. A subscript “d” indicates a 2′-OCH₂(CO)NH(CH₂)₂N(CH₃)₂ (DMAEAc), subscript “ef” indicates a 2′-OCH₂CH₂F (FEt) modified nucleoside, a subscript “f” indicates a 2′-fluoro modified nucleoside, a subscript “m” indicates 2′-O-methyl modified nucleoside, a subscript “e” indicates a 2′-O(CH₂)₂OCH₃ (MOE) modified nucleoside, and a subscript R or S or Rd or Sd or x indicates one of the 5′-modified nucleosides (R or S) or one of the 2′,5′-bis modified nucleosides listed below (Rd, Sd, Rb, Sb, Rc or Sc). In general, each modified nucleoside having an x after it will have the same sugar modification.

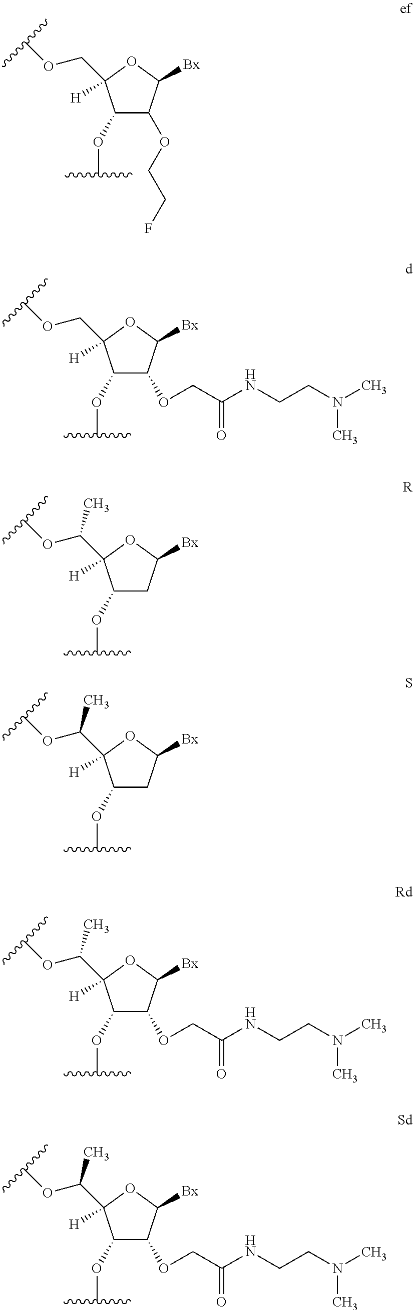

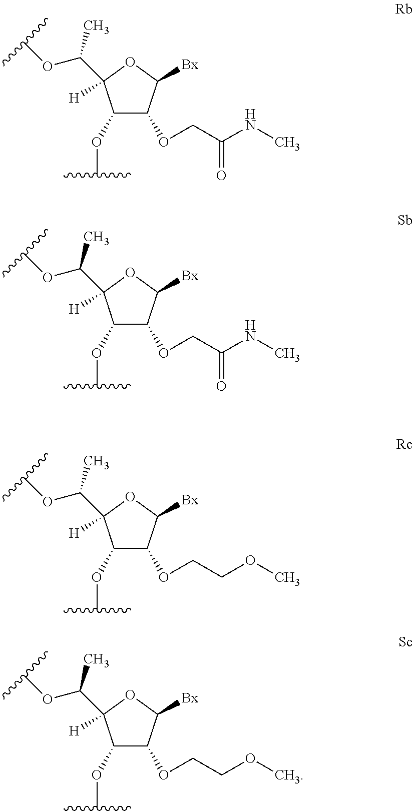

### Example 58

### Gapped Oligomeric Compounds Targeted to PTEN: In Vivo Study

In accordance with the present disclosure, oligomeric compounds are synthesized and tested for their ability to reduce PTEN expression in vivo at doses of 20 and 60 mg/kg. Six week old male Balb/c mice (Jackson Laboratory, Bar Harbor, Me.) are administered a single intraperitoneal (i.p) injection at either 20 or 60 mg/kg of a 2-10-2 gapped oligomer. A 5-10-5 gapped oligomer having 2′-O-MOE modified nucleosides or other modified nucleosides as provided herein in the wings is also included for comparison. Other motifs as disclosed herein are also amenable to in vivo testing.

Each dose group will include four animals. The mice are sacrificed 48 hours following the final administration to determine the PTEN mRNA levels in liver using real-time PCR and RIBOGREEN® RNA quantification reagent (Molecular Probes, Inc. Eugene, Oreg.) according to standard protocols. PTEN mRNA levels are determined relative to total RNA (using Ribogreen), prior to normalization to saline-treated control. The average % inhibition of mRNA expression for each treatment group, normalized to saline-injected control is determined.

Liver transaminase levels, alanine aminotranferease (ALT) and aspartate aminotransferase (AST), in serum are measured relative to saline injected mice.

| SEQ ID | |
|---|---|
| NO | Composition (5′ to 3′) |
| 08 | ^{me}CₓTₓGₓ^{me}CₓTₓAG^{me}C^{me}CT^{me}CTGGATₓTₓTₓGₓAₓ |
| 09 | CₓTₓTAGCACTGGCCₓTₓ |
| 09 | P-CₓTₓTAGCACTGGCCₓTₓ |
| 09 | ^{me}CₓTₓTAGCACTGGC^{me}CₓTₓ |

Each unmodified nucleoside is β-D-2∝-deoxyribonucleoside. Each internucleoside linkage is a phosphorothioate internucleoside linkage. A “P” at the 5′-end indicates a 5′-phosphate group. ^{me}C indicates a 5′-methyl cytosine nucleoside. Each nucleoside having a subscript x is selected from the list at the end of Example 57, e.g., Rb, Sb, Re, Sc, Rd and Sd. In general, each modified nucleoside having an x after it will have the same sugar modification but can have different bases.

### Example 59

### Oligomeric Compounds Targeted to PTEN: In Vitro Study

In accordance with the present disclosure, oligomeric compounds were synthesized and tested for their ability to reduce PTEN expression over a range of doses. Human HeLa cells were treated with either ISIS 447581 or ISIS 404320. A dose comparison was evaluated with dose concentrations of 0.20, 0.62, 1.9, 5.5, 16.7 and 50 nM using methods described herein. Expression levels of PTEN were determined using real-time PCR and normalized to RIBOGREEN™ using methods described herein. The percent inhibition of PTEN mRNA was determined. Resulting dose-response curves were used to determine the EC₅₀. Tm's were assessed in 100 mM phosphate buffer, 0.1 mM EDTA, pH 7, at 260 nm using 4 μM modified oligomers and 4 μM complementary RNA. The EC₅₀s are listed below.

| SEQ ID | ISIS | | |
|---|---|---|---|
| NO. | NO. | Composition (5′ to 3′) | EC₅₀ (nM) |
| 05 | 447581 | P-T_{Rc}U_{f}GₘU_{f}CₘU_{f}CₘU_{f}GₘG_{f}UₘC_{f}Cₘ-U_{f}UₘA_{f}CₘU_{f}UₘAₑAₑ | 0.87 |
| 06 | 404320 | P-U_{f}U_{f}G_{f}U_{f}C_{f}U_{f}C_{f}U_{f}G_{f}G_{f}U_{f}C_{f}C_{f}U_{f}U_{f} A_{f}C_{f}U_{f}U_{f}AₑAₑ | 13.2 |

Each nucleoside is connected to the following nucleoside by a phosphodiester internucleoside linkage except underlined nucleosides which are connected to the following nucleoside by a phosphorothioate internucleoside linkage (going 5′ to 3′). A “P” at the 5′-end indicates a 5′-phosphate group. Nucleosides followed by a subscript f, m or e are sugar modified nucleosides. A subscript “f” indicates a 2′-fluoro modified nucleoside, a subscript “m” indicates 2′-O-methyl modified nucleoside, a subscript “e” indicates a 2′-O(CH₂)₂OCH₃ (MOE) modified nucleoside and a subscript Rc indicates the 2′,5′-bis modified nucleoside listed in Example 57.

### Example 60

### Modified ssRNA 5′-Phosphate Serum Stability Assay

A serum stability assay is useful for evaluating the stability of oligomeric compounds in the presences of nucleases and other enzymes found in serum. For example, the stability of a 5′-terminal phosphate group of an oligomeric compound can be evaluated by assessing the ability of the 5′-terminal phosphate group to remain attached to the oligomeric compound in the presence of serum. Accordingly, a serum stability assay was employed to evaluate the stability of modified ssRNAs having a 5′-terminal phosphate group.

Various modified ssRNAs, shown below, having a 5′-terminal phosphate group (10 μM) were dissolved in 95% of fresh mouse serum and incubated at 37° C. Aliquots of serum (100 μL) were removed after 0, 1, 3, 6 or 24 hours of incubation times. The serum samples were immediately quenched and snap frozen. The samples were extracted by the strong anion exchange (SAX) and octadecylsilyl (C-18) columns. For each incubation time, the amount of full length modified ssRNA having a 5′-terminal phosphate group was determined by LC/MS, and the half-life of the full length modified ssRNA having a 5′ terminal phosphate group was calculated. The results are expressed as half-time (T_{1/2}) in the table below. These data demonstrate that modifications to oligomeric compounds can improve the stability of the 5′-terminal phosphate group.

| SEQ ID | ISIS | | |
|---|---|---|---|
| NO. | NO. | Composition (5′ to 3′) | T_{1/2} (h) |
| 05 | 422391 | P-T_{d}U_{f}G_{f}U_{f}C_{f}U_{f}C_{f}U_{f}G_{f}G_{f}U_{f}C_{f}C_{f}U_{f}U_{f}A_{f}C_{f}U_{f}U_{f}AₑAₑ | 6.5 |
| 05 | 432356 | P-T_{R}U_{f}G_{f}U_{f}C_{f}U_{f}C_{f}U_{f}G_{f}G_{f}U_{f}C_{f}C_{f}U_{f}U_{f}A_{f}C_{f}U_{f}U_{f}AₑAₑ | 8.7 |
| 06 | 404320 | P-U_{f}U_{f}G_{f}U_{f}C_{f}U_{f}C_{f}U_{f}G_{f}G_{f}U_{f}C_{f}C_{f}U_{f}U_{f}A_{f}C_{f}U_{f}U_{f}AₑAₑ | 4 |
| 010 | 398701 | P-U_{Sf}U_{f}G_{f}U_{f}C_{f}U_{f}C_{f}U_{f}G_{f}G_{f}U_{f}C_{f}C_{f}U_{f}U_{f}A_{f}C_{f}U_{f}U_{f} | 18.2 |

Each nucleoside is connected to the following nucleoside by a phosphodiester internucleoside linkage except underlined nucleosides which are connected to the following nucleoside by a phosphorothioate internucleoside linkage (going 5′ to 3′). A “P” at the 5′-end indicates a 5′-phosphate group. Nucleosides followed by a subscript d, e, f, R or Sf are sugar modified nucleosides. A subscript “d” indicates a 2′-O-dimethylaminoethyl acetamide (DMAEAc) modified nucleoside, a subscript “e” indicates a 2′—O(CH₂)₂OCH₃ (MOE) modified nucleoside, a subscript “1” indicates a 2′-fluoro modified nucleoside, a subscript “R” indicates (R)-5′-methyl-2′-deoxyribonucleoside and a subscript Sf indicates the 2′,5′-bis modified nucleoside listed below.

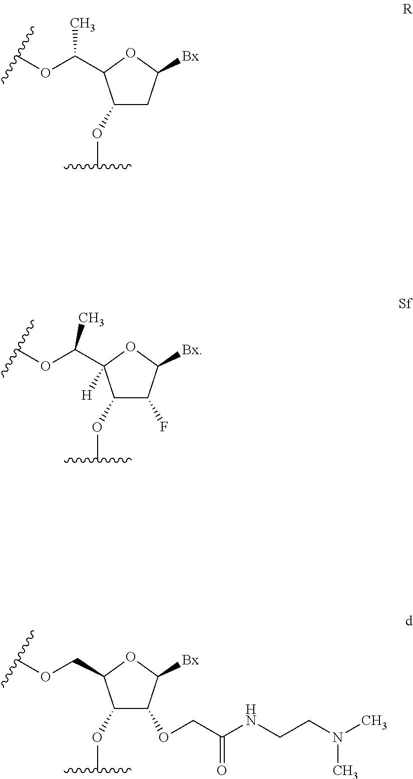

### Example 61

### Design and Screening of Duplexed Antisense Compounds

In accordance with the present invention, a series of nucleic acid duplexes comprising the compounds of the present invention and their complements can be designed. The nucleobase sequence of the antisense strand of the duplex comprises at least a portion of an antisense oligonucleotide targeted to a target sequence as described herein. The ends of the strands may be modified by the addition of one or more natural or modified nucleosides to form an overhang. The sense strand of the dsRNA is then designed and synthesized as the complement of the antisense strand and may also contain modifications or additions to either terminus. For example, in one embodiment, both strands of the dsRNA duplex would be complementary over the central nucleobases, each having overhangs at one or both termini.

For example, a duplex comprising an antisense strand having the sequence CGAGAGGCGGACGGGACCG (SEQ ID NO: 11) and having a two-nucleobase overhang of deoxythymidine(dT) would have the following structure:

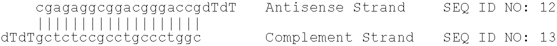

In another embodiment, a duplex comprising an antisense strand having the same sequence CGAGAGGCGGACGGGACCG (SEQ ID NO: 10) may be prepared with blunt ends (no single stranded overhang) as shown:

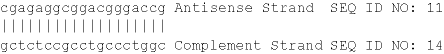

RNA strands of the duplex can be synthesized by methods disclosed herein or purchased from Dharmacon Research Inc., (Lafayette, Colo.). Once synthesized, the complementary strands are annealed. The single strands are aliquoted and diluted to a concentration of 50 μM. Once diluted, 30 μL of each strand is combined with 15 μL of a 5× solution of annealing buffer. The final concentration of the buffer is 100 mM potassium acetate, 30 mM HEPES-KOH pH 7.4, and 2 mM magnesium acetate. The final volume is 75 μL. This solution is incubated for 1 minute at 90° C. and then centrifuged for 15 seconds. The tube is allowed to sit for 1 hour at 37° C. at which time the dsRNA duplexes are used in experimentation. The final concentration of the dsRNA duplex is 20 μM.

Once prepared, the duplexed compounds are evaluated for their ability to modulate target mRNA levels. When cells reach 80% confluency, they are treated with duplexed compounds of the invention. For cells grown in 96-well plates, wells are washed once with 200 μL OPTI-MEM-1™ reduced-serum medium (Gibco BRL) and then treated with 130 μL of OPTI-MEM-1™ containing 5 μg/mL LIPOFECTAMINE 2000™ (Invitrogen Life Technologies, Carlsbad, Calif.) and the duplex antisense compound at the desired final concentration. After about 4 hours of treatment, the medium is replaced with fresh medium. Cells are harvested 16 hours after treatment, at which time RNA is isolated and target reduction measured by quantitative real-time PCR as described herein.

### Example 62

### 5′ and 2′ bis-Substituted Modified Oligomeric Compounds Targeting PTEN—In Vitro Study (ssRNAs vs siRNAs)

A series of 5′ and 2′ bis-substituted modified oligomeric compounds were prepared as single strand RNAs (ssRNAs). The antisense (AS) strands listed below were designed to target human PTEN, and each was also assayed as part of a duplex with the same sense strand (ISIS 341401, shown below) for their ability to reduce PTEN expression levels. HeLa cells were treated with the single stranded or double stranded oligomeric compounds created with the antisense compounds shown below using methods described herein. The IC₅₀'s were calculated using the linear regression equation generated by plotting the normalized mRNA levels to the log of the concentrations used.

| SEQ | | | |
|---|---|---|---|
| ID | | | EC₅₀ (nM) |
| NO. | ISIS NO. | Composition (5′ to 3′) | ssRNA/siRNA |
| 15 | 341401 (S) | AAGUAAGGACCAGAGACAA | ---/--- |
| 05 | 447581 (AS) | P-T_{Rc}U_{f}GₘU_{f}CₘU_{f}CₘU_{f}GₘG_{f}UₘC_{f}CₘU_{f}UₘA_{f}CₘU_{f}UₘAₑAₑ | 1.0/0.4 |
| 05 | 467074 (AS) | P-T_{Sc}U_{f}GₘU_{f}CₘU_{f}CₘU_{f}GₘG_{f}UₘC_{f}CₘU_{f}UₘA_{f}CₘU_{f}UₘAₑAₑ | 2.5/0.1 |
| 05 | 422391 (AS) | P-T_{d}U_{f}G_{f}U_{f}C_{f}U_{f}C_{f}U_{f}G_{f}G_{f}U_{f}C_{f}C_{f}U_{f}U_{f}A_{f}C_{f}U_{f}U_{f}AₑAₑ | 5/0.5 |
| 05 | 432356 (AS) | P-T_{R}U_{f}G_{f}U_{f}C_{f}U_{f}C_{f}U_{f}G_{f}G_{f}U_{f}C_{f}C_{f}U_{f}U_{f}A_{f}C_{f}U_{f}U_{f}AₑAₑ | 3/0.7 |
| 05 | 435397 (AS) | P-T_{d}U_{f}GₘU_{f}CₘU_{f}CₘU_{f}GₘG_{f}UₘC_{f}CₘU_{f}UₘA_{f}CₘU_{f}UₘAₑAₑ | 2/0.4 |
| 06 | 467076 (AS) | Py-^{me}UₘU_{f}GₘU_{f}CₘU_{f}CₘU_{f}GₘG_{f}UₘC_{f}CₘU_{f}UₘA_{f}CₘU_{f}UₘAₑAₑ | 6.0/.05 |
| 06 | 462606 (AS) | Pz-^{me}UₘU_{f}GₘU_{f}CₘU_{f}CₘU_{f}GₘG_{f}UₘC_{f}CₘU_{f}UₘA_{f}CₘU_{f}UₘAₑAₑ | 50/0.4 |
| 06 | 462607 (AS) | Pz-^{me}UₘU_{f}GₘU_{f}CₘU_{f}CₘU_{f}GₘG_{f}UₘC_{f}CₘU_{f}UₘA_{f}CₘU_{f}UₘAₑAₑ | 50/1.0 |
| 06 | 460646 (AS) | Pz-^{me}UₕU_{f}GₘU_{f}CₘU_{f}CₘU_{f}GₘG_{f}UₘC_{f}CₘU_{f}UₘA_{f}CₘU_{f}UₘAₑAₑ | 50/0.8 |
| 06 | 418046(AS) | P-UₘU_{f}GₘU_{f}CₘU_{f}CₘU_{f}GₘG_{f}UₘC_{f}CₘU_{f}UₘA_{f}CₘU_{f}UₘAₑAₑ | 2.0/0.2 |
| 06 | 404320(AS) | P-U_{f}U_{f}G_{f}U_{f}C_{f}U_{f}C_{f}U_{f}G_{f}G_{f}U_{f}C_{f}C_{f}U_{f}U_{f}A_{f}C_{f}U_{f}U_{f}AₑAₑ | 5/0.5 |
| 10 | 359455(AS) | UUGUCUCUGGUCCUUACUU | 50/0.3 |
| 10 | 386187(AS) | P-U_{f}U_{f}G_{f}U_{f}C_{f}U_{f}C_{f}U_{f}G_{f}G_{f}U_{f}C_{f}C_{f}U_{f}U_{f}A_{f}C_{f}U_{f}U_{f} | 15/0.3 |

Each internucleoside linkage is a phosphodiester except that underlined nucleosides are linked to the following nucleoside by a phosphorothioate (going 5′ to 3′). Each nucleoside not followed by a subscript is a ribonucleoside. A “P” at the 5′-end indicates a 5′-phosphate group. A “Py” at the 5′-end indicates a 5′-methylenephosphonate group, (PO(OH)₂CH₂—). A “Pz” at the 5′-end indicates a 5′-difluoromethylenephosphonate group, (PO(OH)₂CF₂—). Nucleosides followed by a subscript indicate modification as follows: subscript “d” indicates a 2′-O-dimethylaminoethyl acetamide (DMAEAc) modified nucleoside; subscript “e” indicates a 2′-O(CH₂)₂OCH₃ (MOE) modified nucleoside, subscript “f” indicates a 2′-fluoro modified nucleoside; subscript “m” indicates 2′-O-methyl modified nucleoside; and subscript “R” indicates a (R)-5′-methyl-2′-deoxyribonucleoside. Superscript “me” indicates a 5-methyl group on the pyrimidine base of the nucleoside. Nucleosides with subscripts “Rc” or “Sc” are shown below.

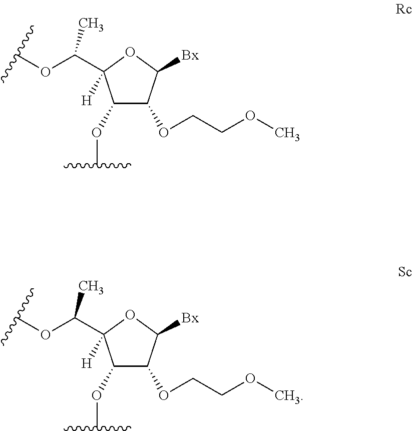

### Example 63

### Modified ssRNAs Targeting PTEN—In Vivo Study

Modified ssRNAs and dsRNAs targeted to PTEN were designed as shown below.

| SEQ ID | | |
|---|---|---|
| NO. | ISIS NO. | Composition (5′ to 3′) |
| 16 | 398239 | 5′-A_{f}AₘG_{f}UₘA_{f}AₘG_{f}GₘA_{f}CₘC_{f}AₘG_{f}AₘG_{f}AₘC_{f}AₘA_{f}UₑUₑ-3′ |
| 06 | 414291 | 3′-AₑAₑUₘU_{f}CₘA_{f}UₘU_{f}CₘC_{f}UₘG_{f}GₘU_{f}CₘU_{f}CₘU_{f}GₘU_{f}Uₘ-5′ |
| 06 | 414291 | P-UₘU_{f}GₘU_{f}CₘU_{f}CₘU_{f}GₘG_{f}UₘC_{f}CₘU_{f}UₘA_{f}CₘU_{f}UₘAₑAₑ |
| 06 | 408874 | P-U_{f}U_{f}G_{f}U_{f}C_{f}UₘCₘU_{f}G_{f}GₘUₘC_{f}C_{f}U_{f}U_{f}A_{f}CₘUₘUₘAₑAₑ |

Phosphorothioate internucleoside linkages are indicated by underlining. Modified nucleosides are indicated by a subscripted letter following the capital letter indicating the nucleoside. In particular, subscript “f” indicates 2′-fluoro; subscript “m” indicates 2′-O-methyl; and subscript “e” indicates 2′-O-methoxyethyl (MOE). For example Uₘ is a modified uridine having a 2′-OCH₃ group. Some of the strands have a 5′-phosphate group designated as “P-”.

### Example 64

### Effect of Modified Internucleoside Linkages on Modified ssRNAs Targeting PTEN—In Vitro Study

A dose response experiment was performed targeting PTEN in human HeLa cells to determine the effects of placement of sugar and internucleoside linkages within ssRNAs. More specifically, the modified ssRNAs were tested for their ability to reduce PTEN mRNA in cultured cells. The modified ssRNAs are shown below, and contain 2′-OMe and 2′-fluoro modified nucleosides, two 2′-O-MOE modified nucleosides at the 3′-terminus, and seven phosphorothioate linkages at the 3′-terminus of the ssRNAs.

HeLa cells were treated with ssRNAs shown below at concentrations of 1.56 nM, 3.13 nM, 6.25 nM, 12.5 nM, 20 nM and 50 nM using methods described herein. Levels of mRNA were determined using real-time PCR methods as described herein. The IC₅₀ for each ssRNA was determined. These data demonstrate that these modified ssRNA exhibit similar activity in decreasing target mRNA levels.

| SEQ ID | ISIS | | |
|---|---|---|---|
| NO. | NO. | Composition (5′ to 3′) | IC_{₅₀} |
| 06 | 404320 | P-U_{f}U_{f}G_{f}U_{f}C_{f}U_{f}C_{f}U_{f}G_{f}G_{f}U_{f}C_{f}C_{f}U_{f}U_{f}A_{f}C_{f}U_{f}U_{f}AₑAₑ | 5.8 |
| 06 | 408874 | P-U_{f}U_{f}G_{f}U_{f}C_{f}UₘCₘU_{f}G_{f}GₘUₘC_{f}C_{f}U_{f}U_{f}A_{f}CₘUₘUₘAₑAₑ | 6.0 |
| 06 | 408877 | P-UₘU_{f}G_{f}U_{f}C_{f}UₘCₘU_{f}G_{f}GₘUₘC_{f}C_{f}U_{f}U_{f}A_{f}CₘUₘUₘAₑAₑ | 7.0 |
| 06 | 409044 | P-UₘU_{f}GₘU_{f}CₘU_{f}CₘU_{f}GₘG_{f}UₘC_{f}CₘU_{f}UₘA_{f}CₘU_{f}UₘAₑAₑ | 10.5 |
| 06 | 407047 | U_{f}U_{f}G_{f}U_{f}C_{f}U_{f}CₘU_{f}GₘG_{f}UₘC_{f}CₘU_{f}UₘA_{f}CₘU_{f}UₘAₑAₑ | 3.5 |
| 17 | 409049 | P-U_{f}U_{f}G_{f}U_{f}C_{f}U_{f}CₘU_{f}GₘG_{f}UₘC_{f}CₘU_{f}UₘA_{f}CₘU_{f}UₘTₑTₑ | 16.2 |
| 17 | 409062 | P-U_{f}U_{f}G_{f}U_{f}C_{f}UₘCₘU_{f}G_{f}GₘUₘC_{f}C_{f}U_{f}U_{f}A_{f}CₘUₘUₘTₑTₑ | 8.6 |

Phosphorothioate internucleoside linkages are indicated by underlining. Modified nucleosides are indicated by a subscripted letter following the capital letter indicating the nucleoside. In particular, subscript “f” indicates 2′-fluoro; subscript “m” indicates 2′-O-methyl; and subscript “e” indicates 2′-O-methoxyethyl (MOE). For example, U_{f} is a modified uridine having a 2′-fluoro group. Some of the strands have a 5′-phosphate group designated as “P-”.

### Example 65

### ssRNAs Stability in Hepatocyte Cell Homogenate Assay—In Vivo Study

The stability of oligomeric compounds can be evaluated in a cell homogenate assay.

Hepatocytes were harvested from bal/c mice in ice-cold hepatocyte wash media (William E Media) with fetal bovine serum, sedimented by centrifugation at 1000 g for 8 minutes and then washed with hepatocyte wash media. Hepatocytes were homogenized with RIPA buffer (50 mM Tris pH 7.5, 10 mM MgCl₂, 150 mM NaCl, 0.5% NP-40 alternative, one tablet of Roche protease inhibitor #11836170001), and centrifuged at 14000 g for 15 minutes at 4° C. and the supernatant was removed and stored in ice. Protein concentration (BSA mg/mL) was determined with Bradford assay and adjusted to a final protein concentration of 2 mg/mL by addition of Ripa buffer volume or cell homogenate volume.

### Phenol/Choroform Extraction.

ssRNA (1 mL, 20 μL) were homogenized in a homogenation buffer (20 mM Tris, pH 8, 20 mM EDTA and 0.1 M NaCl in 0.5% NP-40) at time points 0, 5, 10, 20, 30, 40 and 60 minutes (Exception: 06/408877 at time points 0, 15, 30, 60, 120 and 240 mins, 06/409044, at time points 0, 0.5, 1, 2, 4, 8, and 18 hours). An internal standard (18/355868, a 27-mer, 2′-O-methoxyethyl-modified phosphorothioate oligonucleotide, or 19/116847, a 5-10-5 gappmer, 2′-O-methoxyethyl-modified phosphorothioate oligonucleotide) with concentration at 20 ug/g was added prior to extraction. Tissue samples were extracted with 70 μL of NH₄OH and 240 μL of phenol/chloroform/isoamyl alcohol (25:24:1). The supernatant was removed after centrifugation at 14000 rpm for 2 min. The remaining extractant was vortexed with an additional 5004 of water and the aqueous layer was removed and combined with the supernatant after centrifugation at 14000 rpm for 2 minutes.

### Solid Phase Extraction.

Triethylammonium acetate solution at 1M (5004) was added to the supernatant. The aqueous layer of the mixture was loaded onto the pre-conditioned Biotage™ Phenyl Solid Phase Extraction Plate (SPE plate) after centrifugation at 9000 rpm for 20 minutes. The SPE plate was washed several times with water. The sample was then eluted with 1.5 mL of 1% TEA in 90% MeOH and filtered through the Protein Precipitation Plate (Phenomenex™). The elutent was evaporated to dryness and diluted to 200 μL with 50% quenching buffer (8 M urea, 50 mM EDTA) and water before sample injection.

### LC-MS.

An Agilent 1100 Series LC/MSD system was connected in-line to a mass spectrometry. Mass spectrometer was operated in the electrospray negative ionization mode. The nebulizer nitrogen gas was set at 325 psi and the drying nitrogen gas was set at 12 L/min. The drying temperature was 325° C. Samples (25 μL/well) were introduced via an auto sampler and reversed-phase chromatography was carried out with an XBridge OST C18 2.5 μm 2.1 mm×50 mm HPLC column using a flow rate of 300 μL/min at 55° C. The ion pair buffers consisted of A: 5 mM tributylammonium acetate (TBAA) in 20% acetonitrile and B: 5 nM TBAA in 90% acetonitrile and the loading buffer was 25 mM TBAA in 25% Acetonitrile. Separation was performed on a 30% to 70% B in 9 min and then 80% B in 11 min gradient.

Quantitative analysis of oligonucleotide and internal standard by extracted ion chromatograms of the most abundant ions was performed using MSD ChemStation software. The results are expressed as half-time (T_{1/2}) in the table below. These data demonstrate that modifications to oligomeric compounds improve their stability in a cell homogenate assay.

| | | |
|---|---|---|
| SEQ ID NO./ | T_{1/2} | T_{1/2} |
| ISIS NO. | Test 1 | Test 2 |

| 06/404320 | 4 | min | — | |
|---|---|---|---|---|
| 06/408874 | 22 | min | 18 | min |
| 06/408877 | 30 | min | 24 | min |
| 06/409044 | 4 | hr | 4.3 | hr |

| | | | |
|---|---|---|---|
| 06/407047 | 6 | min | — |
| 17/409049 | 13 | min | — |
| 17/409062 | 17 | min | — |

### Internal Standards:

| SEQ | | |
|---|---|---|
| ID | ISIS | |
| NO. | NO. | Composition (5′ to 3′) |
| 18 | 355868 | Gₑ^{me}CₑGTTTGCTCTTCTTₑ^{me}CₑTₑTₑGₑ^{me}CₑGₑTTTTTₑTₑ |
| 19 | 116847 | ^{me}CₑTₑGₑ^{me}CₑTₑAG^{me}C^{me}CT^{me}CTGGATₑTₑTₑGₑAₑ |

Each internucleoside linkage is a phosphorothioate internucleoside linkage indicated by underlining (going 5′ to 3′). Each unmodified nucleoside is a β-D-2′-deoxyribonucleosides. Nucleosides followed by a subscript “e” indicates a 2′-O(CH₂)₂OCH₃ (MOE) modified nucleoside. Superscript “me” indicates a 5-methyl group on the pyrimidine base of the nucleoside.

### Example 66

### MicroRNA Mimics: Cell Cycle Assay

Oligomeric compounds comprising the nucleobase sequence of a microRNA were synthesized to have certain modifications described herein. These microRNA mimics were tested for their ability to imitate microRNA activity.

A cell cycle assay was used to evaluate the activity of microRNA mimics. A549 cells were plated at a density of approximately 45,000 cells per well of a 24-well plate. The following day, cells were transfected with microRNA mimics and control oligomeric compounds, using RNAIMAX as the transfection reagent. Oligomeric compounds were tested at concentrations ranging from 0.1 nM to 100 nM. Control oligomeric compounds were also tested. Approximately 24 hours following transfection, nocodazole was added to the cells at a concentration ranging from 0.5 to 2.0 μg/ml. Approximately 16 hours later, the cells were harvested, washed, ethanol-fixed and stained with propidium iodide. Cells cycle profiles were generated by subjecting the stained cells to flow cytometry (FACSCAN).

miR-16 Mimics: Cell Cycle Assay

A cell cycle assay was used to test the activity of miR-16 mimics (shown in table below). The addition of a double-stranded miR-16 mimic blocked cells in the G1 phase of the cell cycle. The single stranded miR-16 mimic produced the same phenotype as the double-stranded mimic, blocking cells in the G1 phase of the cell cycle. The single stranded miR-16 mimic exhibited similar efficacy as the double-stranded miR-16 mimic.

| | SEQ | |
|---|---|---|
| | ID | |
| | NO | Composition (5′ to 3′) |
| ss miR-16 | 20 | P-UₘA_{f}G_{f}C_{f}A_{f}G_{f}C_{f}A_{f}CₘGₘU_{f}A_{f}AₘAₘU_{f}A_{f}U_{f}U_{f}G_{f}GₘCₘGₘAₑAₑ |
| ds miR-16 | 21 | UAGCAGCACGUAAAUAUUGGCG |
| | 22 | AAAGCGUCGUGCAUUUAUAACC |

Internucleoside linkage and sugar modifications are indicated as described in previous examples.

miR-34 Mimics: Cell Cycle Assay

A cell cycle assay was used to test the activity of miR-34 mimics. The addition of a double-stranded miR-34 mimic blocked cells in the G1 phase of the cell cycle. The above single stranded miR-34 mimic produced the same phenotype as the double-stranded mimic, blocking cells in the G1 phase of the cell cycle. The single stranded miR-34 mimic exhibited similar efficacy as the double-stranded miR-34 mimic.

In addition to measuring cell cycle progression, cells treated with miR-34 mimics were subjected to microarray analysis to compare the profile of gene expression changes following treatment with microRNA mimics. The microarray analysis is used to evaluate the enrichment of target nucleic acids that comprise a seed match segment in their 3′ untranlated regions from among the pool of nucleic acids that are down-regulated following treatment with a microRNA mimic.

Both the double-stranded miR-34 mimic and single-stranded miR-34 mimic down-regulated miR-34 seed-matched nucleic acids. However, also observed was an enrichment of nucleic acids comprising a seed match segment of the microRNA complement strand (the “passenger strand”) of the double-stranded mimic, thus the microRNA complement strand was also acting an antisense compound. This activity is not specific to miR-34. Accordingly, a single-strand microRNA mimic can provide improved specificity relative to a double-stranded mimic.

These data demonstrate that the oligomeric compounds described herein can be designed as microRNA mimics. Further, single-stranded mimics are effective at imitating microRNA activity.

| | SEQ | |
|---|---|---|
| | ID | |
| | NO | Composition (5′ to 3′) |
| ss miR-34 | 23 | P-UₘG_{f}G_{f}C_{f}A_{f}G_{f}U_{f}G_{f}UₘCₘU_{f}U_{f}AₘGₘC_{f}U_{f}G_{f}G_{f}U_{f}U_{f}G_{f}U_{f}AₑAₑ |

Internucleoside linkage and sugar modifications are indicated as described in previous examples.

Additional miR-34 Mimics: Cell Cycle Assay

Additional single-stranded miR-34 mimics were tested in a cell cycle assay. Each of these oligomeric compounds resulted in a block in the G1 phase of the cell cycle, indicating that these single-stranded microRNA mimics are effective at imitating microRNA activity.

| | SEQ | |
|---|---|---|
| | ID | |
| | NO | Composition (5′ to 3′) |
| ss miR-34 | 23 | P-U_{d}G_{f}G_{f}C_{f}A_{f}G_{f}U_{f}G_{f}U_{f}C_{f}U_{f}U_{f}A_{f}G_{f}C_{f}U_{f}G_{f}G_{f}U_{f}U_{f}G_{f}U_{f}AₑAₑ |
| ss miR-34 | 23 | P-U_{d}G_{f}G_{f}CₘA_{f}GₘU_{f}GₘU_{f}CₘU_{f}UₘA_{f}GₘC_{f}U_{f}G_{f}G_{f}U_{f}U_{f}G_{f}U_{f}AₑAₑ |
| ss miR-34 | 23 | P-UₘG_{f}G_{f}C_{f}A_{f}G_{f}U_{f}GₘUₘC_{f}U_{f}UₘAₘG_{f}C_{f}U_{f}G_{f}G_{f}U_{f}UₘGₘUₘAₑAₑ |
| ss miR-34 | 23 | P-UₑG_{f}G_{f}C_{f}A_{f}G_{f}U_{f}GₘUₘC_{f}U_{f}UₘAₘG_{f}C_{f}U_{f}G_{f}G_{f}U_{f}UₘGₘUₘAₑAₑ |

Internucleoside linkage and sugar modifications are indicated as described in previous examples.

### Example 67

### MicroRNA Mimics: Cytokine Signaling Assay

Oligomeric compounds comprising the nucleobase sequence of a microRNA were synthesized to have certain modifications described herein. These oligomeric compounds were tested for their ability to mimic microRNA activity. A cytokine signaling assay was used to evaluate the activity of microRNA mimics.

miR-146 mimics

miR-146 is known to stimulate the release of cytokines such as IL-8, thus the following assay can be used to measure the activity of miR-146 mimics. A549 cells were treated with the miR-146 mimics shown below. Cells were treated with IL-1B at a concentration ranging from 0.1 to 2.0 ng/ml. After 8 hours and 24 hours, samples were collected for ELISA analysis to measure the release of the cytokine IL-8. Measurement of IL-8 in the cell culture supernatant revealed that single-strand miR-146 mimics decreased the release of IL-8 in a dose-responsive manner in this assay. Accordingly, the single-strand miR-146 mimics shown below exhibit an activity of miR-146.

| | SEQ | |
|---|---|---|
| | ID | |
| | NO | Composition (5′ to 3′) |
| ss miR-146 | 24 | P-UₘG_{f}A_{f}G_{f}A_{f}A_{f}C_{f}U_{f}GₘAₘA_{f}U_{f}UₘCₘC_{f}A_{f}U_{f}G_{f}G_{f}GₘUₘUₘAₑAₑ |
| ss miR-146 | 24 | P-UₘG_{f}A_{f}G_{f}A_{f}A_{f}C_{f}U_{f}G_{f}AₘAₘU_{f}U_{f}CₘCₘA_{f}U_{f}G_{f}G_{f}GₘUₘUₘAₑAₑ |

Additional oligomeric compounds were designed and comprise the nucleobase sequence of miR-146. These oligomeric compounds were shown to mimic miR-146 activity in the IL-8 release assay described above.

| | SEQ | |
|---|---|---|
| | ID | |
| | NO | Composition (5′ to 3′) |
| ss miR-146 | 24 | P-UₘG_{f}A_{f}G_{f}A_{f}A_{f}C_{f}U_{f}GₘAₘA_{f}U_{f}UₘCₘC_{f}A_{f}U_{f}G_{f}G_{f}GₘUₘUₘAₑAₑ |
| ss miR-146 | 24 | P-UₘG_{f}AₘG_{f}A_{f}AₘC_{f}U_{f}GₘAₘA_{f}U_{f}UₘCₘC_{f}A_{f}U_{f}G_{f}G_{f}GₘUₘUₘAₑAₑ |
| ss miR-146 | 24 | P-UₘG_{f}AₘG_{f}AₘA_{f}CₘU_{f}GₘA_{f}AₘU_{f}UₘC_{f}CₘA_{f}UₘG_{f}GₘG_{f}UₘU_{f}AₑAₑ |

Additional oligomeric compounds were designed and comprise the nucleobase sequence of miR-146.

| | SEQ | |
|---|---|---|
| | ID | |
| | NO | Composition (5′ to 3′) |
| ss miR-146 | 24 | P-UₘG_{f}A_{f}G_{f}A_{f}A_{f}C_{f}U_{f}GₘAₘA_{f}U_{f}UₘCₘC_{f}A_{f}U_{f}G_{f}G_{f}GₘUₘUₘAₑAₑ |

miR-155 Mimics

Additional oligomeric compounds were designed and comprise the nucleobase sequence of miR-155.

| | SEQ | |
|---|---|---|
| | ID | |
| | NO | Composition (5′ to 3′) |
| ss miR-155 | 25 | P-UₘU_{f}A_{f}A_{f}U_{f}G_{f}C_{f}U_{f}A_{f}AₘUₘC_{f}G_{f}UₘGₘA_{f}U_{f}A_{f}G_{f}G_{f}GₘGₘUₘAₑAₑ |

Internucleoside linkage and sugar modifications are indicated as described in previous examples.

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents patent application publications, international patent application publications, gene back accession numbers, and the like) cited in the present application is incorporated herein by reference in its entirety.

### Example 68

### Phosphate Stability in Mouse Serum

Single-stranded oligomeric compounds were tested for stability in mouse serum. The single stranded oligomeric compounds and the half lives of full compound with intact phosphorous moiety are provided in the table below.

| SEQ ID NO./ | T_{1/2} full | |
|---|---|---|
| ISIS NO. | length | Sequence |
| 06/404320 | 0.4 hours | 5′-Po-U_{fo}U_{fo}G_{fo}U_{fo}C_{fo}U_{fo}C_{fo}U_{fo}G_{fo}G_{fo}U_{fo}C_{fo}C_{fo}U_{fs}U_{fs}A_{fs}C_{fs}U_{fs}U_{fs}AₑₛAₑ |
| 10/430601 | 3.7 hours | 5′-Ps-U_{d}U_{fo}G_{fo}U_{fo}C_{fo}U_{fo}C_{fo}U_{fo}G_{fo}G_{fo}U_{fo}C_{fo}C_{fo}U_{fs}U_{fs}A_{fs}C_{fs}U_{fs}U_{f} |
| 06/418129 | 5.4 hours | 5′-Po-UᵢU_{fo}G_{fo}U_{fo}C_{fo}U_{fo}C_{fo}U_{fo}G_{fo}G_{fo}U_{fo}C_{fo}C_{fo}U_{fs}U_{fs}A_{fs}C_{fs}U_{fs}U_{fs}AₑₛAₑ |
| 05/418130 | 5.3 hours | 5′-Po-TⱼU_{fo}G_{fo}U_{fo}C_{fo}U_{fo}C_{fo}U_{fo}G_{fo}G_{fo}U_{fo}C_{fo}C_{fo}U_{fs}U_{fs}A_{fs}C_{fs}U_{fs}U_{fs}AₑₛAₑ |
| 05/432356 | 8.7 hours | 5′-Po-UₖU_{fo}G_{fo}U_{fo}C_{fo}U_{fo}C_{fo}U_{fo}G_{fo}G_{fo}U_{fo}C_{fo}C_{fo}U_{fs}U_{fs}A_{fs}C_{fs}U_{fs}U_{fs}AₑₛAₑ |
| 05/422391 | 6.5 hours | 5′-Po-T_{d}U_{fo}G_{fo}U_{fo}C_{fo}U_{fo}C_{fo}U_{fo}G_{fo}G_{fo}U_{fo}C_{fo}C_{fo}U_{fs}U_{fs}A_{fs}C_{fs}U_{fs}U_{fs}AₑₛAₑ |

Subscripts in the Table above: d=DMAEAc; i=N-methoxyamino BNA; J=tcDNA; k=(R) 5′-methyl

Separately, four oligomeric compounds were tested for stability in mouse serum, as summarized in the table below.

| SEQ ID NO./ | T1/2 full | |
|---|---|---|
| ISIS NO. | length | Sequence |
| 06/404320 | 0.4 hours | 5′-Po-U_{fo}U_{fo}G_{fo}U_{fo}C_{fo}U_{fo}C_{fo}U_{fo}G_{fo}G_{fo}U_{fo}C_{fo}C_{fo}U_{fs}U_{fs}A_{fs}C_{fs}U_{fs}U_{fs}AₑₛAₑ |
| 05/422391 | 3.7 hours | 5′-Po-U_{d}U_{fo}G_{fo}U_{fo}C_{fo}U_{fo}C_{fo}U_{fo}G_{fo}G_{fo}U_{fo}C_{fo}C_{fo}U_{fs}U_{fs}A_{fs}C_{fs}U_{fs}U_{f} |
| 05/440141 | 0.3 hours | 5′-Po-TₕU_{fo}G_{fo}U_{fo}C_{fo}U_{fo}C_{fo}U_{fo}G_{fo}G_{fo}U_{fo}C_{fo}C_{fo}U_{fs}U_{fs}A_{fs}C_{fs}U_{fs}U_{fs}AₑₛAₑ |
| 05/435395 | 7.8 hours | 5′-Ps-U_{d}U_{fo}G_{fo}U_{fo}C_{fo}U_{fo}C_{fo}U_{fo}G_{fo}G_{fo}U_{fo}C_{fo}C_{fo}U_{fs}U_{fs}A_{fs}C_{fs}U_{fs}U_{fs}AₑₛAₑ |

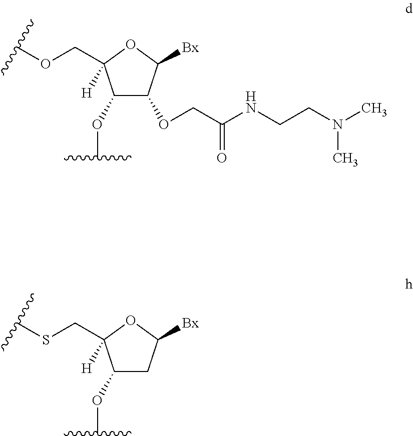

### Example 69

### Modified Oligomeric Compounds Targeting PTEN: In Vitro Study

In accordance with the present disclosure, oligomeric compounds were synthesized and tested for their ability to reduce PTEN expression over a range of doses. Human HeLa cells were treated with either ISIS 447581, 467074, 418046 or 467076. A dose comparison was evaluated with dose concentrations of 0.067, 0.2, 0.62, 1.9, 5.5, 16.7 and 50 nM using methods described herein. Expression levels of PTEN were determined using real-time PCR and normalized to RIBOGREEN™ using methods described herein. The percent inhibition of PTEN mRNA was determined and the resulting dose-response curves were used to determine the EC₅₀. The EC₅₀s are listed below.

| SEQ | | | |
|---|---|---|---|
| ID | ISIS | | EC₅₀ |
| NO. | NO. | Composition (5′ to 3′) | (nM) |
| 05 | 447581 | P-T_{Rc}U_{f}GₘU_{f}CₘU_{f}CₘU_{f}GₘG_{f}UₘC_{f}CₘU_{f}UₘA_{f}CₘU_{f}UₘAₑAₑ | 0.6 |
| 05 | 467074 | P-T_{Sc}U_{f}GₘU_{f}CₘU_{f}CₘU_{f}GₘG_{f}UₘC_{f}CₘU_{f}UₘA_{f}CₘU_{f}UₘAₑAₑ | 2.5 |
| 06 | 418046 | P-UₘU_{f}GₘU_{f}CₘU_{f}CₘU_{f}GₘG_{f}UₘC_{f}CₘU_{f}UₘA_{f}CₘU_{f}UₘAₑAₑ | 0.83 |
| 06 | 467076 | Py-^{me}UₘU_{f}GₘU_{f}CₘU_{f}CₘU_{f}GₘG_{f}UₘC_{f}CₘU_{f}UₘA_{f}CₘU_{f}UₘAₑAₑ | 6.0 |

Each internucleoside linkage is a phosphodiester except that underlined nucleosides are linked to the following nucleoside by a phosphorothioate (going 5′ to 3′). A “P” at the 5′-end indicates a 5′-phosphate group. A “Py” at the 5′-end indicates a 5′-methylenephosphonate group, (PO(OH)₂CH₂—). Nucleosides followed by a subscript e, form indicate modification as follows: subscript “e” indicates a 2′-O(CH₂)₂OCH₃ (MOE) modified nucleoside, subscript “f” indicates a 2′-fluoro modified nucleoside; subscript “m” indicates 2′-O-methyl modified nucleoside. Superscript “me” indicates a 5-methyl group on the pyrimidine base of the nucleoside. Nucleosides with subscript “Rc” or “Sc” are shown below.

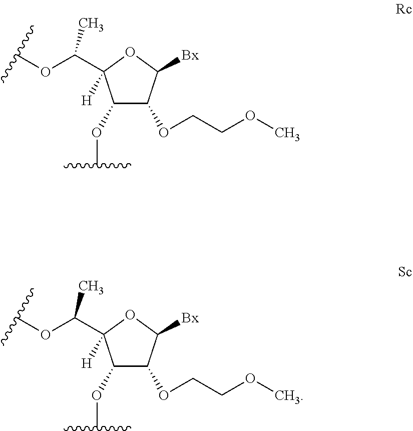

### Example 70

### 5′-Modified Oligomeric Compounds Targeting PTEN: In Vivo Study

Three oligomeric compounds (ISIS 467074, ISIS 467076, ISIS 116847) were synthesized as described above. Sequence and chemistry of the three oligomeric compounds are provided in the table, below. The nucleobase sequence of each oligomeric compound is complementary to PTEN.

| SEQ | | |
|---|---|---|
| ID | ISIS | |
| NO. | NO. | Composition (5′ to 3′) |
| 05 | 467074 | P-T_{Sc}U_{f}GₘU_{f}CₘU_{f}CₘU_{f}GₘG_{f}UₘC_{f}CₘU_{f}UₘA_{f}CₘU_{f}UₘAₑAₑ |
| 06 | 467076 | Py-^{me}UₘU_{f}GₘU_{f}CₘU_{f}CₘU_{f}GₘG_{f}UₘC_{f}CₘU_{f}UₘA_{f}CₘU_{f}UₘAₑAₑ |
| 08 | 116847 | ^{me}CₑTₑGₑ^{me}CₑTₑAG^{me}C^{me}CT^{me}CTGGATₑTₑTₑGₑAₑ |

Each internucleoside linkage is a phosphodiester except that underlined nucleosides are linked to the following nucleoside by a phosphorothioate (going 5′ to 3′). “Py” at the 5′-end indicates a 5′-methylenephosphonate group, (PO(OH)₂CH₂—). Each unmodified nucleoside is a β-D-2′-deoxyribonucleosides. Nucleosides followed by a subscript e, form indicate modification as follows: subscript “e” indicates a 2′-O(CH₂)₂OCH₃ (MOE) modified nucleoside, subscript 1′ indicates a 2′-fluoro modified nucleoside; subscript “m” indicates 2′-O-methyl modified nucleoside. Superscript “me” indicates a 5-methyl group on the pyrimidine base of the nucleoside. Nucleoside with subscript “Sc” is shown below.

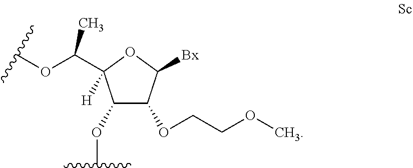

Six-week-old male Balb/c mice (Jackson Laboratory, Bar Harbor, Me.) were injected intraperitenially with a single dose of 75 mg/kg of one of the three oligomeric compounds above or with saline control. Each dose group consisted of four animals. The mice were sacrificed 48 hours following administration. Livers were collected and PTEN mRNA levels were assessed using real-time PCR and RIBOGREEN® RNA quantification reagent (Molecular Probes, Inc. Eugene, Oreg.) according to standard protocols. PTEN mRNA levels were determined relative to total RNA (using Ribogreen), and normalized to the saline-treated control. Results are listed below as the average % inhibition of PTEN mRNA expression for each treatment group, normalized to saline-injected control.

| | SEQ ID NO./ISIS NO | | | |
|---|---|---|---|---|
| | 05/467074 | 06/467076 | 08/116847 | Saline (Control) |
| Dose | 75 mg/kg | 75 mg/kg | 75 mg/kg | 0 mg/kg |
| Time (h) | 48 | 48 | 48 | 48 |
| % inhibition | 11% | 16% | 76% | 0% |

### Example 71

### Stability of 5′-Modified Oligomeric Compounds Targeting PTEN: In Vivo Study

The in vivo stability of the three oligomeric compounds in Example 70 was evaluated. The tissue samples were obtained from the animals in which PTEN was assessed. Tissue samples were collected and prepared using the same technique described in Example 65. Quantitative analysis of the oligonucleotides standard were performed by extracted ion chromatograms in the most abundant charge state (−4) using Chemstation software. The tissue level (μg/g) of intact compound of ISIS 116847, 467074 and 467076 was measured and are provided below:

| SEQ ID NO./ | Dose @ 75 mg/kg (48 h time point) |
|---|---|
| ISIS NO. | Tissue Level of intact compound (μg/g) |
| 05/467074 | none detected |
| 06/467076 | 22.5 |
| 08/116847 | 131.1 |

The 5-10-5 MOE gapmer compound was present at high levels and was a potent inhibitor of PTEN. Intact 467076 was present at a lower concentration and resulted in smaller inhibition of PTEN. Intact 467074 was not detected and resulted in the lowest amount of PTEN reduction. Some 467074 lacking the 5′-phosphate was detected.

### Example 72

### Effect of Modified Internucleoside Linkages on Modified Oligomeric Compounds Targeting PTEN—In Vitro Study

In accordance with the present disclosure, oligomeric compounds were synthesized and tested for their ability to reduce PTEN expression over a range of doses. Human HeLa cells were treated with the following oligomeric compounds. A dose comparison was evaluated with dose concentrations of 0.167, 0.5, 1.5, 5, 15 and 50 nM using methods described herein. Expression levels of PTEN were determined using real-time PCR and normalized to RIBOGREEN™ using methods described herein. The percent inhibition of PTEN mRNA was determined and the resulting dose-response curves were used to determine the IC₅₀. The IC₅₀s are listed below.

| SEQ | | | |
|---|---|---|---|
| ID | ISIS | | IC₅₀ |
| NO. | NO. | Composition (5′ to 3′) | (nM) |
| 05 | 435397 | P-T_{d}U_{f}GₘU_{f}CₘU_{f}CₘU_{f}GₘG_{f}UₘC_{f}CₘU_{f}UₘA_{f}CₘU_{f}UₘAₑAₑ | 2.0 |
| 05 | 435394 | P-T_{d}U_{f}G_{f}U_{f}C_{f}U_{f}C_{f}U_{f}G_{f}G_{f}U_{f}C_{f}C_{f}U_{f}U_{f}A_{f}C_{f}U_{f}U_{f}AₑAₑ | 18.1 |
| 05 | 435399 | P-T_{d}U_{f}GₘU_{f}CₘU_{f}CₘU_{f}GₘG_{f}UₘC_{f}C_{f}U_{f}U_{f}A_{f}C_{f}U_{f}U_{f}AₑAₑ | 2.0 |
| 05 | 418031 | P-TₑU_{f}G_{f}U_{f}C_{f}U_{f}C_{f}U_{f}G_{f}G_{f}U_{f}C_{f}C_{f}U_{f}U_{f}A_{f}C_{f}U_{f}U_{f}AₑAₑ | 3.9 |
| 05 | 418032 | P-T_{ef}U_{f}G_{f}U_{f}C_{f}U_{f}C_{f}U_{f}G_{f}G_{f}U_{f}C_{f}C_{f}U_{f}U_{f}A_{f}C_{f}U_{f}U_{f}AₑAₑ | 2.9 |
| 05 | 418033 | P-T_{ef}U_{f}G_{f}U_{f}C_{f}U_{f}C_{f}U_{f}G_{f}G_{f}U_{f}C_{f}C_{f}U_{f}U_{f}A_{f}C_{f}U_{f}U_{f}AₑAₑ | 11.0 |
| 05 | 418131 | P-TU_{f}G_{f}U_{f}C_{f}U_{f}C_{f}U_{f}G_{f}G_{f}U_{f}C_{f}C_{f}U_{f}U_{f}A_{f}C_{f}U_{f}U_{f}AₑAₑ | 3.4 |
| 06 | 404320 | P-U_{f}U_{f}G_{f}U_{f}C_{f}U_{f}C_{f}U_{f}G_{f}G_{f}U_{f}C_{f}C_{f}U_{f}U_{f}A_{f}C_{f}U_{f}U_{f}AₑAₑ | 7.6 |
| 06 | 414291 | P-UₘU_{f}GₘU_{f}CₘU_{f}CₘU_{f}GₘG_{f}UₘC_{f}CₘU_{f}UₘA_{f}CₘU_{f}UₘAₑAₑ | 13.0 |
| 06 | 416598 | P-UₘU_{f}GₘU_{f}CₘU_{f}CₘU_{f}GₘG_{f}UₘC_{f}CₘU_{f}UₘA_{f}CₘU_{f}UₘAₑAₑ | 6.8 |
| 06 | 418030 | P-UₑU_{f}G_{f}U_{f}C_{f}U_{f}C_{f}U_{f}G_{f}G_{f}U_{f}C_{f}C_{f}U_{f}U_{f}A_{f}C_{f}U_{f}U_{f}AₑAₑ | 8.5 |

Each internucleoside linkage is a phosphodiester except that underlined nucleosides are linked to the following nucleoside by a phosphorothioate (going 5′ to 3′). A “P” at the 5′-end indicates a 5′-phosphate group. Each unmodified nucleoside is a β-D-2′-deoxyribonucleoside. Nucleosides followed by a subscript d, e, f, m or x indicate modification as follows: a subscript “d” indicates a 2′-OCH₂(CO)NH(CH₂)₂N(CH₃)_{2 (DMAEAc), subscript “e” indicates a} 2′-O(CH₂)₂OCH₃ (MOE) modified nucleoside, subscript “f” indicates a 2′-fluoro modified nucleoside subscript “m” indicates 2′-O-methyl modified nucleoside and subscript “ef” indicates a 2′-OCH₂CH₂F (FEt) modified nucleoside.

### Example 73

### Synthesis for precursors of 2,2-Dilinoleyl-4-dimethylaminoethyl-[1,3]-dioxolane

### Synthesis of methanesulfonic acid octadeca-9,12-dienyl ester 2

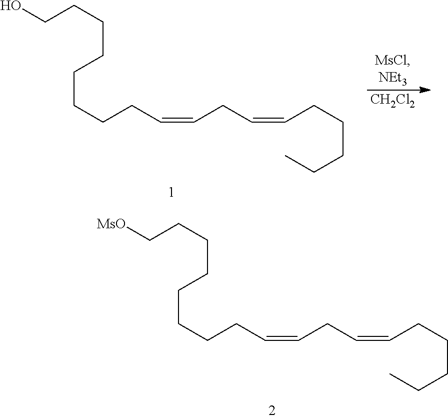

To a solution of the alcohol 1 (26.6 g, 100 mmol) in dichloromethane (100 mL), triethylamine (13.13 g, 130 mmol) was added and this solution was cooled in ice-bath. To this cold solution, a solution of mesyl chloride (12.6 g, 110 mmol) in dichloromethane (60 mL) was added dropwise and after the completion of the addition, the reaction mixture was allowed to warm to ambient temperature and stirred overnight. The TLC of the reaction mixture showed the completion of the reaction. The reaction mixture was diluted with dichloromethane (200 mL), washed with water (200 mL), satd. NaHCO₃ (200 mL), brine (100 mL) and dried (NaSO₄). The organic layer was concentrated to get the crude product which was purified by column chromatography (silica gel) using 0-10% Et₂O iii hexanes. The pure product fractions were combined and concentrated to obtain the pure product 2 as colorless oil (30.6 g, 89%). ¹H NMR (CDCl₃, 400 MHz) δ=5.42-5.21 (m, 4H), 4.20 (t, 2H), 3.06 (s, 3H), 2.79 (t, 2H), 2.19-2.00 (m, 4H), 1.90-1.70 (m, 2H), 1.06-1.18 (m, 18H), 0.88 (t, 3H). ¹³C NMR (CDCl₃) δ=130.76, 130.54, 128.6, 128.4, 70.67, 37.9, 32.05, 30.12, 29.87, 29.85, 29.68, 29.65, 29.53, 27.72, 27.71, 26.15, 25.94, 23.09, 14.60. MS. Molecular weight calculated for C₁₉H₃₆O₃S, Cal. 344.53, Found 343.52 (M−H).

### Synthesis of 18-Bromo-octadeca-6,9-diene 3

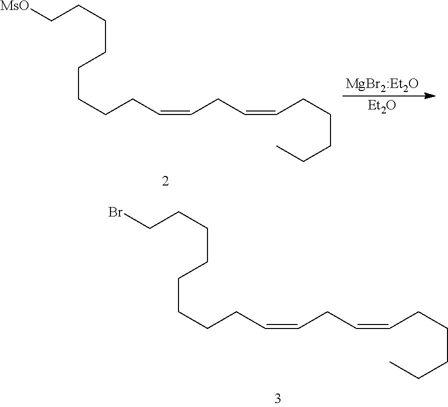

The mesylate (13.44 g, 39 mmol) was dissolved in anhydrous ether (500 mL) and to it the MgBr.Et₂O complex (30.7 g, 118 mmol) was added under argon and the mixture was refluxed under argon for 26 h after which the TLC showed the completion of the reaction. The reaction mixture was diluted with ether (200 mL) and ice-cold water (200 mL) was added to this mixture and the layers were separated. The organic layer was washed with 1% aqueous K₂CO₃ (100 mL), brine (100 mL) and dried (Anhyd. Na₂SO₄). Concentration of the organic layer provided the crude product which was further purified by column chromatography (silica gel) using 0-1% Et₂O in hexanes to isolate the bromide 3 (12.6 g, 94%) as a colorless oil. ¹H NMR (CDCl₃, 400 MHz) δ=5.41-5.29 (m, 4H), 4.20 (d, 2H), 3.40 (t, J=7 Hz, 2H), 2.77 (t, J=6.6 Hz, 2H), 2.09-2.02 (m, 411), 1.88-1.00 (m, 2H), 1.46-1.27 (m, 18H), 0.88 (t, J=3.9 Hz, 3H). ¹³C NMR (CDCl₃) δ=130.41, 130.25, 128.26, 128.12, 34.17, 33.05, 31.75, 29.82, 29.57, 29.54, 29.39, 28.95, 28.38, 27.42, 27.40, 25.84, 22.79, 14.28.

### Synthesis of 18-Cyano-octadeca-6,9-diene 4

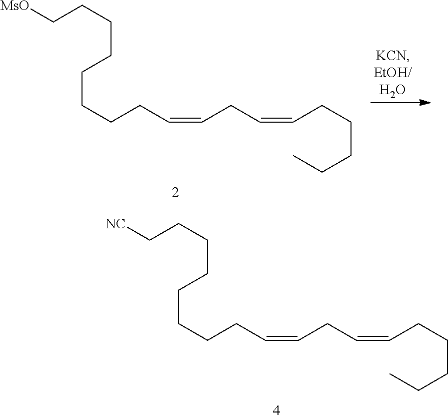 [SMILES: CCCCC/C=C\C/C=C\CCCCCCCCOS(C)(=O)=O ; CCCCC/C=C\C/C=C\CCCCCCCCC#N]

To a solution of the mesylate (3.44 g, 10 mmol) in ethanol (90 mL), a solution of KCN (1.32 g, 20 mmol) in water (10 mL) was added and the mixture was refluxed for 30 min. after which, the TLC of the reaction mixture showed the completion of the reaction after which, ether (200 mL) was added to the reaction mixture followed by the addition of water. The reaction mixture was extracted with ether and the combined organic layers was washed with water (100 mL), brine (200 mL) and dried. Concentration of the organic layer provided the crude which was purified by column chromatography (0-10% Et₂O in hexanes). The pure product 4 was isolated as colorless oil (2 g, 74%). ¹H NMR (CDCl₃, 400 MHz) δ=5.33-5.22 (m, 4H), 2.70 (t, 2H), 2.27-2.23 (m, 2H), 2.00-1.95 (m, 4H), 1.61-1.54 (m, 2H), 1.39-1.20 (m, 1811), 0.82 (t, 3H). ¹³C NMR (CDCl₃) δ=130.20, 129.96, 128.08, 127.87, 119.78, 70.76, 66.02, 32.52, 29.82, 29.57, 29.33, 29.24, 29.19, 29.12, 28.73, 28.65, 27.20, 27.16, 25.62, 25.37, 22.56, 17.10, 14.06. MS. Molecular weight calculated for C₁₉H₃₃N, Cal. 275.47, Found 276.6 (MH⁻).

### Synthesis of Heptatriaconta-6,9,28,31-tetraen-19-one 7

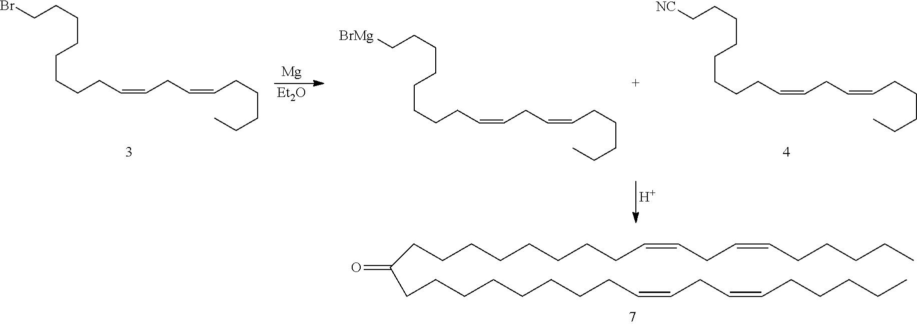

To a flame dried 500 mL 2NRB flask, a freshly activated Mg turnings (0.144 g, 6 mmol) was added and the flask was equipped with a magnetic stir bar and a reflux condenser. This set-up was degassed and flushed with argon and 10 mL of anhydrous ether was added to the flask via syringe. The bromide 3 (26.5 g, 80.47 mmol) was dissolved in anhydrous ether (10 mL) and added dropwise via syringe to the flask. An exothermic reaction was noticed (to confirm/accelerate the Grignard reagent formation, 2 mg of iodine was added and immediate decolorization was observed confirming the formation of the Grignard reagent) and the ether started refluxing. After the completion of the addition the reaction mixture was kept at 35° C. for 1 h and then cooled in ice bath. The cyanide 4 (1.38 g, 5 mmol) was dissolved in anhydrous ether (20 mL) and added dropwise to the reaction mixture with stirring. An exothermic reaction was observed and the reaction mixture was stirred overnight at ambient temperature. The reaction was quenched by adding 10 mL of acetone dropwise followed by ice cold water (60 mL). The reaction mixture was treated with aq. H₂SO₄ (10% by volume, 200 mL) until the solution becomes homogeneous and the layers were separated. The aq. phase was extracted with ether (2×100 mL). The combined ether layers were dried (Na₂SO₄) and concentrated to get the crude product which was purified by column (silica gel, 0-10% ether in hexanes) chromatography. The pure product fractions were evaporated to provide the pure ketone 7 as a colorless oil (2 g, 74%).

In another route, the ketone 7 was synthesized using a two step procedure via the alcohol 6 as follows.

### Synthesis of Heptatriaconta-6,9,28,31-tetraen-19-ol 7

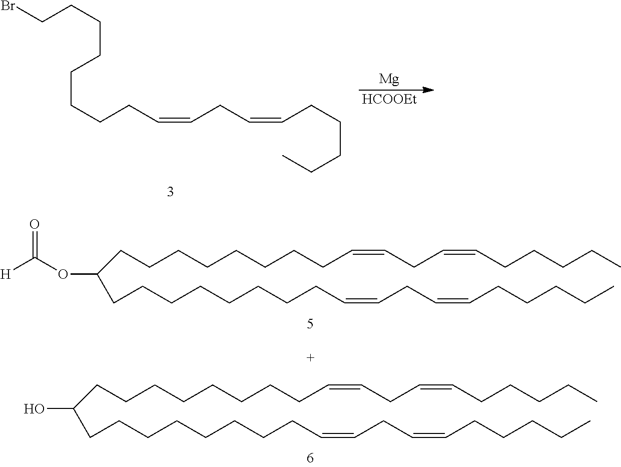

To a flame dried 500 mL RB flask, a freshly activated Mg turnings (2.4 g, 100 mmol) was added and the flask was equipped with a magnetic stir bar, an addition funnel and a reflux condenser. This set-up was degassed and flushed with argon and 10 mL of anhydrous ether was added to the flask via syringe. The bromide 3 (26.5 g, 80.47 mmol) was dissolved in anhydrous ether (50 mL) and added to the addition funnel. About 5 mL of this ether solution was added to the Mg turnings while stirring vigorously. An exothermic reaction was noticed (to confirm/accelerate the Grignard reagent formation, 5 mg of iodine was added and immediate decolorization was observed confirming the formation of the Grignard reagent) and the ether started refluxing. The rest of the solution of the bromide was added dropwise while keeping the reaction under gentle reflux by cooling the flask in water. After the completion of the addition the reaction mixture was kept at 35° C. for 1 h and then cooled in ice bath. Ethyl formate (2.68 g, 36.2 mmol) was dissolved in anhydrous ether (40 mL) and transferred to the addition funnel and added dropwise to the reaction mixture with stirring. An exothermic reaction was observed and the reaction mixture started refluxing. After the initiation of the reaction the rest of the ethereal solution of formate was quickly added as a stream and the reaction mixture was stirred for a further period of 1 h at ambient temperature. The reaction was quenched by adding 10 mL of acetone dropwise followed by ice cold water (60 mL). The reaction mixture was treated with aq. H₂SO₄ (10% by volume, 300 mL) until the solution becomes homogeneous and the layers were separated. The aq. phase was extracted with ether (2×100 mL). The combined ether layers were dried (Na₂SO₄) and concentrated to get the crude product which was purified by column (silica gel, 0-10% ether in hexanes) chromatography. The slightly less polar fractions were concentrated to get the formate 5 (1.9 g) and the pure product fractions were evaporated to provide the pure product 6 as a colorless oil (14.6 g, 78%).

### Synthesis of Heptatriaconta-6,9,28,31-tetraen-19-one 7

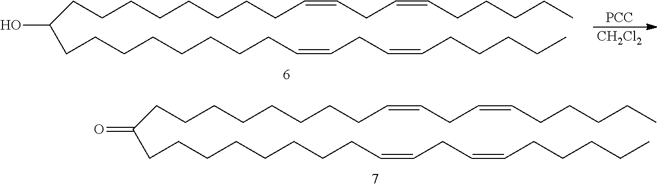

To a solution of the alcohol 6 (3 g, 5.68 mmol) in CH₂Cl₂ (60 mL), a freshly activated 4 A molecular sieves (50 g) was added and to this solution a powdered PCC (4.9 g, 22.7 mmol) was added portionwise over a period of 20 minutes and the mixture was further stirred for 1 hour (Note: careful monitoring of the reaction is necessary in order to get good yields since prolonged reaction times leads to lower yields) and the TLC of the reaction mixture was followed every 10 minutes (5% ether in hexanes) and after the completion of the reaction, the reaction mixture was filtered through a pad of silica gel and the residue was washed with CH₂Cl₂ (400 mL) and the filtrate was concentrated and the thus obtained crude product was further purified by column chromatography (silica gel, 1% Et₂O in hexanes) to isolate the pure product 7 (2.9 g, 97%) as a colorless oil.

Process 1 for preparing 2,2-Dilinoleyl-4-dimethylaminoethyl-[1,3]-dioxolane (5a)

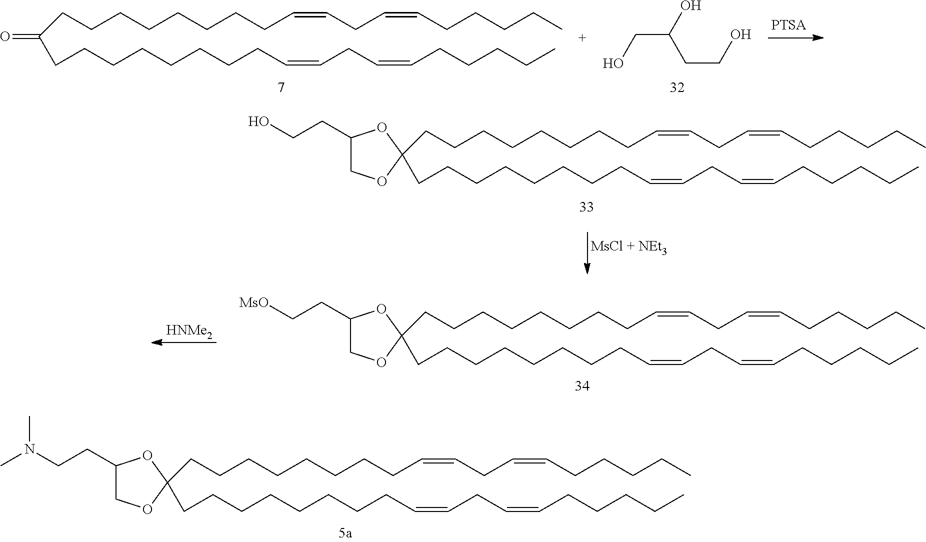

### Preparation of Compound 33

A mixture of compound 32 (10.6 g, 100 mmol), compound 7 (10.54 g, 20 mmol) and PTSA (0.1 eq) was heated under toluene reflux with Soxhlet extractor containing activated 4 Å molecular sieves for 3 h. Removal of solvent then column purification (silica gel, 0-30% EtOAc in hexanes) gave compound 33 (11 g, 90%) as a colorless oil. ¹H NMR (400 MHz, CDCl₃) δ 5.45-5.24 (m, 8H), 4.30-4.17 (m, 1H), 4.08 (dd, J=7.8, 6.1, 1H), 3.80 (dd, J=10.6, 5.0, 3H), 3.53 (t, J=8.0, 1H), 2.77 (t, J=6.4, 5H), 2.29-2.18 (m, 1H), 2.05 (q, J=6.7, 9H), 1.86-1.74 (m, 2H), 1.59 (dd, J=18.3, 9.7, 5H), 1.42-1.18 (m, 43H), 0.89 (t, J=6.8, 6H). ¹³C NMR (101 MHz, CDCl₃) δ 130.39, 130.36, 130.35, 128.14, 112.80, 77.54, 77.22, 76.90, 75.74, 70.14, 61.08, 37.97, 37.50, 35.56, 31.74, 30.14, 30.13, 29.88, 29.80, 29.73, 29.57, 29.53, 27.45, 27.41, 25.84, 24.20, 24.00, 22.79, 14.30.

### Preparation of Compound 34

To an ice-cold solution of compound 33 (10.5 g, 17 mmol) and NEt₃ (5 mL) in DCM (100 mL) a solution of MsCl (2.96 g, 20.5 mmol) in DCM (20 mL) was added dropwise with stirring. After 1 h at r.t., aqueous workup gave a pale yellow oil of 34 which was column purified (silica gel, 0-30% EtOAc in hexanes) to provide the pure mesylate (11.1 g, 94%) as a colorless oil. ¹H NMR (400 MHz, CDCl₃) δ 5.44-5.26 (m, 8H), 4.37 (m, 2H), 4.26-4.13 (m, 1H), 4.10 (m, 1H), 3.53 (m, 1H), 3.02 (s, 3H), 2.76 (d, J=6.4, 4H), 2.05 (d, J=6.9, 10H), 1.55 (s, 4H), 1.29 (d, J=9.8, 34H), 0.88 (t, J=6.9, 6H). Electrospray MS (+ve): Molecular weight for C42H76O5S (M+H)⁺Calc. 693.5, Found 693.4.

### Preparation of Compound 5a (2,2-Dilinoleyl-4-dimethylaminoethyl-[1,3]-dioxolane)

The mesylate 34 (11 g, 15.9 mmol) was dissolved in 400 mL of 2M dimethylamine in THF and the solution was transferred to a Parr pressure reactor and the contents were stirred at 70° C. for 14 h. The reaction mixture was cooled and the TLC of the reaction mixture showed the completion of the reaction. The reaction mixture was concentrated in a rotary evaporator and the thus obtained crude product was purified by column chromatography (silica gel, 0-10% MeOH in dichloromethane) to yield the pure product 5a (9.4 g, 92%) as a colorless oil. ¹H NMR (400 MHz, CDCl₃) δ 5.45-5.24 (m, 8H), 4.07 (dt, J=17.3, 6.4, 2H), 3.48 (t, J=7.3, 1H), 2.77 (t, J=6.4, 4H), 2.47-2.25 (m, 2H), 2.24 (d, J=10.5, 6H), 2.04 (q, J=6.6, 8H), 1.73 (ddd, J=22.8, 14.5, 7.9, 2H), 1.59 (dt, J=20.0, 9.9, 4H), 1.43-1.18 (m, 34H), 0.89 (t, J=6.8, 6H). ¹³C NMR (CDCl₃, 100 MHz) δ=130.2, 130.1, 128.0, 112.1, 74.8, 70.0, 56.3, 45.5, 37.8, 37.5, 31.8, 31.5, 30.0, 30.0, 29.7, 29.6, 29.6, 29.5, 29.5, 29.3, 29.3, 27.2, 27.2, 25.6, 24.0, 23.7, 22.6, 14.0: Electrospray MS (+ve): Molecular weight for C₄₃H₇₉NO₂ (M+H)⁺Calc. 642.6, Found 642.6.

### Example 74

### Synthesis of mPEG2000-1,2-Di-O-alkyl-sn3-carbomoylglyceride

The PEG-lipids, such as mPEG2000-1,2-Di-O-alkyl-sn3-carbomoylglyceride (PEG-DMG) were synthesized using the following procedures:

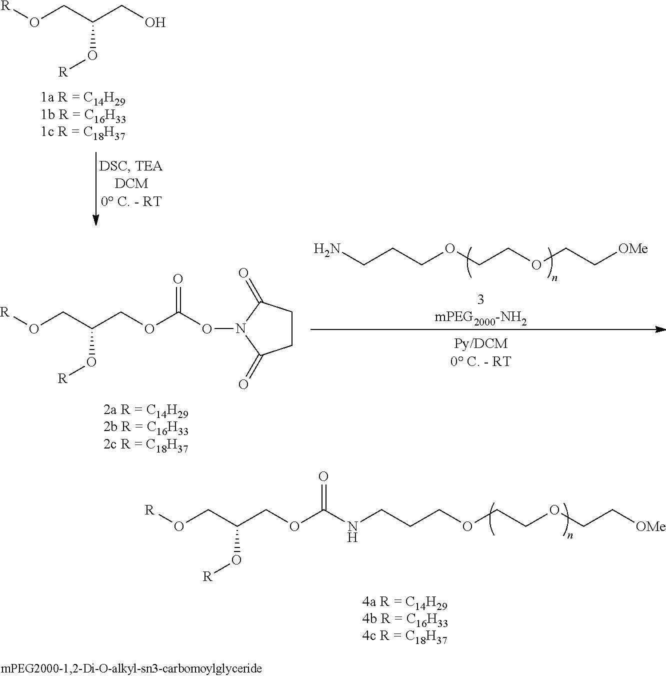

### Preparation of Compound 4a

1,2-Di-O-tetradecyl-sn-glyceride 1a (30 g, 61.80 mmol) and N,N′-succinimidylcarboante (DSC, 23.76 g, 1.5 eq) were taken in dichloromethane (DCM, 500 mL) and stirred over an ice water mixture. Triethylamine (25.30 mL, 3 eq) was added to stirring solution and subsequently the reaction mixture was allowed to stir overnight at ambient temperature. Progress of the reaction was monitored by TLC. The reaction mixture was diluted with DCM (400 mL) and the organic layer was washed with water (2×500 mL), aqueous NaHCO₃ solution (500 mL) followed by standard work-up. Residue obtained was dried at ambient temperature under high vacuum overnight. After drying the crude carbonate 2a thus obtained was dissolved in dichloromethane (500 mL) and stirred over an ice bath. To the stirring solution mPEG₂₀₀₀-NH₂ (3, 103.00 g, 47.20 mmol, purchased from NOF Corporation, Japan) and anhydrous pyridine (80 mL, excess) were added under argon. In some embodiments, the methoxy-(PEG)x-amine has an x=from 45-49, preferably 47-49, and more preferably 49. The reaction mixture was then allowed stir at ambient temperature overnight. Solvents and volatiles were removed under vacuum and the residue was dissolved in DCM (200 mL) and charged on a column of silica gel packed in ethyl acetate. The column was initially eluted with ethyl acetate and subsequently with gradient of 5-10% methanol in dichloromethane to afford the desired PEG-Lipid 4a as a white solid (105.30 g, 83%). NMR (CDCl₃, 400 MHz) δ=5.20-5.12 (m, 1H), 4.18-4.01 (m, 2H), 3.80-3.70 (m, 2H), 3.70-3.20 (m, —O—CH₂—CH₂—O—, PEG-CH₂), 2.10-2.01 (m, 2H), 1.70-1.60 (m, 2H), 1.56-1.45 (m, 4H), 1.31-1.15 (m, 48H), 0.84 (t, J=6.5 Hz, 6H). MS range found: 2660-2836.

### Preparation of 4b

1,2-Di-O-hexadecyl-sn-glyceride 1b (1.00 g, 1.848 mmol) and DSC (0.710 g, 1.5 eq) were taken together in dichloromethane (20 mL) and cooled down to 0° C. in an ice water mixture. Triethylamine (1.00 mL, 3 eq) was added to that and stirred overnight. The reaction was followed by TLC, diluted with DCM, washed with water (2 times), NaHCO₃ solution and dried over sodium sulfate. Solvents were removed under reduced pressure and the residue 2b under high vacuum overnight. This compound was directly used for the next reaction without further purification. MPEG₂₀₀₀-NH₂ 3 (1.50 g, 0.687 mmol, purchased from NOF Corporation, Japan) and compound from previous step 2b (0.702 g, 1.5 eq) were dissolved in dichloromethane (20 mL) under argon. The reaction was cooled to 0° C. Pyridine (1 mL, excess) was added to that and stirred overnight. The reaction was monitored by TLC. Solvents and volatiles were removed under vacuum and the residue was purified by chromatography (first Ethyl acetate then 5-10% MeOH/DCM as a gradient elution) to get the required compound 4b as white solid (1.46 g, 76%). NMR (CDCl₃, 400 MHz) δ=5.17 (t, J=5.5 Hz, 1H), 4.13 (dd, J=4.00 Hz, 11.00 Hz, 1H), 4.05 (dd, J=5.00 Hz, 11.00 Hz, 1H), 3.82-3.75 (m, 2H), 3.70-3.20 (m, —O—CH₂—CH₂—O—, PEG-CH₂), 2.05-1.90 (m, 2H), 1.80-1.70 (m, 2H), 1.61-1.45 (m, 6H), 1.35-1.17 (m, 56H), 0.85 (t, J=6.5 Hz, 6H). MS range found: 2716-2892.

### Preparation of 4c

1,2-Di-O-octadecyl-sn-glyceride 1c (4.00 g, 6.70 mmol) and DSC (2.58 g, 15 eq) were taken together in dichloromethane (60 mL) and cooled down to 0° C. in an ice water mixture. Triethylamine (2.75 mL, 3 eq) was added to that and stirred overnight. The reaction was followed by TLC, diluted with DCM, washed with water (2 times), NaHCO₃ solution and dried over sodium sulfate. Solvents were removed under reduced pressure and the residue under high vacuum overnight. This compound was directly used for the next reaction with further purification. MPEG₂₀₀₀-NH₂ 3 (1.50 g, 0.687 mmol, purchased from NOF Corporation, Japan) and compound from previous step 2c (0.760 g, 1.5 eq) were dissolved in dichloromethane (20 mL) under argon. The reaction was cooled to 0° C. Pyridine (1 mL, excess) was added to that and stirred overnight. The reaction was monitored by TLC. Solvents and volatiles were removed under vacuum and the residue was purified by chromatography (first Ethyl acetate then 5-10% MeOH/DCM as a gradient elution) to get the required compound 4 c as white solid (0.92 g, 48%). ¹H NMR (CDCl₃, 400 MHz) δ=5.22-5.15 (m, 1H), 4.16 (dd, J=4.00 Hz, 11.00 Hz, 1H), 4.06 (dd, J=5.00 Hz, 11.00 Hz, 1H), 3.81-3.75 (m, 2H), 3.70-3.20 (m, —O—CH₂—CH₂—O—, PEG-CH₂), 1.80-1.70 (m, 2H), 1.60-1.48 (m, 4H), 1.31-1.15 (m, 64H), 0.85 (t, J=6.5 Hz, 6H). MS range found: 2774-2948.

### Example 75

### General Protocol for the Extrusion Method

Lipids (Lipid A, DSPC, cholesterol, DMG-PEG) are solubilized and mixed in ethanol according to the desired molar ratio. Liposomes are formed by an ethanol injection method where mixed lipids are added to sodium acetate buffer at pH 5.2. This results in the spontaneous formation of liposomes in 35% ethanol. The liposomes are extruded through a 0.08 μm polycarbonate membrane at least 2 times. A stock siRNA solution is prepared in sodium acetate and 35% ethanol and is added to the liposome to load. The siRNA-liposome solution is incubated at 37° C. for 30 min and, subsequently, diluted. Ethanol is removed and exchanged to PBS buffer by dialysis or tangential flow filtration.

### Example 76

### General Protocol for the in-Line Mixing Method

Individual and separate stock solutions are prepared—one containing lipid and the other siRNA. Lipid stock containing lipid A, DSPC, cholesterol and PEG lipid is prepared by solubilized in 90% ethanol. The remaining 10% is low pH citrate buffer. The concentration of the lipid stock is 4 mg/mL. The pH of this citrate buffer can range between pH 3-5, depending on the type of fusogenic lipid employed. The siRNA is also solubilized in citrate buffer at a concentration of 4 mg/mL. For small scale, 5 mL of each stock solution is prepared.

Stock solutions are completely clear and lipids must be completely solubilized before combining with siRNA. Therefore stock solutions may be heated to completely solubilize the lipids. The siRNAs used in the process may be unmodified oligonucleotides or modified and may be conjugated with lipophilic moieties such as cholesterol.

The individual stocks are combined by pumping each solution to a T-junction. A dual-head Watson-Marlow pump is used to simultaneously control the start and stop of the two streams. A 1.6 mm polypropylene tubing is further downsized to a 0.8 mm tubing in order to increase the linear flow rate. The polypropylene line (ID=0.8 mm) are attached to either side of a T-junction. The polypropylene T has a linear edge of 1.6 mm for a resultant volume of 4.1 mm³. Each of the large ends (1.6 mm) of polypropylene line is placed into test tubes containing either solubilized lipid stock or solubilized siRNA. After the T-junction a single tubing is placed where the combined stream will emit. The tubing is then extending into a container with 2× volume of PBS. The PBS is rapidly stirring. The flow rate for the pump is at a setting of 300 rpm or 110 mL/min. Ethanol is removed and exchanged for PBS by dialysis. The lipid formulations are then concentrated using centrifugation or diafiltration to an appropriate working concentration.

### Example 77

### In Vivo Evaluation of Single Stranded RNA Formulated in Lipid Particle LNP06

In accordance with the present disclosure, oligomeric compounds were synthesized and tested for their ability to reduce PTEN. C57BL/6 mice (Charles River Labs, MA) received either saline or LNP formulated single stranded RNA via tail vein injection at a volume of 0.01 mL/g and the single stranded RNA dose is 4.5 mg/kg. 25 groups of female mice and 9 modified ssRNAs targeting PTEN were formulated in LNP06 and tested. To determine liver mRNA levels of PTEN, 2 days post injection, animals were sacrificed and livers were harvested and snap frozen in liquid nitrogen. Liver lysates were prepared from the frozen tissues and liver mRNA levels of PTEN normalized to GAPDH were quantified using a branched DNA assay (QuantiGene Assay, Panomics, Calif.).

| SEQ | | |
|---|---|---|
| ID | | |
| NO. | ISIS NO. | Composition (5′ to 3′) |
| 05 | 467074 (AS) | P-T_{Sc}U_{f}GₘU_{f}CₘU_{f}CₘU_{f}GₘG_{f}UₘC_{f}CₘU_{f}UₘA_{f}CₘU_{f}UₘAₑAₑ |
| | (A-63801) | |
| 05 | 435397 (AS) | P-T_{d}U_{f}GₘU_{f}CₘU_{f}CₘU_{f}GₘG_{f}UₘC_{f}CₘU_{f}UₘA_{f}CₘU_{f}UₘAₑAₑ |
| | (A-63800) | |
| 06 | 467076 (AS) | Py-^{me}UₘU_{f}GₘU_{f}CₘU_{f}CₘU_{f}GₘG_{f}UₘC_{f}CₘU_{f}UₘA_{f}CₘU_{f}UₘAₑAₑ |
| | (A-63795) | |
| 05 | 467088 (AS) | P-U_{SS}U_{f}GₘU_{f}CₘU_{f}CₘU_{f}GₘG_{f}UₘC_{f}CₘU_{f}UₘA_{f}CₘU_{f}UₘAₑAₑ |
| 05 | A-53286 (AS) | P-TₘₒₑU_{f}GₘU_{f}CₘU_{f}CₘU_{f}GₘG_{f}UₘC_{f}CₘU_{f}UₘA_{f}CₘU_{f}UₘAₑAₑ |
| 05 | A-59889 (AS) | PS-TₘₒₑU_{f}GₘU_{f}CₘU_{f}CₘU_{f}GₘG_{f}UₘC_{f}CₘU_{f}UₘA_{f}CₘU_{f}UₘAₑAₑ |
| 05 | A-59890 (AS) | PS2-TₘₒₑU_{f}GₘU_{f}CₘU_{f}CₘU_{f}GₘG_{f}UₘC_{f}CₘU_{f}UₘA_{f}CₘU_{f}UₘAₑAₑ |

### P-Uss is

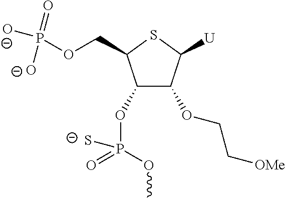

### P-Tₘₒₑ is

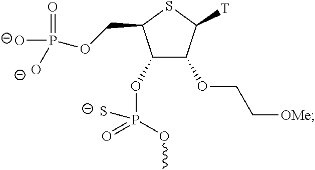

### PS-Tₘₒₑ is

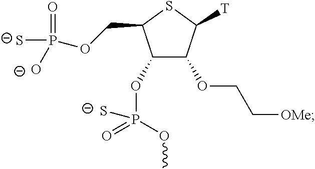

### PS-Tₘₒₑ is

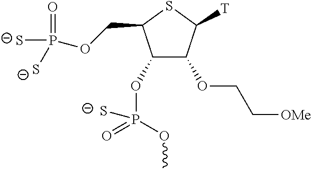

### Results:

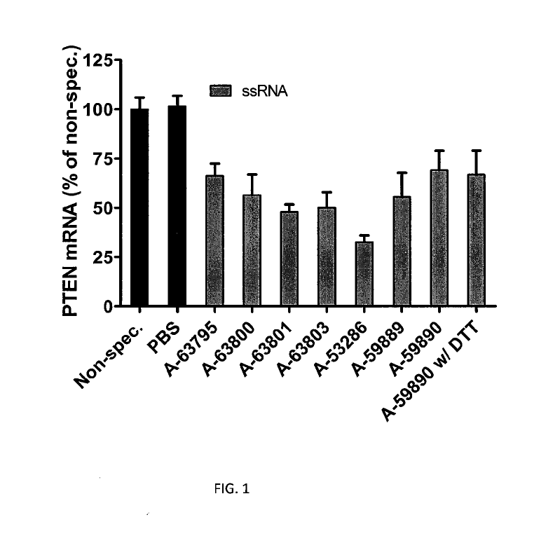

Reduction of the PTEN mRNA was observed with various ssRNA formulated in LNP06 as shown in FIG. 1 and the table below compared with no or low in vivo silencing observed with the corresponding unformulated ssRNA.

| Isis ID | % Kd | SD |
|---|---|---|
| 467076 | 34 | 6.2 |
| 435397 | 44 | 10.5 |
| 467074 | 52 | 3.7 |
| 467088 | 50 | 8.0 |
| A-53286 | 67 | 3.4 |
| A-59889 | 45 | 12.3 |
| A-59890 | 31 | 9.8 |
| A-59890 (DTT) | 33 | 12.2 |

### EQUIVALENTS

Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed.

1. A composition comprising a nucleic acid lipid particle comprising a single stranded RNA, wherein the nucleic acid lipid particle comprises a lipid formulation comprising 45-65 mol % of a cationic lipid, 5 mol % to about 10 mol %, of a non-cationic lipid, 25-40 mol % of a sterol, and 0.5-5 mol % of a PEG or PEG-modified lipid.

2. The composition of claim 1, wherein the cationic lipid comprises formula A wherein formula A is 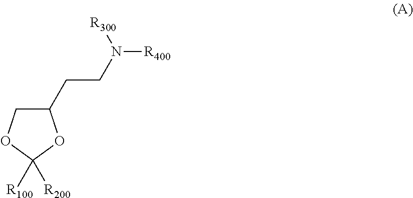
where R₁₀₀ and R₂₀₀ are independently alkyl, alkenyl or alkynyl, each can be optionally substituted, and R₃₀₀ and R₄₀₀ are independently lower alkyl or R₃₀₀ and R₄₀₀ can be taken together to form an optionally substituted heterocyclic ring.

3. The composition of claim 2, wherein the cationic lipid comprises 2,2-Dilinoleyl-4-dimethylaminoethyl-[1,3]-dioxolane.

4. The composition of claim 2, wherein the cationic lipid comprises 2,2-Dilinoleyl-4-dimethylaminoethyl-[1,3]-dioxolane, the non-cationic lipid comprises DSPC, the sterol comprises cholesterol and the PEG lipid comprises PEG-DMG.

5. The composition of claim 4, wherein the cationic lipid comprises 2,2-Dilinoleyl-4-dimethylaminoethyl-[1,3]-dioxolane and the formulation is selected from the group consisting of:

| | |
|---|---|
| LNP05 | Cationic lipid/DSPC/Cholesterol/PEG-DMG |
| | 57.5/7.5/31.5/3.5 |
| | lipid:siRNA ~6:1 |
| LNP06 | Cationic lipid/DSPC/Cholesterol/PEG-DMG |
| | 57.5/7.5/31.5/3.5 |
| | lipid:siRNA ~11:1 |
| LNP07 | Cationic lipid/DSPC/Cholesterol/PEG-DMG |
| | 60/7.5/31/1.5, |
| | lipid:siRNA ~6:1 |
| LNP08 | Cationic lipid/DSPC/Cholesterol/PEG-DMG |
| | 60/7.5/31/1.5, |
| | lipid:siRNA ~11:1 |
| LNP09 | Cationic lipid/DSPC/Cholesterol/PEG-DMG |
| | 50/10/38.5/1.5 |
| | lipid:siRNA ~10:1 |

6. The composition of claim 1, wherein the single stranded RNA comprising a nucleoside having Formula I: 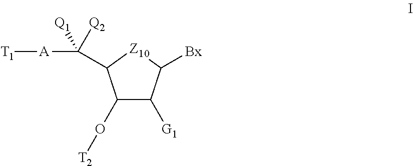
wherein:
Bx is a heterocyclic base moiety;
A is O, S or N(R₁);
Z₁₀ is O, S, N(R₁), or CH₂;
R₁ is H, C₁-C₆ alkyl or substituted C₁-C₆ alkyl;
T₁ is a phosphorus moiety;
T₂ is an internucleoside linking group linking the monomer of Formula I to the remainder of the oligomeric compound;
each of Q₁ and Q₂ is independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;
G₁ is halogen, X₁—V, or O—X₂;
X₁ is O, S or CR₂R₃;
each R₂ and R₃ is, independently, H or C₁-C₆ alkyl;
V is a conjugate group, aryl, (CH₂)₂[O(CH₂)₂]ₜOCH₃, where t is from 1-3, (CH₂)₂F, CH₂COOH, CH₂CONH₂, CH₂CONR₅R₆, CH₂COOCH₂CH₃, CH₂CONH(CH₂)ᵢ—S—R₄ where i is from 1 to 10, CH₂CONH(CH₂)ₖ₃NR₅R₆ where k₃ is from 1 to 6, CH₂CONH[(CH₂)ₖ₁—N(H)]ₖ₂—(CH₂)ₖ₁NH₂ where each k₁ is independently from 2 to 4 and k₂ is from 2 to 10;
R₄ is H, C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, C₆-C₁₄ aryl or a thio protecting group;
R₅ and R₆ are each, independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;
X₂ is [C(R₇)(R₈)]ₙ—[(C═O)ₘX]ⱼ—Z;
each R₇ and R₈ is independently, H, halogen, C₁-C₆ alkyl or substituted C₁-C₆ alkyl;
X is O, S or N(E₁);
Z is H, halogen, C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl or N(E₂)(E₃);
E₁, E₂, and E₃ are each independently H, C₁-C₆ alkyl, or substituted C₁-C₆ alkyl;
n is from 1 to about 6;
m is 0 or 1;
j is 0 or 1;
each substituted group comprises one or more optionally protected substituent groups independently selected from H, halogen, OJ₁, N(J₁)(J₂), ═NJ₁; SJ₁, N₃, CN, OC(=L) J₁, OC(=L)N(J₁)(J₂), C(=L)N(J₁)(J₂), C(=L)N(H)—(CH₂)₂N(J₁)(J₂) or a mono or polycyclic ring system;
L is O, S or NJ₃;
each J₁, J₂ and J₃ is, independently, H or C₁-C₆ alkyl; and
when j is 1, then Z is other than halogen or N(E₂)(E₃).

7. The composition of claim 1, wherein the single stranded RNA comprising a nucleoside having Formula II: 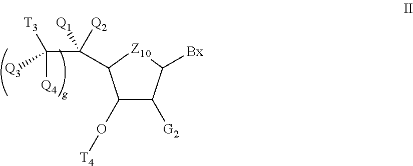
wherein:
Bx is a heterocyclic base moiety;
T₃ is a phosphorus moiety;
Z₁₀ is O, S, N(R₁), or CH₂;
T₄ is an internucleoside linking group linking the monomer of Formula II to the remainder of the oligomeric compound;
Q₁, Q₂, Q₃ and Q₄ are each, independently, H, halogen, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl, hydroxyl, substituted oxy, O—C₁-C₆ alkyl, substituted O—C₁-C₆ alkyl, S—C₁-C₆ alkyl, substituted S—C₁-C₆ alkyl, N(R₁)—C₁-C₆ alkyl or substituted N(R₁)—C₁-C₆ alkyl;
R₁ is H, C₁-C₆ alkyl or substituted C₁-C₆ alkyl;
G₂ is H, OH, halogen, O-aryl or O—[C(R₄)(R₅)]ₙ—[(C═O)ₘX]ⱼ—Z;
each R₄ and R₅ is, independently, H, halogen, C₁-C₆ alkyl or substituted C₁-C₆ alkyl;
X is O, S or N(E₁);
Z is H, halogen, C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl or N(E₂)(E₃);
E₁, E₂, and E₃ are each independently H, C₁-C₆ alkyl, or substituted C₁-C₆ alkyl;
n is from 1 to about 6;
m is 0 or 1;
j is 0 or 1;
each substituted group comprises one or more optionally protected substituent groups independently selected from H, halogen, OJ₁, N(J₁)(J₂), ═NJ₁, SJ₁, N₃, CN, OC(=L)J₁, OC(=L)N(J₁)(J₂), C(=L)N(J₁)(J₂), C(=L)N(H)—(CH₂)₂N(J₁)(J₂), a mono or poly cyclic ring system, a phosphate group or a phosphorus moiety;
L is O, S or NJ₃;
each J₁, J₂ and J₃ is, independently, H or C₁-C₆ alkyl;
when j is 1 then Z is other than halogen or N(E₂)(E₃); and
when Q₁, Q₂, Q₃ and Q₄ are each H, or when Q₁ and Q₂ are H and Q₃ and Q₄ are each F, or when Q₁ and Q₂ are each H and one of Q₃ and Q₄ is H and the other of Q₃ and Q₄ is R₉, then G₂ is other than H, hydroxyl, OR₉, halogen, CF₃, CCl₃, CHCl₂ or CH₂OH, wherein R₉ is alkyl, alkenyl, alkynyl, aryl or alkaryl.

8. The composition of claim 1, wherein the single stranded RNA comprising a nucleoside having Formula III: 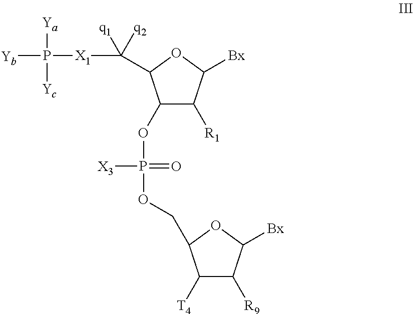
wherein:
each Bx is independently a heterocyclic base moiety;
T₄ is an internucleoside linking group attaching the nucleoside of Formula III to the remainder of the oligonucleotide;
each of q₁ and q₂ is, independently selected from H, C₁-C₆ alkyl, C₂-C₆ alkenyl, C₂-C₆ alkynyl, substituted C₁-C₆ alkyl, substituted C₂-C₆ alkenyl and substituted C₂-C₆ alkynyl;
X₁ is S, NR₁₆, or CR₁₀R₁₁ wherein each R₁₀ and R₁₁ is, independently, H, F, C₁-C₆ haloalkyl, or C₁-C₆ alkyl; and
R₁ is selected from a halogen, X₂—V, and O—X₄;
Or
each of q₁ and q₂ is, independently, selected from H, C₁-C₆ alkyl, C₂-C₆ alkenyl, C₂-C₆ alkynyl, substituted C₁-C₆ alkyl, substituted C₂-C₆ alkenyl and substituted C₂-C₆ alkynyl;
X₁ is O, S, NR₁₆R₁₇, or CR₁₀R₁₁ wherein each R₁₀ and R₁₁ is, independently, H, F, C₁-C₆ haloalkyl, or C₁-C₆ alkyl; and
R₁ is X₂—V;
Or
each of q₁ and q₂ is, independently, selected from C₁-C₆ alkyl, C₂-C₆ alkenyl, C₂-C₆ alkynyl, substituted C₁-C₆ alkyl, substituted C₁-C₆ alkenyl and substituted C₂-C₆ alkynyl;
X₁ is O, S, NR₁₆R₁₇, or CR₁₀R₁₁ wherein each R₁₀ and R₁₁ is, independently, H, F, C₁-C₆ haloalkyl, or C₁-C₆ alkyl; and
R₁ is selected from a halogen, X₂—V, and O—X₄;
X₂ is O, S or CR₇R₈ wherein each R₇ and R₈ is, independently, H or C₁-C₆ alkyl;
V is selected from cholesterol, (CH₂)₂[O(CH₂)₂]ₜOCH₃, where t is from 1-3, (CH₂)₂F, CH₂COOH, CH₂CONH₂, CH₂CONR₅R₆, CH₂COOCH₂CH₃, CH₂CONH(CH₂)ᵢ—S—R₄ where i is from 1 to 10, CH₂CONH(CH₂)_{J}NR₅R₆ where j is from 1 to 6, and CH₂CONH[(CH₂)ₖ₁—N(H)]ₖ—(CH₂)ₖ₁NH₂ where each k₁ is independently from 2 to 4 and k₂ is from 2 to 10;
R₄ is selected from H, C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, C₆-C₁₄ aryl or a thio protecting group;
R₅ and R₆ are each, independently, selected from H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;
R₁₆ is selected from H, C₁-C₆ alkyl, or substituted C₁-C₆ alkyl;
X₄ is [C(Rₐ)(R_{b})]ₙ—[(C═O)ₘX_{c}]ₖ—R_{d} wherein
each Rₐ and R_{b} is independently H or halogen;
X_{c} is O, S or N(E₁);
R_{d} is H, C₁-C₆ alkyl, C₂-C₆ alkenyl, C₂-C₆ alkynyl, substituted C₁-C₆ alkyl, s substituted C₂-C₆ alkenyl, substituted C₂-C₆ alkynyl or NE₂E₃;
each E₁, E₂, and E₃ is independently H, C₁-C₆ alkyl, or substituted C₁-C₆ alkyl;
n is 1 to 6;
m is 0 or 1; and
k is 0 or 1;
X₃ is OH or SH;
Yₐ is O or S;
each Y_{b} and Y_{c} is, independently, selected from OH, SH, alkyl, alkoxy, substituted C₁-C₆ alkyl and substituted C₁-C₆ alkoxy; and
R₉ is selected from a halogen, X₂—V, and O—X₄;
wherein each substituted group is, independently, mono or poly substituted with optionally protected substituent groups independently selected from halogen, oxo, OJ₁, NJ₁J₂, SJ₁, N₃, OC(═O)J₁ and CN, wherein each J₁ and J₂ is, independently, H or C₁-C₆ alkyl; and J₄ is hydrogen, or a protecting group.

9. The composition of claim 8 wherein R₁ is selected from halogen, O-alkyl, O-haloalkyl, O-alkoxy.

10. The composition of claim 8 wherein R₁ is F.

11. The oligomeric compound of claim 8 wherein R₁ is O—C₂-C₄ alkyl or haloalkyl.

12. The oligomeric compound of claim 8 wherein R₁ is OCH₃.

13. The oligomeric compound of claim 8 wherein R₁ is O(CH₂)₂OCH₃.

14. The oligomeric compound of claim 8 wherein R₁ is FCH₂CH₄.

15. The oligomeric compound of claim 8 wherein R₁ is (CH₂)₂[O(CH₂)₂]ₜOCH₃, where t is from 1-3.

16. The oligomeric compound of claim 8 wherein R₁ is selected from, trifluoroalkoxy, azido, aminooxy, S-alkyl, N(J₄)-alkyl, O-alkenyl, S-alkenyl, N(J₄)-alkenyl, O-alkynyl, S-alkynyl, N(J₄)-alkynyl, and X₂—V.

17. The oligomeric compound of claim 8 wherein R₁ is X₂—V.

18. The oligomeric compound of claim 17 wherein V is (CH₂)₂F.

19. The oligomeric compound of claim 17 wherein V is CH₂CONH(CH₂)ᵢ—S—R₄

20. The oligomeric compound of claim 17 wherein V is CH₂CONH[(CH₂)ₖ₁—N(H)]ₖ₂—(CH₂)ₖ₁NH₂.

21. The oligomeric compound of claim 17 wherein V is CH₂CONH—(CH₂)₃—N(H)—(CH₂)₄—N(H)—(CH₂)₃NH₂.

22. The oligomeric compound of claim 17 wherein V is CH₂CONH(CH₂)_{J}NR₅R₆.

23. The oligomeric compound of claim 22 wherein R₅ is methyl and R₆ is methyl.

24. The oligomeric compound of claim 8, wherein X₂ is O.

25. The oligomeric compound of claim 8, wherein X₂ is S.

26. The oligomeric compound of claim 8, wherein X₂ is CR₇R₈.

27. The oligomeric compound of claim 8, wherein at least one of q₁ and q₂ is C₁-C₆ alkyl or substituted C₁-C₆ alkyl.

28. The oligomeric compound of claim 27 wherein at least one of q₁ and q₂ is C₁-C₆ alkyl.

29. The oligomeric compound of claim 6, wherein the phosphorus moiety is P(Yₐ)(Y_{b})(Y_{c}), where Yₐ is O or S, and each Y_{b} and Y_{c} is, independently, selected from OH, SH, alkyl, alkoxy, substituted C₁-C₆ alkyl and substituted C₁-C₆ alkoxy.

30. The oligomeric compound of claim 29 wherein Yₐ is O and Y_{b} and Y_{c} are each OH.

31. The composition of claim 1, further comprising a lipoprotein.

32. The composition of claim 1, further comprising apolipoprotein E (ApoE).
